(19) United States
(12) Patent Application Publication
(10) Pub. No.: US 2026/0009037 A1
(43) Pub. Date: Jan. 8, 2026

(54) RNAi Agents for Inhibiting Expression of Mitochondrial Amidoxime Reducing Component 1 (MARC1), Pharmaceutical Compositions Thereof, and Methods of Use

(71) Applicant: Arrowhead Pharmaceuticals, Inc., Pasadena, CA (US)

(72) Inventors: Jonathan Van Dyke, Burke, WI (US); Zhi-Ming Ding, Waunakee, WI (US); Zhao Xu, San Diego, CA (US); Lindsey Moser, Madison, WI (US); Daniel Braas, Madison, WI (US); Audra Winter, Lake Mills, WI (US); Anthony Nicholas, Oregon, WI (US); Tao Pei, Middleton, WI (US)

(21) Appl. No.: 19/328,832

(22) Filed: Sep. 15, 2025

Related U.S. Application Data
(63) Continuation of application No. PCT/US24/20222, filed on Mar. 15, 2024.
(60) Provisional application No. 63/490,694, filed on Mar. 16, 2023.

(51) Int. Cl. C12N 15/113; A61K 9/00; A61K 47/02; A61P 1/16

(52) U.S. Cl. CPC C12N 15/1137; A61K 9/0019; A61K 47/02; A61P 1/16; C12N 2310/11; C12N 2310/14; C12N 2310/312; C12N 2310/315; C12N 2310/321; C12N 2310/322; C12N 2310/351; C12N 2320/35; C12Y 107/00

(57) ABSTRACT

The present disclosure relates to RNAi agents, e.g., double stranded RNAi agents, able to inhibit mitochondrial amidoxime reducing component 1 (MARC1) gene expression. Also disclosed are pharmaceutical compositions that include MARC1 RNAi agents and methods of use thereof. The MARC1 RNAi agents disclosed herein may be conjugated to targeting ligands, including ligands that comprise N-acetyl-galactosamine, to facilitate the delivery to hepatocyte cells. Delivery of the MARC1 RNAi agents in vivo provides for inhibition of MARC1 gene expression. The RNAi agents can be used in methods of treatment of diseases, disorders, or symptoms mediated in part by MARC1 gene expression, including nonalcoholic steatohepatitis (NASH), nonalcoholic fatty liver disease (NAFLD), alcoholic fatty liver disease, autoimmune hepatitis, hepatic fibrosis, cirrhosis, elevated blood cholesterol levels, hypertriglyceridemia, liver disease, and/or other MARC1-related disease.

### CROSS REFERENCE TO RELATED APPLICATIONS

This application is a continuation application of International Application No. PCT/US2024/020222, filed Mar. 15, 2024, which claims the benefit of priority to U.S. Provisional Patent Application Ser. No. 63/490,694, filed on 16 Mar. 2023, the contents of which are incorporated herein by reference in their entirety.

### SEQUENCE LISTING

This application contains a Sequence Listing (in compliance with Standard ST26), which has been submitted in xml format and is hereby incorporated by reference in its entirety. The xml sequence listing file is named 30716-US1_SeqListing.xml. created September 15. 2025, and is 6,540,386 bytes in size.

### FIELD OF THE INVENTION

The present disclosure relates to RNA interference (RNAi) agents, e.g., double stranded RNAi agents such as chemically modified small (or short) interfering RNA (siRNA), for inhibition mitochondrial amidoxime reducing component 1 (MARC1), pharmaceutical compositions that include MARC1 RNAi agents, and methods of use thereof for the treatment of MARC1-related diseases and disorders.

### BACKGROUND

The mitochondrial amidoxime-reducing component (MARC) protein was first discovered and described as a molybdenum cofactor-containing component in the mitochondrial benzamidoxime prodrug-converting system in 2006. The human genome contains two MARC genes: MTARCI and 2 (commonly known as and referred to as simply MARC1 and 2, respectively), that encode MARC1 and MARC2 proteins which share significant homologies in sequence and function.

Researchers identified a rare missense variant in MARC1 (referred to as p.A165T mutation) that causes a loss-of-function in MARC1 protein, which mutation was associated with the protection against liver cirrhosis, the lowering hepatic fat, and reductions of other various biomarkers for liver disease. (Emdin CA, et al., A missense variant in Mitochondrial Amidoxime Reducing Component 1 gene and protection against liver disease, PLOS Genet. (April 2020): 16 (4): e1008629). Individuals homozygous for this loss-of-function mutation in MARC1 exhibit lower levels of hepatic fat and a decreased likelihood of physician-diagnosed fatty liver. Loss-of-function mutations in MARC1 are also associated with low blood levels of alanine transaminase, alkaline phosphatase, total cholesterol, and LDL-cholesterol.

While the exact mechanism of MARC1 with respect to the progression of liver disease is not yet fully understood, the reported linkage of MARC1 was further validated by recent genome-wide association studies focused on liver disease and autoimmune hepatitis, which further confirmed that the missense mutations in MARC1 and resulting loss-of-function were protective against liver injury and cirrhosis (Janik et al., MARC1 p.A165T variant is associated with decreased markers of liver injury and enhanced antioxidant capacity in autoimmune hepatitis. Sci Rep (2021): 11:24407). These aggregate data suggest that reductions in MARC1 protein may be able to lower blood cholesterol levels and protect against liver cirrhosis, and that inhibition of MARC1 is a potential therapeutic target for the treatment of liver disease.

### SUMMARY

There exists a need for novel RNA interference (RNAi) agents (termed RNAi agents, RNAi triggers, or triggers), e.g., double stranded RNAi agents such as chemically modified siRNAs, that are able to selectively and efficiently inhibit MARC1 gene expression. Further, there exists a need for compositions of novel MARC1-specific RNAi agents for use as a therapeutic or medicament for the treatment of MARC1-related diseases or disorders, such as nonalcoholic steatohepatitis (NASH), nonalcoholic fatty liver disease (NAFLD), alcoholic fatty liver disease, autoimmune hepatitis, hepatic fibrosis, cirrhosis, elevated blood cholesterol levels, hypertriglyceridemia, liver disease, and/or other MARC1-related disease.

The nucleotide sequences and chemical modifications of the MARC1 RNAi agents disclosed herein differ from those previously disclosed or known in the art. The MARC1 RNAi agents disclosed herein provide for highly specific, potent, and efficient in vivo and/or in vitro inhibition of the expression of a MARC1 gene.

In some embodiments, the sense strand comprises a nucleotide sequence of at least 15 contiguous nucleotides differing by 0 or 1 nucleotides from 15 contiguous nucleotides of any one of the sense strand sequences of Table 2, Table 4, Table 5, or Table 6D, and wherein the sense strand has a region of at least 85% complementarity over the 15 contiguous nucleotides to the antisense strand.

In some embodiments, disclosed herein are RNAi agents for inhibiting expression of a MARC1 gene, comprising:

an antisense strand wherein nucleotides 1-19 of the antisense strand comprise nucleotides 1-19 of the antisense strand sequences of Table 2, Table 3, or Table 6D, and a sense strand comprising a nucleotide sequence that is at least partially complementary to the antisense strand, wherein all or substantially all of the nucleotides of the antisense strand and/or the sense strand are modified nucleotides, and the RNAi agent is linked to a targeting ligand that comprises N-acetyl-galactosamine.

In some embodiments, disclosed herein are RNAi agents for inhibiting expression of a MARC1 gene, comprising a sense strand comprising a nucleotide sequence of at least 15 contiguous nucleotides differing by 0 or 1 nucleotides from 15 contiguous nucleotides of any one of the sense strand sequences of Table 2, Table 4, Table 5, or Table 6D, and wherein the sense strand has a region of at least 85% complementarity over the 15 contiguous nucleotides to the antisense strand.

In some embodiments, at least one nucleotide of the MARC1 RNAi agent includes a modified internucleoside linkage.

In some embodiments, the modified nucleotides of the MARC1 RNAi agents disclosed herein are selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.

In other embodiments, all or substantially all of the modified nucleotides of the RNAi agents disclosed herein are 2′-O-methyl nucleotides, 2′-fluoro nucleotides, or combinations thereof.

In some embodiments, the antisense strand consists of, consists essentially of, or comprises the nucleotide sequence of any one of the modified antisense strand sequences of Table 3 or Table 6D.

In some embodiments, the sense strand consists of, consists essentially of, or comprises the nucleotide sequence of any of the modified sense strand sequences of Table 4, Table 5, or Table 6D.

In some embodiments, the antisense strand comprises the nucleotide sequence of any one of the modified sequences of Table 3 or Table 6D and the sense strand comprises the nucleotide sequence of any one of the modified sequences of Table 4 or Table 6D.

The RNAi agents disclosed herein are linked to a targeting ligand that comprises N-acetyl-galactosamine. In further embodiments, the targeting ligand is linked to the sense strand. In some embodiments, the targeting ligand is linked to the 5′ terminal end of the sense strand.

In some embodiments, the sense strand is between 15 and 30 nucleotides in length, and the antisense strand is between 19 and 30 nucleotides in length. In other embodiments, the sense strand and the antisense strand are each between 21 and 27 nucleotides in length. In other embodiments, the sense strand and the antisense strand are each between 21 and 24 nucleotides in length. In still other embodiments, sense strand and the antisense strand are each 21 nucleotides in length.

In some embodiments, the RNAi agents have two blunt ends.

In some embodiments, the sense strand comprises one or two terminal caps. In other embodiments, the sense strand comprises one or two inverted abasic residues.

In some embodiments, the RNAi agents are comprised of a sense strand and an antisense strand that form a duplex sequence of the duplex structures shown in Table 6A, 6B, 6C, or 6D.

In some embodiments, the sense strand further includes inverted abasic residues at the 3′ terminal end of the nucleotide sequence, at the 5′ end of the nucleotide sequence, or at both.

In further embodiments, the targeting ligand comprises or consists of:

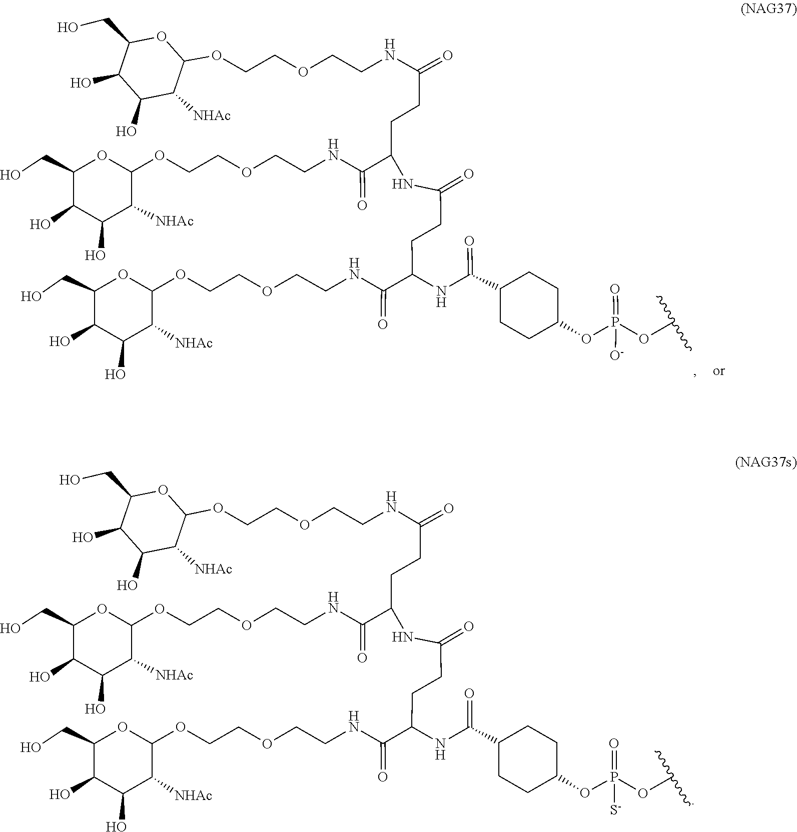

Also disclosed herein are compositions comprising the disclosed RNAi agents, wherein the compositions further comprise a pharmaceutically acceptable excipient.

Additionally, provided herein are methods for inhibiting expression of a MARC1 gene in a hepatocyte cell in a human subject in vivo, the methods comprising introducing into the subject an effective amount of the disclosed MARC1 RNAi agents or the disclosed compositions.

Further provided herein are methods of treating a MARC1-related disease, disorder, or symptom, the methods comprising administering to a human subject in need thereof a therapeutically effective amount of the disclosed compositions.

In some embodiments, the disease is nonalcoholic steatohepatitis (NASH), alcoholic and nonalcoholic fatty liver disease (NAFLD), fatty liver disease, cirrhosis, elevated blood cholesterol levels, hypertriglyceridemia, liver disease, and/or other MARC1-related disease.

In some embodiments, the RNAi agents are administered at a dose of about 0.05 mg/kg to about 5.0 mg/kg of body weight of the human subject. In some embodiments, the MARC1 RNAi agents disclosed herein are administered in a fixed dose of a single injection containing about 50 mg, about 100 mg, about 200 mg, about 300 mg, or about 400 mg of MARC1 RNAi agent.

Also provided herein are usages of the disclosed RNAi agents or the disclosed compositions, for the treatment of a disease, disorder, or symptom that is mediated at least in part by MARC1 gene expression.

Further provided herein are usages of the disclosed RNAi agents or the disclosed compositions, for the preparation of a pharmaceutical compositions for treating a disease, disorder, or symptom that is mediated at least in part by MARC1 gene expression.

### DETAILED DESCRIPTION

The disclosed RNAi agents, compositions thereof, and methods of use may be understood more readily by reference to the following detailed description, which form a part of this disclosure. It is to be understood that the disclosure is not limited to what is specifically described and/or shown herein, and that the terminology used herein is for the purpose of describing particular embodiments by way of example only and is not intended to be limiting.

It is to be appreciated that while certain features of the disclosures included herein are, for clarity, described herein in the context of separate embodiments, they may also be provided in combination in a single embodiment. Conversely, various features of the disclosed methods that are, for brevity, described in the context of a single embodiment, may also be provided separately or in any subcombination.

### Definitions

As used herein, an “RNAi agent” means a chemical composition that contains an RNA or RNA-like (e.g., chemically modified RNA) oligonucleotide molecule that is capable of degrading or inhibiting (e.g., degrades or inhibits under appropriate conditions) translation of messenger RNA (mRNA) transcripts of a target mRNA in a sequence specific manner. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: small (or short) interfering RNAs (siRNAs), double stranded RNAs (dsRNA), micro RNAs (miRNAs), short hairpin RNAs (shRNA), and dicer substrates. The antisense strand of the RNAi agents described herein is at least partially complementary to the mRNA being targeted (i.e. MARC1 mRNA). RNAi agents can include one or more modified nucleotides and/or one or more non-phosphodiester linkages.

As used herein, the terms “silence.” “reduce.” “inhibit,” “down-regulate,” or “knockdown” when referring to expression of a given gene, mean that the expression of the gene, as measured by the level of RNA transcribed from the gene or the level of polypeptide, protein, or protein subunit translated from the mRNA in a cell, group of cells, tissue, organ, or subject in which the gene is transcribed, is reduced when the cell, group of cells, tissue, organ, or subject is treated with the RNAi agents described herein as compared to a second cell, group of cells, tissue, organ, or subject that has not or have not been so treated.

As used herein, the terms “sequence” and “nucleotide sequence” mean a succession or order of nucleobases or nucleotides, described with a succession of letters using standard nomenclature. A nucleic acid molecule can comprise unmodified and/or modified nucleotides. A nucleotide sequence can comprise unmodified and/or modified nucleotides.

As used herein, a “base.” “nucleotide base,” or “nucleobase,” is a heterocyclic pyrimidine or purine compound that is a component of a nucleotide, and includes the primary purine bases adenine and guanine, and the primary pyrimidine bases cytosine, thymine, and uracil. A nucleobase may further be modified to include, without limitation, universal bases, hydrophobic bases, promiscuous bases, size-expanded bases, and fluorinated bases. (See. e.g., Modified Nucleosides in Biochemistry, Biotechnology and Medicine, Herdewijn, P. ed. Wiley-VCH, 2008). The synthesis of such modified nucleobases (including phosphoramidite compounds that include modified nucleobases) is known in the art.

As used herein, the term “nucleotide” has the same meaning as commonly understood in the art. Thus, the term “nucleotide” as used herein, refers to a glycoside comprising a sugar moiety, a base moiety and a covalently linked group (linkage group), such as a phosphate or phosphorothioate internucleoside linkage group, and covers both naturally occurring nucleotides, such as DNA or RNA, and non-naturally occurring nucleotides comprising modified sugar and/or base moieties, which are also referred to as nucleotide analogs herein. Herein, a single nucleotide can be referred to as a monomer or unit.

As used herein, and unless otherwise indicated, the term “complementary,” when used to describe a first nucleobase or nucleotide sequence (e.g., RNAi agent sense strand or targeted mRNA) in relation to a second nucleobase or nucleotide sequence (e.g., RNAi agent antisense strand or a single-stranded antisense oligonucleotide), means the ability of an oligonucleotide or polynucleotide including the first nucleotide sequence to hybridize (form base pair hydrogen bonds under mammalian physiological conditions (or otherwise suitable in vivo or in vitro conditions)) and form a duplex or double helical structure under certain standard conditions with an oligonucleotide that includes the second nucleotide sequence. The person of ordinary skill in the art would be able to select the set of conditions most appropriate for a hybridization test. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af, as defined herein, are complementary to U (or T) and identical to A for the purposes of determining identity or complementarity.

As used herein, “perfectly complementary” or “fully complementary” means that in a hybridized pair of nucleobase or nucleotide sequence molecules, all (100%) of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “partially complementary” means that in a hybridized pair of nucleobase or nucleotide sequence molecules, at least 70%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “substantially complementary” means that in a hybridized pair of nucleobase or nucleotide sequence molecules, at least 85%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, the terms “complementary.” “fully complementary.” “partially complementary.” and “substantially complementary” are used with respect to the nucleobase or nucleotide matching between the sense strand and the antisense strand of an RNAi agent, or between the antisense strand of an RNAi agent and a sequence of a MARC1 mRNA.

As used herein, the term “substantially identical” or “substantial identity,” as applied to a nucleic acid sequence means the nucleotide sequence (or a portion of a nucleotide sequence) has at least about 85% sequence identity or more, e.g., at least 90%, at least 95%, or at least 99% identity, compared to a reference sequence. Percentage of sequence identity is determined by comparing two optimally aligned sequences over a comparison window. The percentage is calculated by determining the number of positions at which the same type of nucleic acid base occurs in both sequences to yield the number of matched positions, dividing the number of matched positions by the total number of positions in the window of comparison and multiplying the result by 100 to yield the percentage of sequence identity. The inventions disclosed herein encompass nucleotide sequences substantially identical to those disclosed herein.

As used herein, the terms “individual”, “patient” and “subject”, are used interchangeably to refer to a member of any animal species including, but not limited to, birds, humans and other primates, and other mammals including commercially relevant mammals or animal models such as mice, rats, monkeys, cattle, pigs, horses, sheep, cats, and dogs. Preferably, the subject is a human.

As used herein, the terms “treat.” “treatment.” and the like, mean the methods or steps taken to provide relief from or alleviation of the number, severity, and/or frequency of one or more symptoms of a disease in a subject. As used herein, “treat” and “treatment” may include the prevention, management, prophylactic treatment, and/or inhibition or reduction of the number, severity, and/or frequency of one or more symptoms of a disease in a subject.

As used herein, the phrase “introducing into a cell,” when referring to an RNAi agent. means functionally delivering the RNAi agent into a cell. The phrase “functional delivery,” means delivering the RNAi agent to the cell in a manner that enables the RNAi agent to have the expected biological activity, e.g., sequence-specific inhibition of gene expression.

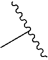

Unless stated otherwise, use of the symbol as used herein means that any group or groups may be linked thereto that is in accordance with the scope of the inventions described herein.

As used herein, the term “isomers” refers to compounds that have identical molecular formulae, but that differ in the nature or the sequence of bonding of their atoms or in the arrangement of their atoms in space. Isomers that differ in the arrangement of their atoms in space are termed “stereoisomers.” Stereoisomers that are not mirror images of one another are termed “diastereoisomers.” and stereoisomers that are non-superimposable mirror images are termed “enantiomers.” or sometimes optical isomers. A carbon atom bonded to four non-identical substituents is termed a “chiral center.”

As used herein, unless specifically identified in a structure as having a particular conformation, for each structure in which asymmetric centers are present and thus give rise to enantiomers, diastereomers, or other stereoisomeric configurations, each structure disclosed herein is intended to represent all such possible isomers, including their optically pure and racemic forms. For example, the structures disclosed herein are intended to cover mixtures of diastereomers as well as single stereoisomers.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N. O. or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art. Correspondingly, compounds described herein with labile protons or basic atoms should also be understood to represent salt forms of the corresponding compound. Compounds described herein may be in a free acid, free base, or salt form. Pharmaceutically acceptable salts of the compounds described herein should be understood to be within the scope of the invention.

As used herein, the term “linked” or “conjugated” when referring to the connection between two compounds or molecules means that two compounds or molecules are joined by a covalent bond. Unless stated, the terms “linked” and “conjugated” as used herein may refer to the connection between a first compound and a second compound either with or without any intervening atoms or groups of atoms.

As used herein, the term “including” is used to herein mean, and is used interchangeably with, the phrase “including but not limited to.” The term “or” is used herein to mean, and is used interchangeably with, the term “and/or.” unless the context clearly indicates otherwise.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Where a value is explicitly recited, it is to be understood that values which are about the same quantity or amount as the recited value are also within the scope of the disclosure. Where a combination is disclosed, each sub-combination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed: more than one element of a disclosure can have such exclusions, and all combinations of elements having such exclusions are hereby disclosed.

Other objects, features, aspects, and advantages of the invention will be apparent from the following detailed description, accompanying figures, and from the claims.

### DETAILED DESCRIPTION

### RNAi Agents

Described herein are RNAi agents for inhibiting expression of a MARC1 gene. Each MARC1 RNAi agent comprises a sense strand and an antisense strand. The sense strand can be 15 to 49 nucleotides in length. The antisense strand can be 19 to 49 nucleotides in length. The sense and antisense strands can be either the same length or they can be different lengths. In some embodiments, the sense and antisense strands are each independently 19 to 27 nucleotides in length. In some embodiments, both the sense and antisense strands are each 21-26 nucleotides in length. In some embodiments, the sense and antisense strands are each 21-24 nucleotides in length. In some embodiments, the sense strand is about 19 nucleotides in length while the antisense strand is about 21 nucleotides in length. In some embodiments, the sense strand is about 21 nucleotides in length while the antisense strand is about 23 nucleotides in length. In some embodiments, a sense strand is 23 nucleotides in length and an antisense strand is 21 nucleotides in length. In some embodiments. both the sense and antisense strands are each 21 nucleotides in length. In some embodiments, the RNAi agent antisense strands are each independently 21, 22. 23. 24, 25, 26, 27. 28, 29, or 30 nucleotides in length. In some embodiments, the RNAi agent sense strands are each independently 15, 16, 17, 18. 19, 20, 21, 22, 23, 24, 25, 26, 27, 28, 29, 30, 31, 32, 33, 34, 35, 36, 37, 38, 39, 40, 41, 42, 43, 44, 45, 46, 47. 48, or 49 nucleotides in length. The sense and antisense strands are annealed to form a duplex, and in some embodiments, a double-stranded RNAi agent has a duplex length of about 15. 16. 17, 18, 19, 20, 21. 22. 23, 24, 25, 26, 27. 28, 29, or 30 nucleotides.

Examples of nucleotide sequences used in forming MARC1 RNAi agents are provided in Tables 2. 3, 4, and 5. Examples of RNAi agent duplexes. that include the sense strand and antisense strand sequences in Tables 2, 3, 4, and 5, are shown in Tables 6A. and 6B.

In some embodiments, the region of perfect, substantial, or partial complementarity between the sense strand and the antisense strand is 15-26 (e.g., 15, 16, 17, 18, 19, 20, 21. 22. 23. 24. 25. or 26) nucleotides in length and occurs at or near the 5′ end of the antisense strand (e.g., this region may be separated from the 5′ end of the antisense strand by 0, 1, 2. 3, or 4 nucleotides that are not perfectly, substantially, or partially complementary).

A sense strand of the MARC1 RNAi agents described herein includes at least 15 consecutive nucleotides that have at least 85% identity to a core stretch sequence (also referred to herein as a “core stretch” or “core sequence”) of the same number of nucleotides in a MARC1 mRNA. In some embodiments, a sense strand core stretch sequence is 100% (perfectly) complementary or at least about 85% (substantially) complementary to a core stretch sequence in the antisense strand, and thus the sense strand core stretch sequence is typically perfectly identical or at least about 85% identical to a nucleotide sequence of the same length (sometimes referred to, e.g., as a target sequence) present in the MARC1 mRNA target. In some embodiments, this sense strand core stretch is 15, 16, 17, 18, 19, 20, 21, 22, or 23 nucleotides in length. In some embodiments, this sense strand core stretch is 17 nucleotides in length. In some embodiments, this sense strand core stretch is 19 nucleotides in length. In some embodiments, this sense strand core stretch is 21 nucleotides in length.

An antisense strand of a MARC1 RNAi agent described herein includes at least 15 consecutive nucleotides that have at least 85% complementarity to a core stretch of the same number of nucleotides in a MARC1 mRNA and to a core stretch of the same number of nucleotides in the corresponding sense strand. In some embodiments, an antisense strand core stretch is 100% (perfectly) complementary or at least about 85% (substantially) complementary to a nucleotide sequence (e.g., target sequence) of the same length present in the MARC1 mRNA target. In some embodiments, this antisense strand core stretch is 15, 16, 17, 18, 19, 20, 21, 22, or 23 nucleotides in length. In some embodiments, this antisense strand core stretch is 21 nucleotides in length. In some embodiments, this antisense strand core stretch is 19 nucleotides in length. A sense strand core stretch sequence can be the same length as a corresponding antisense core sequence or it can be a different length.

The MARC1 RNAi agent sense and antisense strands anneal to form a duplex. A sense strand and an antisense strand of a MARC1 RNAi agent can be partially, substantially, or fully complementary to each other. Within the complementary duplex region, the sense strand core stretch sequence is at least 85% complementary or 100% complementary to the antisense core stretch sequence. In some embodiments, the sense strand core stretch sequence contains a sequence of at least 15, at least 16, at least 17, at least 18, at least 19, at least 20, at least 21, at least 22, at least 23, at least 24, or at least 25 nucleotides that is at least 85% or 100% complementary to a corresponding 15, 16, 17, 18, 19. 20, 21, 22, 23, 24, or 25 nucleotide sequence of the antisense strand core stretch sequence (i.e., the sense and antisense core stretch sequences of a MARC1 RNAi agent have a region of at least 15, at least 16, at least 17, at least 18, at least 19, at least 20, at least 21, at least 22, at least 23, at least 24, or at least 25 nucleotides that is at least 85% base paired or 100% base paired.)

In some embodiments, the antisense strand of a MARC1 RNAi agent disclosed herein differs by 0, 1, 2, or 3 nucleotides from any of the antisense strand sequences in Table 2, or Table 3. In some embodiments, the sense strand of a MARC1 RNAi agent disclosed herein differs by 0, 1, 2, or 3 nucleotides from any of the sense strand sequences in Table 2. Table 4, or Table 5.

In some embodiments, the sense strand and/or the antisense strand can optionally and independently contain an additional 1. 2. 3, 4, 5, or 6 nucleotides (extension) at the 3′ end, the 5′ end, or both the 3′ and 5′ ends of the core stretch sequences. The antisense strand additional nucleotides, if present, may or may not be complementary to the corresponding sequence in the MARC1 mRNA. The sense strand additional nucleotides, if present, may or may not be identical to the corresponding sequence in the MARC1 mRNA. The antisense strand additional nucleotides, if present, may or may not be complementary to the corresponding sense strand's additional nucleotides, if present.

As used herein, an extension comprises 1, 2, 3, 4, 5, or 6 nucleotides at the 5′ and/or 3′ end of the sense strand core stretch sequence and/or antisense strand core stretch sequence. The extension nucleotides on a sense strand may or may not be complementary to nucleotides, either core stretch sequence nucleotides or extension nucleotides, in the corresponding antisense strand. Conversely, the extension nucleotides on an antisense strand may or may not be complementary to nucleotides, either core stretch nucleotides or extension nucleotides, in the corresponding sense strand. In some embodiments, both the sense strand and the antisense strand of an RNAi agent contain 3′ and 5′ extensions. In some embodiments, one or more of the 3′ extension nucleotides of one strand base pairs with one or more 5′ extension nucleotides of the other strand. In other embodiments, one or more of 3′ extension nucleotides of one strand do not base pair with one or more 5′ extension nucleotides of the other strand. In some embodiments, a MARC1 RNAi agent has an antisense strand having a 3′ extension and a sense strand having a 5′ extension. In some embodiments, the extension nucleotide(s) are unpaired and form an overhang. As used herein and in the art, an “overhang” refers to an extension of a stretch of one or more unpaired nucleotides located at a terminal end of either the sense strand or the antisense strand that does not form part of the hybridized or duplexed portion of an RNAi agent disclosed herein.

In some embodiments, a MARC1 RNAi agent comprises an antisense strand having a 3′ extension of 1, 2, 3, 4, 5, or 6 nucleotides in length. In other embodiments, a MARC1 RNAi agent comprises an antisense strand having a 3′ extension of 1, 2, or 3 nucleotides in length. In some embodiments, one or more of the antisense strand extension nucleotides comprise nucleotides that are complementary to the corresponding MARC1 mRNA sequence. In some embodiments, one or more of the antisense strand extension nucleotides comprise nucleotides that are not complementary to the corresponding MARC1 mRNA sequence.

In some embodiments, a MARC1 RNAi agent comprises a sense strand having a 3′ extension of 1, 2, 3, 4, or 5 nucleotides in length. In some embodiments, one or more of the sense strand extension nucleotides comprises adenosine, uracil, or thymidine nucleotides, AT dinucleotide, or nucleotides that correspond to or are the identical to nucleotides in the MARC1 mRNA sequence. In some embodiments, the 3′ sense strand extension includes or consists of one of the following sequences, but is not limited to: T. UT. TT. UU, UUT, TTT, or TTTT (each listed 5′ to 3′).

A sense strand can have a 3′ extension and/or a 5′ extension. In some embodiments, a MARC1 RNAi agent comprises a sense strand having a 5′ extension of 1, 2, 3, 4, 5, or 6 nucleotides in length. In some embodiments, one or more of the sense strand extension nucleotides comprise nucleotides that correspond to or are identical to nucleotides in the MARC1 mRNA sequence.

Examples of sequences used in forming MARC1 RNAi agents are provided in Tables 2, 3, 4, 5, and 6D. In some embodiments, a MARC1 RNAi agent antisense strand includes a sequence of any of the sequences in Tables 2, 3, or 6D. In certain embodiments, a MARC1 RNAi agent antisense strand comprises or consists of any one of the modified sequences in Table 3 or Table 6D. In some embodiments, a MARC1 RNAi agent antisense strand includes the sequence of nucleotides (from 5′ end →3′ end) 1-17. 2-15. 2-17. 1-18. 2-18, 1-19. 2-19, 1-20, 2-20, 1-21. or 2-21. of any of the sequences in Tables 2, 3 or 6D. In some embodiments, a MARC1 RNAi agent sense strand includes the sequence of any of the sequences in Tables 2, 4, 5, or 6D. In some embodiments, a MARC1 RNAi agent sense strand includes the sequence of nucleotides (from 5′ end →3′ end) 1-18. 1-19, 1-20, 1-21. 2-19, 2-20. 2-21. 3-20. 3-21, or 4-21 of any of the sequences in Tables 2, 4, 5, or 6D. In certain embodiments, a MARC1 RNAi agent sense strand comprises or consists of a modified sequence of any one of the modified sequences in Table 4, 5 or 6D.

In some embodiments, the sense and antisense strands of the RNAi agents described herein contain the same number of nucleotides. In some embodiments, the sense and antisense strands of the RNAi agents described herein contain different numbers of nucleotides. In some embodiments, the sense strand 5′ end and the antisense strand 3′ end of an RNAi agent form a blunt end. In some embodiments, the sense strand 3′ end and the antisense strand 5′ end of an RNAi agent form a blunt end. In some embodiments, both ends of an RNAi agent form blunt ends. In some embodiments, neither end of an RNAi agent is blunt-ended. As used herein a “blunt end” refers to an end of a double stranded RNAi agent in which the terminal nucleotides of the two annealed strands are complementary (form a complementary base-pair).

In some embodiments, the sense strand 5′ end and the antisense strand 3′ end of an RNAi agent form a frayed end. In some embodiments, the sense strand 3′ end and the antisense strand 5′ end of an RNAi agent form a frayed end. In some embodiments, both ends of an RNAi agent form a frayed end. In some embodiments, neither end of an RNAi agent is a frayed end. As used herein a frayed end refers to an end of a double stranded RNAi agent in which the terminal nucleotides of the two annealed strands from a pair (i.e., do not form an overhang) but are not complementary (i.e. form a non-complementary pair). In some embodiments, one or more unpaired nucleotides at the end of one strand of a double stranded RNAi agent form an overhang. The unpaired nucleotides may be on the sense strand or the antisense strand, creating either 3′ or 5′ overhangs. In some embodiments, the RNAi agent contains: a blunt end and a frayed end, a blunt end and 5′ overhang end, a blunt end and a 3′ overhang end, a frayed end and a 5′ overhang end, a frayed end and a 3′ overhang end, two 5′ overhang ends, two 3′ overhang ends, a 5′ overhang end and a 3′ overhang end, two frayed ends, or two blunt ends. Typically, when present, overhangs are located at the 3′ terminal ends of the sense strand, the antisense strand, or both the sense strand and the antisense strand.

The MARC1 RNAi agents disclosed herein may also be comprised of one or more modified nucleotides. In some embodiments, substantially all of the nucleotides of the sense strand and substantially all of the nucleotides of the antisense strand of the MARC1 RNAi agent are modified nucleotides. The MARC1 RNAi agents disclosed herein may further be comprised of one or more modified internucleoside linkages, e.g., one or more phosphorothioate linkages. In some embodiments, a MARC1 RNAi agent contains one or more modified nucleotides and one or more modified internucleoside linkages. In some embodiments, a 2′-modified nucleotide is combined with modified internucleoside linkage.

In some embodiments, a MARC1 RNAi agent is prepared or provided as a salt, mixed salt, or a free-acid. In some embodiments, a MARC1 RNAi agent is prepared as a pharmaceutically acceptable salt. In some embodiments, a MARC1 RNAi agent is prepared as a pharmaceutically acceptable sodium salt. Such forms that are well known in the art are within the scope of the inventions disclosed herein.

### Modified Nucleotides

Modified nucleotides, when used in various oligonucleotide constructs, can preserve activity of the compound in cells while at the same time increasing the serum stability of these compounds, and can also minimize the possibility of activating interferon activity in humans upon administering of the oligonucleotide construct.

In some embodiments, a MARC1 RNAi agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides can include, but are not limited to, deoxyribonucleotides, nucleotide mimics, abasic nucleotides, 2′-modified nucleotides, inverted nucleotides, modified nucleobase-comprising nucleotides, bridged nucleotides, peptide nucleic acids (PNAs), 2′,3′-seco nucleotide mimics (unlocked nucleobase analogues), locked nucleotides, 3′-O-methoxy (2′ internucleoside linked) nucleotides, 2′-F-Arabino nucleotides, 5′-Me, 2′-fluoro nucleotide, morpholino nucleotides, vinyl phosphonate deoxyribonucleotides, vinyl phosphonate containing nucleotides, and cyclopropyl phosphonate containing nucleotides. 2′-modified nucleotides (i.e., a nucleotide with a group other than a hydroxyl group at the 2′ position of the five-membered sugar ring) include, but are not limited to, 2′-O-methyl nucleotides (also referred to herein or in the art as 2′-methoxy nucleotides), 2′-fluoro nucleotides (also referred to herein or in the art as 2′-deoxy-2′-fluoro nucleotides), 2′-deoxy nucleotides, 2′-methoxyethyl (2′-O-2-methoxylethyl) nucleotides (also referred herein or in the art as 2′-MOE nucleotides), 2′-amino nucleotides, and 2′-alkyl nucleotides. It is not necessary for all positions in a given compound to be uniformly modified. Conversely, more than one modification can be incorporated in a single MARC1 RNAi agent or even in a single nucleotide thereof. The MARC1 RNAi agent sense strands and antisense strands can be synthesized and/or modified by methods known in the art. Modification at one nucleotide is independent of modification at another nucleotide.

Modified nucleobases include synthetic and natural nucleobases, such as 5-substituted pyrimidines, 6-azapyrimidines and N-2, N-6 and O-6 substituted purines, (e.g., 2-aminopropyladenine, 5-propynyluracil, or 5-propynylcytosine), 5-methylcytosine (5-me-C), 5-hydroxymethyl cytosine, inosine, xanthine, hypoxanthine, 2-aminoadenine, 6-alkyl (e.g., 6-methyl, 6-ethyl. 6-isopropyl. or 6-n-butyl) derivatives of adenine and guanine. 2-alkyl (e.g., 2-methyl, 2-ethyl, 2-isopropyl, or 2-n-butyl) and other alkyl derivatives of adenine and guanine. 2-thiouracil, 2-thiothymine, 2-thiocytosine, 5-halouracil, cytosine, 5-propynyl uracil, 5-propynyl cytosine. 6-azo uracil, 6-azo cytosine. 6-azo thymine, 5-uracil (pseudouracil), 4-thiouracil, 8-halo, 8-amino, 8-sulfhydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine. 8-azaguanine and 8-azaadenine. 7-deazaguanine. 7-deazaadenine, 3-deazaguanine, and 3-deazaadenine.

In some embodiments, the 5′ and/or 3′ end of the antisense strand can include abasic residues (Ab), which can also be referred to as an “abasic site” or “abasic nucleotide.” An abasic residue (Ab) is a nucleotide or nucleoside that lacks a nucleobase at the 1′ position of the sugar moiety. In some embodiments, an abasic residue can be placed internally in a nucleotide sequence. In some embodiments, Ab or AbAb can be added to the 3′ end of the antisense strand. In some embodiments, the 5′ end of the sense strand can include one or more additional abasic residues (e.g., (Ab) or (AbAb)). In some embodiments, UUAb, UAb, or Ab are added to the 3′ end of the sense strand. In some embodiments, an abasic (deoxyribose) residue can be replaced with a ribitol (abasic ribose) residue.

In some embodiments, all or substantially all of the nucleotides of an RNAi agent are modified nucleotides. As used herein, an RNAi agent wherein substantially all of the nucleotides present are modified nucleotides is an RNAi agent having four or fewer (i.e., 0, 1, 2. 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0. 1, or 2) nucleotides in the sense strand being unmodified ribonucleotides. As used herein, an antisense strand wherein substantially all of the nucleotides present are modified nucleotides is an antisense strand having two or fewer (i.e., 0), 1, or 2) nucleotides in the antisense strand being unmodified ribonucleotides. In some embodiments, one or more nucleotides of an RNAi agent is an unmodified ribonucleotide. Chemical structures for certain modified nucleotides are set forth in Table 7 herein.

### Modified Internucleoside Linkages

In some embodiments, one or more nucleotides of a MARC1 RNAi agent are linked by non-standard linkages or backbones (i.e., modified internucleoside linkages or modified backbones). Modified internucleoside linkages or backbones include, but are not limited to, phosphorothioate groups (represented herein as a lower case “s”), chiral phosphorothioates. thiophosphates, phosphorodithioates, phosphotriesters, aminoalkyl-phosphotriesters, alkyl phosphonates (e.g., methyl phosphonates or 3′-alkylene phosphonates), chiral phosphonates. phosphinates. phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified internucleoside linkage or backbone lacks a phosphorus atom. Modified internucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified internucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH₂ components.

In some embodiments, a sense strand of a MARC1 RNAi agent can contain 1, 2, 3, 4, 5, or 6 phosphorothioate linkages, an antisense strand of a MARC1 RNAi agent can contain 1. 2. 3, 4, 5, or 6 phosphorothioate linkages, or both the sense strand and the antisense strand independently can contain 1. 2. 3, 4, 5, or 6 phosphorothioate linkages. In some embodiments, a sense strand of a MARC1 RNAi agent can contain 1, 2, 3, or 4 phosphorothioate linkages, an antisense strand of a MARC1 RNAi agent can contain 1, 2, 3, or 4 phosphorothioate linkages, or both the sense strand and the antisense strand independently can contain 1, 2, 3, or 4 phosphorothioate linkages.

In some embodiments, a MARC1 RNAi agent sense strand contains at least two phosphorothioate internucleoside linkages. In some embodiments, the phosphorothioate internucleoside linkages are between the nucleotides at positions 1-3 from the 3′ end of the sense strand. In some embodiments, one phosphorothioate internucleoside linkage is at the 5′ end of the sense strand nucleotide sequence, and another phosphorothioate linkage is at the 3′ end of the sense strand nucleotide sequence. In some embodiments, two phosphorothioate internucleoside linkages are located at the 5′ end of the sense strand, and another phosphorothioate linkage is at the 3′ end of the sense strand. In some embodiments, the sense strand does not include any phosphorothioate internucleoside linkages between the nucleotides, but contains one, two, or three phosphorothioate linkages between the terminal nucleotides on both the 5′ and 3′ ends and the optionally present inverted abasic residue terminal caps. In some embodiments, the targeting ligand is linked to the sense strand via a phosphorothioate linkage.

In some embodiments, a MARC1 RNAi agent antisense strand contains four phosphorothioate internucleoside linkages. In some embodiments, the four phosphorothioate internucleoside linkages are between the nucleotides at positions 1-3 from the 5′ end of the antisense strand and between the nucleotides at positions 19-21, 20-22, 21-23, 22-24, 23-25, or 24-26 from the 5′ end. In some embodiments, three phosphorothioate internucleoside linkages are located between positions 1˜4 from the 5′ end of the antisense strand, and a fourth phosphorothioate internucleoside linkage is located between positions 20-21 from the 5′ end of the antisense strand. In some embodiments, a MARC1 RNAi agent contains at least three or four phosphorothioate internucleoside linkages in the antisense strand.

### Capping Residues or Moieties

In some embodiments, the sense strand may include one or more capping residues or moieties, sometimes referred to in the art as a “cap.” a “terminal cap.” or a “capping residue.” As used herein, a “capping residue” is a non-nucleotide compound or other moiety that can be incorporated at one or more termini of a nucleotide sequence of an RNAi agent disclosed herein. A capping residue can provide the RNAi agent, in some instances, with certain beneficial properties, such as, for example, protection against exonuclease degradation. In some embodiments, inverted abasic residues (invAb) (also referred to in the art as “inverted abasic sites”) are added as capping residues. (See, e.g., F. Czauderna, Nucleic Acids Res., 2003, 31 (11), 2705-16: U.S. Pat. No. 5,998,203). Capping residues are generally known in the art, and include, for example, inverted abasic residues as well as carbon chains such as a terminal C3H7 (propyl), C6H13 (hexyl), or C12H25 (dodecyl) groups. In some embodiments, a capping residue is present at either the 5′ terminal end, the 3′ terminal end, or both the 5′ and 3′ terminal ends of the sense strand. In some embodiments, the 5′ end and/or the 3′ end of the sense strand may include more than one inverted abasic deoxyribose moiety as a capping residue.

In some embodiments, one or more inverted abasic residues (invAb) are added to the 3′ end of the sense strand. In some embodiments, one or more inverted abasic residues (invAb) are added to the 5′ end of the sense strand. In some embodiments, one or more inverted abasic residues or inverted abasic sites are inserted between the targeting ligand and the nucleotide sequence of the sense strand of the RNAi agent. In some embodiments, the inclusion of one or more inverted abasic residues or inverted abasic sites at or near the terminal end or terminal ends of the sense strand of an RNAi agent allows for enhanced activity or other desired properties of an RNAi agent.

In some embodiments, one or more inverted abasic residues (invAb) are added to the 5′ end of the sense strand. In some embodiments, one or more inverted abasic residues can be inserted between the targeting ligand and the nucleotide sequence of the sense strand of the RNAi agent. The inverted abasic residues may be linked via phosphate, phosphorothioate (e.g., shown herein as (invAb) s)), or other internucleoside linkages. In some embodiments, the inclusion of one or more inverted abasic residues at or near the terminal end or terminal ends of the sense strand of an RNAi agent may allow for enhanced activity or other desired properties of an RNAi agent. In some embodiments, an inverted abasic (deoxyribose) residue can be replaced with an inverted ribitol (abasic ribose) residue. In some embodiments, the 3′ end of the antisense strand core stretch sequence, or the 3′ end of the antisense strand sequence, may include an inverted abasic residue. The chemical structures for inverted abasic deoxyribose residues are shown in Table 7 below.

### MARC1 RNAi Agents

The MARC1 RNAi agents disclosed herein are designed to target specific positions on a MARC1 gene (e.g., SEQ ID NO:1).

Homo sapiens. mitochondrial amidoxime reducing component 1 (MARC1). mRNA transcript (SEQ ID NO: 1) (7287 bases). NCBI Reference Sequence: NM_022746.4:

```
      1 cttgccgccg ccacctcgcg gagaagccag ccatgggcgc cgccggctcc tccgcgctgg
    61 cgcgctttgt cctcctcgcg caatcccggc ccgggtggct cggggttgcc gcgctgggcc
  121 tgaccgcggt ggcgctgggg gctgtcgcct ggcgccgcgc atggcccacg cggcgccggc
  181 ggctgctgca gcaggtgggc acagtggcgc agctctggat ctaccctgtg aaatcctgca
  241 agggggtgcc ggtgagcgag gcggagtgca cggccatggg gctgcgcagc ggcaacctgc
  301 gggacaggtt ttggcttgtg atcaaccagg agggaaacat ggttactgct cgccaggaac
  361 ctcgcctggt cctgatttcc ctgacctgcg atggtgacac cctgactctc agtgcagcct
  421 acacaaagga cctactactg cctatcaaaa cgcccaccac aaatgcagtg cacaagtgca
  481 gagtgcacgg cctggagata gagggcaggg actgtggcga ggccaccgcc cagtggataa
  541 ccagcttcct gaagtcacag ccctaccgcc tggtgcactt cgagcctcac atgcgaccga
  601 gacgtcctca tcaaatagca gacttgttcc gacccaagga ccagattgct tactcagaca
  661 ccagcccatt cttgatcctt tctgaggcgt cgctggcgga tctcaactcc aggctagaga
  721 agaaagttaa agcaaccaac ttcaggccca atattgtaat ttcaggatgc gatgtctatg
  781 cagaggattc ttgggatgag cttcttattg gtgacgtgga actgaaaagg gtgatggctt
  841 gttccagatg cattttaacc acagtggacc cagacaccgg tgtcatgagc aggaaggaac
  901 cgctggaaac actgaagagt tatcgccagt gtgacccttc agaacgaaag ttatatggaa
  961 aatcaccact ctttgggcag tattttgtgc tggaaaaccc agggaccatc aaagtgggag
1021 accctgtgta cctgctgggc cagtaatggg aaccgtatgt cctggaatat tagatgcctt
1081 ttaaaaatgt tctcaaaaat gacaacactt gaagcatggt gtttcagaac tgagacctct
1141 acattttctt taaatttgtg attttcacat ttttcgtctt ttggacttct ggtgtctcaa
1201 tgcttcaatg tcccagtgca aaaagtaaag aaatatagtc tcaataactt agtaggactt
1261 cagtaagtca cttaaatgac aagacaggat tctgaaaact ccccgtttaa ctgattatgg
1321 aatagttctt tctcctgctt ctccgtttat ctaccaagag cgcagacttg catcctgtca
1381 ctaccactcg ttagagaaag agaagaagag aaagaggaag agtgggtggg ctggaagaat
1441 atcctagaat gtgttattgc ccctgttcat gaggtacgca atgaaaatta aattgcaccc
1501 caaatatggc tggaatgcca cttccctttt cttctcaagc cccgggctag cttttgaaat
1561 ggcataaaga ctgaggtgac cttcaggaag cactgcagat attaattttc catagatctg
1621 gatctggccc tgctgcttct cagacagcat tggatttcct aaaggtgctc aggaggatgg
1681 ttgtgtagtc atggaggacc cctggatcct tgccattccc ctcagctaat gacggagtgc
1741 tccttctcca gttccgggtg aaaaagttct gaattctgtg gaggagaaga aaagtgattc
1801 agtgatttca gatagactac tgaaaacctt taaaggggga aaaggaaagc atatgtcagt
1861 tgtttaaaac ccaatatcta ttttttaact gattgtataa ctctaagatc tgatgaagta
1921 tattttttat tgccattttg tcctttgatt atattgggaa gttgactaaa cttgaaaaat
1981 gtttttaaaa ctgtgaataa atggaagcta ctttgactag tttcagatct tactaacttc
2041 ttggcacaaa gttagactgt gaaagctgac tgaggctggg cacaggggct catgcctgta
2101 attccagcac tttgggaggc caaggtggga gaatggcttg agcccaggag tttgagacca
2161 gcccagaaaa tataatggga tcctgtcgct acaaaatgtt tttaaaatgc actcggtgtg
2221 gtggtgtgtg cctgcagtcc tggctatggc tactcgggag gatgaggtag aaggattggt
2281 tgagcccagg agcgggagat tgaggctgca gtgagttatg attgcaccac tacactccag
2341 cctgagtgat agagtgagac cctatctcta aaaaagaaac aggaaaaaaa aagaaagctg
2401 actgaggtga atgggcaaag ccagtaattc tgacacctga ccacagctgg gtcttctgca
2461 taatggacct cctcacccac agcctcccag gcaagcaccc atgtttgaag gactatcaag
2521 tcaacatgct ttttaccaaa agctgcacat ttttcacttt gattttataa aagaggtcag
2581 taatcgctga aatctagctg agccctgaag taaagttctg agcaaagagg tgcatgtgct
2641 tgttttatgg ttggtgaatt attacagttt gttttctgca tgcttggcat gaggtgaata
2701 attacatcaa ttttccagag aacctgggcc atcaccttcc ccaacaagtc cagttgatgt
2761 tgaaactaca gatagattga gacaaagcga agtgttcagc aagtagcatt actaatggga
2821 ccgggggacc cgtgggagag tgagtgtaca caggatttag gaaaccatgt gaatatgggc
2881 tctctgggaa tagccaatag gtagggagca atcagaaacc caaggtttgg tggctcttcc
2941 taggtattta taattagtgg caagtgaaag ccttagtcct gaatttctaa ccacttgtaa
3001 gaactaacag ccacttctct gtgccccgtc cgggcagtaa ccatcattct ccatggacag
3061 gctctcgggg tagctagctc tgcagggcag cacccacgtg gaagggagca cccagaaacc
3121 ctcctcactg ggcagacctg tccttctgtg cctcacagtg tgaggaagat tcctgtttga
3181 agagagaagt tccagtgacc tctagaatct cagagtagtt gccaagcttt ctgtcagtga
3241 gatttaaagg ccatttactt gtgtttattt tatatttaat gagttggtta atgccagaga
3301 caaagctgat atcccattta ttttggatac tgagcatttg cacactattc cacttgaaat
3361 atagaatcag gaatgtaggc catcccagac tttcagatct tacaacagca aatgacagat
3421 gtttgagatc aggccaaaat atccaccctc ggtgggcatc tcctctgtgt ggcaacttat
3481 gctgcagcca cagtggggag tcacaaactc agagctggag gtcttgaaaa ggacaatgtg
3541 ggccaggctc cggaggggct gcctaaaggc ttgcttttgt gactctcctg cagaaaatgt
3601 tagaaacttc caaccgaaag acgagggcag caacttatac acacgaaggc agaaagaaat
3661 tggggaaggg gaggctgttg gaattcaggc cgttgtccta tagggagaaa tactcctcct
3721 ctccttctcc ctttactgat aacggggcat ggtgaggaga tgagcttgtg agggtctgcc
3781 agtttggtaa gagtgcatgg ggaggttggg taaattagac tagccaaatg ggacttcggg
3841 aaaccattta tgaggctgtc accaacagtg atggcaggct gaaattccag gcaagtgctc
3901 ccagcattcc aagagtgtat caaattaaag caacccatga tggtggagaa cagatacatt
3961 aaagttcctt gaaaatgaca gagtggctct cagaccagac cttgattgtg ggtataatcg
4021 gagtgttgct accacaccct aacactgcat ttcccgtgtt ttattggtcc atggaattct
4081 gaaagtttgc ctttcgggat gcttctaaaa acaattccat ggaccagtaa gtttggaaag
4141 tcctgcgtgc ctcacttctc ttcaaaggca aaaggctctg gagaggcctt catgaagaca
4201 tctgtgttta atgctgccct tcccaaaggt ctgtttttga ctgtcttttg agaaatgatc
4261 ctctgatctc taggcagaat gccagtgagc caaggaatcc cagttagcag gaggggtgca
4321 ctcatgggaa gactgaagaa gttaaaagtt cccgccaagt gaaggagacc tatcttggga
4381 cacttcccct tgtcctctcc cttgcccctc ttgctggagt aaaaggatgg aactgggact
4441 tgataggtta aaggaggtgt ggagaagtgt cttagaccag ctctcctgtt gtgggcctta
4501 gggagaagca ctctctttct tcgggatcat tttccaaaca tgcatttttg gatggatagg
4561 gtggatcagg gtgagggaag ggaaaccaaa ctctctctaa ccttgccctt acagcaatac
4621 ctgtgatgta agttacaaaa ccacctgtga tgaaagtgct ccaggatgct tcatgcacca
4681 gggaggggtg ccctgtttct cttctgctag cttctccttt cttttttttt tttcttcttt
4741 tttttgagac agtgtctcac tctgttgcca ggctggagtg cagtggtgag atctcagctc
4801 actgcagcct ctgcctccca ggttcaagca attcttctgc ctcagcctcc cgagtagctg
4861 gtgtgtctgg agttggttcc ttctggtggg ttcttggtct cgctgacttc aagaatgaag
4921 ccacagacct tcgcagtgag tgttacagct cttaaaggtg gcacggaccc aaagtgagca
4981 gtagcaagat ttattgtgga gagcgaaaga acaaagcttc ggaaggggac ccaaatgggc
5041 tgctgctgct ggctggggtg gccacctttt attcccttat ttgtccctgc ccatgtcctg
5101 ctgattgctc cattttacag agtgctgatt ggtccatttt acagagtgct gattggtgca
5161 tttacaatcc tttagctaga cacagagtgc cgattggtga gtttttacag tgctgattgg
5221 tgcatttaca atcctttagc tagacacaga acactgactg gtgcatttat aatcctctag
5281 ctagaaagaa aagttctcca agtccccact agacccagga agtccagctg gcttcacctc
5341 tcactgggac tacaggtgca caccaccaca cccagctaat ttttgtattt ttagtagaga
5401 cggggtttca ccatgttgtt caggatggtc tcgaactctt gatctcgtga tctgcccgcc
5461 tcggcctccc aaagtgctgg gattacagtt gtgagccacc acgcccggcc ctagcttttc
5521 ctttctgttg caagtcctct caactagtgt tgccttccac cctacaaagc agaattacct
5581 cagaagtcct atggccctga ctctatctat gtctgcacaa agcactactg tgctttgctg
5641 tctgcaagaa cagagattgt ttgcttcaac cactttctct gaatggatga atgagttatg
5701 atgatatcta aagttaccca atttcaagca agaggaagaa tctggctcgg taccacagat
5761 gttcttggaa ttgggatagt aaaaaagtcc ctgaggcatc ccttggtctg ctctgaccac
5821 actctcttca caggaagagg cttgggccac agctctgact ataactctgc tcttcctcca
5881 aacacagctg aggaattggg tggtggggca cctgctccca tgctctgtgg cctggctcag
5941 agagaagagt tgccttaatt acattattat tcttcctgga caggctgtag gttgtgtaaa
6001 gtaacaaaaa ggactgagaa gtgacttccc attcagcctc ttccaaggcc atttttgata
6061 ggcaggtcaa attcactcac atttggttat ttgttggcca gtctagtgca ttcacccttg
6121 ctggtcctca gtcatgctcc tttaccttta cagagcatcc tagactgctc ttcctcttac
6181 cttccttgtg aaacccacaa cccctagtcc ctccccttcc ctggcatttg ttatgccctc
6241 taccaatccc tgacctggta ttggtcagtc tccaatcctg gtggatccct gtgggaacta
6301 agttaagtct aacttttgtc tccctcttta gaatttactg ggagtactgt aaataaacta
6361 ttgttgttat aattatttct gattaacatt tttacaccta acaaagtctc agagagattg
6421 aatttactgg gttgaaggga ggagcacctt ccacatgacc tgcccagcaa ttaaagccgc
6481 ttgttagtcc gaggcccagg acggccgagg acagctggag agctcttcgt tgcaggcagc
6541 tctggttaac atcaaccggg aaagctcttt gtaaacacat gaataattga tcgtccagcg
6601 ctcacatagc taccgcggat ctgagcccgt atgactcatt tgcgagccat tcctgtcgtc
6661 tggatgccat aacattggag gaatgatgat cgtttcttgg aggttcttct gtggccagag
6721 ttgccaagac caaggctgta atggtttgtt atgatgacct ttgttattcc attaggctca
6781 attgctttaa aaaatgatgt gtgcatactt taggaacgtt tttacccttt atgttgacct
6841 gacatcatag tttatattat aaaatgtatt aatgacagaa gagtgttttc atgtcccaag
6901 gacaaatttt aacaaccata atctgccctc agtcatcata aatataaatg tattggtcaa
6961 acagatctcg ttaatgtggc caagataaat gcaagtctat attttaaggc agtcgaagtc
7021 ctagagaata tatctggagc ttttgtgggg ctaagagatc ttgtatatat gctatcaaaa
7081 ggctgagaaa attaacatgt tcccccctct gattttgcat tggacagata taaatgtctt
7141 ggggatgtca agtaagattg ttcacatagt ttctggacac cattaatgcc tgatggggtg
7201 aatcttagtt cttaaagcta tattctgctc attatgctca cagggctttt gaaaagagaa
7261 caaaataaag atttcaagtc ttagcaa
```

As defined herein, an antisense strand sequence is designed to target a MARC1 gene at a given position on the gene when the 5′ terminal nucleobase of the antisense strand is aligned with a position that is 21 nucleotides downstream (towards the 3′ end) from the position on the gene when base pairing to the gene. For example, as illustrated in Tables 1 and 2 herein, an antisense strand sequence designed to target a MARC1 gene at position 1275 requires that when base pairing to the gene, the 5′ terminal nucleobase of the antisense strand is aligned with position 1295 of the MARC1 gene.

As provided herein, a MARC1 RNAi agent does not require that the nucleobase at position 1 (5′→3′) of the antisense strand be complementary to the gene, provided that there is at least 85% complementarity (e.g., at least 85, 86, 87, 88, 89, 90, 91, 92, 93, 94, 95, 96, 97, 98, 99, or 100% complementarity) of the antisense strand and the gene across a core stretch sequence of at least 15 consecutive nucleotides. For example, for a MARC1 RNAi agent disclosed herein that is designed to target position 1275 of a MARC1 gene, the 5′ terminal nucleobase of the antisense strand of the of the MARC1 RNAi agent must be aligned with position 1295 of the gene; however, the 5′ terminal nucleobase of the antisense strand may be, but is not required to be, complementary to position 1295 of a MARC1 gene, provided that there is at least 85% complementarity (e.g., at least 85, 86, 87, 88, 89, 90, 91, 92, 93, 94, 95, 96, 97, 98, 99, or 100% complementarity) of the antisense strand and the gene across a core stretch sequence of at least 15 consecutive nucleotides. As shown by, among other things, the examples disclosed herein and as is well known in the art, the specific site of binding of the gene by the antisense strand of the MARC1 RNAi agent (e.g., whether the MARC1 RNAi agent is designed to target a MARC1 gene at position 1275, at position 1190, or at some other position) is important to the level of inhibition achieved by the MARC1 RNAi agent as well as the toxicity profile achieved by the molecule. (See. e.g., Kamola et al., The siRNA Non-seed Region and Its Target Sequences are Auxiliary Determinants of Off-Target Effects. PLOS Computational Biology, 11 (12), FIG. 1 (2015)).

In some embodiments, the MARC1 RNAi agents disclosed herein target a MARC1 gene at or near the positions of the MARC1 gene sequence shown in Table 1. In some embodiments, the antisense strand of a MARC1 RNAi agent disclosed herein includes a core stretch sequence that is fully, substantially, or at least partially complementary to a target MARC1 19-mer sequence disclosed in Table 1.

TABLE 1
MARC1 19-mer mRNA Target Sequences (taken from homo sapiens MARC1,
mRNA, GenBank NM_022746.4 (SEQ ID NO: 1))

| | | Corresponding | Targeted Gene |
|---|---|---|---|
| | MARC1 19-mer | Positions of | Position |
| SEQ ID | Target Sequences | Sequence | (as referred |
| No. | (5′→3′) | on SEQ ID NO: 1 | to herein) |
| 2 | GGUUUUGGCUUGUGAUCAA | 307-325 | 305 |
| 3 | GUUUUGGCUUGUGAUCAAC | 308-326 | 306 |
| 4 | UUUUGGCUUGUGAUCAACC | 309-327 | 307 |
| 5 | CAGGAGGGAAACAUGGUUA | 327-345 | 325 |
| 6 | UCUCAGUGCAGCCUACACA | 407-425 | 405 |
| 7 | CUCAGUGCAGCCUACACAA | 408-426 | 406 |
| 8 | AGUGCAGCCUACACAAAGG | 411-429 | 409 |
| 9 | UGCAGCCUACACAAAGGAC | 413-431 | 411 |
| 10 | ACACAAAGGACCUACUACU | 421-439 | 419 |
| 11 | UAACCAGCUUCCUGAAGUC | 538-556 | 536 |
| 12 | CCGCCUGGUGCACUUCGAG | 566-584 | 564 |
| 13 | CGCCUGGUGCACUUCGAGC | 567-585 | 565 |
| 14 | GCCUGGUGCACUUCGAGCC | 568-586 | 566 |
| 15 | CCUGGUGCACUUCGAGCCU | 569-587 | 567 |
| 16 | CGUCCUCAUCAAAUAGCAG | 603-621 | 601 |
| 17 | UCAAAUAGCAGACUUGUUC | 611-629 | 609 |
| 18 | CAAAUAGCAGACUUGUUCC | 612-630 | 610 |
| 19 | AAAUAGCAGACUUGUUCCG | 613-631 | 611 |
| 20 | GGACCAGAUUGCUUACUCA | 638-656 | 636 |
| 21 | CAGAUUGCUUACUCAGACA | 642-660 | 640 |
| 22 | UUGCUUACUCAGACACCAG | 646-664 | 644 |
| 23 | GGCUAGAGAAGAAAGUUAA | 712-730 | 710 |
| 24 | AGAGAAGAAAGUUAAAGCA | 716-734 | 714 |
| 25 | AGAAGAAAGUUAAAGCAAC | 718-736 | 716 |
| 26 | GCCCAAUAUUGUAAUUUCA | 746-764 | 744 |
| 27 | CAGGAUGCGAUGUCUAUGC | 763-781 | 761 |
| 28 | UCCAGAUGCAUUUUAACCA | 843-861 | 841 |
| 29 | CUGGAAACACUGAAGAGUU | 903-921 | 901 |
| 30 | ACCCUUCAGAACGAAAGUU | 934-952 | 932 |
| 31 | CCCUUCAGAACGAAAGUUA | 935-953 | 933 |
| 32 | CAGAACGAAAGUUAUAUGG | 940-958 | 938 |
| 33 | GAACGAAAGUUAUAUGGAA | 942-960 | 940 |
| 34 | AAAGUUAUAUGGAAAAUCA | 947-965 | 945 |
| 35 | UGGAAAAUCACCACUCUUU | 956-974 | 954 |
| 36 | GAAAAUCACCACUCUUUGG | 958-976 | 956 |
| 37 | CAAAGUGGGAGACCCUGUG | 1010-1028 | 1008 |
| 38 | AAAGUGGGAGACCCUGUGU | 1011-1029 | 1009 |
| 39 | GUGGGAGACCCUGUGUACC | 1014-1032 | 1012 |
| 40 | UGGGAGACCCUGUGUACCU | 1015-1033 | 1013 |
| 41 | GGGAGACCCUGUGUACCUG | 1016-1034 | 1014 |
| 42 | GUCCUGGAAUAUUAGAUGC | 1059-1077 | 1057 |
| 43 | UCCUGGAAUAUUAGAUGCC | 1060-1078 | 1058 |
| 44 | CCUGGAAUAUUAGAUGCCU | 1061-1079 | 1059 |
| 45 | CUGGAAUAUUAGAUGCCUU | 1062-1080 | 1060 |
| 46 | UCUCAAAAAUGACAACACU | 1091-1109 | 1089 |
| 47 | UGACAACACUUGAAGCAUG | 1100-1118 | 1098 |
| 48 | AACACUUGAAGCAUGGUGU | 1104-1122 | 1102 |
| 49 | GAAGCAUGGUGUUUCAGAA | 1111-1129 | 1109 |
| 50 | AGCAUGGUGUUUCAGAACU | 1113-1131 | 1111 |
| 51 | AGAACUGAGACCUCUACAU | 1126-1144 | 1124 |
| 52 | AGACCUCUACAUUUUCUUU | 1133-1151 | 1131 |
| 53 | UGAUUUUCACAUUUUUCGU | 1159-1177 | 1157 |
| 54 | GUGUCUCAAUGCUUCAAUG | 1192-1210 | 1190 |
| 55 | UCUCAAUGCUUCAAUGUCC | 1195-1213 | 1193 |
| 56 | CUUAGUAGGACUUCAGUAA | 1248-1266 | 1246 |
| 57 | AUGACAAGACAGGAUUCUG | 1276-1294 | 1274 |
| 58 | UGACAAGACAGGAUUCUGA | 1277-1295 | 1275 |
| 59 | GACAAGACAGGAUUCUGAA | 1278-1296 | 1276 |
| 60 | ACAGGAUUCUGAAAACUCC | 1284-1302 | 1282 |
| 61 | CGUUUAACUGAUUAUGGAA | 1304-1322 | 1302 |
| 62 | UGAUUAUGGAAUAGUUCUU | 1312-1330 | 1310 |
| 63 | UUAUGGAAUAGUUCUUUCU | 1315-1333 | 1313 |
| 64 | UAUGGAAUAGUUCUUUCUC | 1316-1334 | 1314 |
| 65 | CCUGCUUCUCCGUUUAUCU | 1334-1352 | 1332 |
| 66 | GAAGAAUAUCCUAGAAUGU | 1434-1452 | 1432 |
| 67 | UUUCCAUAGAUCUGGAUCU | 1607-1625 | 1605 |
| 68 | GCUUCUCAGACAGCAUUGG | 1635-1653 | 1633 |
| 69 | UUCUCAGACAGCAUUGGAU | 1637-1655 | 1635 |
| 70 | ACAGCAUUGGAUUUCCUAA | 1644-1662 | 1642 |
| 71 | CAUUGGAUUUCCUAAAGGU | 1648-1666 | 1646 |
| 72 | UUGGAUUUCCUAAAGGUGC | 1650-1668 | 1648 |
| 73 | ACUGAAAACCUUUAAAGGG | 1819-1837 | 1817 |
| 74 | GGAAAGCAUAUGUCAGUUG | 1844-1862 | 1842 |
| 75 | UGUCAGUUGUUUAAAACCC | 1854-1872 | 1852 |
| 76 | AACUCUAAGAUCUGAUGAA | 1899-1917 | 1897 |
| 77 | ACUCUAAGAUCUGAUGAAG | 1900-1918 | 1898 |
| 78 | UCUAAGAUCUGAUGAAGUA | 1902-1920 | 1900 |
| 79 | AUUGCCAUUUUGUCCUUUG | 1929-1947 | 1927 |
| 80 | CCAUUUUGUCCUUUGAUUA | 1933-1951 | 1931 |
| 81 | GGGAAGUUGACUAAACUUG | 1956-1974 | 1954 |
| 82 | GGAAGUUGACUAAACUUGA | 1957-1975 | 1955 |
| 83 | UGUGAAUAAAUGGAAGCUA | 1992-2010 | 1990 |
| 84 | ACUAGUUUCAGAUCUUACU | 2016-2034 | 2014 |
| 85 | GUUUCAGAUCUUACUAACU | 2020-2038 | 2018 |
| 86 | UUCAGAUCUUACUAACUUC | 2022-2040 | 2020 |
| 87 | GCAUGUGCUUGUUUUAUGG | 2632-2650 | 2630 |
| 88 | GACAAAGCGAAGUGUUCAG | 2781-2799 | 2779 |
| 89 | CCAGUGACCUCUAGAAUCU | 3192-3210 | 3190 |
| 90 | GCAUUUGCACACUAUUCCA | 3334-3352 | 3332 |
| 91 | GACAGAUGUUUGAGAUCAG | 3414-3432 | 3412 |
| 92 | UGUGUGGCAACUUAUGCUG | 3466-3484 | 3464 |
| 93 | GACCAGACCUUGAUUGUGG | 3993-4011 | 3991 |
| 94 | GGAGAAGCACUCUCUUUCU | 4502-4520 | 4500 |
| 95 | AACCAAACUCUCUCUAACC | 4584-4602 | 4582 |
| 96 | CAAACUCUCUCUAACCUUG | 4587-4605 | 4585 |
| 97 | GAUGUAAGUUACAAAACCA | 4625-4643 | 4623 |
| 98 | CCACCUGUGAUGAAAGUGC | 4641-4659 | 4639 |
| 99 | ACUGGUGCAUUUAUAAUCC | 5257-5275 | 5255 |
| 100 | AGCUCUGACUAUAACUCUG | 5851-5869 | 5849 |
| 101 | CCUUUACAGAGCAUCCUAG | 6145-6163 | 6143 |
| 102 | AACUAAGUUAAGUCUAACU | 6296-6314 | 6294 |
| 103 | CAGAGAGAUUGAAUUUACU | 6410-6428 | 6408 |
| 104 | GCUCUGGUUAACAUCAACC | 6539-6557 | 6537 |
| 105 | UGACCUGACAUCAUAGUUU | 6835-6853 | 6833 |
| 106 | AUAAAAUGUAUUAAUGACA | 6959-6977 | 6957 |
| 107 | UCUUAAAGCUAUAUUCUGC | 7210-7228 | 7208 |

In some embodiments, a MARC1 RNAi agent includes an antisense strand wherein position 19 of the antisense strand (5′>3′) is capable of forming a base pair with position 1 of a 19-mer target sequence disclosed in Table 1. In some embodiments, a MARC1 RNAi agent includes an antisense strand wherein position 1 of the antisense strand (5′→3′) is capable of forming a base pair with position 19 of the 19-mer target sequence disclosed in Table 1.

In some embodiments, a MARC1 RNAi agent includes an antisense strand wherein position 2 of the antisense strand (5′→3′) is capable of forming a base pair with position 18 of the 19-mer target sequence disclosed in Table 1. In some embodiments, a MARC1 RNAi agent includes an antisense strand wherein positions 2 through 18 of the antisense strand (5′→3′) are capable of forming base pairs with each of the respective complementary bases located at positions 18 through 2 of the 19-mer target sequence disclosed in Table 1.

For the RNAi agents disclosed herein, the nucleotide at position 1 of the antisense strand (from 5′ end->3′ end) can be perfectly complementary to the MARC1 gene, or can be non-complementary to the MARC1 gene. In some embodiments, the nucleotide at position 1 of the antisense strand (from 5′ end->3′ end) is a U, A, or dT. In some embodiments, the nucleotide at position 1 of the antisense strand (from 5′ end →3′ end) forms an A: U or U: A base pair with the sense strand.

In some embodiments, a MARC1 RNAi agent antisense strand comprises the sequence of nucleotides (from 5′ end →3′ end) 2-18, 2-19, 2-20, or 2-21 of any of the antisense strand sequences in Table 2 or Table 3. In some embodiments, a MARC1 RNAi sense strand comprises the sequence of nucleotides (from 5′ end->3′ end) 3-21, 2-21, 1-21, 3-20, 2-20, 1-20, 3-19, 2-19, 1-19, 3-18, 2-18, or 1-18 of any of the sense strand sequences in Table 2, Table 4, Table 5, or Table 6D.

In some embodiments, a MARC1 RNAi agent antisense strand comprises the sequence of nucleotides (from 5′ end->3′ end) 2-18, 2-19, 2-20, or 2-21 of any of the antisense strand sequences of Table 2, or Table 3. In some embodiments, a MARC1 RNAi sense strand comprises the sequence of nucleotides (from 5′ end->3′ end) 3-21, 2-21, 1-21, 3-20, 2-20, 1-20, 3-19, 2-19, 1-19, 3-18, 2-18, or 1-18 of any of the sense strand sequences of Table 2, Table 4, Table 5, or Table 6D.

In some embodiments, a MARC1 RNAi agent is comprised of (i) an antisense strand comprising the sequence of nucleotides (from 5′ end →3′ end) 2-18 or 2-19 of any of the antisense strand sequences in Table 2, Table 3, or Table 6D, and (ii) a sense strand comprising the sequence of nucleotides (from 5′ end->3′ end) 3-21, 2-21, 1-21, 3-20, 2-20, 1-20, 3-19, 2-19, 1-19, 3-18, 2-18, or 1-18 of any of the sense strand sequences in Table 2, Table 4, Table 5, or Table 6D.

In some embodiments, a MARC1 RNAi agent is comprised of (i) an antisense strand comprising the sequence of nucleotides (from 5′ end->3′ end) 2-18 or 2-19 of any of the antisense strand sequences of Table 2, Table 3, or Table 6D, and (ii) a sense strand comprising the sequence of nucleotides (from 5′ end →3′ end) 3-21, 2-21, 1-21, 3-20, 2-20, 1-20, 3-19, 2-19, 1-19, 3-18, 2-18, or 1-18 of any of the sense strand sequences of Table 2, Table 4, Table 5, or Table 6D.

In some embodiments, the MARC1 RNAi agents include core 19-mer nucleotide sequences shown in the following Table 2.

TABLE 2
MARC1 RNAi agent Antisense Strand and Sense Strand Core Stretch Base Sequences
(N = any nucleobase)

| | | | | Corresponding | |
|---|---|---|---|---|---|
| | Antisense Strand Base | | Sense Strand Base | Positions of | |
| SEQ | Sequence (5′→3′) | SEQ | Sequence (5′→3′) | Identified | Targeted |
| ID | (Shown as an Unmodified | ID | (Shown as an Unmodified | Sequence on | Genome |
| NO:. | Nucleotide Sequence) | NO:. | Nucleotide Sequence) | SEQ ID NO: 1 | Position |
| 108 | UUGAUCACAAGCCAAAACC | 582 | GGUUUUGGCUUGUGAUCAA | 307-325 | 305 |
| 109 | AUGAUCACAAGCCAAAACC | 583 | GGUUUUGGCUUGUGAUCAU | 307-325 | 305 |
| 110 | NUGAUCACAAGCCAAAACC | 584 | GGUUUUGGCUUGUGAUCAN | 307-325 | 305 |
| 111 | NUGAUCACAAGCCAAAACN | 585 | NGUUUUGGCUUGUGAUCAN | 307-325 | 305 |
| 112 | GUUGAUCACAAGCCAAAAC | 586 | GUUUUGGCUUGUGAUCAAC | 308-326 | 306 |
| 113 | UUUGAUCACAAGCCAAAAC | 587 | GUUUUGGCUUGUGAUCAAA | 308-326 | 306 |
| 114 | AUUGAUCACAAGCCAAAAC | 588 | GUUUUGGCUUGUGAUCAAU | 308-326 | 306 |
| 115 | NUUGAUCACAAGCCAAAAC | 589 | GUUUUGGCUUGUGAUCAAN | 308-326 | 306 |
| 116 | NUUGAUCACAAGCCAAAAN | 590 | NUUUUGGCUUGUGAUCAAN | 308-326 | 306 |
| 117 | GGUUGAUCACAAGCCAAAA | 591 | UUUUGGCUUGUGAUCAACC | 309-327 | 307 |
| 118 | UGUUGAUCACAAGCCAAAA | 592 | UUUUGGCUUGUGAUCAACA | 309-327 | 307 |
| 119 | AGUUGAUCACAAGCCAAAA | 593 | UUUUGGCUUGUGAUCAACU | 309-327 | 307 |
| 120 | NGUUGAUCACAAGCCAAAA | 594 | UUUUGGCUUGUGAUCAACN | 309-327 | 307 |
| 121 | NGUUGAUCACAAGCCAAAN | 595 | NUUUGGCUUGUGAUCAACN | 309-327 | 307 |
| 122 | UAACCAUGUUUCCCUCCUG | 596 | CAGGAGGGAAACAUGGUUA | 327-345 | 325 |
| 123 | AAACCAUGUUUCCCUCCUG | 597 | CAGGAGGGAAACAUGGUUU | 327-345 | 325 |
| 124 | NAACCAUGUUUCCCUCCUG | 598 | CAGGAGGGAAACAUGGUUN | 327-345 | 325 |
| 125 | NAACCAUGUUUCCCUCCUN | 599 | NAGGAGGGAAACAUGGUUN | 327-345 | 325 |
| 126 | UGUGUAGGCUGCACUGAGA | 600 | UCUCAGUGCAGCCUACACA | 407-425 | 405 |
| 127 | AGUGUAGGCUGCACUGAGA | 601 | UCUCAGUGCAGCCUACACU | 407-425 | 405 |
| 128 | NGUGUAGGCUGCACUGAGA | 602 | UCUCAGUGCAGCCUACACN | 407-425 | 405 |
| 129 | NGUGUAGGCUGCACUGAGN | 603 | NCUCAGUGCAGCCUACACN | 407-425 | 405 |
| 130 | UUGUGUAGGCUGCACUGAG | 604 | CUCAGUGCAGCCUACACAA | 408-426 | 406 |
| 131 | AUGUGUAGGCUGCACUGAG | 605 | CUCAGUGCAGCCUACACAU | 408-426 | 406 |
| 132 | NUGUGUAGGCUGCACUGAG | 606 | CUCAGUGCAGCCUACACAN | 408-426 | 406 |
| 133 | NUGUGUAGGCUGCACUGAN | 607 | NUCAGUGCAGCCUACACAN | 408-426 | 406 |
| 134 | CCUUUGUGUAGGCUGCACU | 608 | AGUGCAGCCUACACAAAGG | 411-429 | 409 |
| 135 | UCUUUGUGUAGGCUGCACU | 609 | AGUGCAGCCUACACAAAGA | 411-429 | 409 |
| 136 | ACUUUGUGUAGGCUGCACU | 610 | AGUGCAGCCUACACAAAGU | 411-429 | 409 |
| 137 | NCUUUGUGUAGGCUGCACU | 611 | AGUGCAGCCUACACAAAGN | 411-429 | 409 |
| 138 | NCUUUGUGUAGGCUGCACN | 612 | NGUGCAGCCUACACAAAGN | 411-429 | 409 |
| 139 | GUCCUUUGUGUAGGCUGCA | 613 | UGCAGCCUACACAAAGGAC | 413-431 | 411 |
| 140 | UUCCUUUGUGUAGGCUGCA | 614 | UGCAGCCUACACAAAGGAA | 413-431 | 411 |
| 141 | AUCCUUUGUGUAGGCUGCA | 615 | UGCAGCCUACACAAAGGAU | 413-431 | 411 |
| 142 | NUCCUUUGUGUAGGCUGCA | 616 | UGCAGCCUACACAAAGGAN | 413-431 | 411 |
| 143 | NUCCUUUGUGUAGGCUGCN | 617 | NGCAGCCUACACAAAGGAN | 413-431 | 411 |
| 144 | AGUAGUAGGUCCUUUGUGU | 618 | ACACAAAGGACCUACUACU | 421-439 | 419 |
| 145 | UGUAGUAGGUCCUUUGUGU | 619 | ACACAAAGGACCUACUACA | 421-439 | 419 |
| 146 | NGUAGUAGGUCCUUUGUGU | 620 | ACACAAAGGACCUACUACN | 421-439 | 419 |
| 147 | NGUAGUAGGUCCUUUGUGN | 621 | NCACAAAGGACCUACUACN | 421-439 | 419 |
| 148 | GACUUCAGGAAGCUGGUUA | 622 | UAACCAGCUUCCUGAAGUC | 538-556 | 536 |
| 149 | UACUUCAGGAAGCUGGUUA | 623 | UAACCAGCUUCCUGAAGUA | 538-556 | 536 |
| 150 | AACUUCAGGAAGCUGGUUA | 624 | UAACCAGCUUCCUGAAGUU | 538-556 | 536 |
| 151 | NACUUCAGGAAGCUGGUUA | 625 | UAACCAGCUUCCUGAAGUN | 538-556 | 536 |
| 152 | NACUUCAGGAAGCUGGUUN | 626 | NAACCAGCUUCCUGAAGUN | 538-556 | 536 |
| 153 | CUCGAAGUGCACCAGGCGG | 627 | CCGCCUGGUGCACUUCGAG | 566-584 | 564 |
| 154 | UUCGAAGUGCACCAGGCGG | 628 | CCGCCUGGUGCACUUCGAA | 566-584 | 564 |
| 155 | AUCGAAGUGCACCAGGCGG | 629 | CCGCCUGGUGCACUUCGAU | 566-584 | 564 |
| 156 | NUCGAAGUGCACCAGGCGG | 630 | CCGCCUGGUGCACUUCGAN | 566-584 | 564 |
| 157 | NUCGAAGUGCACCAGGCGN | 631 | NCGCCUGGUGCACUUCGAN | 566-584 | 564 |
| 158 | GCUCGAAGUGCACCAGGCG | 632 | CGCCUGGUGCACUUCGAGC | 567-585 | 565 |
| 159 | UCUCGAAGUGCACCAGGCG | 633 | CGCCUGGUGCACUUCGAGA | 567-585 | 565 |
| 160 | ACUCGAAGUGCACCAGGCG | 634 | CGCCUGGUGCACUUCGAGU | 567-585 | 565 |
| 161 | NCUCGAAGUGCACCAGGCG | 635 | CGCCUGGUGCACUUCGAGN | 567-585 | 565 |
| 162 | NCUCGAAGUGCACCAGGCN | 636 | NGCCUGGUGCACUUCGAGN | 567-585 | 565 |
| 163 | GGCUCGAAGUGCACCAGGC | 637 | GCCUGGUGCACUUCGAGCC | 568-586 | 566 |
| 164 | UGCUCGAAGUGCACCAGGC | 638 | GCCUGGUGCACUUCGAGCA | 568-586 | 566 |
| 165 | AGCUCGAAGUGCACCAGGC | 639 | GCCUGGUGCACUUCGAGCU | 568-586 | 566 |
| 166 | NGCUCGAAGUGCACCAGGC | 640 | GCCUGGUGCACUUCGAGCN | 568-586 | 566 |
| 167 | NGCUCGAAGUGCACCAGGN | 641 | NCCUGGUGCACUUCGAGCN | 568-586 | 566 |
| 168 | AGGCUCGAAGUGCACCAGG | 642 | CCUGGUGCACUUCGAGCCU | 569-587 | 567 |
| 169 | UGGCUCGAAGUGCACCAGG | 643 | CCUGGUGCACUUCGAGCCA | 569-587 | 567 |
| 170 | NGGCUCGAAGUGCACCAGG | 644 | CCUGGUGCACUUCGAGCCN | 569-587 | 567 |
| 171 | NGGCUCGAAGUGCACCAGN | 645 | NCUGGUGCACUUCGAGCCN | 569-587 | 567 |
| 172 | CUGCUAUUUGAUGAGGACG | 646 | CGUCCUCAUCAAAUAGCAG | 603-621 | 601 |
| 173 | UUGCUAUUUGAUGAGGACG | 647 | CGUCCUCAUCAAAUAGCAA | 603-621 | 601 |
| 174 | AUGCUAUUUGAUGAGGACG | 648 | CGUCCUCAUCAAAUAGCAU | 603-621 | 601 |
| 175 | NUGCUAUUUGAUGAGGACG | 649 | CGUCCUCAUCAAAUAGCAN | 603-621 | 601 |
| 176 | NUGCUAUUUGAUGAGGACN | 650 | NGUCCUCAUCAAAUAGCAN | 603-621 | 601 |
| 177 | GAACAAGUCUGCUAUUUGA | 651 | UCAAAUAGCAGACUUGUUC | 611-629 | 609 |
| 178 | UAACAAGUCUGCUAUUUGA | 652 | UCAAAUAGCAGACUUGUUA | 611-629 | 609 |
| 179 | AAACAAGUCUGCUAUUUGA | 653 | UCAAAUAGCAGACUUGUUU | 611-629 | 609 |
| 180 | NAACAAGUCUGCUAUUUGA | 654 | UCAAAUAGCAGACUUGUUN | 611-629 | 609 |
| 181 | NAACAAGUCUGCUAUUUGN | 655 | NCAAAUAGCAGACUUGUUN | 611-629 | 609 |
| 182 | GGAACAAGUCUGCUAUUUG | 656 | CAAAUAGCAGACUUGUUCC | 612-630 | 610 |
| 183 | UGAACAAGUCUGCUAUUUG | 657 | CAAAUAGCAGACUUGUUCA | 612-630 | 610 |
| 184 | AGAACAAGUCUGCUAUUUG | 658 | CAAAUAGCAGACUUGUUCU | 612-630 | 610 |
| 185 | NGAACAAGUCUGCUAUUUG | 659 | CAAAUAGCAGACUUGUUCN | 612-630 | 610 |
| 186 | NGAACAAGUCUGCUAUUUN | 660 | NAAAUAGCAGACUUGUUCN | 612-630 | 610 |
| 187 | CGGAACAAGUCUGCUAUUU | 661 | AAAUAGCAGACUUGUUCCG | 613-631 | 611 |
| 188 | UGGAACAAGUCUGCUAUUU | 662 | AAAUAGCAGACUUGUUCCA | 613-631 | 611 |
| 189 | AGGAACAAGUCUGCUAUUU | 663 | AAAUAGCAGACUUGUUCCU | 613-631 | 611 |
| 190 | NGGAACAAGUCUGCUAUUU | 664 | AAAUAGCAGACUUGUUCCN | 613-631 | 611 |
| 191 | NGGAACAAGUCUGCUAUUN | 665 | NAAUAGCAGACUUGUUCCN | 613-631 | 611 |
| 192 | UGAGUAAGCAAUCUGGUCC | 666 | GGACCAGAUUGCUUACUCA | 638-656 | 636 |
| 193 | AGAGUAAGCAAUCUGGUCC | 667 | GGACCAGAUUGCUUACUCU | 638-656 | 636 |
| 194 | NGAGUAAGCAAUCUGGUCC | 668 | GGACCAGAUUGCUUACUCN | 638-656 | 636 |
| 195 | NGAGUAAGCAAUCUGGUCN | 669 | NGACCAGAUUGCUUACUCN | 638-656 | 636 |
| 196 | UGUCUGAGUAAGCAAUCUG | 670 | CAGAUUGCUUACUCAGACA | 642-660 | 640 |
| 197 | AGUCUGAGUAAGCAAUCUG | 671 | CAGAUUGCUUACUCAGACU | 642-660 | 640 |
| 198 | NGUCUGAGUAAGCAAUCUG | 672 | CAGAUUGCUUACUCAGACN | 642-660 | 640 |
| 199 | NGUCUGAGUAAGCAAUCUN | 673 | NAGAUUGCUUACUCAGACN | 642-660 | 640 |
| 200 | CUGGUGUCUGAGUAAGCAA | 674 | UUGCUUACUCAGACACCAG | 646-664 | 644 |
| 201 | UUGGUGUCUGAGUAAGCAA | 675 | UUGCUUACUCAGACACCAA | 646-664 | 644 |
| 202 | AUGGUGUCUGAGUAAGCAA | 676 | UUGCUUACUCAGACACCAU | 646-664 | 644 |
| 203 | NUGGUGUCUGAGUAAGCAA | 677 | UUGCUUACUCAGACACCAN | 646-664 | 644 |
| 204 | NUGGUGUCUGAGUAAGCAN | 678 | NUGCUUACUCAGACACCAN | 646-664 | 644 |
| 205 | UUAACUUUCUUCUCUAGCC | 679 | GGCUAGAGAAGAAAGUUAA | 712-730 | 710 |
| 206 | AUAACUUUCUUCUCUAGCC | 680 | GGCUAGAGAAGAAAGUUAU | 712-730 | 710 |
| 207 | NUAACUUUCUUCUCUAGCC | 681 | GGCUAGAGAAGAAAGUUAN | 712-730 | 710 |
| 208 | NUAACUUUCUUCUCUAGCN | 682 | NGCUAGAGAAGAAAGUUAN | 712-730 | 710 |
| 209 | UGCUUUAACUUUCUUCUCU | 683 | AGAGAAGAAAGUUAAAGCA | 716-734 | 714 |
| 210 | AGCUUUAACUUUCUUCUCU | 684 | AGAGAAGAAAGUUAAAGCU | 716-734 | 714 |
| 211 | NGCUUUAACUUUCUUCUCU | 685 | AGAGAAGAAAGUUAAAGCN | 716-734 | 714 |
| 212 | NGCUUUAACUUUCUUCUCN | 686 | NGAGAAGAAAGUUAAAGCN | 716-734 | 714 |
| 213 | GUUGCUUUAACUUUCUUCU | 687 | AGAAGAAAGUUAAAGCAAC | 718-736 | 716 |
| 214 | UUUGCUUUAACUUUCUUCU | 688 | AGAAGAAAGUUAAAGCAAA | 718-736 | 716 |
| 215 | AUUGCUUUAACUUUCUUCU | 689 | AGAAGAAAGUUAAAGCAAU | 718-736 | 716 |
| 216 | NUUGCUUUAACUUUCUUCU | 690 | AGAAGAAAGUUAAAGCAAN | 718-736 | 716 |
| 217 | NUUGCUUUAACUUUCUUCN | 691 | NGAAGAAAGUUAAAGCAAN | 718-736 | 716 |
| 218 | UGAAAUUACAAUAUUGGGC | 692 | GCCCAAUAUUGUAAUUUCA | 746-764 | 744 |
| 219 | AGAAAUUACAAUAUUGGGC | 693 | GCCCAAUAUUGUAAUUUCU | 746-764 | 744 |
| 220 | NGAAAUUACAAUAUUGGGC | 694 | GCCCAAUAUUGUAAUUUCN | 746-764 | 744 |
| 221 | NGAAAUUACAAUAUUGGGN | 695 | NCCCAAUAUUGUAAUUUCN | 746-764 | 744 |
| 222 | GCAUAGACAUCGCAUCCUG | 696 | CAGGAUGCGAUGUCUAUGC | 763-781 | 761 |
| 223 | UCAUAGACAUCGCAUCCUG | 697 | CAGGAUGCGAUGUCUAUGA | 763-781 | 761 |
| 224 | ACAUAGACAUCGCAUCCUG | 698 | CAGGAUGCGAUGUCUAUGU | 763-781 | 761 |
| 225 | NCAUAGACAUCGCAUCCUG | 699 | CAGGAUGCGAUGUCUAUGN | 763-781 | 761 |
| 226 | NCAUAGACAUCGCAUCCUN | 700 | NAGGAUGCGAUGUCUAUGN | 763-781 | 761 |
| 227 | UGGUUAAAAUGCAUCUGGA | 701 | UCCAGAUGCAUUUUAACCA | 843-861 | 841 |
| 228 | AGGUUAAAAUGCAUCUGGA | 702 | UCCAGAUGCAUUUUAACCU | 843-861 | 841 |
| 229 | NGGUUAAAAUGCAUCUGGA | 703 | UCCAGAUGCAUUUUAACCN | 843-861 | 841 |
| 230 | NGGUUAAAAUGCAUCUGGN | 704 | NCCAGAUGCAUUUUAACCN | 843-861 | 841 |
| 231 | AACUCUUCAGUGUUUCCAG | 705 | CUGGAAACACUGAAGAGUU | 903-921 | 901 |
| 232 | UACUCUUCAGUGUUUCCAG | 706 | CUGGAAACACUGAAGAGUA | 903-921 | 901 |
| 233 | NACUCUUCAGUGUUUCCAG | 707 | CUGGAAACACUGAAGAGUN | 903-921 | 901 |
| 234 | NACUCUUCAGUGUUUCCAN | 708 | NUGGAAACACUGAAGAGUN | 903-921 | 901 |
| 235 | AACUUUCGUUCUGAAGGGU | 709 | ACCCUUCAGAACGAAAGUU | 934-952 | 932 |
| 236 | UACUUUCGUUCUGAAGGGU | 710 | ACCCUUCAGAACGAAAGUA | 934-952 | 932 |
| 237 | NACUUUCGUUCUGAAGGGU | 711 | ACCCUUCAGAACGAAAGUN | 934-952 | 932 |
| 238 | NACUUUCGUUCUGAAGGGN | 712 | NCCCUUCAGAACGAAAGUN | 934-952 | 932 |
| 239 | UAACUUUCGUUCUGAAGGG | 713 | CCCUUCAGAACGAAAGUUA | 935-953 | 933 |
| 240 | AAACUUUCGUUCUGAAGGG | 714 | CCCUUCAGAACGAAAGUUU | 935-953 | 933 |
| 241 | NAACUUUCGUUCUGAAGGG | 715 | CCCUUCAGAACGAAAGUUN | 935-953 | 933 |
| 242 | NAACUUUCGUUCUGAAGGN | 716 | NCCUUCAGAACGAAAGUUN | 935-953 | 933 |
| 243 | CCAUAUAACUUUCGUUCUG | 717 | CAGAACGAAAGUUAUAUGG | 940-958 | 938 |
| 244 | UCAUAUAACUUUCGUUCUG | 718 | CAGAACGAAAGUUAUAUGA | 940-958 | 938 |
| 245 | ACAUAUAACUUUCGUUCUG | 719 | CAGAACGAAAGUUAUAUGU | 940-958 | 938 |
| 246 | NCAUAUAACUUUCGUUCUG | 720 | CAGAACGAAAGUUAUAUGN | 940-958 | 938 |
| 247 | NCAUAUAACUUUCGUUCUN | 721 | NAGAACGAAAGUUAUAUGN | 940-958 | 938 |
| 248 | UUCCAUAUAACUUUCGUUC | 722 | GAACGAAAGUUAUAUGGAA | 942-960 | 940 |
| 249 | AUCCAUAUAACUUUCGUUC | 723 | GAACGAAAGUUAUAUGGAU | 942-960 | 940 |
| 250 | NUCCAUAUAACUUUCGUUC | 724 | GAACGAAAGUUAUAUGGAN | 942-960 | 940 |
| 251 | NUCCAUAUAACUUUCGUUN | 725 | NAACGAAAGUUAUAUGGAN | 942-960 | 940 |
| 252 | UGAUUUUCCAUAUAACUUU | 726 | AAAGUUAUAUGGAAAAUCA | 947-965 | 945 |
| 253 | AGAUUUUCCAUAUAACUUU | 727 | AAAGUUAUAUGGAAAAUCU | 947-965 | 945 |
| 254 | NGAUUUUCCAUAUAACUUU | 728 | AAAGUUAUAUGGAAAAUCN | 947-965 | 945 |
| 255 | NGAUUUUCCAUAUAACUUN | 729 | NAAGUUAUAUGGAAAAUCN | 947-965 | 945 |
| 256 | AAAGAGUGGUGAUUUUCCA | 730 | UGGAAAAUCACCACUCUUU | 956-974 | 954 |
| 257 | UAAGAGUGGUGAUUUUCCA | 731 | UGGAAAAUCACCACUCUUA | 956-974 | 954 |
| 258 | NAAGAGUGGUGAUUUUCCA | 732 | UGGAAAAUCACCACUCUUN | 956-974 | 954 |
| 259 | NAAGAGUGGUGAUUUUCCN | 733 | NGGAAAAUCACCACUCUUN | 956-974 | 954 |
| 260 | CCAAAGAGUGGUGAUUUUC | 734 | GAAAAUCACCACUCUUUGG | 958-976 | 956 |
| 261 | UCAAAGAGUGGUGAUUUUC | 735 | GAAAAUCACCACUCUUUGA | 958-976 | 956 |
| 262 | ACAAAGAGUGGUGAUUUUC | 736 | GAAAAUCACCACUCUUUGU | 958-976 | 956 |
| 263 | NCAAAGAGUGGUGAUUUUC | 737 | GAAAAUCACCACUCUUUGN | 958-976 | 956 |
| 264 | NCAAAGAGUGGUGAUUUUN | 738 | NAAAAUCACCACUCUUUGN | 958-976 | 956 |
| 265 | CACAGGGUCUCCCACUUUG | 739 | CAAAGUGGGAGACCCUGUG | 1010-1028 | 1008 |
| 266 | UACAGGGUCUCCCACUUUG | 740 | CAAAGUGGGAGACCCUGUA | 1010-1028 | 1008 |
| 267 | AACAGGGUCUCCCACUUUG | 741 | CAAAGUGGGAGACCCUGUU | 1010-1028 | 1008 |
| 268 | NACAGGGUCUCCCACUUUG | 742 | CAAAGUGGGAGACCCUGUN | 1010-1028 | 1008 |
| 269 | NACAGGGUCUCCCACUUUN | 743 | NAAAGUGGGAGACCCUGUN | 1010-1028 | 1008 |
| 270 | ACACAGGGUCUCCCACUUU | 744 | AAAGUGGGAGACCCUGUGU | 1011-1029 | 1009 |
| 271 | UCACAGGGUCUCCCACUUU | 745 | AAAGUGGGAGACCCUGUGA | 1011-1029 | 1009 |
| 272 | NCACAGGGUCUCCCACUUU | 746 | AAAGUGGGAGACCCUGUGN | 1011-1029 | 1009 |
| 273 | NCACAGGGUCUCCCACUUN | 747 | NAAGUGGGAGACCCUGUGN | 1011-1029 | 1009 |
| 274 | GGUACACAGGGUCUCCCAC | 748 | GUGGGAGACCCUGUGUACC | 1014-1032 | 1012 |
| 275 | UGUACACAGGGUCUCCCAC | 749 | GUGGGAGACCCUGUGUACA | 1014-1032 | 1012 |
| 276 | AGUACACAGGGUCUCCCAC | 750 | GUGGGAGACCCUGUGUACU | 1014-1032 | 1012 |
| 277 | NGUACACAGGGUCUCCCAC | 751 | GUGGGAGACCCUGUGUACN | 1014-1032 | 1012 |
| 278 | NGUACACAGGGUCUCCCAN | 752 | NUGGGAGACCCUGUGUACN | 1014-1032 | 1012 |
| 279 | AGGUACACAGGGUCUCCCA | 753 | UGGGAGACCCUGUGUACCU | 1015-1033 | 1013 |
| 280 | UGGUACACAGGGUCUCCCA | 754 | UGGGAGACCCUGUGUACCA | 1015-1033 | 1013 |
| 281 | NGGUACACAGGGUCUCCCA | 755 | UGGGAGACCCUGUGUACCN | 1015-1033 | 1013 |
| 282 | NGGUACACAGGGUCUCCCN | 756 | NGGGAGACCCUGUGUACCN | 1015-1033 | 1013 |
| 283 | CAGGUACACAGGGUCUCCC | 757 | GGGAGACCCUGUGUACCUG | 1016-1034 | 1014 |
| 284 | UAGGUACACAGGGUCUCCC | 758 | GGGAGACCCUGUGUACCUA | 1016-1034 | 1014 |
| 285 | AAGGUACACAGGGUCUCCC | 759 | GGGAGACCCUGUGUACCUU | 1016-1034 | 1014 |
| 286 | NAGGUACACAGGGUCUCCC | 760 | GGGAGACCCUGUGUACCUN | 1016-1034 | 1014 |
| 287 | NAGGUACACAGGGUCUCCN | 761 | NGGAGACCCUGUGUACCUN | 1016-1034 | 1014 |
| 288 | GCAUCUAAUAUUCCAGGAC | 762 | GUCCUGGAAUAUUAGAUGC | 1059-1077 | 1057 |
| 289 | UCAUCUAAUAUUCCAGGAC | 763 | GUCCUGGAAUAUUAGAUGA | 1059-1077 | 1057 |
| 290 | ACAUCUAAUAUUCCAGGAC | 764 | GUCCUGGAAUAUUAGAUGU | 1059-1077 | 1057 |
| 291 | NCAUCUAAUAUUCCAGGAC | 765 | GUCCUGGAAUAUUAGAUGN | 1059-1077 | 1057 |
| 292 | NCAUCUAAUAUUCCAGGAN | 766 | NUCCUGGAAUAUUAGAUGN | 1059-1077 | 1057 |
| 293 | GGCAUCUAAUAUUCCAGGA | 767 | UCCUGGAAUAUUAGAUGCC | 1060-1078 | 1058 |
| 294 | AGCAUCUAAUAUUCCAGGA | 768 | UCCUGGAAUAUUAGAUGCU | 1060-1078 | 1058 |
| 295 | UGCAUCUAAUAUUCCAGGA | 769 | UCCUGGAAUAUUAGAUGCA | 1060-1078 | 1058 |
| 296 | NGCAUCUAAUAUUCCAGGA | 770 | UCCUGGAAUAUUAGAUGCN | 1060-1078 | 1058 |
| 297 | NGCAUCUAAUAUUCCAGGN | 771 | NCCUGGAAUAUUAGAUGEN | 1060-1078 | 1058 |
| 298 | AGGCAUCUAAUAUUCCAGG | 772 | CCUGGAAUAUUAGAUGCCU | 1061-1079 | 1059 |
| 299 | UGGCAUCUAAUAUUCCAGG | 773 | CCUGGAAUAUUAGAUGCCA | 1061-1079 | 1059 |
| 300 | NGGCAUCUAAUAUUCCAGG | 774 | CCUGGAAUAUUAGAUGCCN | 1061-1079 | 1059 |
| 301 | NGGCAUCUAAUAUUCCAGN | 775 | NCUGGAAUAUUAGAUGCCN | 1061-1079 | 1059 |
| 302 | AAGGCAUCUAAUAUUCCAG | 776 | CUGGAAUAUUAGAUGCCUU | 1062-1080 | 1060 |
| 303 | UAGGCAUCUAAUAUUCCAG | 777 | CUGGAAUAUUAGAUGCCUA | 1062-1080 | 1060 |
| 304 | NAGGCAUCUAAUAUUCCAG | 778 | CUGGAAUAUUAGAUGCCUN | 1062-1080 | 1060 |
| 305 | NAGGCAUCUAAUAUUCCAN | 779 | NUGGAAUAUUAGAUGCCUN | 1062-1080 | 1060 |
| 306 | AGUGUUGUCAUUUUUGAGA | 780 | UCUCAAAAAUGACAACACU | 1091-1109 | 1089 |
| 307 | UGUGUUGUCAUUUUUGAGA | 781 | UCUCAAAAAUGACAACACA | 1091-1109 | 1089 |
| 308 | NGUGUUGUCAUUUUUGAGA | 782 | UCUCAAAAAUGACAACACN | 1091-1109 | 1089 |
| 309 | NGUGUUGUCAUUUUUGAGN | 783 | NCUCAAAAAUGACAACACN | 1091-1109 | 1089 |
| 310 | CAUGCUUCAAGUGUUGUCA | 784 | UGACAACACUUGAAGCAUG | 1100-1118 | 1098 |
| 311 | UAUGCUUCAAGUGUUGUCA | 785 | UGACAACACUUGAAGCAUA | 1100-1118 | 1098 |
| 312 | AAUGCUUCAAGUGUUGUCA | 786 | UGACAACACUUGAAGCAUU | 1100-1118 | 1098 |
| 313 | NAUGCUUCAAGUGUUGUCA | 787 | UGACAACACUUGAAGCAUN | 1100-1118 | 1098 |
| 314 | NAUGCUUCAAGUGUUGUCN | 788 | NGACAACACUUGAAGCAUN | 1100-1118 | 1098 |
| 315 | ACACCAUGCUUCAAGUGUU | 789 | AACACUUGAAGCAUGGUGU | 1104-1122 | 1102 |
| 316 | UCACCAUGCUUCAAGUGUU | 790 | AACACUUGAAGCAUGGUGA | 1104-1122 | 1102 |
| 317 | NCACCAUGCUUCAAGUGUU | 791 | AACACUUGAAGCAUGGUGN | 1104-1122 | 1102 |
| 318 | NCACCAUGCUUCAAGUGUN | 792 | AACACUUGAAGCAUGGUGN | 1104-1122 | 1102 |
| 319 | UUCUGAAACACCAUGCUUC | 793 | GAAGCAUGGUGUUUCAGAA | 1111-1129 | 1109 |
| 320 | AUCUGAAACACCAUGCUUC | 794 | GAAGCAUGGUGUUUCAGAU | 1111-1129 | 1109 |
| 321 | NUCUGAAACACCAUGCUUC | 795 | GAAGCAUGGUGUUUCAGAN | 1111-1129 | 1109 |
| 322 | NUCUGAAACACCAUGCUUN | 796 | NAAGCAUGGUGUUUCAGAN | 1111-1129 | 1109 |
| 323 | AGUUCUGAAACACCAUGCU | 797 | AGCAUGGUGUUUCAGAACU | 1113-1131 | 1111 |
| 324 | UGUUCUGAAACACCAUGCU | 798 | AGCAUGGUGUUUCAGAACA | 1113-1131 | 1111 |
| 325 | NGUUCUGAAACACCAUGCU | 799 | AGCAUGGUGUUUCAGAACN | 1113-1131 | 1111 |
| 326 | NGUUCUGAAACACCAUGEN | 800 | NGCAUGGUGUUUCAGAACN | 1113-1131 | 1111 |
| 327 | AUGUAGAGGUCUCAGUUCU | 801 | AGAACUGAGACCUCUACAU | 1126-1144 | 1124 |
| 328 | UUGUAGAGGUCUCAGUUCU | 802 | AGAACUGAGACCUCUACAA | 1126-1144 | 1124 |
| 329 | NUGUAGAGGUCUCAGUUCU | 803 | AGAACUGAGACCUCUACAN | 1126-1144 | 1124 |
| 330 | NUGUAGAGGUCUCAGUUCN | 804 | NGAACUGAGACCUCUACAN | 1126-1144 | 1124 |
| 331 | AAAGAAAAUGUAGAGGUCU | 805 | AGACCUCUACAUUUUCUUU | 1133-1151 | 1131 |
| 332 | UAAGAAAAUGUAGAGGUCU | 806 | AGACCUCUACAUUUUCUUA | 1133-1151 | 1131 |
| 333 | NAAGAAAAUGUAGAGGUCU | 807 | AGACCUCUACAUUUUCUUN | 1133-1151 | 1131 |
| 334 | NAAGAAAAUGUAGAGGUCN | 808 | NGACCUCUACAUUUUCUUN | 1133-1151 | 1131 |
| 335 | ACGAAAAAUGUGAAAAUCA | 809 | UGAUUUUCACAUUUUUCGU | 1159-1177 | 1157 |
| 336 | UCGAAAAAUGUGAAAAUCA | 810 | UGAUUUUCACAUUUUUCGA | 1159-1177 | 1157 |
| 337 | NCGAAAAAUGUGAAAAUCA | 811 | UGAUUUUCACAUUUUUCGN | 1159-1177 | 1157 |
| 338 | NCGAAAAAUGUGAAAAUCN | 812 | NGAUUUUCACAUUUUUCGN | 1159-1177 | 1157 |
| 339 | CAUUGAAGCAUUGAGACAC | 813 | GUGUCUCAAUGCUUCAAUG | 1192-1210 | 1190 |
| 340 | UAUUGAAGCAUUGAGACAC | 814 | GUGUCUCAAUGCUUCAAUA | 1192-1210 | 1190 |
| 341 | AAUUGAAGCAUUGAGACAC | 815 | GUGUCUCAAUGCUUCAAUU | 1192-1210 | 1190 |
| 342 | NAUUGAAGCAUUGAGACAC | 816 | GUGUCUCAAUGCUUCAAUN | 1192-1210 | 1190 |
| 343 | NAUUGAAGCAUUGAGACAN | 817 | NUGUCUCAAUGCUUCAAUN | 1192-1210 | 1190 |
| 344 | GGACAUUGAAGCAUUGAGA | 818 | UCUCAAUGCUUCAAUGUCC | 1195-1213 | 1193 |
| 345 | UGACAUUGAAGCAUUGAGA | 819 | UCUCAAUGCUUCAAUGUCA | 1195-1213 | 1193 |
| 346 | AGACAUUGAAGCAUUGAGA | 820 | UCUCAAUGCUUCAAUGUCU | 1195-1213 | 1193 |
| 347 | NGACAUUGAAGCAUUGAGA | 821 | UCUCAAUGCUUCAAUGUCN | 1195-1213 | 1193 |
| 348 | NGACAUUGAAGCAUUGAGN | 822 | NCUCAAUGCUUCAAUGUCN | 1195-1213 | 1193 |
| 349 | UUACUGAAGUCCUACUAAG | 823 | CUUAGUAGGACUUCAGUAA | 1248-1266 | 1246 |
| 350 | AUACUGAAGUCCUACUAAG | 824 | CUUAGUAGGACUUCAGUAU | 1248-1266 | 1246 |
| 351 | NUACUGAAGUCCUACUAAG | 825 | CUUAGUAGGACUUCAGUAN | 1248-1266 | 1246 |
| 352 | NUACUGAAGUCCUACUAAN | 826 | NUUAGUAGGACUUCAGUAN | 1248-1266 | 1246 |
| 353 | CAGAAUCCUGUCUUGUCAU | 827 | AUGACAAGACAGGAUUCUG | 1276-1294 | 1274 |
| 354 | UAGAAUCCUGUCUUGUCAU | 828 | AUGACAAGACAGGAUUCUA | 1276-1294 | 1274 |
| 355 | AAGAAUCCUGUCUUGUCAU | 829 | AUGACAAGACAGGAUUCUU | 1276-1294 | 1274 |
| 356 | NAGAAUCCUGUCUUGUCAU | 830 | AUGACAAGACAGGAUUCUN | 1276-1294 | 1274 |
| 357 | NAGAAUCCUGUCUUGUCAN | 831 | NUGACAAGACAGGAUUCUN | 1276-1294 | 1274 |
| 358 | UCAGAAUCCUGUCUUGUCA | 832 | UGACAAGACAGGAUUCUGA | 1277-1295 | 1275 |
| 359 | ACAGAAUCCUGUCUUGUCA | 833 | UGACAAGACAGGAUUCUGU | 1277-1295 | 1275 |
| 360 | NCAGAAUCCUGUCUUGUCA | 834 | UGACAAGACAGGAUUCUGN | 1277-1295 | 1275 |
| 361 | NCAGAAUCCUGUCUUGUCN | 835 | NGACAAGACAGGAUUCUGN | 1277-1295 | 1275 |
| 362 | UUCAGAAUCCUGUCUUGUC | 836 | GACAAGACAGGAUUCUGAA | 1278-1296 | 1276 |
| 363 | AUCAGAAUCCUGUCUUGUC | 837 | GACAAGACAGGAUUCUGAU | 1278-1296 | 1276 |
| 364 | NUCAGAAUCCUGUCUUGUC | 838 | GACAAGACAGGAUUCUGAN | 1278-1296 | 1276 |
| 365 | NUCAGAAUCCUGUCUUGUN | 839 | NACAAGACAGGAUUCUGAN | 1278-1296 | 1276 |
| 366 | GGAGUUUUCAGAAUCCUGU | 840 | ACAGGAUUCUGAAAACUCC | 1284-1302 | 1282 |
| 367 | UGAGUUUUCAGAAUCCUGU | 841 | ACAGGAUUCUGAAAACUCA | 1284-1302 | 1282 |
| 368 | AGAGUUUUCAGAAUCCUGU | 842 | ACAGGAUUCUGAAAACUCU | 1284-1302 | 1282 |
| 369 | NGAGUUUUCAGAAUCCUGU | 843 | ACAGGAUUCUGAAAACUCN | 1284-1302 | 1282 |
| 370 | NGAGUUUUCAGAAUCCUGN | 844 | NCAGGAUUCUGAAAACUCN | 1284-1302 | 1282 |
| 371 | UUCCAUAAUCAGUUAAACG | 845 | CGUUUAACUGAUUAUGGAA | 1304-1322 | 1302 |
| 372 | AUCCAUAAUCAGUUAAACG | 846 | CGUUUAACUGAUUAUGGAU | 1304-1322 | 1302 |
| 373 | NUCCAUAAUCAGUUAAACG | 847 | CGUUUAACUGAUUAUGGAN | 1304-1322 | 1302 |
| 374 | NUCCAUAAUCAGUUAAACN | 848 | NGUUUAACUGAUUAUGGAN | 1304-1322 | 1302 |
| 375 | AAGAACUAUUCCAUAAUCA | 849 | UGAUUAUGGAAUAGUUCUU | 1312-1330 | 1310 |
| 376 | UAGAACUAUUCCAUAAUCA | 850 | UGAUUAUGGAAUAGUUCUA | 1312-1330 | 1310 |
| 377 | NAGAACUAUUCCAUAAUCA | 851 | UGAUUAUGGAAUAGUUCUN | 1312-1330 | 1310 |
| 378 | NAGAACUAUUCCAUAAUCN | 852 | NGAUUAUGGAAUAGUUCUN | 1312-1330 | 1310 |
| 379 | AGAAAGAACUAUUCCAUAA | 853 | UUAUGGAAUAGUUCUUUCU | 1315-1333 | 1313 |
| 380 | UGAAAGAACUAUUCCAUAA | 854 | UUAUGGAAUAGUUCUUUCA | 1315-1333 | 1313 |
| 381 | NGAAAGAACUAUUCCAUAA | 855 | UUAUGGAAUAGUUCUUUCN | 1315-1333 | 1313 |
| 382 | NGAAAGAACUAUUCCAUAN | 856 | NUAUGGAAUAGUUCUUUCN | 1315-1333 | 1313 |
| 383 | GAGAAAGAACUAUUCCAUA | 857 | UAUGGAAUAGUUCUUUCUC | 1316-1334 | 1314 |
| 384 | UAGAAAGAACUAUUCCAUA | 858 | UAUGGAAUAGUUCUUUCUA | 1316-1334 | 1314 |
| 385 | AAGAAAGAACUAUUCCAUA | 859 | UAUGGAAUAGUUCUUUCUU | 1316-1334 | 1314 |
| 386 | NAGAAAGAACUAUUCCAUA | 860 | UAUGGAAUAGUUCUUUCUN | 1316-1334 | 1314 |
| 387 | NAGAAAGAACUAUUCCAUN | 861 | NAUGGAAUAGUUCUUUCUN | 1316-1334 | 1314 |
| 388 | AGAUAAACGGAGAAGCAGG | 862 | CCUGCUUCUCCGUUUAUCU | 1334-1352 | 1332 |
| 389 | UGAUAAACGGAGAAGCAGG | 863 | CCUGCUUCUCCGUUUAUCA | 1334-1352 | 1332 |
| 390 | NGAUAAACGGAGAAGCAGG | 864 | CCUGCUUCUCCGUUUAUCN | 1334-1352 | 1332 |
| 391 | NGAUAAACGGAGAAGCAGN | 865 | NCUGCUUCUCCGUUUAUCN | 1334-1352 | 1332 |
| 392 | ACAUUCUAGGAUAUUCUUC | 866 | GAAGAAUAUCCUAGAAUGU | 1434-1452 | 1432 |
| 393 | UCAUUCUAGGAUAUUCUUC | 867 | GAAGAAUAUCCUAGAAUGA | 1434-1452 | 1432 |
| 394 | NCAUUCUAGGAUAUUCUUC | 868 | GAAGAAUAUCCUAGAAUGN | 1434-1452 | 1432 |
| 395 | NCAUUCUAGGAUAUUCUUN | 869 | NAAGAAUAUCCUAGAAUGN | 1434-1452 | 1432 |
| 396 | AGAUCCAGAUCUAUGGAAA | 870 | UUUCCAUAGAUCUGGAUCU | 1607-1625 | 1605 |
| 397 | UGAUCCAGAUCUAUGGAAA | 871 | UUUCCAUAGAUCUGGAUCA | 1607-1625 | 1605 |
| 398 | NGAUCCAGAUCUAUGGAAA | 872 | UUUCCAUAGAUCUGGAUCN | 1607-1625 | 1605 |
| 399 | NGAUCCAGAUCUAUGGAAN | 873 | NUUCCAUAGAUCUGGAUCN | 1607-1625 | 1605 |
| 400 | CCAAUGCUGUCUGAGAAGC | 874 | GCUUCUCAGACAGCAUUGG | 1635-1653 | 1633 |
| 401 | UCAAUGCUGUCUGAGAAGC | 875 | GCUUCUCAGACAGCAUUGA | 1635-1653 | 1633 |
| 402 | ACAAUGCUGUCUGAGAAGC | 876 | GCUUCUCAGACAGCAUUGU | 1635-1653 | 1633 |
| 403 | NCAAUGCUGUCUGAGAAGC | 877 | GCUUCUCAGACAGCAUUGN | 1635-1653 | 1633 |
| 404 | NCAAUGCUGUCUGAGAAGN | 878 | NCUUCUCAGACAGCAUUGN | 1635-1653 | 1633 |
| 405 | AUCCAAUGCUGUCUGAGAA | 879 | UUCUCAGACAGCAUUGGAU | 1637-1655 | 1635 |
| 406 | UUCCAAUGCUGUCUGAGAA | 880 | UUCUCAGACAGCAUUGGAA | 1637-1655 | 1635 |
| 407 | NUCCAAUGCUGUCUGAGAA | 881 | UUCUCAGACAGCAUUGGAN | 1637-1655 | 1635 |
| 408 | NUCCAAUGCUGUCUGAGAN | 882 | NUCUCAGACAGCAUUGGAN | 1637-1655 | 1635 |
| 409 | UUAGGAAAUCCAAUGCUGU | 883 | ACAGCAUUGGAUUUCCUAA | 1644-1662 | 1642 |
| 410 | AUAGGAAAUCCAAUGCUGU | 884 | ACAGCAUUGGAUUUCCUAU | 1644-1662 | 1642 |
| 411 | NUAGGAAAUCCAAUGCUGU | 885 | ACAGCAUUGGAUUUCCUAN | 1644-1662 | 1642 |
| 412 | NUAGGAAAUCCAAUGCUGN | 886 | NCAGCAUUGGAUUUCCUAN | 1644-1662 | 1642 |
| 413 | ACCUUUAGGAAAUCCAAUG | 887 | CAUUGGAUUUCCUAAAGGU | 1648-1666 | 1646 |
| 414 | UCCUUUAGGAAAUCCAAUG | 888 | CAUUGGAUUUCCUAAAGGA | 1648-1666 | 1646 |
| 415 | NCCUUUAGGAAAUCCAAUG | 889 | CAUUGGAUUUCCUAAAGGN | 1648-1666 | 1646 |
| 416 | NCCUUUAGGAAAUCCAAUN | 890 | NAUUGGAUUUCCUAAAGGN | 1648-1666 | 1646 |
| 417 | GCACCUUUAGGAAAUCCAA | 891 | UUGGAUUUCCUAAAGGUGC | 1650-1668 | 1648 |
| 418 | UCACCUUUAGGAAAUCCAA | 892 | UUGGAUUUCCUAAAGGUGA | 1650-1668 | 1648 |
| 419 | ACACCUUUAGGAAAUCCAA | 893 | UUGGAUUUCCUAAAGGUGU | 1650-1668 | 1648 |
| 420 | NCACCUUUAGGAAAUCCAA | 894 | UUGGAUUUCCUAAAGGUGN | 1650-1668 | 1648 |
| 421 | NCACCUUUAGGAAAUCCAN | 895 | NUGGAUUUCCUAAAGGUGN | 1650-1668 | 1648 |
| 422 | CCCUUUAAAGGUUUUCAGU | 896 | ACUGAAAACCUUUAAAGGG | 1819-1837 | 1817 |
| 423 | UCCUUUAAAGGUUUUCAGU | 897 | ACUGAAAACCUUUAAAGGA | 1819-1837 | 1817 |
| 424 | ACCUUUAAAGGUUUUCAGU | 898 | ACUGAAAACCUUUAAAGGU | 1819-1837 | 1817 |
| 425 | NCCUUUAAAGGUUUUCAGU | 899 | ACUGAAAACCUUUAAAGGN | 1819-1837 | 1817 |
| 426 | NCCUUUAAAGGUUUUCAGN | 900 | NCUGAAAACCUUUAAAGGN | 1819-1837 | 1817 |
| 427 | CAACUGACAUAUGCUUUCC | 901 | GGAAAGCAUAUGUCAGUUG | 1844-1862 | 1842 |
| 428 | UAACUGACAUAUGCUUUCC | 902 | GGAAAGCAUAUGUCAGUUA | 1844-1862 | 1842 |
| 429 | AAACUGACAUAUGCUUUCC | 903 | GGAAAGCAUAUGUCAGUUU | 1844-1862 | 1842 |
| 430 | NAACUGACAUAUGCUUUCC | 904 | GGAAAGCAUAUGUCAGUUN | 1844-1862 | 1842 |
| 431 | NAACUGACAUAUGCUUUCN | 905 | NGAAAGCAUAUGUCAGUUN | 1844-1862 | 1842 |
| 432 | GGGUUUUAAACAACUGACA | 906 | UGUCAGUUGUUUAAAACCC | 1854-1872 | 1852 |
| 433 | UGGUUUUAAACAACUGACA | 907 | UGUCAGUUGUUUAAAACCA | 1854-1872 | 1852 |
| 434 | AGGUUUUAAACAACUGACA | 908 | UGUCAGUUGUUUAAAACCU | 1854-1872 | 1852 |
| 435 | NGGUUUUAAACAACUGACA | 909 | UGUCAGUUGUUUAAAACCN | 1854-1872 | 1852 |
| 436 | NGGUUUUAAACAACUGACN | 910 | NGUCAGUUGUUUAAAACCN | 1854-1872 | 1852 |
| 437 | UUCAUCAGAUCUUAGAGUU | 911 | AACUCUAAGAUCUGAUGAA | 1899-1917 | 1897 |
| 438 | AUCAUCAGAUCUUAGAGUU | 912 | AACUCUAAGAUCUGAUGAU | 1899-1917 | 1897 |
| 439 | NUCAUCAGAUCUUAGAGUU | 913 | AACUCUAAGAUCUGAUGAN | 1899-1917 | 1897 |
| 440 | NUCAUCAGAUCUUAGAGUN | 914 | NACUCUAAGAUCUGAUGAN | 1899-1917 | 1897 |
| 441 | CUUCAUCAGAUCUUAGAGU | 915 | ACUCUAAGAUCUGAUGAAG | 1900-1918 | 1898 |
| 442 | UUUCAUCAGAUCUUAGAGU | 916 | ACUCUAAGAUCUGAUGAAA | 1900-1918 | 1898 |
| 443 | AUUCAUCAGAUCUUAGAGU | 917 | ACUCUAAGAUCUGAUGAAU | 1900-1918 | 1898 |
| 444 | NUUCAUCAGAUCUUAGAGU | 918 | ACUCUAAGAUCUGAUGAAN | 1900-1918 | 1898 |
| 445 | NUUCAUCAGAUCUUAGAGN | 919 | NCUCUAAGAUCUGAUGAAN | 1900-1918 | 1898 |
| 446 | UACUUCAUCAGAUCUUAGA | 920 | UCUAAGAUCUGAUGAAGUA | 1902-1920 | 1900 |
| 447 | AACUUCAUCAGAUCUUAGA | 921 | UCUAAGAUCUGAUGAAGUU | 1902-1920 | 1900 |
| 448 | NACUUCAUCAGAUCUUAGA | 922 | UCUAAGAUCUGAUGAAGUN | 1902-1920 | 1900 |
| 449 | NACUUCAUCAGAUCUUAGN | 923 | NCUAAGAUCUGAUGAAGUN | 1902-1920 | 1900 |
| 450 | CAAAGGACAAAAUGGCAAU | 924 | AUUGCCAUUUUGUCCUUUG | 1929-1947 | 1927 |
| 451 | UAAAGGACAAAAUGGCAAU | 925 | AUUGCCAUUUUGUCCUUUA | 1929-1947 | 1927 |
| 452 | AAAAGGACAAAAUGGCAAU | 926 | AUUGCCAUUUUGUCCUUUU | 1929-1947 | 1927 |
| 453 | NAAAGGACAAAAUGGCAAU | 927 | AUUGCCAUUUUGUCCUUUN | 1929-1947 | 1927 |
| 454 | NAAAGGACAAAAUGGCAAN | 928 | NUUGCCAUUUUGUCCUUUN | 1929-1947 | 1927 |
| 455 | UAAUCAAAGGACAAAAUGG | 929 | CCAUUUUGUCCUUUGAUUA | 1933-1951 | 1931 |
| 456 | AAAUCAAAGGACAAAAUGG | 930 | CCAUUUUGUCCUUUGAUUU | 1933-1951 | 1931 |
| 457 | NAAUCAAAGGACAAAAUGG | 931 | CCAUUUUGUCCUUUGAUUN | 1933-1951 | 1931 |
| 458 | NAAUCAAAGGACAAAAUGN | 932 | NCAUUUUGUCCUUUGAUUN | 1933-1951 | 1931 |
| 459 | CAAGUUUAGUCAACUUCCC | 933 | GGGAAGUUGACUAAACUUG | 1956-1974 | 1954 |
| 460 | UAAGUUUAGUCAACUUCCC | 934 | GGGAAGUUGACUAAACUUA | 1956-1974 | 1954 |
| 461 | AAAGUUUAGUCAACUUCCC | 935 | GGGAAGUUGACUAAACUUU | 1956-1974 | 1954 |
| 462 | NAAGUUUAGUCAACUUCCC | 936 | GGGAAGUUGACUAAACUUN | 1956-1974 | 1954 |
| 463 | NAAGUUUAGUCAACUUCCN | 937 | NGGAAGUUGACUAAACUUN | 1956-1974 | 1954 |
| 464 | UCAAGUUUAGUCAACUUCC | 938 | GGAAGUUGACUAAACUUGA | 1957-1975 | 1955 |
| 465 | ACAAGUUUAGUCAACUUCC | 939 | GGAAGUUGACUAAACUUGU | 1957-1975 | 1955 |
| 466 | NCAAGUUUAGUCAACUUCC | 940 | GGAAGUUGACUAAACUUGN | 1957-1975 | 1955 |
| 467 | NCAAGUUUAGUCAACUUCN | 941 | NGAAGUUGACUAAACUUGN | 1957-1975 | 1955 |
| 468 | UAGCUUCCAUUUAUUCACA | 942 | UGUGAAUAAAUGGAAGCUA | 1992-2010 | 1990 |
| 469 | AAGCUUCCAUUUAUUCACA | 943 | UGUGAAUAAAUGGAAGCUU | 1992-2010 | 1990 |
| 470 | NAGCUUCCAUUUAUUCACA | 944 | UGUGAAUAAAUGGAAGCUN | 1992-2010 | 1990 |
| 471 | NAGCUUCCAUUUAUUCACN | 945 | NGUGAAUAAAUGGAAGCUN | 1992-2010 | 1990 |
| 472 | AGUAAGAUCUGAAACUAGU | 946 | ACUAGUUUCAGAUCUUACU | 2016-2034 | 2014 |
| 473 | UGUAAGAUCUGAAACUAGU | 947 | ACUAGUUUCAGAUCUUACA | 2016-2034 | 2014 |
| 474 | NGUAAGAUCUGAAACUAGU | 948 | ACUAGUUUCAGAUCUUACN | 2016-2034 | 2014 |
| 475 | NGUAAGAUCUGAAACUAGN | 949 | NCUAGUUUCAGAUCUUACN | 2016-2034 | 2014 |
| 476 | AGUUAGUAAGAUCUGAAAC | 950 | GUUUCAGAUCUUACUAACU | 2020-2038 | 2018 |
| 477 | UGUUAGUAAGAUCUGAAAC | 951 | GUUUCAGAUCUUACUAACA | 2020-2038 | 2018 |
| 478 | NGUUAGUAAGAUCUGAAAC | 952 | GUUUCAGAUCUUACUAACN | 2020-2038 | 2018 |
| 479 | NGUUAGUAAGAUCUGAAAN | 953 | NUUUCAGAUCUUACUAACN | 2020-2038 | 2018 |
| 480 | GAAGUUAGUAAGAUCUGAA | 954 | UUCAGAUCUUACUAACUUC | 2022-2040 | 2020 |
| 481 | UAAGUUAGUAAGAUCUGAA | 955 | UUCAGAUCUUACUAACUUA | 2022-2040 | 2020 |
| 482 | AAAGUUAGUAAGAUCUGAA | 956 | UUCAGAUCUUACUAACUUU | 2022-2040 | 2020 |
| 483 | NAAGUUAGUAAGAUCUGAA | 957 | UUCAGAUCUUACUAACUUN | 2022-2040 | 2020 |
| 484 | NAAGUUAGUAAGAUCUGAN | 958 | NUCAGAUCUUACUAACUUN | 2022-2040 | 2020 |
| 485 | CCAUAAAACAAGCACAUGC | 959 | GCAUGUGCUUGUUUUAUGG | 2632-2650 | 2630 |
| 486 | UCAUAAAACAAGCACAUGC | 960 | GCAUGUGCUUGUUUUAUGA | 2632-2650 | 2630 |
| 487 | ACAUAAAACAAGCACAUGC | 961 | GCAUGUGCUUGUUUUAUGU | 2632-2650 | 2630 |
| 488 | NCAUAAAACAAGCACAUGC | 962 | GCAUGUGCUUGUUUUAUGN | 2632-2650 | 2630 |
| 489 | NCAUAAAACAAGCACAUGN | 963 | NCAUGUGCUUGUUUUAUGN | 2632-2650 | 2630 |
| 490 | CUGAACACUUCGCUUUGUC | 964 | GACAAAGCGAAGUGUUCAG | 2781-2799 | 2779 |
| 491 | UUGAACACUUCGCUUUGUC | 965 | GACAAAGCGAAGUGUUCAA | 2781-2799 | 2779 |
| 492 | AUGAACACUUCGCUUUGUC | 966 | GACAAAGCGAAGUGUUCAU | 2781-2799 | 2779 |
| 493 | NUGAACACUUCGCUUUGUC | 967 | GACAAAGCGAAGUGUUCAN | 2781-2799 | 2779 |
| 494 | NUGAACACUUCGCUUUGUN | 968 | NACAAAGCGAAGUGUUCAN | 2781-2799 | 2779 |
| 495 | AGAUUCUAGAGGUCACUGG | 969 | CCAGUGACCUCUAGAAUCU | 3192-3210 | 3190 |
| 496 | UGAUUCUAGAGGUCACUGG | 970 | CCAGUGACCUCUAGAAUCA | 3192-3210 | 3190 |
| 497 | NGAUUCUAGAGGUCACUGG | 971 | CCAGUGACCUCUAGAAUCN | 3192-3210 | 3190 |
| 498 | NGAUUCUAGAGGUCACUGN | 972 | NCAGUGACCUCUAGAAUCN | 3192-3210 | 3190 |
| 499 | UGGAAUAGUGUGCAAAUGC | 973 | GCAUUUGCACACUAUUCCA | 3334-3352 | 3332 |
| 500 | AGGAAUAGUGUGCAAAUGC | 974 | GCAUUUGCACACUAUUCCU | 3334-3352 | 3332 |
| 501 | NGGAAUAGUGUGCAAAUGC | 975 | GCAUUUGCACACUAUUCCN | 3334-3352 | 3332 |
| 502 | NGGAAUAGUGUGCAAAUGN | 976 | NCAUUUGCACACUAUUCCN | 3334-3352 | 3332 |
| 503 | CUGAUCUCAAACAUCUGUC | 977 | GACAGAUGUUUGAGAUCAG | 3414-3432 | 3412 |
| 504 | UUGAUCUCAAACAUCUGUC | 978 | GACAGAUGUUUGAGAUCAA | 3414-3432 | 3412 |
| 505 | AUGAUCUCAAACAUCUGUC | 979 | GACAGAUGUUUGAGAUCAU | 3414-3432 | 3412 |
| 506 | NUGAUCUCAAACAUCUGUC | 980 | GACAGAUGUUUGAGAUCAN | 3414-3432 | 3412 |
| 507 | NUGAUCUCAAACAUCUGUN | 981 | NACAGAUGUUUGAGAUCAN | 3414-3432 | 3412 |
| 508 | CAGCAUAAGUUGCCACACA | 982 | UGUGUGGCAACUUAUGCUG | 3466-3484 | 3464 |
| 509 | UAGCAUAAGUUGCCACACA | 983 | UGUGUGGCAACUUAUGCUA | 3466-3484 | 3464 |
| 510 | AAGCAUAAGUUGCCACACA | 984 | UGUGUGGCAACUUAUGCUU | 3466-3484 | 3464 |
| 511 | NAGCAUAAGUUGCCACACA | 985 | UGUGUGGCAACUUAUGCUN | 3466-3484 | 3464 |
| 512 | NAGCAUAAGUUGCCACACN | 986 | NGUGUGGCAACUUAUGCUN | 3466-3484 | 3464 |
| 513 | CCACAAUCAAGGUCUGGUC | 987 | GACCAGACCUUGAUUGUGG | 3993-4011 | 3991 |
| 514 | UCACAAUCAAGGUCUGGUC | 988 | GACCAGACCUUGAUUGUGA | 3993-4011 | 3991 |
| 515 | ACACAAUCAAGGUCUGGUC | 989 | GACCAGACCUUGAUUGUGU | 3993-4011 | 3991 |
| 516 | NCACAAUCAAGGUCUGGUC | 990 | GACCAGACCUUGAUUGUGN | 3993-4011 | 3991 |
| 517 | NCACAAUCAAGGUCUGGUN | 991 | NACCAGACCUUGAUUGUGN | 3993-4011 | 3991 |
| 518 | AGAAAGAGAGUGCUUCUCC | 992 | GGAGAAGCACUCUCUUUCU | 4502-4520 | 4500 |
| 519 | UGAAAGAGAGUGCUUCUCC | 993 | GGAGAAGCACUCUCUUUCA | 4502-4520 | 4500 |
| 520 | NGAAAGAGAGUGCUUCUCC | 994 | GGAGAAGCACUCUCUUUCN | 4502-4520 | 4500 |
| 521 | NGAAAGAGAGUGCUUCUCN | 995 | NGAGAAGCACUCUCUUUCN | 4502-4520 | 4500 |
| 522 | GGUUAGAGAGAGUUUGGUU | 996 | AACCAAACUCUCUCUAACC | 4584-4602 | 4582 |
| 523 | UGUUAGAGAGAGUUUGGUU | 997 | AACCAAACUCUCUCUAACA | 4584-4602 | 4582 |
| 524 | AGUUAGAGAGAGUUUGGUU | 998 | AACCAAACUCUCUCUAACU | 4584-4602 | 4582 |
| 525 | NGUUAGAGAGAGUUUGGUU | 999 | AACCAAACUCUCUCUAACN | 4584-4602 | 4582 |
| 526 | NGUUAGAGAGAGUUUGGUN | 1000 | NACCAAACUCUCUCUAACN | 4584-4602 | 4582 |
| 527 | CAAGGUUAGAGAGAGUUUG | 1001 | CAAACUCUCUCUAACCUUG | 4587-4605 | 4585 |
| 528 | UAAGGUUAGAGAGAGUUUG | 1002 | CAAACUCUCUCUAACCUUA | 4587-4605 | 4585 |
| 529 | AAAGGUUAGAGAGAGUUUG | 1003 | CAAACUCUCUCUAACCUUU | 4587-4605 | 4585 |
| 530 | NAAGGUUAGAGAGAGUUUG | 1004 | CAAACUCUCUCUAACCUUN | 4587-4605 | 4585 |
| 531 | NAAGGUUAGAGAGAGUUUN | 1005 | NAAACUCUCUCUAACCUUN | 4587-4605 | 4585 |
| 532 | UGGUUUUGUAACUUACAUC | 1006 | GAUGUAAGUUACAAAACCA | 4625-4643 | 4623 |
| 533 | AGGUUUUGUAACUUACAUC | 1007 | GAUGUAAGUUACAAAACCU | 4625-4643 | 4623 |
| 534 | NGGUUUUGUAACUUACAUC | 1008 | GAUGUAAGUUACAAAACCN | 4625-4643 | 4623 |
| 535 | NGGUUUUGUAACUUACAUN | 1009 | NAUGUAAGUUACAAAACCN | 4625-4643 | 4623 |
| 536 | GCACUUUCAUCACAGGUGG | 1010 | CCACCUGUGAUGAAAGUGC | 4641-4659 | 4639 |
| 537 | UCACUUUCAUCACAGGUGG | 1011 | CCACCUGUGAUGAAAGUGA | 4641-4659 | 4639 |
| 538 | ACACUUUCAUCACAGGUGG | 1012 | CCACCUGUGAUGAAAGUGU | 4641-4659 | 4639 |
| 539 | NCACUUUCAUCACAGGUGG | 1013 | CCACCUGUGAUGAAAGUGN | 4641-4659 | 4639 |
| 540 | NCACUUUCAUCACAGGUGN | 1014 | NCACCUGUGAUGAAAGUGN | 4641-4659 | 4639 |
| 541 | GGAUUAUAAAUGCACCAGU | 1015 | ACUGGUGCAUUUAUAAUCC | 5257-5275 | 5255 |
| 542 | UGAUUAUAAAUGCACCAGU | 1016 | ACUGGUGCAUUUAUAAUCA | 5257-5275 | 5255 |
| 543 | AGAUUAUAAAUGCACCAGU | 1017 | ACUGGUGCAUUUAUAAUCU | 5257-5275 | 5255 |
| 544 | NGAUUAUAAAUGCACCAGU | 1018 | ACUGGUGCAUUUAUAAUCN | 5257-5275 | 5255 |
| 545 | NGAUUAUAAAUGCACCAGN | 1019 | ACUGGUGCAUUUAUAAUCN | 5257-5275 | 5255 |
| 546 | CAGAGUUAUAGUCAGAGCU | 1020 | AGCUCUGACUAUAACUCUG | 5851-5869 | 5849 |
| 547 | UAGAGUUAUAGUCAGAGCU | 1021 | AGCUCUGACUAUAACUCUA | 5851-5869 | 5849 |
| 548 | AAGAGUUAUAGUCAGAGCU | 1022 | AGCUCUGACUAUAACUCUU | 5851-5869 | 5849 |
| 549 | NAGAGUUAUAGUCAGAGCU | 1023 | AGCUCUGACUAUAACUCUN | 5851-5869 | 5849 |
| 550 | NAGAGUUAUAGUCAGAGCN | 1024 | NGCUCUGACUAUAACUCUN | 5851-5869 | 5849 |
| 551 | CUAGGAUGCUCUGUAAAGG | 1025 | CCUUUACAGAGCAUCCUAG | 6145-6163 | 6143 |
| 552 | UUAGGAUGCUCUGUAAAGG | 1026 | CCUUUACAGAGCAUCCUAA | 6145-6163 | 6143 |
| 553 | AUAGGAUGCUCUGUAAAGG | 1027 | CCUUUACAGAGCAUCCUAU | 6145-6163 | 6143 |
| 554 | NUAGGAUGCUCUGUAAAGG | 1028 | CCUUUACAGAGCAUCCUAN | 6145-6163 | 6143 |
| 555 | NUAGGAUGCUCUGUAAAGN | 1029 | NCUUUACAGAGCAUCCUAN | 6145-6163 | 6143 |
| 556 | AGUUAGACUUAACUUAGUU | 1030 | AACUAAGUUAAGUCUAACU | 6296-6314 | 6294 |
| 557 | UGUUAGACUUAACUUAGUU | 1031 | AACUAAGUUAAGUCUAACA | 6296-6314 | 6294 |
| 558 | NGUUAGACUUAACUUAGUU | 1032 | AACUAAGUUAAGUCUAACN | 6296-6314 | 6294 |
| 559 | NGUUAGACUUAACUUAGUN | 1033 | NACUAAGUUAAGUCUAACN | 6296-6314 | 6294 |
| 560 | AGUAAAUUCAAUCUCUCUG | 1034 | CAGAGAGAUUGAAUUUACU | 6410-6428 | 6408 |
| 561 | UGUAAAUUCAAUCUCUCUG | 1035 | CAGAGAGAUUGAAUUUACA | 6410-6428 | 6408 |
| 562 | NGUAAAUUCAAUCUCUCUG | 1036 | CAGAGAGAUUGAAUUUACN | 6410-6428 | 6408 |
| 563 | NGUAAAUUCAAUCUCUCUN | 1037 | NAGAGAGAUUGAAUUUACN | 6410-6428 | 6408 |
| 564 | GGUUGAUGUUAACCAGAGC | 1038 | GCUCUGGUUAACAUCAACC | 6539-6557 | 6537 |
| 565 | UGUUGAUGUUAACCAGAGC | 1039 | GCUCUGGUUAACAUCAACA | 6539-6557 | 6537 |
| 566 | AGUUGAUGUUAACCAGAGC | 1040 | GCUCUGGUUAACAUCAACU | 6539-6557 | 6537 |
| 567 | NGUUGAUGUUAACCAGAGC | 1041 | GCUCUGGUUAACAUCAACN | 6539-6557 | 6537 |
| 568 | NGUUGAUGUUAACCAGAGN | 1042 | NCUCUGGUUAACAUCAACN | 6539-6557 | 6537 |
| 569 | AAACUAUGAUGUCAGGUCA | 1043 | UGACCUGACAUCAUAGUUU | 6835-6853 | 6833 |
| 570 | UAACUAUGAUGUCAGGUCA | 1044 | UGACCUGACAUCAUAGUUA | 6835-6853 | 6833 |
| 571 | NAACUAUGAUGUCAGGUCA | 1045 | UGACCUGACAUCAUAGUUN | 6835-6853 | 6833 |
| 572 | NAACUAUGAUGUCAGGUCN | 1046 | NGACCUGACAUCAUAGUUN | 6835-6853 | 6833 |
| 573 | ACAUUAACGAGAUCUGUUU | 1047 | AAACAGAUCUCGUUAAUGU | 6959-6977 | 6957 |
| 574 | UCAUUAACGAGAUCUGUUU | 1048 | AAACAGAUCUCGUUAAUGA | 6959-6977 | 6957 |
| 575 | NCAUUAACGAGAUCUGUUU | 1049 | AAACAGAUCUCGUUAAUGN | 6959-6977 | 6957 |
| 576 | NCAUUAACGAGAUCUGUUN | 1050 | NAACAGAUCUCGUUAAUGN | 6959-6977 | 6957 |
| 577 | GCAGAAUAUAGCUUUAAGA | 1051 | UCUUAAAGCUAUAUUCUGC | 7210-7228 | 7208 |
| 578 | UCAGAAUAUAGCUUUAAGA | 1052 | UCUUAAAGCUAUAUUCUGA | 7210-7228 | 7208 |
| 579 | ACAGAAUAUAGCUUUAAGA | 1053 | UCUUAAAGCUAUAUUCUGU | 7210-7228 | 7208 |
| 580 | NCAGAAUAUAGCUUUAAGA | 1054 | UCUUAAAGCUAUAUUCUGN | 7210-7228 | 7208 |
| 581 | NCAGAAUAUAGCUUUAAGN | 1055 | NCUUAAAGCUAUAUUCUGN | 7210-7228 | 7208 |

The MARC1 RNAi agent sense strands and antisense strands that comprise or consist of the sequences in Table 2 can be modified nucleotides or unmodified nucleotides. In some embodiments, the MARC1 RNAi agents having the sense and antisense strand sequences that comprise or consist of the sequences in Table 2 are all or substantially all modified nucleotides.

In some embodiments, the antisense strand of a MARC1 RNAi agent disclosed herein differs by 0. 1. 2, or 3 nucleotides from any of the antisense strand sequences in Table 2. In some embodiments, the sense strand of a MARC1 RNAi agent disclosed herein differs by 0, 1, 2, or 3 nucleotides from any of the sense strand sequences in Table 2.

As used herein, each N listed in a sequence disclosed in Table 2 may be independently selected from any and all nucleobases (including those found on both modified and unmodified nucleotides). In some embodiments, an N nucleotide listed in a sequence disclosed in Table 2 has a nucleobase that is complementary to the N nucleotide at the corresponding position on the other strand. In some embodiments, an N nucleotide listed in a sequence disclosed in Table 2 has a nucleobase that is not complementary to the N nucleotide at the corresponding position on the other strand. In some embodiments, an N nucleotide listed in a sequence disclosed in Table 2 has a nucleobase that is the same as the N nucleotide at the corresponding position on the other strand. In some embodiments. an N nucleotide listed in a sequence disclosed in Table 2 has a nucleobase that is different from the N nucleotide at the corresponding position on the other strand.

Certain modified MARC1 RNAi agent antisense strands, as well as their underlying unmodified nucleobase sequences, are provided in Table 3. Certain modified MARC1 RNAi agent sense strands, as well as their underlying unmodified nucleobase sequences, are provided in Table 4. In forming MARC1 RNAi agents, each of the nucleotides in each of the underlying base sequences listed in Tables 3 and 4. as well as in Table 2, above, can be a modified nucleotide.

The MARC1 RNAi agents described herein are formed by annealing an antisense strand with a sense strand. A sense strand containing a sequence listed in Table 2, Table 4, or Table 6D, can be hybridized to any antisense strand containing a sequence listed in Table 2 or Table 3 or Table 6D, provided the two sequences have a region of at least 85% complementarity over a contiguous 15, 16, 17, 18, 19, 20, or 21 nucleotide sequence.

In some embodiments, a MARC1 RNAi agent antisense strand comprises a nucleotide sequence of any of the sequences in Table 2 or Table 3 or Table 6D.

In some embodiments, a MARC1 RNAi agent comprises or consists of a duplex having the nucleobase sequences of the sense strand and the antisense strand of any of the sequences in Table 2, Table 3, Table 4, or Table 6D. In some embodiments, a MARC1 RNAi agent comprises or consists of a duplex sequence prepared or provided as a sodium salt, mixed salt, or a free-acid.

Examples of antisense strands containing modified nucleotides are provided in Table 3 and Table 6D. Examples of sense strands containing modified nucleotides are provided in Table 4, Table 5, and Table 6D.

As used in Tables 3, 4, and 5, the following notations are used to indicate modified nucleotides and linking groups:
A=adenosine-3′-phosphate:
C=cytidine-3′-phosphate:
G=guanosine-3′-phosphate:
U=uridine-3′-phosphate
I=inosine-3′-phosphate
a=2′-O-methyladenosine-3′-phosphate
as =2′-O-methyladenosine-3′-phosphorothioate
C=2′-O-methylcytidine-3′-phosphate
CS: 2′-O-methylcytidine-3′-phosphorothioate
g 2′-O-methylguanosine-3′-phosphate
gs: 2′-O-methylguanosine-3′-phosphorothioate
t=2′-O-methyl-5-methyluridine-3′-phosphate
ts=2′-O-methyl-5-methyluridine-3′-phosphorothioate
u=2′-O-methyluridine-3′-phosphate
us=2′-O-methyluridine-3′-phosphorothioate
i=2′-O-methylinosine-3′-phosphate
is =2′-O-methylinosine-3′-phosphorothioate
Af=2′-fluoroadenosine-3′-phosphate
Afs=2′-fluoroadenosine-3′-phosporothioate
Cf 2′-fluorocytidine-3′-phosphate
Cfs=2′-fluorocytidine-3′-phosphorothioate
Gf: 2′-fluoroguanosine-3′-phosphate
Gfs=2′-fluoroguanosine-3′-phosphorothioate
Tf=2′-fluoro-5′-methyluridine-3′-phosphate
Tfs=2′-fluoro-5′-methyluridine-3′-phosphorothioate
Uf=2′-fluorouridine-3′-phosphate
Ufs=2′-fluorouridine-3′-phosphorothioate
A_{UNA}=2′,3′-seco-adenosine-3′-phosphate, see Table 7
A_{UNAS}=2′,3′-seco-adenosine-3′-phosphorothioate, see Table 7
C_{UNA}=2′, 3′-seco-cytidine-3′-phosphate, see Table 7
C_{UNAS}=2′,3′-seco-cytidine-3′-phosphorothioate, see Table 7
G_{UNA} 2′,3′-seco-guanosine-3′-phosphate, see Table 7
G_{UNAS} 2′,3′-seco-guanosine-3′-phosphorothioate, see Table 7
U_{UNA} 2′,3′-seco-uridine-3′-phosphate, see Table 7
U_{UNAS} 2′,3′-seco-uridine-3′-phosphorothioate, see Table 7
a 2N=2′-O-methyl-2-aminoadenosine-3′-phosphate, see Table 7
a 2Ns=2′-O-methyl-2-aminoadenosine-3′-phosphorothioate, see Table 7
(invAb)=inverted abasic deoxyribonucleotide, see Table 7
(invAb)s=inverted abasic deoxyribonucleotide-5′-phosphorothioate, see Table 7
cPrpa=5′-cyclopropyl phosphonate-2′-O-methyladenosine-3′-phosphate (see Table 7)
cPrpas=5′-cyclopropyl phosphonate-2′-O-methyladenosine-3′-phosphorothioate (see Table 7)
cPrpu 5′-cyclopropyl phosphonate-2′-O-methyluridine-3′-phosphate (see Table 7)
cPrpus=5′-cyclopropyl phosphonate-2′-O-methyluridine-3′-phosphorothioate (see Table 7)

As the person of ordinary skill in the art would readily understand, unless otherwise indicated by the sequence (such as, for example. by a phosphorothioate linkage “s”). when present in an oligonucleotide, the nucleotide monomers are mutually linked by 5′-3′-phosphodiester bonds. As the person of ordinary skill in the art would clearly understand, the inclusion of a phosphorothioate linkage as shown in the modified nucleotide sequences disclosed herein replaces the phosphodiester linkage typically present in oligonucleotides. Further, the person of ordinary skill in the art would readily understand that the terminal nucleotide at the 3′ end of a given oligonucleotide sequence would typically have a hydroxyl (—OH) group at the respective 3′ position of the given monomer instead of a phosphate moiety ex vivo. Additionally, for the embodiments disclosed herein, when viewing the respective strand 5′→3′, the inverted abasic residues are inserted such that the 3′ position of the deoxyribose is linked at the 3′ end of the preceding monomer on the respective strand (see. e.g., Table 7). Moreover, as the person of ordinary skill would readily understand and appreciate, while the phosphorothioate chemical structures depicted herein typically show the anion on the sulfur atom, the inventions disclosed herein encompass all phosphorothioate tautomers and resonance structures (e.g., where the sulfur atom has a double-bond and the anion is on an oxygen atom). Unless expressly indicated otherwise herein, such understandings of the person of ordinary skill in the art are used when describing the MARC1 RNAi agents and compositions of MARC1 RNAi agents disclosed herein.

Certain examples of targeting ligands, targeting groups, and linking groups used with the MARC1 RNAi agents disclosed herein are provided below in Table 7. More specifically, targeting groups and linking groups (which together can form a targeting ligand) include (NAG37) and (NAG37) s, for which their chemical structures are provided below in Table 7. Each sense strand and/or antisense strand can have any targeting ligands, targeting groups, or linking groups listed herein, as well as other groups, conjugated to the 5′ and/or 3′ end of the sequence.

TABLE 3
MARC1 RNAi agent Antisense Strand Sequences

| | | | Underlying Base Sequence | |
|---|---|---|---|---|
| | | SEQ | (5′ → 3′) | SEQ |
| AS Strand | Modified Antisense | ID | (Shown as an Unmodified | ID |
| ID | Strand (5′ → 3′) | NO. | Nucleotide Sequence) | NO. |
| AM16712-AS | usAfsasCfcAfuguuuCfcCfuCfcUfggsu | 1056 | UAACCAUGUUUCCCUCCUGGU | 1542 |
| AM16714-AS | usAfscsUfuCfaggaaGfcUfgGfuUfausc | 1057 | UACUUCAGGAAGCUGGUUAUC | 1543 |
| AM16716-AS | usAfsasCfaAfgucugCfuAfuUfuGfausg | 1058 | UAACAAGUCUGCUAUUUGAUG | 1544 |
| AM16718-AS | usGfsgsAfaCfaagucUfgCfuAfuUfugsg | 1059 | UGGAACAAGUCUGCUAUUUGG | 1545 |
| AM16720-AS | usGfsasGfuAfagcaaUfcUfgGfuCfcusc | 1060 | UGAGUAAGCAAUCUGGUCCUC | 1546 |
| AM16722-AS | usGfsusCfuGfaguaaGfcAfaUfcUfggsu | 1061 | UGUCUGAGUAAGCAAUCUGGU | 1547 |
| AM16724-AS | usUfsgsGfuGfU_{UNA}cugaGfuAfaGfcAfausc | 1062 | UUGGUGUCUGAGUAAGCAAUC | 1548 |
| AM16726-AS | usUfsasAfcUfuucuuCfuCfuAfgCfcusg | 1063 | UUAACUUUCUUCUCUAGCCUG | 1549 |
| AM16728-AS | usGfsgsUfuAfaaaugCfaUfcUfgGfaasc | 1064 | UGGUUAAAAUGCAUCUGGAAC | 1550 |
| AM16730-AS | asAfscsUfuUfcguucUfgAfaGfgGfucsa | 1065 | AACUUUCGUUCUGAAGGGUCA | 1551 |
| AM16732-AS | usUfscsCfaUfauaacUfuUfcGfuUfcusg | 1066 | UUCCAUAUAACUUUCGUUCUG | 1552 |
| AM16734-AS | usGfsasUfuUfuccauAfuAfaCfuUfucsg | 1067 | UGAUUUUCCAUAUAACUUUCG | 1553 |
| AM16736-AS | asAfsasGfaGfuggugAfuUfuUfcCfausg | 1068 | AAAGAGUGGUGAUUUUCCAUG | 1554 |
| AM16738-AS | usCfsasUfcUfaauauUfcCfaGfgAfcasc | 1069 | UCAUCUAAUAUUCCAGGACAC | 1555 |
| AM16740-AS | asGfsusGfuUfgucauUfuUfuGfaGfaasc | 1070 | AGUGUUGUCAUUUUUGAGAAC | 1556 |
| AM16742-AS | usAfsusGfcUfucaagUfgUfuGfuCfausc | 1071 | UAUGCUUCAAGUGUUGUCAUC | 1557 |
| AM16744-AS | asCfsasCfcAfU_{UNA}gcuuCfaAfgUfgUfugsc | 1072 | ACACCAUGCUUCAAGUGUUGC | 1558 |
| AM16746-AS | asGfsusUfcUfgaaacAfcCfaUfgCfuusc | 1073 | AGUUCUGAAACACCAUGCUUC | 1559 |
| AM16748-AS | usAfsusUfgAfagcauUfgAfgAfcAfccsa | 1074 | UAUUGAAGCAUUGAGACACCA | 1560 |
| AM16750-AS | usGfsasCfaUfugaagCfaUfuGfaGfacsg | 1075 | UGACAUUGAAGCAUUGAGACG | 1561 |
| AM16752-AS | usGfsasGfuUfuucagAfaUfcCfuGfucsc | 1076 | UGAGUUUUCAGAAUCCUGUCC | 1562 |
| AM16754-AS | asAfsgsAfaCfuauucCfaUfaAfuCfagsc | 1077 | AAGAACUAUUCCAUAAUCAGC | 1563 |
| AM16756-AS | asGfsasAfaGfaacuaUfuCfcAfuAfausc | 1078 | AGAAAGAACUAUUCCAUAAUC | 1564 |
| AM16758-AS | asGfsasUfcCfagaucUfaUfgGfaAfaasc | 1079 | AGAUCCAGAUCUAUGGAAAAC | 1565 |
| AM16760-AS | asUfscsCfaAfugcugUfcUfgAfgAfagsc | 1080 | AUCCAAUGCUGUCUGAGAAGC | 1566 |
| AM16762-AS | asCfscsUfuUfaggaaAfuCfcAfaUfgcsc | 1081 | ACCUUUAGGAAAUCCAAUGCC | 1567 |
| AM16764-AS | usCfsasCfcUfuuaggAfaAfuCfcAfausg | 1082 | UCACCUUUAGGAAAUCCAAUG | 1568 |
| AM16766-AS | usGfsgsUfuUfuaaacAfaCfuGfaCfausg | 1083 | UGGUUUUAAACAACUGACAUG | 1569 |
| AM16768-AS | usUfscsAfuCfagaucUfuAfgAfgUfuasc | 1084 | UUCAUCAGAUCUUAGAGUUAC | 1570 |
| AM16770-AS | usUfsusCfaUfcagauCfuUfaGfaGfuusg | 1085 | UUUCAUCAGAUCUUAGAGUUG | 1571 |
| AM16772-AS | usCfsasAfgUfuuaguCfaAfcUfuCfccsa | 1086 | UCAAGUUUAGUCAACUUCCCA | 1572 |
| AM16774-AS | usAfsgsCfuUfccauuUfaUfuCfaCfagsc | 1087 | UAGCUUCCAUUUAUUCACAGC | 1573 |
| AM17404-AS | asGfsusguugucauUfuUfuGfagaasc | 1088 | AGUGUUGUCAUUUUUGAGAAC | 1556 |
| AM17405-AS | cPrpasGfsusguugucauUfuUfuGfagaasc | 1089 | AGUGUUGUCAUUUUUGAGAAC | 1556 |
| AM17406-AS | usAfsusugaagcauUfgAfgAfcaccsa | 1090 | UAUUGAAGCAUUGAGACACCA | 1560 |
| AM17407-AS | cPrpusAfsusugaagcauUfgAfgAfcaccsa | 1091 | UAUUGAAGCAUUGAGACACCA | 1560 |
| AM17408-AS | asAfsgsaacuauucCfaUfaAfucagsc | 1092 | AAGAACUAUUCCAUAAUCAGC | 1563 |
| AM17409-AS | cPrpasAfsgsaacuauucCfaUfaAfucagsc | 1093 | AAGAACUAUUCCAUAAUCAGC | 1563 |
| AM17410-AS | asGfsasaagaacuaUfuCfcAfuaausc | 1094 | AGAAAGAACUAUUCCAUAAUC | 1564 |
| AM17411-AS | cPrpasGfsasaagaacuaUfuCfcAfuaausc | 1095 | AGAAAGAACUAUUCCAUAAUC | 1564 |
| AM17488-AS | usUfsgsAfuCfacaagCfcAfaAfaCfcusg | 1096 | UUGAUCACAAGCCAAAACCUG | 1574 |
| AM17490-AS | usCfsasUfaGfacaucGfcAfuCfcUfgasa | 1097 | UCAUAGACAUCGCAUCCUGAA | 1575 |
| AM17492-AS | usCfsasAfaGfaguggUfgAfuUfuUfccsa | 1098 | UCAAAGAGUGGUGAUUUUCCA | 1576 |
| AM17494-AS | usUfscsUfgAfaacacCfaUfgCfuUfcasa | 1099 | UUCUGAAACACCAUGCUUCAA | 1577 |
| AM17496-AS | usCfsasGfaAfuccugUfcUfuGfuCfausu | 1100 | UCAGAAUCCUGUCUUGUCAUU | 1578 |
| AM17498-AS | usCfsasAfuGfcugucUfgAfgAfaGfcasg | 1101 | UCAAUGCUGUCUGAGAAGCAG | 1579 |
| AM17500-AS | usCfscsUfuUfaaaggUfuUfuCfaGfuasg | 1102 | UCCUUUAAAGGUUUUCAGUAG | 1580 |
| AM17502-AS | usAfscsUfuCfaucagAfuCfuUfaGfagsu | 1103 | UACUUCAUCAGAUCUUAGAGU | 1581 |
| AM17504-AS | usAfsasGfuUfuagucAfaCfuUfcCfcasa | 1104 | UAAGUUUAGUCAACUUCCCAA | 1582 |
| AM17808-AS | usGfsusGfuAfggcugCfaCfuGfaGfagsu | 1105 | UGUGUAGGCUGCACUGAGAGU | 1583 |
| AM17810-AS | usUfsgsUfgUfaggcuGfcAfcUfgAfgasg | 1106 | UUGUGUAGGCUGCACUGAGAG | 1584 |
| AM17812-AS | usCfsusUfuGfuguagGfcUfgCfaCfugsa | 1107 | UCUUUGUGUAGGCUGCACUGA | 1585 |
| AM17814-AS | usUfscsCfuUfuguguAfgGfcUfgCfacsu | 1108 | UUCCUUUGUGUAGGCUGCACU | 1586 |
| AM17816-AS | usUfscsGfaAfgugcaCfcAfgGfcGfgusa | 1109 | UUCGAAGUGCACCAGGCGGUA | 1587 |
| AM17818-AS | usCfsusCfgAfagugcAfcCfaGfgCfggsu | 1110 | UCUCGAAGUGCACCAGGCGGU | 1588 |
| AM17820-AS | usGfscsUfcGfaagugCfaCfcAfgGfcgsg | 1111 | UGCUCGAAGUGCACCAGGCGG | 1589 |
| AM17822-AS | asGfsgsCfuCfgaaguGfcAfcCfaGfgcsg | 1112 | AGGCUCGAAGUGCACCAGGCG | 1590 |
| AM17824-AS | asAfscsUfcUfucaguGfuUfuCfcAfgcsg | 1113 | AACUCUUCAGUGUUUCCAGCG | 1591 |
| AM17826-AS | usAfscsAfgGfgucucCfcAfcUfuUfgasu | 1114 | UACAGGGUCUCCCACUUUGAU | 1592 |
| AM17828-AS | asCfsasCfaGfggucuCfcCfaCfuUfugsa | 1115 | ACACAGGGUCUCCCACUUUGA | 1593 |
| AM17830-AS | usGfsusAfcAfcagggUfcUfcCfcAfcusu | 1116 | UGUACACAGGGUCUCCCACUU | 1594 |
| AM17832-AS | asGfsgsUfaCfacaggGfuCfuCfcCfacsu | 1117 | AGGUACACAGGGUCUCCCACU | 1595 |
| AM17834-AS | usAfsgsGfuAfcacagGfgUfcUfcCfcasc | 1118 | UAGGUACACAGGGUCUCCCAC | 1596 |
| AM17864-AS | usGfsasAfcAfagucuGfcUfaUfuUfgasu | 1119 | UGAACAAGUCUGCUAUUUGAU | 1597 |
| AM17866-AS | usGfscsUfuUfaacuuUfcUfuCfuCfuasg | 1120 | UGCUUUAACUUUCUUCUCUAG | 1598 |
| AM17868-AS | usUfsusGfcUfuuaacUfuUfcUfuCfucsu | 1121 | UUUGCUUUAACUUUCUUCUCU | 1599 |
| AM17870-AS | usGfsasAfaUfuacaaUfaUfuGfgGfccsu | 1122 | UGAAAUUACAAUAUUGGGCCU | 1600 |
| AM17872-AS | usAfsasCfuUfucguuCfuGfaAfgGfgusc | 1123 | UAACUUUCGUUCUGAAGGGUC | 1601 |
| AM17874-AS | usCfsasUfaUfaacuuUfcGfuUfcUfgasa | 1124 | UCAUAUAACUUUCGUUCUGAA | 1602 |
| AM17876-AS | usUfscsCfaUfaaucaGfuUfaAfaCfggsg | 1125 | UUCCAUAAUCAGUUAAACGGG | 1603 |
| AM17878-AS | usUfsasGfgAfaauccAfaUfgCfuGfucsu | 1126 | UUAGGAAAUCCAAUGCUGUCU | 1604 |
| AM17880-AS | usAfsasAfgGfacaaaAfuGfgCfaAfuasa | 1127 | UAAAGGACAAAAUGGCAAUAA | 1605 |
| AM17882-AS | usAfsasUfcAfaaggaCfaAfaAfuGfgcsa | 1128 | UAAUCAAAGGACAAAAUGGCA | 1606 |
| AM18384-AS | usUfsgsauCfacaagCfcAfaAfaccusg | 1129 | UUGAUCACAAGCCAAAACCUG | 1574 |
| AM18385-AS | cPrpusUfsgsauCfacaagCfcAfaAfaccusg | 1130 | UUGAUCACAAGCCAAAACCUG | 1574 |
| AM18386-AS | usUfsgsaucacaagCfcAfaAfaccusg | 1131 | UUGAUCACAAGCCAAAACCUG | 1574 |
| AM18387-AS | cPrpusUfsgsaucacaagCfcAfaAfaccusg | 1132 | UUGAUCACAAGCCAAAACCUG | 1574 |
| AM18388-AS | usCfscsuuUfaaaggUfuUfuCfaguasg | 1133 | UCCUUUAAAGGUUUUCAGUAG | 1580 |
| AM18389-AS | cPrpusCfscsuuUfaaaggUfuUfuCfaguasg | 1134 | UCCUUUAAAGGUUUUCAGUAG | 1580 |
| AM18390-AS | usCfscsuuuaaaggUfuUfuCfaguasg | 1135 | UCCUUUAAAGGUUUUCAGUAG | 1580 |
| AM18391-AS | cPrpusCfscsuuuaaaggUfuUfuCfaguasg | 1136 | UCCUUUAAAGGUUUUCAGUAG | 1580 |
| AM18393-AS | usCfscsuuuaaaggUfuUfuCfagugsg | 1137 | UCCUUUAAAGGUUUUCAGUGG | 1607 |
| AM18394-AS | cPrpusCfscsuuuaaaggUfuUfuCfagugsg | 1138 | UCCUUUAAAGGUUUUCAGUGG | 1607 |
| AM18398-AS | asGfsasaaGfaacuaUfuCfcAfuaausc | 1139 | AGAAAGAACUAUUCCAUAAUC | 1564 |
| AM18399-AS | cPrpasGfsasaaGfaacuaUfuCfcAfuaausc | 1140 | AGAAAGAACUAUUCCAUAAUC | 1564 |
| AM18401-AS | usGfsasaaGfaacuaUfuCfcAfuaausc | 1141 | UGAAAGAACUAUUCCAUAAUC | 1608 |
| AM18402-AS | cPrpusGfsasaaGfaacuaUfuCfcAfuaausc | 1142 | UGAAAGAACUAUUCCAUAAUC | 1608 |
| AM18403-AS | cPrpusGfaaaGfaacuaUfuCfcAfuaausc | 1143 | UGAAAGAACUAUUCCAUAAUC | 1608 |
| AM18404-AS | asGfsusguUfgucauUfuUfuGfagaasc | 1144 | AGUGUUGUCAUUUUUGAGAAC | 1556 |
| AM18405-AS | cPrpasGfsusguUfgucauUfuUfuGfagaasc | 1145 | AGUGUUGUCAUUUUUGAGAAC | 1556 |
| AM18407-AS | usGfsusguUfgucauUfuUfuGfagaasc | 1146 | UGUGUUGUCAUUUUUGAGAAC | 1609 |
| AM18408-AS | cPrpusGfsusguUfgucauUfuUfuGfagaasc | 1147 | UGUGUUGUCAUUUUUGAGAAC | 1609 |
| AM18409-AS | cPrpusGfsusguU_{UNA}gucauUfuUfuGfagaasc | 1148 | UGUGUUGUCAUUUUUGAGAAC | 1609 |
| AM18410-AS | cPrpusGfuguUfgucauUfuUfuGfagaasc | 1149 | UGUGUUGUCAUUUUUGAGAAC | 1609 |
| AM18411-AS | usGfsusguugucauUfuUfuGfagaasc | 1150 | UGUGUUGUCAUUUUUGAGAAC | 1609 |
| AM18412-AS | cPrpusGfsusguugucauUfuUfuGfagaasc | 1151 | UGUGUUGUCAUUUUUGAGAAC | 1609 |
| AM18413-AS | cPrpusGfuguugucauUfuUfuGfagaasc | 1152 | UGUGUUGUCAUUUUUGAGAAC | 1609 |
| AM18606-AS | usCfsasgaAfuccugUfcUfuGfucausu | 1153 | UCAGAAUCCUGUCUUGUCAUU | 1578 |
| AM18607-AS | cPrpusCfsasgaAfuccugUfcUfuGfucausu | 1154 | UCAGAAUCCUGUCUUGUCAUU | 1578 |
| AM18608-AS | usCfsasgaauccugUfcUfuGfucausu | 1155 | UCAGAAUCCUGUCUUGUCAUU | 1578 |
| AM18609-AS | cPrpusCfsasgaauccugUfcUfuGfucausu | 1156 | UCAGAAUCCUGUCUUGUCAUU | 1578 |
| AM18610-AS | cPrpusCfagaauccugUfcUfuGfucausu | 1157 | UCAGAAUCCUGUCUUGUCAUU | 1578 |
| AM18611-AS | usAfsasguuuagucAfaCfuUfcccasa | 1158 | UAAGUUUAGUCAACUUCCCAA | 1582 |
| AM18612-AS | cPrpusAfsasguuuagucAfaCfuUfcccasa | 1159 | UAAGUUUAGUCAACUUCCCAA | 1582 |
| AM18614-AS | usUfsusGfaUfcacaaGfcCfaAfaAfccsu | 1160 | UUUGAUCACAAGCCAAAACCU | 1610 |
| AM18616-AS | usGfsusUfgAfucacaAfgCfcAfaAfacsc | 1161 | UGUUGAUCACAAGCCAAAACC | 1611 |
| AM18629-AS | asGfsasaAfgaacuaUfuCfcAfuaausc | 1162 | AGAAAGAACUAUUCCAUAAUC | 1564 |
| AM18632-AS | asGfsasaagAfacuaUfuCfcAfuaausc | 1163 | AGAAAGAACUAUUCCAUAAUC | 1564 |
| AM18634-AS | usAfsgsaaAfgaacuAfuUfcCfauaasu | 1164 | UAGAAAGAACUAUUCCAUAAU | 1612 |
| AM18636-AS | usAfsgsaaAfgaacuAfuUfcCfauagsu | 1165 | UAGAAAGAACUAUUCCAUAGU | 1613 |
| AM18638-AS | usAfsgsaaAfgaacuAfuUfcCfauagsc | 1166 | UAGAAAGAACUAUUCCAUAGC | 1614 |
| AM18878-AS | asGfsusAfaGfaucugAfaAfcUfaGfucsa | 1167 | AGUAAGAUCUGAAACUAGUCA | 1615 |
| AM18880-AS | asGfsusUfaGfuaagaUfcUfgAfaAfcusa | 1168 | AGUUAGUAAGAUCUGAAACUA | 1616 |
| AM18882-AS | usAfsasGfuUfaguaaGfaUfcUfgAfaasc | 1169 | UAAGUUAGUAAGAUCUGAAAC | 1617 |
| AM18884-AS | usCfsasUfaAfaacaaGfcAfcAfuGfcasc | 1170 | UCAUAAAACAAGCACAUGCAC | 1618 |
| AM18886-AS | usUfsgsAfaCfacuucGfcUfuUfgUfcusc | 1171 | UUGAACACUUCGCUUUGUCUC | 1619 |
| AM18888-AS | asGfsasUfuCfuagagGfuCfaCfuGfgasa | 1172 | AGAUUCUAGAGGUCACUGGAA | 1620 |
| AM18890-AS | usGfsgsAfaUfaguguGfcAfaAfuGfcusc | 1173 | UGGAAUAGUGUGCAAAUGCUC | 1621 |
| AM18892-AS | usUfsgsAfuCfucaaaCfaUfcUfgUfcasu | 1174 | UUGAUCUCAAACAUCUGUCAU | 1622 |
| AM18894-AS | usAfsgsCfaUfaaguuGfcCfaCfaCfagsa | 1175 | UAGCAUAAGUUGCCACACAGA | 1623 |
| AM18896-AS | usCfsasCfaAfucaagGfuCfuGfgUfcusg | 1176 | UCACAAUCAAGGUCUGGUCUG | 1624 |
| AM18898-AS | asGfsasAfaGfagaguGfcUfuCfuCfccsu | 1177 | AGAAAGAGAGUGCUUCUCCCU | 1625 |
| AM18900-AS | usGfsusUfaGfagagaGfuUfuGfgUfuusc | 1178 | UGUUAGAGAGAGUUUGGUUUC | 1626 |
| AM18902-AS | usAfsasGfgUfuagagAfgAfgUfuUfggsu | 1179 | UAAGGUUAGAGAGAGUUUGGU | 1627 |
| AM18904-AS | usGfsgsUfuUfuguaaCfuUfaCfaUfcasc | 1180 | UGGUUUUGUAACUUACAUCAC | 1628 |
| AM18906-AS | usCfsasCfuUfucaucAfcAfgGfuGfgusu | 1181 | UCACUUUCAUCACAGGUGGUU | 1629 |
| AM18908-AS | usGfsasUfuAfuaaauGfcAfcCfaGfucsa | 1182 | UGAUUAUAAAUGCACCAGUCA | 1630 |
| AM18910-AS | usAfsgsAfgUfuauagUfcAfgAfgCfugsu | 1183 | UAGAGUUAUAGUCAGAGCUGU | 1631 |
| AM18912-AS | usUfsasGfgAfugcucUfgUfaAfaGfgusa | 1184 | UUAGGAUGCUCUGUAAAGGUA | 1632 |
| AM18914-AS | asGfsusUfaGfacuuaAfcUfuAfgUfucsc | 1185 | AGUUAGACUUAACUUAGUUCC | 1633 |
| AM18916-AS | asGfsusAfaAfuucaaUfcUfcUfcUfgasg | 1186 | AGUAAAUUCAAUCUCUCUGAG | 1634 |
| AM18918-AS | usGfsusUfgAfuguuaAfcCfaGfaGfcusg | 1187 | UGUUGAUGUUAACCAGAGCUG | 1635 |
| AM18920-AS | asAfsasCfuAfugaugUfcAfgGfuCfaasc | 1188 | AAACUAUGAUGUCAGGUCAAC | 1636 |
| AM18922-AS | asCfsasUfuAfacgagAfuCfuGfuUfugsa | 1189 | ACAUUAACGAGAUCUGUUUGA | 1637 |
| AM18924-AS | usCfsasGfaAfuauagCfuUfuAfaGfaasc | 1190 | UCAGAAUAUAGCUUUAAGAAC | 1638 |
| AM18944-AS | asGfsusAfgUfaggucCfuUfuGfuGfuasg | 1191 | AGUAGUAGGUCCUUUGUGUAG | 1639 |
| AM18946-AS | usUfsgsCfuAfuuugaUfgAfgGfaCfgusc | 1192 | UUGCUAUUUGAUGAGGACGUC | 1640 |
| AM18948-AS | asUfsgsUfaGfaggucUfcAfgUfuCfugsa | 1193 | AUGUAGAGGUCUCAGUUCUGA | 1641 |
| AM18950-AS | asAfsasGfaAfaauguAfgAfgGfuCfucsa | 1194 | AAAGAAAAUGUAGAGGUCUCA | 1642 |
| AM18952-AS | asCfsgsAfaAfaauguGfaAfaAfuCfacsa | 1195 | ACGAAAAAUGUGAAAAUCACA | 1643 |
| AM18954-AS | usUfsasCfuGfaagucCfuAfcUfaAfgusu | 1196 | UUACUGAAGUCCUACUAAGUU | 1644 |
| AM18956-AS | asGfsasUfaAfacggaGfaAfgCfaGfgasg | 1197 | AGAUAAACGGAGAAGCAGGAG | 1645 |
| AM18958-AS | asCfsasUfuCfuaggaUfaUfuCfuUfccsa | 1198 | ACAUUCUAGGAUAUUCUUCCA | 1646 |
| AM18960-AS | usAfsasCfuGfacauaUfgCfuUfuCfcusu | 1199 | UAACUGACAUAUGCUUUCCUU | 1647 |
| AM19120-AS | cPrpusCfscsUfuUfaaaggUfuUfuCfaGfuasg | 1200 | UCCUUUAAAGGUUUUCAGUAG | 1580 |
| AM19121-AS | usCfscsUfuuaaaggUfuUfuCfaguasg | 1201 | UCCUUUAAAGGUUUUCAGUAG | 1580 |
| AM19122-AS | cPrpusCfscsUfuuaaaggUfuUfuCfaguasg | 1202 | UCCUUUAAAGGUUUUCAGUAG | 1580 |
| AM19123-AS | usCfscsuuuAfaaggUfuUfuCfaguasg | 1203 | UCCUUUAAAGGUUUUCAGUAG | 1580 |
| AM19125-AS | usAfsgsGfuacacagGfgUfcUfcccasc | 1204 | UAGGUACACAGGGUCUCCCAC | 1596 |
| AM19126-AS | usAfsgsguAfcacagGfgUfcUfcccasc | 1205 | UAGGUACACAGGGUCUCCCAC | 1596 |
| AM19127-AS | usAfsgsguaCfacagGfgUfcUfcccasc | 1206 | UAGGUACACAGGGUCUCCCAC | 1596 |
| AM19128-AS | cPrpusAfsgsguaCfacagGfgUfcUfcccasc | 1207 | UAGGUACACAGGGUCUCCCAC | 1596 |
| AM19131-AS | usAfsgsguaCfacagGfgUfcUfcccasu | 1208 | UAGGUACACAGGGUCUCCCAU | 1648 |
| AM19132-AS | usAfsgsguaCfacagGfgUfcUfccuasc | 1209 | UAGGUACACAGGGUCUCCUAC | 1649 |
| AM19181-AS | cPrpusCfaGfaauccugUfcUfuGfucausu | 1210 | UCAGAAUCCUGUCUUGUCAUU | 1578 |
| AM19182-AS | cPrpusCfagAfauccugUfcUfuGfucausu | 1211 | UCAGAAUCCUGUCUUGUCAUU | 1578 |
| AM19183-AS | cPrpusCfagaaUfccugUfcUfuGfucausu | 1212 | UCAGAAUCCUGUCUUGUCAUU | 1578 |
| AM19185-AS | cPrpasCfagaauccugUfcUfuGfucausu | 1213 | ACAGAAUCCUGUCUUGUCAUU | 1650 |
| AM19186-AS | asCfagaauccugUfcUfuGfucausu | 1214 | ACAGAAUCCUGUCUUGUCAUU | 1650 |
| AM19189-AS | cPrpusCfagaauccugUfcUfuGfucgusu | 1215 | UCAGAAUCCUGUCUUGUCGUU | 1651 |
| AM19191-AS | cPrpusCfagaauccugUfcUfuGfucausc | 1216 | UCAGAAUCCUGUCUUGUCAUC | 1652 |
| AM19193-AS | cPrpasCfaGfaauccugUfcUfuGfucausc | 1217 | ACAGAAUCCUGUCUUGUCAUC | 1653 |
| AM19219-AS | asGfsgsCfaUfcuaauAfuUfcCfaGfgasc | 1218 | AGGCAUCUAAUAUUCCAGGAC | 1654 |
| AM19221-AS | asGfscsAfuCfuaauaUfuCfcAfgGfacsa | 1219 | AGCAUCUAAUAUUCCAGGACA | 1655 |
| AM19223-AS | asAfsgsGfcAfucuaaUfaUfuCfcAfggsa | 1220 | AAGGCAUCUAAUAUUCCAGGA | 1656 |
| AM19405-AS | cPrpusGfuguuGfucauUfuUfuGfagaasc | 1221 | UGUGUUGUCAUUUUUGAGAAC | 1609 |
| AM19406-AS | cPrpusGfuGfuugucauUfuUfuGfagaasc | 1222 | UGUGUUGUCAUUUUUGAGAAC | 1609 |
| AM19407-AS | usAfsasucaAfaggaCfaAfaAfuggcsa | 1223 | UAAUCAAAGGACAAAAUGGCA | 1606 |
| AM19408-AS | cPrpusAfaucaAfaggaCfaAfaAfuggcsa | 1224 | UAAUCAAAGGACAAAAUGGCA | 1606 |
| AM19410-AS | usAfsasUfcaaaggaCfaAfaAfuggcsa | 1225 | UAAUCAAAGGACAAAAUGGCA | 1606 |
| AM19411-AS | cPrpusAfaUfcaaaggaCfaAfaAfuggcsa | 1226 | UAAUCAAAGGACAAAAUGGCA | 1606 |
| AM19536-AS | asCfagAfauccugUfcUfuGfucausu | 1227 | ACAGAAUCCUGUCUUGUCAUU | 1650 |
| AM19538-AS | asCfagAfauccugUfcUfuGfucgusu | 1228 | ACAGAAUCCUGUCUUGUCGUU | 1657 |
| AM19538.1-AS | isCfagAfauccugUfcUfuGfucgusu | 1229 | ICAGAAUCCUGUCUUGUCGUU | 1658 |
| AM19539-AS | asCfagAfauccugUfcUfuGfucausc | 1230 | ACAGAAUCCUGUCUUGUCAUC | 1653 |
| AM19656-AS | usUfsasggaAfauccAfaUfgCfugucsu | 1231 | UUAGGAAAUCCAAUGCUGUCU | 1604 |
| AM19657-AS | cPrpusUfsasggaAfauccAfaUfgCfugucsu | 1232 | UUAGGAAAUCCAAUGCUGUCU | 1604 |
| AM19658-AS | usAfsusugaAfgcauUfgAfgAfcaccsa | 1233 | UAUUGAAGCAUUGAGACACCA | 1560 |
| AM19659-AS | cPrpusAfuugaAfgcauUfgAfgAfcaccsa | 1234 | UAUUGAAGCAUUGAGACACCA | 1560 |
| AM19661-AS | usAfsusugaAfgcauUfgAfgAfcaccsg | 1235 | UAUUGAAGCAUUGAGACACCG | 1659 |
| AM19663-AS | usAfsusugaAfgcauUfgAfgAfcaucsg | 1236 | UAUUGAAGCAUUGAGACAUCG | 1660 |
| CA004443 | cPrpusCfagAfauccugUfcUfuGfucgusu | 1237 | UCAGAAUCCUGUCUUGUCGUU | 1651 |
| CA004444 | cPrpusCfagAfauccugUfcUfuGfucausc | 1238 | UCAGAAUCCUGUCUUGUCAUC | 1652 |
| CA004446 | cPrpusCfagAfauccugUfcUfuGfucgusc | 1239 | UCAGAAUCCUGUCUUGUCGUC | 1661 |
| CA004447 | cPrpusCfagAfaU_{UNA}ccugUfcUfuGfucgusu | 1240 | UCAGAAUCCUGUCUUGUCGUU | 1651 |
| CA004449 | usUfuaAfcuuucuUfcUfcUfagccsu | 1241 | UUUAACUUUCUUCUCUAGCCU | 1662 |
| CA004481 | usAfsgsaAfuccuguCfuUfgUfcauusc | 1242 | UAGAAUCCUGUCUUGUCAUUC | 1663 |
| CA004483 | usUfscsaGfaauccuGfuCfuUfgucasc | 1243 | UUCAGAAUCCUGUCUUGUCAC | 1664 |
| CA004798 | isCfagAfauccugUfcUfuGfucgusu | 1244 | ICAGAAUCCUGUCUUGUCGUU | 1658 |
| CA005198 | asGfsgcauCfuaauauUfcCfaggsusu | 1245 | AGGCAUCUAAUAUUCCAGGUU | 1665 |
| CA008056 | usCfuuUfguguagGfcUfgCfacugsa | 1246 | UCUUUGUGUAGGCUGCACUGA | 1585 |
| CA008057 | asAfcuCfuucaguGfuUfuCfcagcsg | 1247 | AACUCUUCAGUGUUUCCAGCG | 1591 |
| CA008058 | usAfggUfacacagGfgUfcUfcccasc | 1248 | UAGGUACACAGGGUCUCCCAC | 1596 |
| CA008429 | cPrpusGfaaagaacuaguCfcauaausc | 1249 | UGAAAGAACUAGUCCAUAAUC | 1666 |
| CA008430 | cPrpusGfaaaGfaacuaUfuCfcAfuaasusc | 1250 | UGAAAGAACUAUUCCAUAAUC | 1608 |
| CA915324 | asCfagAfauccugUfcUfuGfucgusu | 1251 | ACAGAAUCCUGUCUUGUCGUU | 1657 |

TABLE 4
MARC1 RNAi Agent Sense Strand Sequences (Shown Without Linkers,
Conjugates, or Capping Moieties).

| | | | Underlying Base Sequence | |
|---|---|---|---|---|
| | | SEQ | (5′ → 3′) | SEQ |
| MARCI | Modified Sense Strand | ID | (Shown as an Unmodified | ID |
| Strand ID | (5′ → 3′) | NO. | Nucleotide Sequence) | NO. |
| AM16711-SS-NL | accaggagGfGfAfaacaugguua | 1252 | ACCAGGAGGGAAACAUGGUUA | 1667 |
| AM16713-SS-NL | gauaaccaGfCfUfuccugaagua | 1253 | GAUAACCAGCUUCCUGAAGUA | 1668 |
| AM16715-SS-NL | caucaaauAfGfCfagacuuguua | 1254 | CAUCAAAUAGCAGACUUGUUA | 1669 |
| AM16717-SS-NL | ccaaauagCfAfGfacuuiuucca | 1255 | CCAAAUAGCAGACUUIUUCCA | 1670 |
| AM16719-SS-NL | gaggaccaGfAfUfugcuuacuca | 1256 | GAGGACCAGAUUGCUUACUCA | 1671 |
| AM16721-SS-NL | accagauuGfCfUfuacucagaca | 1257 | ACCAGAUUGCUUACUCAGACA | 1672 |
| AM16723-SS-NL | gauugcuuAfCfUfcagacaccaa | 1258 | GAUUGCUUACUCAGACACCAA | 1673 |
| AM16725-SS-NL | caggcuagAfGfAfagaaaguuaa | 1259 | CAGGCUAGAGAAGAAAGUUAA | 1674 |
| AM16727-SS-NL | guuccagaUfGfCfauuuuaacca | 1260 | GUUCCAGAUGCAUUUUAACCA | 1675 |
| AM16729-SS-NL | ugacccuuCfAfGfaacgaaaguu | 1261 | UGACCCUUCAGAACGAAAGUU | 1676 |
| AM16731-SS-NL | cagaacgaAfAfGfuuauauggaa | 1262 | CAGAACGAAAGUUAUAUGGAA | 1677 |
| AM16733-SS-NL | cgaaaguuAfUfAfuggaaaauca | 1263 | CGAAAGUUAUAUGGAAAAUCA | 1678 |
| AM16735-SS-NL | cauggaaaAfUfCfaccacucuuu | 1264 | CAUGGAAAAUCACCACUCUUU | 1679 |
| AM16737-SS-NL | guguccugGfAfAfuauuagauga | 1265 | GUGUCCUGGAAUAUUAGAUGA | 1680 |
| AM16739-SS-NL | guucucaaAfAfAfugacaacacu | 1266 | GUUCUCAAAAAUGACAACACU | 1681 |
| AM16741-SS-NL | gaugacaaCfAfCfuugaagcaua | 1267 | GAUGACAACACUUGAAGCAUA | 1682 |
| AM16743-SS-NL | gcaacacuUfGfAfagcauggugu | 1268 | GCAACACUUGAAGCAUGGUGU | 1683 |
| AM16745-SS-NL | gaagcaugGfUfGfuuucagaacu | 1269 | GAAGCAUGGUGUUUCAGAACU | 1684 |
| AM16747-SS-NL | uggugucuCfAfAfugcuucaaua | 1270 | UGGUGUCUCAAUGCUUCAAUA | 1685 |
| AM16749-SS-NL | cgucucaaUfGfCfuucaauguca | 1271 | CGUCUCAAUGCUUCAAUGUCA | 1686 |
| AM16751-SS-NL | ggacaggaUfUfCfugaaaacuca | 1272 | GGACAGGAUUCUGAAAACUCA | 1687 |
| AM16753-SS-NL | gcugauuaUfGfGfaauaguucuu | 1273 | GCUGAUUAUGGAAUAGUUCUU | 1688 |
| AM16755-SS-NL | gauuauggAfAfUfaguucuuucu | 1274 | GAUUAUGGAAUAGUUCUUUCU | 1689 |
| AM16757-SS-NL | guuuuccaUfAfGfaucuigaucu | 1275 | GUUUUCCAUAGAUCUIGAUCU | 1690 |
| AM16759-SS-NL | gcuucucaGfAfCfagcauuggau | 1276 | GCUUCUCAGACAGCAUUGGAU | 1691 |
| AM16761-SS-NL | ggcauuggAfUfUfuccuaaaggu | 1277 | GGCAUUGGAUUUCCUAAAGGU | 1692 |
| AM16763-SS-NL | cauuggauUfUfCfcuaaagguia | 1278 | CAUUGGAUUUCCUAAAGGUIA | 1693 |
| AM16765-SS-NL | caugucagUfUfGfuuuaaaacca | 1279 | CAUGUCAGUUGUUUAAAACCA | 1694 |
| AM16767-SS-NL | guaacucuAfAfGfaucugaugaa | 1280 | GUAACUCUAAGAUCUGAUGAA | 1695 |
| AM16769-SS-NL | caacucuaAfGfAfucugaugaaa | 1281 | CAACUCUAAGAUCUGAUGAAA | 1696 |
| AM16771-SS-NL | ugggaaguUfGfAfcuaaacuuga | 1282 | UGGGAAGUUGACUAAACUUGA | 1697 |
| AM16773-SS-NL | gcugugaaUfAfAfauggaagcua | 1283 | GCUGUGAAUAAAUGGAAGCUA | 1698 |
| AM17487-SS-NL | cagguuuuGfGfCfuugugaucaa | 1284 | CAGGUUUUGGCUUGUGAUCAA | 1699 |
| AM17489-SS-NL | uucaggauGfCfGfaugucuauga | 1285 | UUCAGGAUGCGAUGUCUAUGA | 1700 |
| AM17491-SS-NL | uggaaaauCfAfCfcacucuuuga | 1286 | UGGAAAAUCACCACUCUUUGA | 1701 |
| AM17493-SS-NL | uugaagcaUfGfGfuguuucagaa | 1287 | UUGAAGCAUGGUGUUUCAGAA | 1702 |
| AM17495-SS-NL | aaugacaaGfAfCfaggauucuga | 1288 | AAUGACAAGACAGGAUUCUGA | 1703 |
| AM17497-SS-NL | cugcuucuCfAfGfacagcauuga | 1289 | CUGCUUCUCAGACAGCAUUGA | 1704 |
| AM17499-SS-NL | cuacugaaAfAfCfcuuuaaagga | 1290 | CUACUGAAAACCUUUAAAGGA | 1705 |
| AM17501-SS-NL | acucuaagAfUfCfugaugaagua | 1291 | ACUCUAAGAUCUGAUGAAGUA | 1706 |
| AM17503-SS-NL | uugggaagUfUfGfacuaaacuua | 1292 | UUGGGAAGUUGACUAAACUUA | 1707 |
| AM17505-SS-NL | cugcuucuCfAfGfacaguauuga | 1293 | CUGCUUCUCAGACAGUAUUGA | 1708 |
| AM17807-SS-NL | acucucagUfGfCfagccuacaca | 1294 | ACUCUCAGUGCAGCCUACACA | 1709 |
| AM17809-SS-NL | cucucaguGfCfAfgccuacacaa | 1295 | CUCUCAGUGCAGCCUACACAA | 1710 |
| AM17811-SS-NL | ucagugcaGfCfCfuacacaaaga | 1296 | UCAGUGCAGCCUACACAAAGA | 1711 |
| AM17813-SS-NL | agugcagcCfUfAfcacaaaggaa | 1297 | AGUGCAGCCUACACAAAGGAA | 1712 |
| AM17815-SS-NL | uaccgccuGfGfUfgcacuucgaa | 1298 | UACCGCCUGGUGCACUUCGAA | 1713 |
| AM17817-SS-NL | accgccugGfUfGfcacuucgaga | 1299 | ACCGCCUGGUGCACUUCGAGA | 1714 |
| AM17819-SS-NL | ccgccuggUfGfCfacuucgagca | 1300 | CCGCCUGGUGCACUUCGAGCA | 1715 |
| AM17821-SS-NL | cgccugguGfCfAfcuucgagccu | 1301 | CGCCUGGUGCACUUCGAGCCU | 1716 |
| AM17823-SS-NL | cgcuggaaAfCfAfcugaagaguu | 1302 | CGCUGGAAACACUGAAGAGUU | 1717 |
| AM17825-SS-NL | aucaaaguGfGfGfagacccugua | 1303 | AUCAAAGUGGGAGACCCUGUA | 1718 |
| AM17827-SS-NL | ucaaagugGfGfAfgacccugugu | 1304 | UCAAAGUGGGAGACCCUGUGU | 1719 |
| AM17829-SS-NL | aagugggaGfAfCfccuguguaca | 1305 | AAGUGGGAGACCCUGUGUACA | 1720 |
| AM17831-SS-NL | agugggagAfCfCfcuguguaccu | 1306 | AGUGGGAGACCCUGUGUACCU | 1721 |
| AM17833-SS-NL | gugggagaCfCfCfuguguaccua | 1307 | GUGGGAGACCCUGUGUACCUA | 1722 |
| AM17863-SS-NL | aucaaauaGfCfAfgacuuguuca | 1308 | AUCAAAUAGCAGACUUGUUCA | 1723 |
| AM17865-SS-NL | cuagagaaGfAfAfaguuaaagca | 1309 | CUAGAGAAGAAAGUUAAAGCA | 1724 |
| AM17867-SS-NL | agagaagaAfAfGfuuaaagcaaa | 1310 | AGAGAAGAAAGUUAAAGCAAA | 1725 |
| AM17869-SS-NL | aggcccaaUfAfUfuguaauuuca | 1311 | AGGCCCAAUAUUGUAAUUUCA | 1726 |
| AM17871-SS-NL | gacccuucAfGfAfacgaaaguua | 1312 | GACCCUUCAGAACGAAAGUUA | 1727 |
| AM17873-SS-NL | uucagaacGfAfAfaguuauauga | 1313 | UUCAGAACGAAAGUUAUAUGA | 1728 |
| AM17875-SS-NL | cccguuuaAfCfUfgauuauggaa | 1314 | CCCGUUUAACUGAUUAUGGAA | 1729 |
| AM17877-SS-NL | agacagcaUfUfGfgauuuccuaa | 1315 | AGACAGCAUUGGAUUUCCUAA | 1730 |
| AM17879-SS-NL | uuauugccAfUfUfuuguccuuua | 1316 | UUAUUGCCAUUUUGUCCUUUA | 1731 |
| AM17881-SS-NL | ugccauuuUfGfUfccuuugauua | 1317 | UGCCAUUUUGUCCUUUGAUUA | 1732 |
| AM18392-SS-NL | ccacugaaAfAfCfcuuuaaagga | 1318 | CCACUGAAAACCUUUAAAGGA | 1733 |
| AM18400-SS-NL | gauuauggAfAfUfaguucuuuca | 1319 | GAUUAUGGAAUAGUUCUUUCA | 1734 |
| AM18406-SS-NL | guucucaaAfAfAfugacaacaca | 1320 | GUUCUCAAAAAUGACAACACA | 1735 |
| AM18613-SS-NL | agguuuugGfCfUfugugaucaaa | 1321 | AGGUUUUGGCUUGUGAUCAAA | 1736 |
| AM18615-SS-NL | gguuuuggCfUfUfgugaucaaca | 1322 | GGUUUUGGCUUGUGAUCAACA | 1737 |
| AM18630-SS-NL | gauuauggAfaUfaGfuucuuucu | 1323 | GAUUAUGGAAUAGUUCUUUCU | 1689 |
| AM18631-SS-NL | gauuauggAfaUfaguucuuucu | 1324 | GAUUAUGGAAUAGUUCUUUCU | 1689 |
| AM18633-SS-NL | auuauggaAfUfAfguucuuucua | 1325 | AUUAUGGAAUAGUUCUUUCUA | 1738 |
| AM18635-SS-NL | acuauggaAfUfAfguucuuucua | 1326 | ACUAUGGAAUAGUUCUUUCUA | 1739 |
| AM18637-SS-NL | gcuauggaAfUfAfguucuuucua | 1327 | GCUAUGGAAUAGUUCUUUCUA | 1740 |
| AM18877-SS-NL | ugacuaguUfUfCfagaucuuacu | 1328 | UGACUAGUUUCAGAUCUUACU | 1741 |
| AM18879-SS-NL | uaguuucaGfAfUfcuuacuaacu | 1329 | UAGUUUCAGAUCUUACUAACU | 1742 |
| AM18881-SS-NL | guuucagaUfCfUfuacuaacuua | 1330 | GUUUCAGAUCUUACUAACUUA | 1743 |
| AM18883-SS-NL | gugcauguGfCfUfuguuuuauga | 1331 | GUGCAUGUGCUUGUUUUAUGA | 1744 |
| AM18885-SS-NL | gagacaaaGfCfGfaaguguucaa | 1332 | GAGACAAAGCGAAGUGUUCAA | 1745 |
| AM18887-SS-NL | uuccagugAfCfCfucuagaaucu | 1333 | UUCCAGUGACCUCUAGAAUCU | 1746 |
| AM18889-SS-NL | gagcauuuGfCfAfcacuauucca | 1334 | GAGCAUUUGCACACUAUUCCA | 1747 |
| AM18891-SS-NL | augacagaUfGfUfuugagaucaa | 1335 | AUGACAGAUGUUUGAGAUCAA | 1748 |
| AM18893-SS-NL | ucugugugGfCfAfacuuaugcua | 1336 | UCUGUGUGGCAACUUAUGCUA | 1749 |
| AM18895-SS-NL | cagaccagAfCfCfuugauuguga | 1337 | CAGACCAGACCUUGAUUGUGA | 1750 |
| AM18897-SS-NL | agggagaaGfCfAfcucucuuucu | 1338 | AGGGAGAAGCACUCUCUUUCU | 1751 |
| AM18899-SS-NL | gaaaccaaAfCfUfcucucuaaca | 1339 | GAAACCAAACUCUCUCUAACA | 1752 |
| AM18901-SS-NL | accaaacuCfUfCfucuaaccuua | 1340 | ACCAAACUCUCUCUAACCUUA | 1753 |
| AM18903-SS-NL | gugauguaAfGfUfuacaaaacca | 1341 | GUGAUGUAAGUUACAAAACCA | 1754 |
| AM18905-SS-NL | aaccaccuGfUfGfaugaaaguga | 1342 | AACCACCUGUGAUGAAAGUGA | 1755 |
| AM18907-SS-NL | ugacugguGfCfAfuuuauaauca | 1343 | UGACUGGUGCAUUUAUAAUCA | 1756 |
| AM18909-SS-NL | acagcucuGfAfCfuauaacucua | 1344 | ACAGCUCUGACUAUAACUCUA | 1757 |
| AM18911-SS-NL | uaccuuuaCfAfGfagcauccuaa | 1345 | UACCUUUACAGAGCAUCCUAA | 1758 |
| AM18913-SS-NL | ggaacuaaGfUfUfaagucuaacu | 1346 | GGAACUAAGUUAAGUCUAACU | 1759 |
| AM18915-SS-NL | cucagagaGfAfUfugaauuuacu | 1347 | CUCAGAGAGAUUGAAUUUACU | 1760 |
| AM18917-SS-NL | cagcucugGfUfUfaacaucaaca | 1348 | CAGCUCUGGUUAACAUCAACA | 1761 |
| AM18919-SS-NL | guugaccuGfAfCfaucauaguuu | 1349 | GUUGACCUGACAUCAUAGUUU | 1762 |
| AM18921-SS-NL | ucaaacagAfUfCfucguuaaugu | 1350 | UCAAACAGAUCUCGUUAAUGU | 1763 |
| AM18923-SS-NL | guucuuaaAfGfCfuauauucuga | 1351 | GUUCUUAAAGCUAUAUUCUGA | 1764 |
| AM18943-SS-NL | cuacacaaAfGfGfaccuacuacu | 1352 | CUACACAAAGGACCUACUACU | 1765 |
| AM18945-SS-NL | gacguccuCfAfUfcaaauagcaa | 1353 | GACGUCCUCAUCAAAUAGCAA | 1766 |
| AM18947-SS-NL | ucagaacuGfAfGfaccucuacau | 1354 | UCAGAACUGAGACCUCUACAU | 1767 |
| AM18949-SS-NL | ugagaccuCfUfAfcauuuucuuu | 1355 | UGAGACCUCUACAUUUUCUUU | 1768 |
| AM18951-SS-NL | ugugauuuUfCfAfcauuuuucgu | 1356 | UGUGAUUUUCACAUUUUUCGU | 1769 |
| AM18953-SS-NL | aacuuaguAfGfGfacuucaguaa | 1357 | AACUUAGUAGGACUUCAGUAA | 1770 |
| AM18955-SS-NL | cuccugcuUfCfUfccguuuaucu | 1358 | CUCCUGCUUCUCCGUUUAUCU | 1771 |
| AM18957-SS-NL | uggaagaaUfAfUfccuagaaugu | 1359 | UGGAAGAAUAUCCUAGAAUGU | 1772 |
| AM18959-SS-NL | aaggaaagCfAfUfaugucaguua | 1360 | AAGGAAAGCAUAUGUCAGUUA | 1773 |
| AM19119-SS-NL | cuacugaaAfAfCfcuuuaaaiga | 1361 | CUACUGAAAACCUUUAAAIGA | 1774 |
| AM19124-SS-NL | cuacugaaAfaCfcUfuuaaagga | 1362 | CUACUGAAAACCUUUAAAGGA | 1705 |
| AM19129-SS-NL | gugggagaCfcCfuGfuguaccua | 1363 | GUGGGAGACCCUGUGUACCUA | 1722 |
| AM19130-SS-NL | gugggagaCfCfCfuguguaucua | 1364 | GUGGGAGACCCUGUGUAUCUA | 1775 |
| AM19184-SS-NL | aaugacaaGfAfCfaggauucugu | 1365 | AAUGACAAGACAGGAUUCUGU | 1776 |
| AM19187-SS-NL | aaugacaaGfaCfaGfgauucuga | 1366 | AAUGACAAGACAGGAUUCUGA | 1703 |
| AM19188-SS-NL | aacgacaaGfAfCfaggauucuga | 1367 | AACGACAAGACAGGAUUCUGA | 1777 |
| AM19190-SS-NL | gaugacaaGfAfCfaggauucuga | 1368 | GAUGACAAGACAGGAUUCUGA | 1778 |
| AM19192-SS-NL | gaugacaaGfaCfaGfgauucugu | 1369 | GAUGACAAGACAGGAUUCUGU | 1779 |
| AM19194-SS-NL | gaugacaaGfAfCfaggauucugu | 1370 | GAUGACAAGACAGGAUUCUGU | 1779 |
| AM19218-SS-NL | guccuggaAfUfAfuuagaugccu | 1371 | GUCCUGGAAUAUUAGAUGCCU | 1780 |
| AM19220-SS-NL | uguccuggAfAfUfauuagaugcu | 1372 | UGUCCUGGAAUAUUAGAUGCU | 1781 |
| AM19222-SS-NL | uccuggaaUfAfUfuagaugccuu | 1373 | UCCUGGAAUAUUAGAUGCCUU | 1782 |
| AM19404-SS-NL | guucucaaAfaAfuGfacaacaca | 1374 | GUUCUCAAAAAUGACAACACA | 1735 |
| AM19409-SS-NL | ugccauuuUfgUfcCfuuugauua | 1375 | UGCCAUUUUGUCCUUUGAUUA | 1732 |
| AM19537-SS-NL | aacgacaaGfAfCfaggauucugu | 1376 | AACGACAAGACAGGAUUCUGU | 1783 |
| AM19660-SS-NL | cggugucuCfAfAfugcuucaaua | 1377 | CGGUGUCUCAAUGCUUCAAUA | 1784 |
| AM19662-SS-NL | cgaugucuCfAfAfugcuucaaua | 1378 | CGAUGUCUCAAUGCUUCAAUA | 1785 |
| CS004445-NL | gacgacaaGfAfCfaggauucuga | 1379 | GACGACAAGACAGGAUUCUGA | 1786 |
| CS004448-NL | aggcuagaGfAfAfgaaaguuaaa | 1380 | AGGCUAGAGAAGAAAGUUAAA | 1787 |
| CS004480-NL | gaaugacaAfGfAfcaggauucua | 1381 | GAAUGACAAGACAGGAUUCUA | 1788 |
| CS004482-NL | gugacaagAfCfAfggauucugaa | 1382 | GUGACAAGACAGGAUUCUGAA | 1789 |
| CS005197-NL | ccuggaAfuAfUfUfAfgaugccuu | 1383 | CCUGGAAUAUUAGAUGCCUU | 1790 |
| CS008428-NL | gauuauggAfAfUfaggucuuuca | 1389 | GAUUAUGGAAUAGGUCUUUCA | 1791 |
| CS913717-NL | ucagugcaGfCfCfuacacaaaga | 1390 | UCAGUGCAGCCUACACAAAGA | 1711 |
| CS913729-NL | cgcuggaaAfCfAfcugaagaguu | 1391 | CGCUGGAAACACUGAAGAGUU | 1717 |
| CS913739-NL | gugggagaCfCfCfuguguaccua | 1392 | GUGGGAGACCCUGUGUACCUA | 1722 |
| CS914289-NL | gauuauggAfAfUfaguucuuuca | 1393 | GAUUAUGGAAUAGUUCUUUCA | 1734 |
| CS914989-NL | aacgacaaGfAfCfaggauucuga | 1394 | AACGACAAGACAGGAUUCUGA | 1777 |
| CS914991-NL | gaugacaaGfAfCfaggauucuga | 1395 | GAUGACAAGACAGGAUUCUGA | 1778 |
| CS915323-NL | aacgacaaGfAfCfaggauucugu | 1396 | AACGACAAGACAGGAUUCUGU | 1783 |

(A^{2N})=2-aminoadenosine nucleotide; I = hypoxanthine (inosine) nucleotide

TABLE 5
MARC1 RNAi Agent Sense Strand Sequences (Shown With (NAG37) Targeting Ligand (see Table 7 for structure information)).

| | | | Underlying Base Sequence (5′→3′) | |
|---|---|---|---|---|
| | | | (Shown as an Unmodified | |
| Strand ID | Modified Sense Strand (5′→3′) | SEQ ID NO. | Nucleotide Sequence) | SEQ ID NO. |
| AM16711-SS | (NAG37)s(invAb)saccaggagGfGfAfaacaugguuas(invAb) | 1397 | ACCAGGAGGGAAACAUGGUUA | 1667 |
| AM16713-SS | (NAG37)s(invAb)sgauaaccaGfCfUfuccugaaguas(invAb) | 1398 | GAUAACCAGCUUCCUGAAGUA | 1668 |
| AM16715-SS | (NAG37)s(invAb)scaucaaauAfGfCfagacuuguuas(invAb) | 1399 | CAUCAAAUAGCAGACUUGUUA | 1669 |
| AM16717-SS | (NAG37)s(invAb)sccaaauagCfAfGfacuuiuuccas(invAb) | 1400 | CCAAAUAGCAGACUUIUUCCA | 1670 |
| AM16719-SS | (NAG37)s(invAb)sgaggaccaGfAfUfugcuuacucas(invAb) | 1401 | GAGGACCAGAUUGCUUACUCA | 1671 |
| AM16721-SS | (NAG37)s(invAb)saccagauuGfCfUfuacucagacas(invAb) | 1402 | ACCAGAUUGCUUACUCAGACA | 1672 |
| AM16723-SS | (NAG37)s(invAb)sgauugcuuAfCfUfcagacaccaas(invAb) | 1403 | GAUUGCUUACUCAGACACCAA | 1673 |
| AM16725-SS | (NAG37)s(invAb)scaggcuagAfGfAfagaaaguuaas(invAb) | 1404 | CAGGCUAGAGAAGAAAGUUAA | 1674 |
| AM16727-SS | (NAG37)s(invAb)sguuccagaUfGfCfauuuuaaccas(invAb) | 1405 | GUUCCAGAUGCAUUUUAACCA | 1675 |
| AM16729-SS | (NAG37)s(invAb)sugacccuuCfAfGfaacgaaaguus(invAb) | 1406 | UGACCCUUCAGAACGAAAGUU | 1676 |
| AM16731-SS | (NAG37)s(invAb)scagaacgaAfAfGfuuauauggaas(invAb) | 1407 | CAGAACGAAAGUUAUAUGGAA | 1677 |
| AM16733-SS | (NAG37)s(invAb)scgaaaguuAfUfAfuggaaaaucas(invAb) | 1408 | CGAAAGUUAUAUGGAAAAUCA | 1678 |
| AM16735-SS | (NAG37)s(invAb)scauggaaaAfUfCfaccacucuuus(invAb) | 1409 | CAUGGAAAAUCACCACUCUUU | 1679 |
| AM16737-SS | (NAG37)s(invAb)sguguccugGfAfAfuauuagaugas(invAb) | 1410 | GUGUCCUGGAAUAUUAGAUGA | 1680 |
| AM16739-SS | (NAG37)s(invAb)sguucucaaAfAfAfugacaacacus(invAb) | 1411 | GUUCUCAAAAAUGACAACACU | 1681 |
| AM16741-SS | (NAG37)s(invAb)sgaugacaaCfAfCfuugaagcauas(invAb) | 1412 | GAUGACAACACUUGAAGCAUA | 1682 |
| AM16743-SS | (NAG37)s(invAb)sgcaacacuUfGfAfagcauggugus(invAb) | 1413 | GCAACACUUGAAGCAUGGUGU | 1683 |
| AM16745-SS | (NAG37)s(invAb)sgaagcaugGfUfGfuuucagaacus(invAb) | 1414 | GAAGCAUGGUGUUUCAGAACU | 1684 |
| AM16747-SS | (NAG37)s(invAb)suggugucuCfAfAfugcuucaauas(invAb) | 1415 | UGGUGUCUCAAUGCUUCAAUA | 1685 |
| AM16749-SS | (NAG37)s(invAb)scgucucaaUfGfCfuucaaugucas(invAb) | 1416 | CGUCUCAAUGCUUCAAUGUCA | 1686 |
| AM16751-SS | (NAG37)s(invAb)sggacaggaUfUfCfugaaaacucas(invAb) | 1417 | GGACAGGAUUCUGAAAACUCA | 1687 |
| AM16753-SS | (NAG37)s(invAb)sgcugauuaUfGfGfaauaguucuus(invAb) | 1418 | GCUGAUUAUGGAAUAGUUCUU | 1688 |
| AM16755-SS | (NAG37)s(invAb)sgauuauggAfAfUfaguucuuucus(invAb) | 1419 | GAUUAUGGAAUAGUUCUUUCU | 1689 |
| AM16757-SS | (NAG37)s(invAb)sguuuuccaUfAfGfaucuigaucus(invAb) | 1420 | GUUUUCCAUAGAUCUIGAUCU | 1690 |
| AM16759-SS | (NAG37)s(invAb)sgcuucucaGfAfCfagcauuggaus(invAb) | 1421 | GCUUCUCAGACAGCAUUGGAU | 1691 |
| AM16761-SS | (NAG37)s(invAb)sggcauuggAfUfUfuccuaaaggus(invAb) | 1422 | GGCAUUGGAUUUCCUAAAGGU | 1692 |
| AM16763-SS | (NAG37)s(invAb)scauuggauUfUfCfcuaaagguias(invAb) | 1423 | CAUUGGAUUUCCUAAAGGUIA | 1693 |
| AM16765-SS | (NAG37)s(invAb)scaugucagUfUfGfuuuaaaaccas(invAb) | 1424 | CAUGUCAGUUGUUUAAAACCA | 1694 |
| AM16767-SS | (NAG37)s(invAb)sguaacucuAfAfGfaucugaugaas(invAb) | 1425 | GUAACUCUAAGAUCUGAUGAA | 1695 |
| AM16769-SS | (NAG37)s(invAb)scaacucuaAfGfAfucugaugaaas(invAb) | 1426 | CAACUCUAAGAUCUGAUGAAA | 1696 |
| AM16771-SS | (NAG37)s(invAb)sugggaaguUfGfAfcuaaacuugas(invAb) | 1427 | UGGGAAGUUGACUAAACUUGA | 1697 |
| AM16773-SS | (NAG37)s(invAb)sgcugugaaUfAfAfauggaagcuas(invAb) | 1428 | GCUGUGAAUAAAUGGAAGCUA | 1698 |
| AM17487-SS | (NAG37)s(invAb)scagguuuuGfGfCfuugugaucaas(invAb) | 1429 | CAGGUUUUGGCUUGUGAUCAA | 1699 |
| AM17489-SS | (NAG37)s(invAb)suucaggauGfCfGfaugucuaugas(invAb) | 1430 | UUCAGGAUGCGAUGUCUAUGA | 1700 |
| AM17491-SS | (NAG37)s(invAb)suggaaaauCfAfCfcacucuuugas(invAb) | 1431 | UGGAAAAUCACCACUCUUUGA | 1701 |
| AM17493-SS | (NAG37)s(invAb)suugaagcaUfGfGfuguuucagaas(invAb) | 1432 | UUGAAGCAUGGUGUUUCAGAA | 1702 |
| AM17495-SS | (NAG37)s(invAb)saaugacaaGfAfCfaggauucugas(invAb) | 1433 | AAUGACAAGACAGGAUUCUGA | 1703 |
| AM17497-SS | (NAG37)s(invAb)scugcuucuCfAfGfacagcauugas(invAb) | 1434 | CUGCUUCUCAGACAGCAUUGA | 1704 |
| AM17499-SS | (NAG37)s(invAb)scuacugaaAfAfCfcuuuaaaggas(invAb) | 1435 | CUACUGAAAACCUUUAAAGGA | 1705 |
| AM17501-SS | (NAG37)s(invAb)sacucuaagAfUfCfugaugaaguas(invAb) | 1436 | ACUCUAAGAUCUGAUGAAGUA | 1706 |
| AM17503-SS | (NAG37)s(invAb)suugggaagUfUfGfacuaaacuuas(invAb) | 1437 | UUGGGAAGUUGACUAAACUUA | 1707 |
| AM17505-SS | (NAG37)s(invAb)scugcuucuCfAfGfacaguauugas(invAb) | 1438 | CUGCUUCUCAGACAGUAUUGA | 1708 |
| AM17807-SS | (NAG37)s(invAb)sacucucagUfGfCfagccuacacas(invAb) | 1439 | ACUCUCAGUGCAGCCUACACA | 1709 |
| AM17809-SS | (NAG37)s(invAb)scucucaguGfCfAfgccuacacaas(invAb) | 1440 | CUCUCAGUGCAGCCUACACAA | 1710 |
| AM17811-SS | (NAG37)s(invAb)sucagugcaGfCfCfuacacaaagas(invAb) | 1441 | UCAGUGCAGCCUACACAAAGA | 1711 |
| AM17813-SS | (NAG37)s(invAb)sagugcagcCfUfAfcacaaaggaas(invAb) | 1442 | AGUGCAGCCUACACAAAGGAA | 1712 |
| AM17815-SS | (NAG37)s(invAb)suaccgccuGfGfUfgcacuucgaas(invAb) | 1443 | UACCGCCUGGUGCACUUCGAA | 1713 |
| AM17817-SS | (NAG37)s(invAb)saccgccugGfUfGfcacuucgagas(invAb) | 1444 | ACCGCCUGGUGCACUUCGAGA | 1714 |
| AM17819-SS | (NAG37)s(invAb)sccgccuggUfGfCfacuucgagcas(invAb) | 1445 | CCGCCUGGUGCACUUCGAGCA | 1715 |
| AM17821-SS | (NAG37)s(invAb)scgccugguGfCfAfcuucgagccus(invAb) | 1446 | CGCCUGGUGCACUUCGAGCCU | 1716 |
| AM17823-SS | (NAG37)s(invAb)scgcuggaaAfCfAfcugaagaguus(invAb) | 1447 | CGCUGGAAACACUGAAGAGUU | 1717 |
| AM17825-SS | (NAG37)s(invAb)saucaaaguGfGfGfagacccuguas(invAb) | 1448 | AUCAAAGUGGGAGACCCUGUA | 1718 |
| AM17827-SS | (NAG37)s(invAb)sucaaagugGfGfAfgacccugugus(invAb) | 1449 | UCAAAGUGGGAGACCCUGUGU | 1719 |
| AM17829-SS | (NAG37)s(invAb)saagugggaGfAfCfccuguguacas(invAb) | 1450 | AAGUGGGAGACCCUGUGUACA | 1720 |
| AM17831-SS | (NAG37)s(invAb)sagugggagAfCfCfcuguguaccus(invAb) | 1451 | AGUGGGAGACCCUGUGUACCU | 1721 |
| AM17833-SS | (NAG37)s(invAb)sgugggagaCfCfCfuguguaccuas(invAb) | 1452 | GUGGGAGACCCUGUGUACCUA | 1722 |
| AM17863-SS | (NAG37)s(invAb)saucaaauaGfCfAfgacuuguucas(invAb) | 1453 | AUCAAAUAGCAGACUUGUUCA | 1723 |
| AM17865-SS | (NAG37)s(invAb)scuagagaaGfAfAfaguuaaagcas(invAb) | 1454 | CUAGAGAAGAAAGUUAAAGCA | 1724 |
| AM17867-SS | (NAG37)s(invAb)sagagaagaAfAfGfuuaaagcaaas(invAb) | 1455 | AGAGAAGAAAGUUAAAGCAAA | 1725 |
| AM17869-SS | (NAG37)s(invAb)saggcccaaUfAfUfuguaauuucas(invAb) | 1456 | AGGCCCAAUAUUGUAAUUUCA | 1726 |
| AM17871-SS | (NAG37)s(invAb)sgacccuucAfGfAfacgaaaguuas(invAb) | 1457 | GACCCUUCAGAACGAAAGUUA | 1727 |
| AM17873-SS | (NAG37)s(invAb)suucagaacGfAfAfaguuauaugas(invAb) | 1458 | UUCAGAACGAAAGUUAUAUGA | 1728 |
| AM17875-SS | (NAG37)s(invAb)scccguuuaAfCfUfgauuauggaas(invAb) | 1459 | CCCGUUUAACUGAUUAUGGAA | 1729 |
| AM17877-SS | (NAG37)s(invAb)sagacagcaUfUfGfgauuuccuaas(invAb) | 1460 | AGACAGCAUUGGAUUUCCUAA | 1730 |
| AM17879-SS | (NAG37)s(invAb)suuauugccAfUfUfuuguccuuuas(invAb) | 1461 | UUAUUGCCAUUUUGUCCUUUA | 1731 |
| AM17881-SS | (NAG37)s(invAb)sugccauuuUfGfUfccuuugauuas(invAb) | 1462 | UGCCAUUUUGUCCUUUGAUUA | 1732 |
| AM18392-SS | (NAG37)s(invAb)sccacugaaAfAfCfcuuuaaaggas(invAb) | 1463 | CCACUGAAAACCUUUAAAGGA | 1733 |
| AM18400-SS | (NAG37)s(invAb)sgauuauggAfAfUfaguucuuucas(invAb) | 1464 | GAUUAUGGAAUAGUUCUUUCA | 1734 |
| AM18406-SS | (NAG37)s(invAb)sguucucaaAfAfAfugacaacacas(invAb) | 1465 | GUUCUCAAAAAUGACAACACA | 1735 |
| AM18613-SS | (NAG37)s(invAb)sagguuuugGfCfUfugugaucaaas(invAb) | 1466 | AGGUUUUGGCUUGUGAUCAAA | 1736 |
| AM18615-SS | (NAG37)s(invAb)sgguuuuggCfUfUfgugaucaacas(invAb) | 1467 | GGUUUUGGCUUGUGAUCAACA | 1737 |
| AM18630-SS | (NAG37)s(invAb)sgauuauggAfaUfaGfuucuuucus(invAb) | 1468 | GAUUAUGGAAUAGUUCUUUCU | 1689 |
| AM18631-SS | (NAG37)s(invAb)sgauuauggAfaUfaguucuuucus(invAb) | 1469 | GAUUAUGGAAUAGUUCUUUCU | 1689 |
| AM18633-SS | (NAG37)s(invAb)sauuauggaAfUfAfguucuuucuas(invAb) | 1470 | AUUAUGGAAUAGUUCUUUCUA | 1738 |
| AM18635-SS | (NAG37)s(invAb)sacuauggaAfUfAfguucuuucuas(invAb) | 1471 | ACUAUGGAAUAGUUCUUUCUA | 1739 |
| AM18637-SS | (NAG37)s(invAb)sgcuauggaAfUfAfguucuuucuas(invAb) | 1472 | GCUAUGGAAUAGUUCUUUCUA | 1740 |
| AM18877-SS | (NAG37)s(invAb)sugacuaguUfUfCfagaucuuacus(invAb) | 1473 | UGACUAGUUUCAGAUCUUACU | 1741 |
| AM18879-SS | (NAG37)s(invAb)suaguuucaGfAfUfcuuacuaacus(invAb) | 1474 | UAGUUUCAGAUCUUACUAACU | 1742 |
| AM18881-SS | (NAG37)s(invAb)sguuucagaUfCfUfuacuaacuuas(invAb) | 1475 | GUUUCAGAUCUUACUAACUUA | 1743 |
| AM18883-SS | (NAG37)s(invAb)sgugcauguGfCfUfuguuuuaugas(invAb) | 1476 | GUGCAUGUGCUUGUUUUAUGA | 1744 |
| AM18885-SS | (NAG37)s(invAb)sgagacaaaGfCfGfaaguguucaas(invAb) | 1477 | GAGACAAAGCGAAGUGUUCAA | 1745 |
| AM18887-SS | (NAG37)s(invAb)suuccagugAfCfCfucuagaaucus(invAb) | 1478 | UUCCAGUGACCUCUAGAAUCU | 1746 |
| AM18889-SS | (NAG37)s(invAb)sgagcauuuGfCfAfcacuauuccas(invAb) | 1479 | GAGCAUUUGCACACUAUUCCA | 1747 |
| AM18891-SS | (NAG37)s(invAb)saugacagaUfGfUfuugagaucaas(invAb) | 1480 | AUGACAGAUGUUUGAGAUCAA | 1748 |
| AM18893-SS | (NAG37)s(invAb)sucugugugGfCfAfacuuaugcuas(invAb) | 1481 | UCUGUGUGGCAACUUAUGCUA | 1749 |
| AM18895-SS | (NAG37)s(invAb)scagaccagAfCfCfuugauugugas(invAb) | 1482 | CAGACCAGACCUUGAUUGUGA | 1750 |
| AM18897-SS | (NAG37)s(invAb)sagggagaaGfCfAfcucucuuucus(invAb) | 1483 | AGGGAGAAGCACUCUCUUUCU | 1751 |
| AM18899-SS | (NAG37)s(invAb)sgaaaccaaAfCfUfcucucuaacas(invAb) | 1484 | GAAACCAAACUCUCUCUAACA | 1752 |
| AM18901-SS | (NAG37)s(invAb)saccaaacuCfUfCfucuaaccuuas(invAb) | 1485 | ACCAAACUCUCUCUAACCUUA | 1753 |
| AM18903-SS | (NAG37)s(invAb)sgugauguaAfGfUfuacaaaaccas(invAb) | 1486 | GUGAUGUAAGUUACAAAACCA | 1754 |
| AM18905-SS | (NAG37)s(invAb)saaccaccuGfUfGfaugaaagugas(invAb) | 1487 | AACCACCUGUGAUGAAAGUGA | 1755 |
| AM18907-SS | (NAG37)s(invAb)sugacugguGfCfAfuuuauaaucas(invAb) | 1488 | UGACUGGUGCAUUUAUAAUCA | 1756 |
| AM18909-SS | (NAG37)s(invAb)sacagcucuGfAfCfuauaacucuas(invAb) | 1489 | ACAGCUCUGACUAUAACUCUA | 1757 |
| AM18911-SS | (NAG37)s(invAb)suaccuuuaCfAfGfagcauccuaas(invAb) | 1490 | UACCUUUACAGAGCAUCCUAA | 1758 |
| AM18913-SS | (NAG37)s(invAb)sggaacuaaGfUfUfaagucuaacus(invAb) | 1491 | GGAACUAAGUUAAGUCUAACU | 1759 |
| AM18915-SS | (NAG37)s(invAb)scucagagaGfAfUfugaauuuacus(invAb) | 1492 | CUCAGAGAGAUUGAAUUUACU | 1760 |
| AM18917-SS | (NAG37)s(invAb)scagcucugGfUfUfaacaucaacas(invAb) | 1493 | CAGCUCUGGUUAACAUCAACA | 1761 |
| AM18919-SS | (NAG37)s(invAb)sguugaccuGfAfCfaucauaguuus(invAb) | 1494 | GUUGACCUGACAUCAUAGUUU | 1762 |
| AM18921-SS | (NAG37)s(invAb)sucaaacagAfUfCfucguuaaugus(invAb) | 1495 | UCAAACAGAUCUCGUUAAUGU | 1763 |
| AM18923-SS | (NAG37)s(invAb)sguucuuaaAfGfCfuauauucugas(invAb) | 1496 | GUUCUUAAAGCUAUAUUCUGA | 1764 |
| AM18943-SS | (NAG37)s(invAb)scuacacaaAfGfGfaccuacuacus(invAb) | 1497 | CUACACAAAGGACCUACUACU | 1765 |
| AM18945-SS | (NAG37)s(invAb)sgacguccuCfAfUfcaaauagcaas(invAb) | 1498 | GACGUCCUCAUCAAAUAGCAA | 1766 |
| AM18947-SS | (NAG37)s(invAb)sucagaacuGfAfGfaccucuacaus(invAb) | 1499 | UCAGAACUGAGACCUCUACAU | 1767 |
| AM18949-SS | (NAG37)s(invAb)sugagaccuCfUfAfcauuuucuuus(invAb) | 1500 | UGAGACCUCUACAUUUUCUUU | 1768 |
| AM18951-SS | (NAG37)s(invAb)sugugauuuUfCfAfcauuuuucgus(invAb) | 1501 | UGUGAUUUUCACAUUUUUCGU | 1769 |
| AM18953-SS | (NAG37)s(invAb)saacuuaguAfGfGfacuucaguaas(invAb) | 1502 | AACUUAGUAGGACUUCAGUAA | 1770 |
| AM18955-SS | (NAG37)s(invAb)scuccugcuUfCfUfccguuuaucus(invAb) | 1503 | CUCCUGCUUCUCCGUUUAUCU | 1771 |
| AM18957-SS | (NAG37)s(invAb)suggaagaaUfAfUfccuagaaugus(invAb) | 1504 | UGGAAGAAUAUCCUAGAAUGU | 1772 |
| AM18959-SS | (NAG37)s(invAb)saaggaaagCfAfUfaugucaguuas(invAb) | 1505 | AAGGAAAGCAUAUGUCAGUUA | 1773 |
| AM19119-SS | (NAG37)s(invAb)scuacugaaAfAfCfcuuuaaaigas(invAb) | 1506 | CUACUGAAAACCUUUAAAIGA | 1774 |
| AM19124-SS | (NAG37)s(invAb)scuacugaaAfaCfcUfuuaaaggas(invAb) | 1507 | CUACUGAAAACCUUUAAAGGA | 1705 |
| AM19129-SS | (NAG37)s(invAb)sgugggagaCfcCfuGfuguaccuas(invAb) | 1508 | GUGGGAGACCCUGUGUACCUA | 1722 |
| AM19130-SS | (NAG37)s(invAb)sgugggagaCfCfCfuguguaucuas(invAb) | 1509 | GUGGGAGACCCUGUGUAUCUA | 1775 |
| AM19184-SS | (NAG37)s(invAb)saaugacaaGfAfCfaggauucugus(invAb) | 1510 | AAUGACAAGACAGGAUUCUGU | 1776 |
| AM19187-SS | (NAG37)s(invAb)saaugacaaGfaCfaGfgauucugas(invAb) | 1511 | AAUGACAAGACAGGAUUCUGA | 1703 |
| AM19188-SS | (NAG37)s(invAb)saacgacaaGfAfCfaggauucugas(invAb) | 1512 | AACGACAAGACAGGAUUCUGA | 1777 |
| AM19190-SS | (NAG37)s(invAb)sgaugacaaGfAfCfaggauucugas(invAb) | 1513 | GAUGACAAGACAGGAUUCUGA | 1778 |
| AM19192-SS | (NAG37)s(invAb)sgaugacaaGfaCfaGfgauucugus(invAb) | 1514 | GAUGACAAGACAGGAUUCUGU | 1779 |
| AM19194-SS | (NAG37)s(invAb)sgaugacaaGfAfCfaggauucugus(invAb) | 1515 | GAUGACAAGACAGGAUUCUGU | 1779 |
| AM19218-SS | (NAG37)s(invAb)sguccuggaAfUfAfuuagaugccus(invAb) | 1516 | GUCCUGGAAUAUUAGAUGCCU | 1780 |
| AM19220-SS | (NAG37)s(invAb)suguccuggAfAfUfauuagaugcus(invAb) | 1517 | UGUCCUGGAAUAUUAGAUGCU | 1781 |
| AM19222-SS | (NAG37)s(invAb)succuggaaUfAfUfuagaugccuus(invAb) | 1518 | UCCUGGAAUAUUAGAUGCCUU | 1782 |
| AM19404-SS | (NAG37)s(invAb)sguucucaaAfaAfuGfacaacacas(invAb) | 1519 | GUUCUCAAAAAUGACAACACA | 1735 |
| AM19409-SS | (NAG37)s(invAb)sugccauuuUfgUfcCfuuugauuas(invAb) | 1520 | UGCCAUUUUGUCCUUUGAUUA | 1732 |
| AM19537-SS | (NAG37)s(invAb)saacgacaaGfAfCfaggauucugus(invAb) | 1521 | AACGACAAGACAGGAUUCUGU | 1783 |
| AM19660-SS | (NAG37)s(invAb)scggugucuCfAfAfugcuucaauas(invAb) | 1522 | CGGUGUCUCAAUGCUUCAAUA | 1784 |
| AM19662-SS | (NAG37)s(invAb)scgaugucuCfAfAfugcuucaauas(invAb) | 1523 | CGAUGUCUCAAUGCUUCAAUA | 1785 |
| CS004445 | (NAG37)s(invAb)sgacgacaaGfAfCfaggauucugas(invAb) | 1524 | GACGACAAGACAGGAUUCUGA | 1786 |
| CS004448 | (NAG37)s(invAb)saggcuagaGfAfAfgaaaguuaaas(invAb) | 1525 | AGGCUAGAGAAGAAAGUUAAA | 1787 |
| CS004480 | (NAG37)s(invAb)sgaaugacaAfGfAfcaggauucuas(invAb) | 1526 | GAAUGACAAGACAGGAUUCUA | 1788 |
| CS004482 | (NAG37)s(invAb)sgugacaagAfCfAfggauucugaas(invAb) | 1527 | GUGACAAGACAGGAUUCUGAA | 1789 |
| CS005197 | (NAG37)sccuggaAfuAfUfUfAfgaugccuus(invAb) | 1528 | CCUGGAAUAUUAGAUGCCUU | 1790 |
| CS008428 | (NAG37)s(invAb)sgauuauggAfAfUfaggucuuucas(invAb) | 1534 | GAUUAUGGAAUAGGUCUUUCA | 1791 |
| CS913717 | (NAG37)s(invAb)sucagugcaGfCfCfuacacaaagas(invAb) | 1535 | UCAGUGCAGCCUACACAAAGA | 1711 |
| CS913729 | (NAG37)s(invAb)scgcuggaaAfCfAfcugaagaguus(invAb) | 1536 | CGCUGGAAACACUGAAGAGUU | 1717 |
| CS913739 | (NAG37)s(invAb)sgugggagaCfCfCfuguguaccuas(invAb) | 1537 | GUGGGAGACCCUGUGUACCUA | 1722 |
| CS914289 | (NAG37)s(invAb)sgauuauggAfAfUfaguucuuucas(invAb) | 1538 | GAUUAUGGAAUAGUUCUUUCA | 1734 |
| CS914989 | (NAG37)s(invAb)saacgacaaGfAfCfaggauucugas(invAb) | 1539 | AACGACAAGACAGGAUUCUGA | 1777 |
| CS914991 | (NAG37)s(invAb)sgaugacaaGfAfCfaggauucugas(invAb) | 1540 | GAUGACAAGACAGGAUUCUGA | 1778 |
| CS915323 | (NAG37)s(invAb)saacgacaaGfAfCfaggauucugus(invAb) | 1541 | AACGACAAGACAGGAUUCUGU | 1783 |

(A^{2N})=2-aminoadenosine nucleotide; I = hypoxanthine (inosine) nucleotide

The MARC1 RNAi agents described herein are formed by annealing an antisense strand with a sense strand. A sense strand containing a sequence listed in Table 2, Table 4, or Table 5 can be hybridized to any antisense strand containing a sequence listed in Table 2, or Table 3 provided the two sequences have a region of at least 85% complementarity over a contiguous 15, 16, 17, 18, 19, 20, or 21 nucleotide sequence.

In some embodiments, the antisense strand of a MARC1 RNAi agent disclosed herein differs by 0. 1. 2, or 3 nucleotides from any of the antisense strand sequences in Table 3. In some embodiments, the sense strand of a MARC1 RNAi agent disclosed herein differs by 0, 1, 2, or 3 nucleotides from any of the sense strand sequences in Table 4 or Table 5.

In some embodiments, a MARC1 RNAi agent antisense strand comprises a nucleotide sequence of any of the sequences in Table 2, or Table 3. In some embodiments, a MARC1 RNAi agent antisense strand comprises the sequence of nucleotides (from 5′ end->3′ end) 1-17, 2-17, 1-18, 2-18, 1-19, 2-19, 1-20, 2-20, 1-21, or 2-21, of any of the sequences in Table 2 or Table 3. In certain embodiments, a MARC1 RNAi agent antisense strand comprises or consists of a modified sequence of any one of the modified sequences in Table 3.

In some embodiments, a MARC1 RNAi agent sense strand comprises the nucleotide sequence of any of the sequences in Table 2, Table 4, or Table 5. In some embodiments, a MARC1 RNAi agent sense strand comprises the sequence of nucleotides (from 5′ end->3′ end) 1-17, 2-17, 3-17, 4-17, 1-18, 2-18, 3-18, 4-18, 1-19, 2-19, 3-19, 4-19, 1-20, 2-20, 3-20, 4-20, 1-21, 2-21, 3-21, or 4-21, of any of the sequences in Table 2, Table 4, or Table 5. In certain embodiments, a MARC1 RNAi agent sense strand comprises or consists of a modified sequence of any one of the modified sequences in Table 4 or Table 5.

For the MARC1 RNAi agents disclosed herein, the nucleotide at position 1 of the antisense strand (from 5′ end->3′ end) can be perfectly complementary to a MARC1 gene, or can be non-complementary to a MARC1 gene. In some embodiments, the nucleotide at position 1 of the antisense strand (from 5′ end->3′ end) is a U, A, or dT (or a modified version thereof). In some embodiments, the nucleotide at position 1 of the antisense strand (from 5′ end →3′ end) forms an A: U or U: A base pair with the sense strand.

A sense strand containing a sequence listed in Table 2, Table 4, or Table 5 can be hybridized to any antisense strand containing a sequence listed in Table 2, or Table 3 provided the two sequences have a region of at least 85% complementarity over a contiguous 15. 16, 17, 18, 19, 20, or 21 nucleotide sequence. In some embodiments, the MARC1 RNAi agent has a sense strand consisting of the modified sequence of any of the modified sequences in Table 4 or Table 5, and an antisense strand consisting of the modified sequence of any of the modified sequences in Table 3. Certain representative sequence pairings are exemplified by the Duplex ID Nos. shown in Tables 6A or 6B.

In some embodiments, a MARC1 RNAi agent comprises, consists of, or consists essentially of a duplex represented by any one of the Duplex ID Nos. presented herein. In some embodiments, a MARC1 RNAi agent comprises the sense strand and antisense strand nucleotide sequences of any of the duplexes represented by any of the Duplex ID Nos. presented herein. In some embodiments, a MARC1 RNAi agent comprises the sense strand and antisense strand nucleotide sequences of any of the duplexes represented by any of the Duplex ID Nos. presented herein and a targeting group and/or linking group wherein the targeting group and/or linking group is covalently linked (i.e., conjugated) to the sense strand or the antisense strand. In some embodiments, a MARC1 RNAi agent includes the sense strand and antisense strand modified nucleotide sequences of any of the Duplex ID Nos. presented herein. In some embodiments, a MARC1 RNAi agent comprises the sense strand and antisense strand modified nucleotide sequences of any of the Duplex ID Nos. presented herein and a targeting group and/or linking group, wherein the targeting group and/or linking group is covalently linked to the sense strand or the antisense strand.

In some embodiments, a MARC1 RNAi agent comprises an antisense strand and a sense strand having the nucleotide sequences of any of the antisense strand/sense strand duplexes of Table 2 or Tables 6A and 6B, and further comprises a targeting group or targeting ligand. In some embodiments, a MARC1 RNAi agent comprises an antisense strand and a sense strand having the nucleotide sequences of any of the antisense strand/sense strand duplexes of Table 2 or Tables 6A and 6B, and further comprises an asialoglycoprotein receptor ligand targeting group.

A targeting group. with or without a linker, can be linked to the 5′ or 3′ end of any of the sense and/or antisense strands disclosed in Tables 2, 3, 4, or 5. A linker, with or without a targeting group, can be attached to the 5′ or 3′ end of any of the sense and/or antisense strands disclosed in Tables 2, 3, 4, and 5.

In some embodiments, a MARC1 RNAi agent comprises an antisense strand and a sense strand having the nucleotide sequences of any of the antisense strand/sense strand duplexes of Table 2. Table 6A, or Table 6B, and further comprises a targeting ligand selected from the group consisting of: (NAG37) and (NAG37) s, each as defined in Table 7.

In some embodiments, a MARC1 RNAi agent comprises an antisense strand and a sense strand having the modified nucleotide sequence of any of the antisense strand and/or sense strand nucleotide sequences in Table 3 or Table 4.

In some embodiments, a MARC1 RNAi agent comprises an antisense strand and a sense strand having a modified nucleotide sequence of any of the antisense strand and/or sense strand nucleotide sequences of any of the duplexes Tables 6A and 6B, and further comprises an asialoglycoprotein receptor ligand targeting group.

In some embodiments, a MARC1 RNAi agent comprises, consists of, or consists essentially of any of the duplexes of Tables 6A and 6B.

TABLE 6A
MARC1 RNAi Agents Duplexes with Corresponding Sense and
Antisense Strand ID Numbers and Sequence ID numbers
for the modified and unmodified nucleotide sequences.

| | AS | AS | | SS | SS |
|---|---|---|---|---|---|
| | modified | unmodified | | modified | unmodified |
| | SEQ ID | SEQ ID | | SEQ ID | SEQ ID |
| AS ID | NO: | NO: | SS ID | NO: | NO: |
| AM16712-AS | 1056 | 1542 | AM16711-SS-NL | 1252 | 1667 |
| AM16714-AS | 1057 | 1543 | AM16713-SS-NL | 1253 | 1668 |
| AM16716-AS | 1058 | 1544 | AM16715-SS-NL | 1254 | 1669 |
| AM16718-AS | 1059 | 1545 | AM16717-SS-NL | 1255 | 1670 |
| AM16720-AS | 1060 | 1546 | AM16719-SS-NL | 1256 | 1671 |
| AM16722-AS | 1061 | 1547 | AM16721-SS-NL | 1257 | 1672 |
| AM16724-AS | 1062 | 1548 | AM16723-SS-NL | 1258 | 1673 |
| AM16726-AS | 1063 | 1549 | AM16725-SS-NL | 1259 | 1674 |
| AM16728-AS | 1064 | 1550 | AM16727-SS-NL | 1260 | 1675 |
| AM16730-AS | 1065 | 1551 | AM16729-SS-NL | 1261 | 1676 |
| AM16732-AS | 1066 | 1552 | AM16731-SS-NL | 1262 | 1677 |
| AM16734-AS | 1067 | 1553 | AM16733-SS-NL | 1263 | 1678 |
| AM16736-AS | 1068 | 1554 | AM16735-SS-NL | 1264 | 1679 |
| AM16738-AS | 1069 | 1555 | AM16737-SS-NL | 1265 | 1680 |
| AM16740-AS | 1070 | 1556 | AM16739-SS-NL | 1266 | 1681 |
| AM16742-AS | 1071 | 1557 | AM16741-SS-NL | 1267 | 1682 |
| AM16744-AS | 1072 | 1558 | AM16743-SS-NL | 1268 | 1683 |
| AM16746-AS | 1073 | 1559 | AM16745-SS-NL | 1269 | 1684 |
| AM16748-AS | 1074 | 1560 | AM16747-SS-NL | 1270 | 1685 |
| AM16750-AS | 1075 | 1561 | AM16749-SS-NL | 1271 | 1686 |
| AM16752-AS | 1076 | 1562 | AM16751-SS-NL | 1272 | 1687 |
| AM16754-AS | 1077 | 1563 | AM16753-SS-NL | 1273 | 1688 |
| AM16756-AS | 1078 | 1564 | AM16755-SS-NL | 1274 | 1689 |
| AM16758-AS | 1079 | 1565 | AM16757-SS-NL | 1275 | 1690 |
| AM16760-AS | 1080 | 1566 | AM16759-SS-NL | 1276 | 1691 |
| AM16762-AS | 1081 | 1567 | AM16761-SS-NL | 1277 | 1692 |
| AM16764-AS | 1082 | 1568 | AM16763-SS-NL | 1278 | 1693 |
| AM16766-AS | 1083 | 1569 | AM16765-SS-NL | 1279 | 1694 |
| AM16768-AS | 1084 | 1570 | AM16767-SS-NL | 1280 | 1695 |
| AM16770-AS | 1085 | 1571 | AM16769-SS-NL | 1281 | 1696 |
| AM16772-AS | 1086 | 1572 | AM16771-SS-NL | 1282 | 1697 |
| AM16774-AS | 1087 | 1573 | AM16773-SS-NL | 1283 | 1698 |
| AM17404-AS | 1088 | 1556 | AM16739-SS-NL | 1266 | 1681 |
| AM17405-AS | 1089 | 1556 | AM16739-SS-NL | 1266 | 1681 |
| AM17406-AS | 1090 | 1560 | AM16747-SS-NL | 1270 | 1685 |
| AM17407-AS | 1091 | 1560 | AM16747-SS-NL | 1270 | 1685 |
| AM17408-AS | 1092 | 1563 | AM16753-SS-NL | 1273 | 1688 |
| AM17409-AS | 1093 | 1563 | AM16753-SS-NL | 1273 | 1688 |
| AM17410-AS | 1094 | 1564 | AM16755-SS-NL | 1274 | 1689 |
| AM17411-AS | 1095 | 1564 | AM16755-SS-NL | 1274 | 1689 |
| AM17488-AS | 1096 | 1574 | AM17487-SS-NL | 1284 | 1699 |
| AM17490-AS | 1097 | 1575 | AM17489-SS-NL | 1285 | 1700 |
| AM17492-AS | 1098 | 1576 | AM17491-SS-NL | 1286 | 1701 |
| AM17494-AS | 1099 | 1577 | AM17493-SS-NL | 1287 | 1702 |
| AM17496-AS | 1100 | 1578 | AM17495-SS-NL | 1288 | 1703 |
| AM17498-AS | 1101 | 1579 | AM17497-SS-NL | 1289 | 1704 |
| AM17500-AS | 1102 | 1580 | AM17499-SS-NL | 1290 | 1705 |
| AM17502-AS | 1103 | 1581 | AM17501-SS-NL | 1291 | 1706 |
| AM17504-AS | 1104 | 1582 | AM17503-SS-NL | 1292 | 1707 |
| AM17498-AS | 1101 | 1579 | AM17505-SS-NL | 1293 | 1708 |
| AM17808-AS | 1105 | 1583 | AM17807-SS-NL | 1294 | 1709 |
| AM17810-AS | 1106 | 1584 | AM17809-SS-NL | 1295 | 1710 |
| AM17812-AS | 1107 | 1585 | AM17811-SS-NL | 1296 | 1711 |
| AM17814-AS | 1108 | 1586 | AM17813-SS-NL | 1297 | 1712 |
| AM17816-AS | 1109 | 1587 | AM17815-SS-NL | 1298 | 1713 |
| AM17818-AS | 1110 | 1588 | AM17817-SS-NL | 1299 | 1714 |
| AM17820-AS | 1111 | 1589 | AM17819-SS-NL | 1300 | 1715 |
| AM17822-AS | 1112 | 1590 | AM17821-SS-NL | 1301 | 1716 |
| AM17824-AS | 1113 | 1591 | AM17823-SS-NL | 1302 | 1717 |
| AM17826-AS | 1114 | 1592 | AM17825-SS-NL | 1303 | 1718 |
| AM17828-AS | 1115 | 1593 | AM17827-SS-NL | 1304 | 1719 |
| AM17830-AS | 1116 | 1594 | AM17829-SS-NL | 1305 | 1720 |
| AM17832-AS | 1117 | 1595 | AM17831-SS-NL | 1306 | 1721 |
| AM17834-AS | 1118 | 1596 | AM17833-SS-NL | 1307 | 1722 |
| AM17864-AS | 1119 | 1597 | AM17863-SS-NL | 1308 | 1723 |
| AM17866-AS | 1120 | 1598 | AM17865-SS-NL | 1309 | 1724 |
| AM17868-AS | 1121 | 1599 | AM17867-SS-NL | 1310 | 1725 |
| AM17870-AS | 1122 | 1600 | AM17869-SS-NL | 1311 | 1726 |
| AM17872-AS | 1123 | 1601 | AM17871-SS-NL | 1312 | 1727 |
| AM17874-AS | 1124 | 1602 | AM17873-SS-NL | 1313 | 1728 |
| AM17876-AS | 1125 | 1603 | AM17875-SS-NL | 1314 | 1729 |
| AM17878-AS | 1126 | 1604 | AM17877-SS-NL | 1315 | 1730 |
| AM17880-AS | 1127 | 1605 | AM17879-SS-NL | 1316 | 1731 |
| AM17882-AS | 1128 | 1606 | AM17881-SS-NL | 1317 | 1732 |
| AM18384-AS | 1129 | 1574 | AM17487-SS-NL | 1284 | 1699 |
| AM18385-AS | 1130 | 1574 | AM17487-SS-NL | 1284 | 1699 |
| AM18386-AS | 1131 | 1574 | AM17487-SS-NL | 1284 | 1699 |
| AM18387-AS | 1132 | 1574 | AM17487-SS-NL | 1284 | 1699 |
| AM18388-AS | 1133 | 1580 | AM17499-SS-NL | 1290 | 1705 |
| AM18389-AS | 1134 | 1580 | AM17499-SS-NL | 1290 | 1705 |
| AM18390-AS | 1135 | 1580 | AM17499-SS-NL | 1290 | 1705 |
| AM18391-AS | 1136 | 1580 | AM17499-SS-NL | 1290 | 1705 |
| AM18393-AS | 1137 | 1607 | AM18392-SS-NL | 1318 | 1733 |
| AM18394-AS | 1138 | 1607 | AM18392-SS-NL | 1318 | 1733 |
| AM18398-AS | 1139 | 1564 | AM16755-SS-NL | 1274 | 1689 |
| AM18399-AS | 1140 | 1564 | AM16755-SS-NL | 1274 | 1689 |
| AM18401-AS | 1141 | 1608 | AM18400-SS-NL | 1319 | 1734 |
| AM18402-AS | 1142 | 1608 | AM18400-SS-NL | 1319 | 1734 |
| AM18403-AS | 1143 | 1608 | AM18400-SS-NL | 1319 | 1734 |
| AM18404-AS | 1144 | 1556 | AM16739-SS-NL | 1266 | 1681 |
| AM18405-AS | 1145 | 1556 | AM16739-SS-NL | 1266 | 1681 |
| AM18407-AS | 1146 | 1609 | AM18406-SS-NL | 1320 | 1735 |
| AM18408-AS | 1147 | 1609 | AM18406-SS-NL | 1320 | 1735 |
| AM18409-AS | 1148 | 1609 | AM18406-SS-NL | 1320 | 1735 |
| AM18410-AS | 1149 | 1609 | AM18406-SS-NL | 1320 | 1735 |
| AM18411-AS | 1150 | 1609 | AM18406-SS-NL | 1320 | 1735 |
| AM18412-AS | 1151 | 1609 | AM18406-SS-NL | 1320 | 1735 |
| AM18413-AS | 1152 | 1609 | AM18406-SS-NL | 1320 | 1735 |
| AM18606-AS | 1153 | 1578 | AM17495-SS-NL | 1288 | 1703 |
| AM18607-AS | 1154 | 1578 | AM17495-SS-NL | 1288 | 1703 |
| AM18608-AS | 1155 | 1578 | AM17495-SS-NL | 1288 | 1703 |
| AM18609-AS | 1156 | 1578 | AM17495-SS-NL | 1288 | 1703 |
| AM18610-AS | 1157 | 1578 | AM17495-SS-NL | 1288 | 1703 |
| AM18611-AS | 1158 | 1582 | AM17503-SS-NL | 1292 | 1707 |
| AM18612-AS | 1159 | 1582 | AM17503-SS-NL | 1292 | 1707 |
| AM18614-AS | 1160 | 1610 | AM18613-SS-NL | 1321 | 1736 |
| AM18616-AS | 1161 | 1611 | AM18615-SS-NL | 1322 | 1737 |
| AM18629-AS | 1162 | 1564 | AM16755-SS-NL | 1274 | 1689 |
| AM18629-AS | 1162 | 1564 | AM18630-SS-NL | 1323 | 1689 |
| AM18629-AS | 1162 | 1564 | AM18631-SS-NL | 1324 | 1689 |
| AM18632-AS | 1163 | 1564 | AM16755-SS-NL | 1274 | 1689 |
| AM18632-AS | 1163 | 1564 | AM18630-SS-NL | 1323 | 1689 |
| AM18632-AS | 1163 | 1564 | AM18631-SS-NL | 1324 | 1689 |
| AM18634-AS | 1164 | 1612 | AM18633-SS-NL | 1325 | 1738 |
| AM18636-AS | 1165 | 1613 | AM18635-SS-NL | 1326 | 1739 |
| AM18638-AS | 1166 | 1614 | AM18637-SS-NL | 1327 | 1740 |
| AM18878-AS | 1167 | 1615 | AM18877-SS-NL | 1328 | 1741 |
| AM18880-AS | 1168 | 1616 | AM18879-SS-NL | 1329 | 1742 |
| AM18882-AS | 1169 | 1617 | AM18881-SS-NL | 1330 | 1743 |
| AM18884-AS | 1170 | 1618 | AM18883-SS-NL | 1331 | 1744 |
| AM18886-AS | 1171 | 1619 | AM18885-SS-NL | 1332 | 1745 |
| AM18888-AS | 1172 | 1620 | AM18887-SS-NL | 1333 | 1746 |
| AM18890-AS | 1173 | 1621 | AM18889-SS-NL | 1334 | 1747 |
| AM18892-AS | 1174 | 1622 | AM18891-SS-NL | 1335 | 1748 |
| AM18894-AS | 1175 | 1623 | AM18893-SS-NL | 1336 | 1749 |
| AM18896-AS | 1176 | 1624 | AM18895-SS-NL | 1337 | 1750 |
| AM18898-AS | 1177 | 1625 | AM18897-SS-NL | 1338 | 1751 |
| AM18900-AS | 1178 | 1626 | AM18899-SS-NL | 1339 | 1752 |
| AM18902-AS | 1179 | 1627 | AM18901-SS-NL | 1340 | 1753 |
| AM18904-AS | 1180 | 1628 | AM18903-SS-NL | 1341 | 1754 |
| AM18906-AS | 1181 | 1629 | AM18905-SS-NL | 1342 | 1755 |
| AM18908-AS | 1182 | 1630 | AM18907-SS-NL | 1343 | 1756 |
| AM18910-AS | 1183 | 1631 | AM18909-SS-NL | 1344 | 1757 |
| AM18912-AS | 1184 | 1632 | AM18911-SS-NL | 1345 | 1758 |
| AM18914-AS | 1185 | 1633 | AM18913-SS-NL | 1346 | 1759 |
| AM18916-AS | 1186 | 1634 | AM18915-SS-NL | 1347 | 1760 |
| AM18918-AS | 1187 | 1635 | AM18917-SS-NL | 1348 | 1761 |
| AM18920-AS | 1188 | 1636 | AM18919-SS-NL | 1349 | 1762 |
| AM18922-AS | 1189 | 1637 | AM18921-SS-NL | 1350 | 1763 |
| AM18924-AS | 1190 | 1638 | AM18923-SS-NL | 1351 | 1764 |
| AM18944-AS | 1191 | 1639 | AM18943-SS-NL | 1352 | 1765 |
| AM18946-AS | 1192 | 1640 | AM18945-SS-NL | 1353 | 1766 |
| AM18948-AS | 1193 | 1641 | AM18947-SS-NL | 1354 | 1767 |
| AM18950-AS | 1194 | 1642 | AM18949-SS-NL | 1355 | 1768 |
| AM18952-AS | 1195 | 1643 | AM18951-SS-NL | 1356 | 1769 |
| AM18954-AS | 1196 | 1644 | AM18953-SS-NL | 1357 | 1770 |
| AM18956-AS | 1197 | 1645 | AM18955-SS-NL | 1358 | 1771 |
| AM18958-AS | 1198 | 1646 | AM18957-SS-NL | 1359 | 1772 |
| AM18960-AS | 1199 | 1647 | AM18959-SS-NL | 1360 | 1773 |
| AM17500-AS | 1102 | 1580 | AM19119-SS-NL | 1361 | 1774 |
| AM19120-AS | 1200 | 1580 | AM19119-SS-NL | 1361 | 1774 |
| AM19121-AS | 1201 | 1580 | AM19119-SS-NL | 1361 | 1774 |
| AM19122-AS | 1202 | 1580 | AM19119-SS-NL | 1361 | 1774 |
| AM19123-AS | 1203 | 1580 | AM17499-SS-NL | 1290 | 1705 |
| AM19123-AS | 1203 | 1580 | AM19124-SS-NL | 1362 | 1705 |
| AM19121-AS | 1201 | 1580 | AM17499-SS-NL | 1290 | 1705 |
| AM19121-AS | 1201 | 1580 | AM19124-SS-NL | 1362 | 1705 |
| AM19125-AS | 1204 | 1596 | AM17833-SS-NL | 1307 | 1722 |
| AM19126-AS | 1205 | 1596 | AM17833-SS-NL | 1307 | 1722 |
| AM19127-AS | 1206 | 1596 | AM17833-SS-NL | 1307 | 1722 |
| AM19128-AS | 1207 | 1596 | AM17833-SS-NL | 1307 | 1722 |
| AM19127-AS | 1206 | 1596 | AM19129-SS-NL | 1363 | 1722 |
| AM19127-AS | 1206 | 1596 | AM19130-SS-NL | 1364 | 1775 |
| AM19131-AS | 1208 | 1648 | AM17833-SS-NL | 1307 | 1722 |
| AM19132-AS | 1209 | 1649 | AM17833-SS-NL | 1307 | 1722 |
| AM19181-AS | 1210 | 1578 | AM17495-SS-NL | 1288 | 1703 |
| AM19182-AS | 1211 | 1578 | AM17495-SS-NL | 1288 | 1703 |
| AM19183-AS | 1212 | 1578 | AM17495-SS-NL | 1288 | 1703 |
| AM19185-AS | 1213 | 1650 | AM19184-SS-NL | 1365 | 1776 |
| AM19186-AS | 1214 | 1650 | AM19184-SS-NL | 1365 | 1776 |
| AM18610-AS | 1157 | 1578 | AM19187-SS-NL | 1366 | 1703 |
| AM19189-AS | 1215 | 1651 | AM19188-SS-NL | 1367 | 1777 |
| AM19191-AS | 1216 | 1652 | AM19190-SS-NL | 1368 | 1778 |
| AM19193-AS | 1217 | 1653 | AM19192-SS-NL | 1369 | 1779 |
| AM19193-AS | 1217 | 1653 | AM19194-SS-NL | 1370 | 1779 |
| AM19219-AS | 1218 | 1654 | AM19218-SS-NL | 1371 | 1780 |
| AM19221-AS | 1219 | 1655 | AM19220-SS-NL | 1372 | 1781 |
| AM19223-AS | 1220 | 1656 | AM19222-SS-NL | 1373 | 1782 |
| AM18410-AS | 1149 | 1609 | AM19404-SS-NL | 1374 | 1735 |
| AM19405-AS | 1221 | 1609 | AM18406-SS-NL | 1320 | 1735 |
| AM19406-AS | 1222 | 1609 | AM18406-SS-NL | 1320 | 1735 |
| AM19407-AS | 1223 | 1606 | AM17881-SS-NL | 1317 | 1732 |
| AM19408-AS | 1224 | 1606 | AM17881-SS-NL | 1317 | 1732 |
| AM19408-AS | 1224 | 1606 | AM19409-SS-NL | 1375 | 1732 |
| AM19410-AS | 1225 | 1606 | AM17881-SS-NL | 1317 | 1732 |
| AM19411-AS | 1226 | 1606 | AM17881-SS-NL | 1317 | 1732 |
| AM19536-AS | 1227 | 1650 | AM19184-SS-NL | 1365 | 1776 |
| AM19538-AS | 1228 | 1657 | AM19537-SS-NL | 1376 | 1783 |
| AM19538.1-AS | 1229 | 1658 | AM19537-SS-NL | 1376 | 1783 |
| AM19539-AS | 1230 | 1653 | AM19194-SS-NL | 1370 | 1779 |
| AM19656-AS | 1231 | 1604 | AM17877-SS-NL | 1315 | 1730 |
| AM19657-AS | 1232 | 1604 | AM17877-SS-NL | 1315 | 1730 |
| AM19658-AS | 1233 | 1560 | AM16747-SS-NL | 1270 | 1685 |
| AM19659-AS | 1234 | 1560 | AM16747-SS-NL | 1270 | 1685 |
| AM19661-AS | 1235 | 1659 | AM19660-SS-NL | 1377 | 1784 |
| AM19663-AS | 1236 | 1660 | AM19662-SS-NL | 1378 | 1785 |
| CA004443 | 1237 | 1651 | CS914989-NL | 1394 | 1777 |
| CA004444 | 1238 | 1652 | CS914991-NL | 1395 | 1778 |
| CA004446 | 1239 | 1661 | CS004445-NL | 1379 | 1786 |
| CA004447 | 1240 | 1651 | CS914989-NL | 1394 | 1777 |
| CA004449 | 1241 | 1662 | CS004448-NL | 1380 | 1787 |
| CA004481 | 1242 | 1663 | CS004480-NL | 1381 | 1788 |
| CA004483 | 1243 | 1664 | CS004482-NL | 1382 | 1789 |
| CA004798 | 1244 | 1658 | CS915323-NL | 1396 | 1783 |
| CA005198 | 1245 | 1665 | CS005197-NL | 1383 | 1790 |
| CA008056 | 1246 | 1585 | CS913717-NL | 1390 | 1711 |
| CA008057 | 1247 | 1591 | CS913729-NL | 1391 | 1717 |
| CA008058 | 1248 | 1596 | CS913739-NL | 1392 | 1722 |
| CA008429 | 1249 | 1666 | CS008428-NL | 1389 | 1791 |
| CA008430 | 1250 | 1608 | CS914289-NL | 1393 | 1734 |

TABLE 6B
MARC1 RNAi Agents Duplexes with Corresponding Sense and Antisense Strand ID Numbers
and Sequence ID numbers for the modified and unmodified nucleotide sequences.

| | | AS | AS | | SS | SS |
|---|---|---|---|---|---|---|
| | | modified | unmodified | | modified | unmodified |
| | | SEQ ID | SEQ ID | | SEQ ID | SEQ ID |
| Duplex | AS ID | NO: | NO: | SS ID | NO: | NO: |
| AD11764 | AM16712-AS | 1056 | 1542 | AM16711-SS | 1397 | 1667 |
| AD11765 | AM16714-AS | 1057 | 1543 | AM16713-SS | 1398 | 1668 |
| AD11766 | AM16716-AS | 1058 | 1544 | AM16715-SS | 1399 | 1669 |
| AD11767 | AM16718-AS | 1059 | 1545 | AM16717-SS | 1400 | 1670 |
| AD11768 | AM16720-AS | 1060 | 1546 | AM16719-SS | 1401 | 1671 |
| AD11769 | AM16722-AS | 1061 | 1547 | AM16721-SS | 1402 | 1672 |
| AD11770 | AM16724-AS | 1062 | 1548 | AM16723-SS | 1403 | 1673 |
| AD11771 | AM16726-AS | 1063 | 1549 | AM16725-SS | 1404 | 1674 |
| AD11772 | AM16728-AS | 1064 | 1550 | AM16727-SS | 1405 | 1675 |
| AD11773 | AM16730-AS | 1065 | 1551 | AM16729-SS | 1406 | 1676 |
| AD11774 | AM16732-AS | 1066 | 1552 | AM16731-SS | 1407 | 1677 |
| AD11775 | AM16734-AS | 1067 | 1553 | AM16733-SS | 1408 | 1678 |
| AD11776 | AM16736-AS | 1068 | 1554 | AM16735-SS | 1409 | 1679 |
| AD11777 | AM16738-AS | 1069 | 1555 | AM16737-SS | 1410 | 1680 |
| AD11778 | AM16740-AS | 1070 | 1556 | AM16739-SS | 1411 | 1681 |
| AD11779 | AM16742-AS | 1071 | 1557 | AM16741-SS | 1412 | 1682 |
| AD11780 | AM16744-AS | 1072 | 1558 | AM16743-SS | 1413 | 1683 |
| AD11781 | AM16746-AS | 1073 | 1559 | AM16745-SS | 1414 | 1684 |
| AD11782 | AM16748-AS | 1074 | 1560 | AM16747-SS | 1415 | 1685 |
| AD11783 | AM16750-AS | 1075 | 1561 | AM16749-SS | 1416 | 1686 |
| AD11784 | AM16752-AS | 1076 | 1562 | AM16751-SS | 1417 | 1687 |
| AD11785 | AM16754-AS | 1077 | 1563 | AM16753-SS | 1418 | 1688 |
| AD11786 | AM16756-AS | 1078 | 1564 | AM16755-SS | 1419 | 1689 |
| AD11787 | AM16758-AS | 1079 | 1565 | AM16757-SS | 1420 | 1690 |
| AD11788 | AM16760-AS | 1080 | 1566 | AM16759-SS | 1421 | 1691 |
| AD11789 | AM16762-AS | 1081 | 1567 | AM16761-SS | 1422 | 1692 |
| AD11790 | AM16764-AS | 1082 | 1568 | AM16763-SS | 1423 | 1693 |
| AD11791 | AM16766-AS | 1083 | 1569 | AM16765-SS | 1424 | 1694 |
| AD11792 | AM16768-AS | 1084 | 1570 | AM16767-SS | 1425 | 1695 |
| AD11793 | AM16770-AS | 1085 | 1571 | AM16769-SS | 1426 | 1696 |
| AD11794 | AM16772-AS | 1086 | 1572 | AM16771-SS | 1427 | 1697 |
| AD11795 | AM16774-AS | 1087 | 1573 | AM16773-SS | 1428 | 1698 |
| AD12285 | AM17404-AS | 1088 | 1556 | AM16739-SS | 1411 | 1681 |
| AD12286 | AM17405-AS | 1089 | 1556 | AM16739-SS | 1411 | 1681 |
| AD12287 | AM17406-AS | 1090 | 1560 | AM16747-SS | 1415 | 1685 |
| AD12288 | AM17407-AS | 1091 | 1560 | AM16747-SS | 1415 | 1685 |
| AD12289 | AM17408-AS | 1092 | 1563 | AM16753-SS | 1418 | 1688 |
| AD12290 | AM17409-AS | 1093 | 1563 | AM16753-SS | 1418 | 1688 |
| AD12291 | AM17410-AS | 1094 | 1564 | AM16755-SS | 1419 | 1689 |
| AD12292 | AM17411-AS | 1095 | 1564 | AM16755-SS | 1419 | 1689 |
| AD12363 | AM17488-AS | 1096 | 1574 | AM17487-SS | 1429 | 1699 |
| AD12364 | AM17490-AS | 1097 | 1575 | AM17489-SS | 1430 | 1700 |
| AD12365 | AM17492-AS | 1098 | 1576 | AM17491-SS | 1431 | 1701 |
| AD12366 | AM17494-AS | 1099 | 1577 | AM17493-SS | 1432 | 1702 |
| AD12367 | AM17496-AS | 1100 | 1578 | AM17495-SS | 1433 | 1703 |
| AD12368 | AM17498-AS | 1101 | 1579 | AM17497-SS | 1434 | 1704 |
| AD12369 | AM17500-AS | 1102 | 1580 | AM17499-SS | 1435 | 1705 |
| AD12370 | AM17502-AS | 1103 | 1581 | AM17501-SS | 1436 | 1706 |
| AD12371 | AM17504-AS | 1104 | 1582 | AM17503-SS | 1437 | 1707 |
| AD12372 | AM17498-AS | 1101 | 1579 | AM17505-SS | 1438 | 1708 |
| AD12583 | AM17808-AS | 1105 | 1583 | AM17807-SS | 1439 | 1709 |
| AD12584 | AM17810-AS | 1106 | 1584 | AM17809-SS | 1440 | 1710 |
| AD12585 | AM17812-AS | 1107 | 1585 | AM17811-SS | 1441 | 1711 |
| AD12586 | AM17814-AS | 1108 | 1586 | AM17813-SS | 1442 | 1712 |
| AD12587 | AM17816-AS | 1109 | 1587 | AM17815-SS | 1443 | 1713 |
| AD12588 | AM17818-AS | 1110 | 1588 | AM17817-SS | 1444 | 1714 |
| AD12589 | AM17820-AS | 1111 | 1589 | AM17819-SS | 1445 | 1715 |
| AD12590 | AM17822-AS | 1112 | 1590 | AM17821-SS | 1446 | 1716 |
| AD12591 | AM17824-AS | 1113 | 1591 | AM17823-SS | 1447 | 1717 |
| AD12592 | AM17826-AS | 1114 | 1592 | AM17825-SS | 1448 | 1718 |
| AD12593 | AM17828-AS | 1115 | 1593 | AM17827-SS | 1449 | 1719 |
| AD12594 | AM17830-AS | 1116 | 1594 | AM17829-SS | 1450 | 1720 |
| AD12595 | AM17832-AS | 1117 | 1595 | AM17831-SS | 1451 | 1721 |
| AD12596 | AM17834-AS | 1118 | 1596 | AM17833-SS | 1452 | 1722 |
| AD12625 | AM17864-AS | 1119 | 1597 | AM17863-SS | 1453 | 1723 |
| AD12626 | AM17866-AS | 1120 | 1598 | AM17865-SS | 1454 | 1724 |
| AD12627 | AM17868-AS | 1121 | 1599 | AM17867-SS | 1455 | 1725 |
| AD12628 | AM17870-AS | 1122 | 1600 | AM17869-SS | 1456 | 1726 |
| AD12629 | AM17872-AS | 1123 | 1601 | AM17871-SS | 1457 | 1727 |
| AD12630 | AM17874-AS | 1124 | 1602 | AM17873-SS | 1458 | 1728 |
| AD12631 | AM17876-AS | 1125 | 1603 | AM17875-SS | 1459 | 1729 |
| AD12632 | AM17878-AS | 1126 | 1604 | AM17877-SS | 1460 | 1730 |
| AD12633 | AM17880-AS | 1127 | 1605 | AM17879-SS | 1461 | 1731 |
| AD12634 | AM17882-AS | 1128 | 1606 | AM17881-SS | 1462 | 1732 |
| AD12953 | AM18384-AS | 1129 | 1574 | AM17487-SS | 1429 | 1699 |
| AD12954 | AM18385-AS | 1130 | 1574 | AM17487-SS | 1429 | 1699 |
| AD12955 | AM18386-AS | 1131 | 1574 | AM17487-SS | 1429 | 1699 |
| AD12956 | AM18387-AS | 1132 | 1574 | AM17487-SS | 1429 | 1699 |
| AD12957 | AM18388-AS | 1133 | 1580 | AM17499-SS | 1435 | 1705 |
| AD12958 | AM18389-AS | 1134 | 1580 | AM17499-SS | 1435 | 1705 |
| AD12959 | AM18390-AS | 1135 | 1580 | AM17499-SS | 1435 | 1705 |
| AD12960 | AM18391-AS | 1136 | 1580 | AM17499-SS | 1435 | 1705 |
| AD12961 | AM18393-AS | 1137 | 1607 | AM18392-SS | 1463 | 1733 |
| AD12962 | AM18394-AS | 1138 | 1607 | AM18392-SS | 1463 | 1733 |
| AD12972 | AM18398-AS | 1139 | 1564 | AM16755-SS | 1419 | 1689 |
| AD12973 | AM18399-AS | 1140 | 1564 | AM16755-SS | 1419 | 1689 |
| AD12974 | AM18401-AS | 1141 | 1608 | AM18400-SS | 1464 | 1734 |
| AD12975 | AM18402-AS | 1142 | 1608 | AM18400-SS | 1464 | 1734 |
| AD12976 | AM18403-AS | 1143 | 1608 | AM18400-SS | 1464 | 1734 |
| AD12977 | AM18404-AS | 1144 | 1556 | AM16739-SS | 1411 | 1681 |
| AD12978 | AM18405-AS | 1145 | 1556 | AM16739-SS | 1411 | 1681 |
| AD12979 | AM18407-AS | 1146 | 1609 | AM18406-SS | 1465 | 1735 |
| AD12980 | AM18408-AS | 1147 | 1609 | AM18406-SS | 1465 | 1735 |
| AD12981 | AM18409-AS | 1148 | 1609 | AM18406-SS | 1465 | 1735 |
| AD12982 | AM18410-AS | 1149 | 1609 | AM18406-SS | 1465 | 1735 |
| AD12983 | AM18411-AS | 1150 | 1609 | AM18406-SS | 1465 | 1735 |
| AD12984 | AM18412-AS | 1151 | 1609 | AM18406-SS | 1465 | 1735 |
| AD12985 | AM18413-AS | 1152 | 1609 | AM18406-SS | 1465 | 1735 |
| AD13113 | AM18606-AS | 1153 | 1578 | AM17495-SS | 1433 | 1703 |
| AD13114 | AM18607-AS | 1154 | 1578 | AM17495-SS | 1433 | 1703 |
| AD13115 | AM18608-AS | 1155 | 1578 | AM17495-SS | 1433 | 1703 |
| AD13116 | AM18609-AS | 1156 | 1578 | AM17495-SS | 1433 | 1703 |
| AD13117 | AM18610-AS | 1157 | 1578 | AM17495-SS | 1433 | 1703 |
| AD13118 | AM18611-AS | 1158 | 1582 | AM17503-SS | 1437 | 1707 |
| AD13119 | AM18612-AS | 1159 | 1582 | AM17503-SS | 1437 | 1707 |
| AD13120 | AM18614-AS | 1160 | 1610 | AM18613-SS | 1466 | 1736 |
| AD13121 | AM18616-AS | 1161 | 1611 | AM18615-SS | 1467 | 1737 |
| AD13137 | AM18629-AS | 1162 | 1564 | AM16755-SS | 1419 | 1689 |
| AD13138 | AM18629-AS | 1162 | 1564 | AM18630-SS | 1468 | 1689 |
| AD13139 | AM18629-AS | 1162 | 1564 | AM18631-SS | 1469 | 1689 |
| AD13140 | AM18632-AS | 1163 | 1564 | AM16755-SS | 1419 | 1689 |
| AD13141 | AM18632-AS | 1163 | 1564 | AM18630-SS | 1468 | 1689 |
| AD13142 | AM18632-AS | 1163 | 1564 | AM18631-SS | 1469 | 1689 |
| AD13143 | AM18634-AS | 1164 | 1612 | AM18633-SS | 1470 | 1738 |
| AD13144 | AM18636-AS | 1165 | 1613 | AM18635-SS | 1471 | 1739 |
| AD13145 | AM18638-AS | 1166 | 1614 | AM18637-SS | 1472 | 1740 |
| AD13278 | AM18878-AS | 1167 | 1615 | AM18877-SS | 1473 | 1741 |
| AD13279 | AM18880-AS | 1168 | 1616 | AM18879-SS | 1474 | 1742 |
| AD13280 | AM18882-AS | 1169 | 1617 | AM18881-SS | 1475 | 1743 |
| AD13281 | AM18884-AS | 1170 | 1618 | AM18883-SS | 1476 | 1744 |
| AD13282 | AM18886-AS | 1171 | 1619 | AM18885-SS | 1477 | 1745 |
| AD13283 | AM18888-AS | 1172 | 1620 | AM18887-SS | 1478 | 1746 |
| AD13284 | AM18890-AS | 1173 | 1621 | AM18889-SS | 1479 | 1747 |
| AD13285 | AM18892-AS | 1174 | 1622 | AM18891-SS | 1480 | 1748 |
| AD13286 | AM18894-AS | 1175 | 1623 | AM18893-SS | 1481 | 1749 |
| AD13287 | AM18896-AS | 1176 | 1624 | AM18895-SS | 1482 | 1750 |
| AD13288 | AM18898-AS | 1177 | 1625 | AM18897-SS | 1483 | 1751 |
| AD13289 | AM18900-AS | 1178 | 1626 | AM18899-SS | 1484 | 1752 |
| AD13290 | AM18902-AS | 1179 | 1627 | AM18901-SS | 1485 | 1753 |
| AD13291 | AM18904-AS | 1180 | 1628 | AM18903-SS | 1486 | 1754 |
| AD13292 | AM18906-AS | 1181 | 1629 | AM18905-SS | 1487 | 1755 |
| AD13293 | AM18908-AS | 1182 | 1630 | AM18907-SS | 1488 | 1756 |
| AD13294 | AM18910-AS | 1183 | 1631 | AM18909-SS | 1489 | 1757 |
| AD13295 | AM18912-AS | 1184 | 1632 | AM18911-SS | 1490 | 1758 |
| AD13296 | AM18914-AS | 1185 | 1633 | AM18913-SS | 1491 | 1759 |
| AD13297 | AM18916-AS | 1186 | 1634 | AM18915-SS | 1492 | 1760 |
| AD13298 | AM18918-AS | 1187 | 1635 | AM18917-SS | 1493 | 1761 |
| AD13299 | AM18920-AS | 1188 | 1636 | AM18919-SS | 1494 | 1762 |
| AD13300 | AM18922-AS | 1189 | 1637 | AM18921-SS | 1495 | 1763 |
| AD13301 | AM18924-AS | 1190 | 1638 | AM18923-SS | 1496 | 1764 |
| AD13322 | AM18944-AS | 1191 | 1639 | AM18943-SS | 1497 | 1765 |
| AD13323 | AM18946-AS | 1192 | 1640 | AM18945-SS | 1498 | 1766 |
| AD13324 | AM18948-AS | 1193 | 1641 | AM18947-SS | 1499 | 1767 |
| AD13325 | AM18950-AS | 1194 | 1642 | AM18949-SS | 1500 | 1768 |
| AD13326 | AM18952-AS | 1195 | 1643 | AM18951-SS | 1501 | 1769 |
| AD13327 | AM18954-AS | 1196 | 1644 | AM18953-SS | 1502 | 1770 |
| AD13328 | AM18956-AS | 1197 | 1645 | AM18955-SS | 1503 | 1771 |
| AD13329 | AM18958-AS | 1198 | 1646 | AM18957-SS | 1504 | 1772 |
| AD13330 | AM18960-AS | 1199 | 1647 | AM18959-SS | 1505 | 1773 |
| AD13444 | AM17500-AS | 1102 | 1580 | AM19119-SS | 1506 | 1774 |
| AD13445 | AM19120-AS | 1200 | 1580 | AM19119-SS | 1506 | 1774 |
| AD13446 | AM19121-AS | 1201 | 1580 | AM19119-SS | 1506 | 1774 |
| AD13447 | AM19122-AS | 1202 | 1580 | AM19119-SS | 1506 | 1774 |
| AD13448 | AM19123-AS | 1203 | 1580 | AM17499-SS | 1435 | 1705 |
| AD13449 | AM19123-AS | 1203 | 1580 | AM19124-SS | 1507 | 1705 |
| AD13450 | AM19121-AS | 1201 | 1580 | AM17499-SS | 1435 | 1705 |
| AD13451 | AM19121-AS | 1201 | 1580 | AM19124-SS | 1507 | 1705 |
| AD13452 | AM19125-AS | 1204 | 1596 | AM17833-SS | 1452 | 1722 |
| AD13453 | AM19126-AS | 1205 | 1596 | AM17833-SS | 1452 | 1722 |
| AD13454 | AM19127-AS | 1206 | 1596 | AM17833-SS | 1452 | 1722 |
| AD13455 | AM19128-AS | 1207 | 1596 | AM17833-SS | 1452 | 1722 |
| AD13456 | AM19127-AS | 1206 | 1596 | AM19129-SS | 1508 | 1722 |
| AD13457 | AM19127-AS | 1206 | 1596 | AM19130-SS | 1509 | 1775 |
| AD13458 | AM19131-AS | 1208 | 1648 | AM17833-SS | 1452 | 1722 |
| AD13459 | AM19132-AS | 1209 | 1649 | AM17833-SS | 1452 | 1722 |
| AD13507 | AM19181-AS | 1210 | 1578 | AM17495-SS | 1433 | 1703 |
| AD13508 | AM19182-AS | 1211 | 1578 | AM17495-SS | 1433 | 1703 |
| AD13509 | AM19183-AS | 1212 | 1578 | AM17495-SS | 1433 | 1703 |
| AD13510 | AM19185-AS | 1213 | 1650 | AM19184-SS | 1510 | 1776 |
| AD13511 | AM19186-AS | 1214 | 1650 | AM19184-SS | 1510 | 1776 |
| AD13512 | AM18610-AS | 1157 | 1578 | AM19187-SS | 1511 | 1703 |
| AD13513 | AM19189-AS | 1215 | 1651 | AM19188-SS | 1512 | 1777 |
| AD13514 | AM19191-AS | 1216 | 1652 | AM19190-SS | 1513 | 1778 |
| AD13515 | AM19193-AS | 1217 | 1653 | AM19192-SS | 1514 | 1779 |
| AD13516 | AM19193-AS | 1217 | 1653 | AM19194-SS | 1515 | 1779 |
| AD13535 | AM19219-AS | 1218 | 1654 | AM19218-SS | 1516 | 1780 |
| AD13536 | AM19221-AS | 1219 | 1655 | AM19220-SS | 1517 | 1781 |
| AD13537 | AM19223-AS | 1220 | 1656 | AM19222-SS | 1518 | 1782 |
| AD13705 | AM18410-AS | 1149 | 1609 | AM19404-SS | 1519 | 1735 |
| AD13706 | AM19405-AS | 1221 | 1609 | AM18406-SS | 1465 | 1735 |
| AD13707 | AM19406-AS | 1222 | 1609 | AM18406-SS | 1465 | 1735 |
| AD13708 | AM19407-AS | 1223 | 1606 | AM17881-SS | 1462 | 1732 |
| AD13709 | AM19408-AS | 1224 | 1606 | AM17881-SS | 1462 | 1732 |
| AD13710 | AM19408-AS | 1224 | 1606 | AM19409-SS | 1520 | 1732 |
| AD13711 | AM19410-AS | 1225 | 1606 | AM17881-SS | 1462 | 1732 |
| AD13712 | AM19411-AS | 1226 | 1606 | AM17881-SS | 1462 | 1732 |
| AD13804 | AM19536-AS | 1227 | 1650 | AM19184-SS | 1510 | 1776 |
| AD13805 | AM19538-AS | 1228 | 1657 | AM19537-SS | 1521 | 1783 |
| AD13805.1 | AM19538.1-AS | 1229 | 1658 | AM19537-SS | 1521 | 1783 |
| AD13806 | AM19539-AS | 1230 | 1653 | AM19194-SS | 1515 | 1779 |
| AD13921 | AM19656-AS | 1231 | 1604 | AM17877-SS | 1460 | 1730 |
| AD13922 | AM19657-AS | 1232 | 1604 | AM17877-SS | 1460 | 1730 |
| AD13923 | AM19658-AS | 1233 | 1560 | AM16747-SS | 1415 | 1685 |
| AD13924 | AM19659-AS | 1234 | 1560 | AM16747-SS | 1415 | 1685 |
| AD13925 | AM19661-AS | 1235 | 1659 | AM19660-SS | 1522 | 1784 |
| AD13926 | AM19663-AS | 1236 | 1660 | AM19662-SS | 1523 | 1785 |
| AC003589 | CA004443 | 1237 | 1651 | CS914989 | 1539 | 1777 |
| AC003590 | CA004444 | 1238 | 1652 | CS914991 | 1540 | 1778 |
| AC003591 | CA004446 | 1239 | 1661 | CS004445 | 1524 | 1786 |
| AC003592 | CA004447 | 1240 | 1651 | CS914989 | 1539 | 1777 |
| AC003593 | CA004449 | 1241 | 1662 | CS004448 | 1525 | 1787 |
| AC003625 | CA004481 | 1242 | 1663 | CS004480 | 1526 | 1788 |
| AC003626 | CA004483 | 1243 | 1664 | CS004482 | 1527 | 1789 |
| AC003891 | CA004798 | 1244 | 1658 | CS915323 | 1541 | 1783 |
| AC004186 | CA005198 | 1245 | 1665 | CS005197 | 1528 | 1790 |
| AC006749 | CA008056 | 1246 | 1585 | CS913717 | 1535 | 1711 |
| AC006750 | CA008057 | 1247 | 1591 | CS913729 | 1536 | 1717 |
| AC006751 | CA008058 | 1248 | 1596 | CS913739 | 1537 | 1722 |
| AC007084 | CA008429 | 1249 | 1666 | CS008428 | 1534 | 1791 |
| AC007085 | CA008430 | 1250 | 1608 | CS914289 | 1538 | 1734 |

TABLE 6C
MARC1 RNAi Agents Duplexes with Corresponding Sense
and Antisense Strand ID Numbers Referencing Position
Targeted on MARC1 Gene (SEQ ID NO: 1)

| | | | Targeted MARC1 |
|---|---|---|---|
| | Antisense | Sense | Gene Position |
| Duplex ID | Strand ID | Strand ID | (Of SEQ ID NO: 1) |
| AD11764 | AM16712-AS | AM16711-SS | 325 |
| AD11765 | AM16714-AS | AM16713-SS | 536 |
| AD11766 | AM16716-AS | AM16715-SS | 609 |
| AD11767 | AM16718-AS | AM16717-SS | 611 |
| AD11768 | AM16720-AS | AM16719-SS | 636 |
| AD11769 | AM16722-AS | AM16721-SS | 640 |
| AD11770 | AM16724-AS | AM16723-SS | 644 |
| AD11771 | AM16726-AS | AM16725-SS | 710 |
| AD11772 | AM16728-AS | AM16727-SS | 841 |
| AD11773 | AM16730-AS | AM16729-SS | 932 |
| AD11774 | AM16732-AS | AM16731-SS | 940 |
| AD11775 | AM16734-AS | AM16733-SS | 945 |
| AD11776 | AM16736-AS | AM16735-SS | 954 |
| AD11777 | AM16738-AS | AM16737-SS | 1057 |
| AD11778 | AM16740-AS | AM16739-SS | 1089 |
| AD11779 | AM16742-AS | AM16741-SS | 1098 |
| AD11780 | AM16744-AS | AM16743-SS | 1102 |
| AD11781 | AM16746-AS | AM16745-SS | 1111 |
| AD11782 | AM16748-AS | AM16747-SS | 1190 |
| AD11783 | AM16750-AS | AM16749-SS | 1193 |
| AD11784 | AM16752-AS | AM16751-SS | 1282 |
| AD11785 | AM16754-AS | AM16753-SS | 1310 |
| AD11786 | AM16756-AS | AM16755-SS | 1313 |
| AD11787 | AM16758-AS | AM16757-SS | 1605 |
| AD11788 | AM16760-AS | AM16759-SS | 1635 |
| AD11789 | AM16762-AS | AM16761-SS | 1646 |
| AD11790 | AM16764-AS | AM16763-SS | 1648 |
| AD11791 | AM16766-AS | AM16765-SS | 1852 |
| AD11792 | AM16768-AS | AM16767-SS | 1897 |
| AD11793 | AM16770-AS | AM16769-SS | 1898 |
| AD11794 | AM16772-AS | AM16771-SS | 1955 |
| AD11795 | AM16774-AS | AM16773-SS | 1990 |
| AD12285 | AM17404-AS | AM16739-SS | 1089 |
| AD12286 | AM17405-AS | AM16739-SS | 1089 |
| AD12287 | AM17406-AS | AM16747-SS | 1190 |
| AD12288 | AM17407-AS | AM16747-SS | 1190 |
| AD12289 | AM17408-AS | AM16753-SS | 1310 |
| AD12290 | AM17409-AS | AM16753-SS | 1310 |
| AD12291 | AM17410-AS | AM16755-SS | 1313 |
| AD12292 | AM17411-AS | AM16755-SS | 1313 |
| AD12363 | AM17488-AS | AM17487-SS | 305 |
| AD12364 | AM17490-AS | AM17489-SS | 761 |
| AD12365 | AM17492-AS | AM17491-SS | 956 |
| AD12366 | AM17494-AS | AM17493-SS | 1109 |
| AD12367 | AM17496-AS | AM17495-SS | 1275 |
| AD12368 | AM17498-AS | AM17497-SS | 1633 |
| AD12369 | AM17500-AS | AM17499-SS | 1817 |
| AD12370 | AM17502-AS | AM17501-SS | 1900 |
| AD12371 | AM17504-AS | AM17503-SS | 1954 |
| AD12372 | AM17498-AS | AM17505-SS | 1633 |
| AD12583 | AM17808-AS | AM17807-SS | 405 |
| AD12584 | AM17810-AS | AM17809-SS | 406 |
| AD12585 | AM17812-AS | AM17811-SS | 409 |
| AD12586 | AM17814-AS | AM17813-SS | 411 |
| AD12587 | AM17816-AS | AM17815-SS | 564 |
| AD12588 | AM17818-AS | AM17817-SS | 565 |
| AD12589 | AM17820-AS | AM17819-SS | 566 |
| AD12590 | AM17822-AS | AM17821-SS | 567 |
| AD12591 | AM17824-AS | AM17823-SS | 901 |
| AD12592 | AM17826-AS | AM17825-SS | 1008 |
| AD12593 | AM17828-AS | AM17827-SS | 1009 |
| AD12594 | AM17830-AS | AM17829-SS | 1012 |
| AD12595 | AM17832-AS | AM17831-SS | 1013 |
| AD12596 | AM17834-AS | AM17833-SS | 1014 |
| AD12625 | AM17864-AS | AM17863-SS | 610 |
| AD12626 | AM17866-AS | AM17865-SS | 714 |
| AD12627 | AM17868-AS | AM17867-SS | 716 |
| AD12628 | AM17870-AS | AM17869-SS | 744 |
| AD12629 | AM17872-AS | AM17871-SS | 933 |
| AD12630 | AM17874-AS | AM17873-SS | 938 |
| AD12631 | AM17876-AS | AM17875-SS | 1302 |
| AD12632 | AM17878-AS | AM17877-SS | 1642 |
| AD12633 | AM17880-AS | AM17879-SS | 1927 |
| AD12634 | AM17882-AS | AM17881-SS | 1931 |
| AD12953 | AM18384-AS | AM17487-SS | 305 |
| AD12954 | AM18385-AS | AM17487-SS | 305 |
| AD12955 | AM18386-AS | AM17487-SS | 305 |
| AD12956 | AM18387-AS | AM17487-SS | 305 |
| AD12957 | AM18388-AS | AM17499-SS | 1817 |
| AD12958 | AM18389-AS | AM17499-SS | 1817 |
| AD12959 | AM18390-AS | AM17499-SS | 1817 |
| AD12960 | AM18391-AS | AM17499-SS | 1817 |
| AD12961 | AM18393-AS | AM18392-SS | 1817 |
| AD12962 | AM18394-AS | AM18392-SS | 1817 |
| AD12972 | AM18398-AS | AM16755-SS | 1313 |
| AD12973 | AM18399-AS | AM16755-SS | 1313 |
| AD12974 | AM18401-AS | AM18400-SS | 1313 |
| AD12975 | AM18402-AS | AM18400-SS | 1313 |
| AD12976 | AM18403-AS | AM18400-SS | 1313 |
| AD12977 | AM18404-AS | AM16739-SS | 1089 |
| AD12978 | AM18405-AS | AM16739-SS | 1089 |
| AD12979 | AM18407-AS | AM18406-SS | 1089 |
| AD12980 | AM18408-AS | AM18406-SS | 1089 |
| AD12981 | AM18409-AS | AM18406-SS | 1089 |
| AD12982 | AM18410-AS | AM18406-SS | 1089 |
| AD12983 | AM18411-AS | AM18406-SS | 1089 |
| AD12984 | AM18412-AS | AM18406-SS | 1089 |
| AD12985 | AM18413-AS | AM18406-SS | 1089 |
| AD13113 | AM18606-AS | AM17495-SS | 1275 |
| AD13114 | AM18607-AS | AM17495-SS | 1275 |
| AD13115 | AM18608-AS | AM17495-SS | 1275 |
| AD13116 | AM18609-AS | AM17495-SS | 1275 |
| AD13117 | AM18610-AS | AM17495-SS | 1275 |
| AD13118 | AM18611-AS | AM17503-SS | 1954 |
| AD13119 | AM18612-AS | AM17503-SS | 1954 |
| AD13120 | AM18614-AS | AM18613-SS | 306 |
| AD13121 | AM18616-AS | AM18615-SS | 307 |
| AD13137 | AM18629-AS | AM16755-SS | 1313 |
| AD13138 | AM18629-AS | AM18630-SS | 1313 |
| AD13139 | AM18629-AS | AM18631-SS | 1313 |
| AD13140 | AM18632-AS | AM16755-SS | 1313 |
| AD13141 | AM18632-AS | AM18630-SS | 1313 |
| AD13142 | AM18632-AS | AM18631-SS | 1313 |
| AD13143 | AM18634-AS | AM18633-SS | 1314 |
| AD13144 | AM18636-AS | AM18635-SS | 1314 |
| AD13145 | AM18638-AS | AM18637-SS | 1314 |
| AD13278 | AM18878-AS | AM18877-SS | 2014 |
| AD13279 | AM18880-AS | AM18879-SS | 2018 |
| AD13280 | AM18882-AS | AM18881-SS | 2020 |
| AD13281 | AM18884-AS | AM18883-SS | 2630 |
| AD13282 | AM18886-AS | AM18885-SS | 2779 |
| AD13283 | AM18888-AS | AM18887-SS | 3190 |
| AD13284 | AM18890-AS | AM18889-SS | 3332 |
| AD13285 | AM18892-AS | AM18891-SS | 3412 |
| AD13286 | AM18894-AS | AM18893-SS | 3464 |
| AD13287 | AM18896-AS | AM18895-SS | 3991 |
| AD13288 | AM18898-AS | AM18897-SS | 4500 |
| AD13289 | AM18900-AS | AM18899-SS | 4582 |
| AD13290 | AM18902-AS | AM18901-SS | 4585 |
| AD13291 | AM18904-AS | AM18903-SS | 4623 |
| AD13292 | AM18906-AS | AM18905-SS | 4639 |
| AD13293 | AM18908-AS | AM18907-SS | 5255 |
| AD13294 | AM18910-AS | AM18909-SS | 5849 |
| AD13295 | AM18912-AS | AM18911-SS | 6143 |
| AD13296 | AM18914-AS | AM18913-SS | 6294 |
| AD13297 | AM18916-AS | AM18915-SS | 6408 |
| AD13298 | AM18918-AS | AM18917-SS | 6537 |
| AD13299 | AM18920-AS | AM18919-SS | 6833 |
| AD13300 | AM18922-AS | AM18921-SS | 6957 |
| AD13301 | AM18924-AS | AM18923-SS | 7208 |
| AD13322 | AM18944-AS | AM18943-SS | 419 |
| AD13323 | AM18946-AS | AM18945-SS | 601 |
| AD13324 | AM18948-AS | AM18947-SS | 1124 |
| AD13325 | AM18950-AS | AM18949-SS | 1131 |
| AD13326 | AM18952-AS | AM18951-SS | 1157 |
| AD13327 | AM18954-AS | AM18953-SS | 1246 |
| AD13328 | AM18956-AS | AM18955-SS | 1332 |
| AD13329 | AM18958-AS | AM18957-SS | 1432 |
| AD13330 | AM18960-AS | AM18959-SS | 1842 |
| AD13444 | AM17500-AS | AM19119-SS | 1817 |
| AD13445 | AM19120-AS | AM19119-SS | 1817 |
| AD13446 | AM19121-AS | AM19119-SS | 1817 |
| AD13447 | AM19122-AS | AM19119-SS | 1817 |
| AD13448 | AM19123-AS | AM17499-SS | 1817 |
| AD13449 | AM19123-AS | AM19124-SS | 1817 |
| AD13450 | AM19121-AS | AM17499-SS | 1817 |
| AD13451 | AM19121-AS | AM19124-SS | 1817 |
| AD13452 | AM19125-AS | AM17833-SS | 1014 |
| AD13453 | AM19126-AS | AM17833-SS | 1014 |
| AD13454 | AM19127-AS | AM17833-SS | 1014 |
| AD13455 | AM19128-AS | AM17833-SS | 1014 |
| AD13456 | AM19127-AS | AM19129-SS | 1014 |
| AD13457 | AM19127-AS | AM19130-SS | 1014 |
| AD13458 | AM19131-AS | AM17833-SS | 1014 |
| AD13459 | AM19132-AS | AM17833-SS | 1014 |
| AD13507 | AM19181-AS | AM17495-SS | 1275 |
| AD13508 | AM19182-AS | AM17495-SS | 1275 |
| AD13509 | AM19183-AS | AM17495-SS | 1275 |
| AD13510 | AM19185-AS | AM19184-SS | 1275 |
| AD13511 | AM19186-AS | AM19184-SS | 1275 |
| AD13512 | AM18610-AS | AM19187-SS | 1275 |
| AD13513 | AM19189-AS | AM19188-SS | 1275 |
| AD13514 | AM19191-AS | AM19190-SS | 1275 |
| AD13515 | AM19193-AS | AM19192-SS | 1275 |
| AD13516 | AM19193-AS | AM19194-SS | 1275 |
| AD13535 | AM19219-AS | AM19218-SS | 1059 |
| AD13536 | AM19221-AS | AM19220-SS | 1058 |
| AD13537 | AM19223-AS | AM19222-SS | 1060 |
| AD13705 | AM18410-AS | AM19404-SS | 1089 |
| AD13706 | AM19405-AS | AM18406-SS | 1089 |
| AD13707 | AM19406-AS | AM18406-SS | 1089 |
| AD13708 | AM19407-AS | AM17881-SS | 1931 |
| AD13709 | AM19408-AS | AM17881-SS | 1931 |
| AD13710 | AM19408-AS | AM19409-SS | 1931 |
| AD13711 | AM19410-AS | AM17881-SS | 1931 |
| AD13712 | AM19411-AS | AM17881-SS | 1931 |
| AD13804 | AM19536-AS | AM19184-SS | 1275 |
| AD13805 | AM19538-AS | AM19537-SS | 1275 |
| AD13805.1 | AM19538.1-AS | AM19537-SS | 1275 |
| AD13806 | AM19539-AS | AM19194-SS | 1275 |
| AD13921 | AM19656-AS | AM17877-SS | 1642 |
| AD13922 | AM19657-AS | AM17877-SS | 1642 |
| AD13923 | AM19658-AS | AM16747-SS | 1190 |
| AD13924 | AM19659-AS | AM16747-SS | 1190 |
| AD13925 | AM19661-AS | AM19660-SS | 1190 |
| AD13926 | AM19663-AS | AM19662-SS | 1190 |
| AC003589 | CA004443 | CS914989 | 1275 |
| AC003590 | CA004444 | CS914991 | 1275 |
| AC003591 | CA004446 | CS004445 | 1275 |
| AC003592 | CA004447 | CS914989 | 1275 |
| AC003593 | CA004449 | CS004448 | 711 |
| AC003625 | CA004481 | CS004480 | 1274 |
| AC003626 | CA004483 | CS004482 | 1276 |
| AC003891 | CA004798 | CS915323 | 1275 |
| AC004186 | CA005198 | CS005197 | 1059 |
| AC006749 | CA008056 | CS913717 | 409 |
| AC006750 | CA008057 | CS913729 | 901 |
| AC006751 | CA008058 | CS913739 | 1014 |
| AC007084 | CA008429 | CS008428 | 1313 |
| AC007085 | CA008430 | CS914289 | 1313 |

TABLE 6D
MARC1 RNAi Agent Duplexes Showing Chemically Modified Antisense Strand and Sense Strand Sequences

| Duplex ID: | Modified Antisense Strand (5′→3′) | SEQ ID NO. | Modified Sense Strand (5′→3′) | SEQ ID NO. |
|---|---|---|---|---|
| AD11764 | usAfsasCfcAfuguuuCfcCfuCfcUfggsu | 1056 | (NAG37)s(invAb)saccaggagGfGfAfaacaugguuas(invAb) | 1397 |
| AD11765 | usAfscsUfuCfaggaaGfcUfgGfuUfausc | 1057 | (NAG37)s(invAb)sgauaaccaGfCfUfuccugaaguas(invAb) | 1398 |
| AD11766 | usAfsasCfaAfgucugCfuAfuUfuGfausg | 1058 | (NAG37)s(invAb)scaucaaauAfGfCfagacuuguuas(invAb) | 1399 |
| AD11767 | usGfsgsAfaCfaagucUfgCfuAfuUfugsg | 1059 | (NAG37)s(invAb)sccaaauagCfAfGfacuuiuuccas(invAb) | 1400 |
| AD11768 | usGfsasGfuAfagcaaUfcUfgGfuCfcusc | 1060 | (NAG37)s(invAb)sgaggaccaGfAfUfugcuuacucas(invAb) | 1401 |
| AD11769 | usGfsusCfuGfaguaaGfcAfaUfcUfggsu | 1061 | (NAG37)s(invAb)saccagauuGfCfUfuacucagacas(invAb) | 1402 |
| AD11770 | usUfsgsGfuGfU_{UNA}cugaGfuAfaGfcAfausc | 1062 | (NAG37)s(invAb)sgauugcuuAfCfUfcagacaccaas(invAb) | 1403 |
| AD11771 | usUfsasAfcUfuucuuCfuCfuAfgCfcusg | 1063 | (NAG37)s(invAb)scaggcuagAfGfAfagaaaguuaas(invAb) | 1404 |
| AD11772 | usGfsgsUfuAfaaaugCfaUfcUfgGfaasc | 1064 | (NAG37)s(invAb)sguuccagaUfGfCfauuuuaaccas(invAb) | 1405 |
| AD11773 | asAfscsUfuUfcguucUfgAfaGfgGfucsa | 1065 | (NAG37)s(invAb)sugacccuuCfAfGfaacgaaaguus(invAb) | 1406 |
| AD11774 | usUfscsCfaUfauaacUfuUfcGfuUfcusg | 1066 | (NAG37)s(invAb)scagaacgaAfAfGfuuauauggaas(invAb) | 1407 |
| AD11775 | usGfsasUfuUfuccauAfuAfaCfuUfucsg | 1067 | (NAG37)s(invAb)scgaaaguuAfUfAfuggaaaaucas(invAb) | 1408 |
| AD11776 | asAfsasGfaGfuggugAfuUfuUfcCfausg | 1068 | (NAG37)s(invAb)scauggaaaAfUfCfaccacucuuus(invAb) | 1409 |
| AD11777 | usCfsasUfcUfaauauUfcCfaGfgAfcasc | 1069 | (NAG37)s(invAb)sguguccugGfAfAfuauuagaugas(invAb) | 1410 |
| AD11778 | asGfsusGfuUfgucauUfuUfuGfaGfaasc | 1070 | (NAG37)s(invAb)sguucucaaAfAfAfugacaacacus(invAb) | 1411 |
| AD11779 | usAfsusGfcUfucaagUfgUfuGfuCfausc | 1071 | (NAG37)s(invAb)sgaugacaaCfAfCfuugaagcauas(invAb) | 1412 |
| AD11780 | asCfsasCfcAfU_{UNA}gcuuCfaAfgUfgUfugsc | 1072 | (NAG37)s(invAb)sgcaacacuUfGfAfagcauggugus(invAb) | 1413 |
| AD11781 | asGfsusUfcUfgaaacAfcCfaUfgCfuusc | 1073 | (NAG37)s(invAb)sgaagcaugGfUfGfuuucagaacus(invAb) | 1414 |
| AD11782 | usAfsusUfgAfagcauUfgAfgAfcAfccsa | 1074 | (NAG37)s(invAb)suggugucuCfAfAfugcuucaauas(invAb) | 1415 |
| AD11783 | usGfsasCfaUfugaagCfaUfuGfaGfacsg | 1075 | (NAG37)s(invAb)scgucucaaUfGfCfuucaaugucas(invAb) | 1416 |
| AD11784 | usGfsasGfuUfuucagAfaUfcCfuGfucsc | 1076 | (NAG37)s(invAb)sggacaggaUfUfCfugaaaacucas(invAb) | 1417 |
| AD11785 | asAfsgsAfaCfuauucCfaUfaAfuCfagsc | 1077 | (NAG37)s(invAb)sgcugauuaUfGfGfaauaguucuus(invAb) | 1418 |
| AD11786 | asGfsasAfaGfaacuaUfuCfcAfuAfausc | 1078 | (NAG37)s(invAb)sgauuauggAfAfUfaguucuuucus(invAb) | 1419 |
| AD11787 | asGfsasUfcCfagaucUfaUfgGfaAfaasc | 1079 | (NAG37)s(invAb)sguuuuccaUfAfGfaucuigaucus(invAb) | 1420 |
| AD11788 | asUfscsCfaAfugcugUfcUfgAfgAfagsc | 1080 | (NAG37)s(invAb)sgcuucucaGfAfCfagcauuggaus(invAb) | 1421 |
| AD11789 | asCfscsUfuUfaggaaAfuCfcAfaUfgcsc | 1081 | NAG37)s(invAb)sggcauuggAfUfUfuccuaaaggus(invAb) | 1422 |
| AD11790 | usCfsasCfcUfuuaggAfaAfuCfcAfausg | 1082 | (NAG37)s(invAb)scauuggauUfUfCfcuaaagguias(invAb) | 1423 |
| AD11791 | usGfsgsUfuUfuaaacAfaCfuGfaCfausg | 1083 | (NAG37)s(invAb)scaugucagUfUfGfuuuaaaaccas(invAb) | 1424 |
| AD11792 | usUfscsAfuCfagaucUfuAfgAfgUfuasc | 1084 | (NAG37)s(invAb)sguaacucuAfAfGfaucugaugaas(invAb) | 1425 |
| AD11793 | usUfsusCfaUfcagauCfuUfaGfaGfuusg | 1085 | (NAG37)s(invAb)scaacucuaAfGfAfucugaugaaas(invAb) | 1426 |
| AD11794 | usCfsasAfgUfuuaguCfaAfcUfuCfccsa | 1086 | (NAG37)s(invAb)sugggaaguUfGfAfcuaaacuugas(invAb) | 1427 |
| AD11795 | usAfsgsCfuUfccauuUfaUfuCfaCfagsc | 1087 | (NAG37)s(invAb)sgcugugaaUfAfAfauggaagcuas(invAb) | 1428 |
| AD12285 | asGfsusguugucauUfuUfuGfagaasc | 1088 | (NAG37)s(invAb)sguucucaaAfAfAfugacaacacus(invAb) | 1411 |
| AD12286 | cPrpasGfsusguugucauUfuUfuGfagaasc | 1089 | (NAG37)s(invAb)sguucucaaAfAfAfugacaacacus(invAb) | 1411 |
| AD12287 | usAfsusugaagcauUfgAfgAfcaccsa | 1090 | (NAG37)s(invAb)suggugucuCfAfAfugcuucaauas(invAb) | 1415 |
| AD12288 | cPrpusAfsusugaagcauUfgAfgAfcaccsa | 1091 | (NAG37)s(invAb)suggugucuCfAfAfugcuucaauas(invAb) | 1415 |
| AD12289 | asAfsgsaacuauucCfaUfaAfucagsc | 1092 | (NAG37)s(invAb)sgcugauuaUfGfGfaauaguucuus(invAb) | 1418 |
| AD12290 | cPrpasAfsgsaacuauucCfaUfaAfucagsc | 1093 | (NAG37)s(invAb)sgcugauuaUfGfGfaauaguucuus(invAb) | 1418 |
| AD12291 | asGfsasaagaacuaUfuCfcAfuaausc | 1094 | (NAG37)s(invAb)sgauuauggAfAfUfaguucuuucus(invAb) | 1419 |
| AD12292 | cPrpasGfsasaagaacuaUfuCfcAfuaausc | 1095 | (NAG37)s(invAb)sgauuauggAfAfUfaguucuuucus(invAb) | 1419 |
| AD12363 | usUfsgsAfuCfacaagCfcAfaAfaCfcusg | 1096 | (NAG37)s(invAb)scagguuuuGfGfCfuugugaucaas(invAb) | 1429 |
| AD12364 | usCfsasUfaGfacaucGfcAfuCfcUfgasa | 1097 | (NAG37)s(invAb)suucaggauGfCfGfaugucuaugas(invAb) | 1430 |
| AD12365 | usCfsasAfaGfaguggUfgAfuUfuUfccsa | 1098 | (NAG37)s(invAb)suggaaaauCfAfCfcacucuuugas(invAb) | 1431 |
| AD12366 | usUfscsUfgAfaacacCfaUfgCfuUfcasa | 1099 | (NAG37)s(invAb)suugaagcaUfGfGfuguuucagaas(invAb) | 1432 |
| AD12367 | usCfsasGfaAfuccugUfcUfuGfuCfausu | 1100 | (NAG37)s(invAb)saaugacaaGfAfCfaggauucugas(invAb) | 1433 |
| AD12368 | usCfsasAfuGfcugucUfgAfgAfaGfcasg | 1101 | (NAG37)s(invAb)scugcuucuCfAfGfacagcauugas(invAb) | 1434 |
| AD12369 | usCfscsUfuUfaaaggUfuUfuCfaGfuasg | 1102 | (NAG37)s(invAb)scuacugaaAfAfCfcuuuaaaggas(invAb) | 1435 |
| AD12370 | usAfscsUfuCfaucagAfuCfuUfaGfagsu | 1103 | (NAG37)s(invAb)sacucuaagAfUfCfugaugaaguas(invAb) | 1436 |
| AD12371 | usAfsasGfuUfuagucAfaCfuUfcCfcasa | 1104 | (NAG37)s(invAb)suugggaagUfUfGfacuaaacuuas(invAb) | 1437 |
| AD12372 | usCfsasAfuGfcugucUfgAfgAfaGfcasg | 1101 | (NAG37)s(invAb)scugcuucuCfAfGfacaguauugas(invAb) | 1438 |
| AD12583 | usGfsusGfuAfggcugCfaCfuGfaGfagsu | 1105 | (NAG37)s(invAb)sacucucagUfGfCfagccuacacas(invAb) | 1439 |
| AD12584 | usUfsgsUfgUfaggcuGfcAfcUfgAfgasg | 1106 | (NAG37)s(invAb)scucucaguGfCfAfgccuacacaas(invAb) | 1440 |
| AD12585 | usCfsusUfuGfuguagGfcUfgCfaCfugsa | 1107 | (NAG37)s(invAb)sucagugcaGfCfCfuacacaaagas(invAb) | 1441 |
| AD12586 | usUfscsCfuUfuguguAfgGfcUfgCfacsu | 1108 | (NAG37)s(invAb)sagugcagcCfUfAfcacaaaggaas(invAb) | 1442 |
| AD12587 | usUfscsGfaAfgugcaCfcAfgGfcGfgusa | 1109 | (NAG37)s(invAb)suaccgccuGfGfUfgcacuucgaas(invAb) | 1443 |
| AD12588 | usCfsusCfgAfagugcAfcCfaGfgCfggsu | 1110 | (NAG37)s(invAb)saccgccugGfUfGfcacuucgagas(invAb) | 1444 |
| AD12589 | usGfscsUfcGfaagugCfaCfcAfgGfcgsg | 1111 | (NAG37)s(invAb)sccgccuggUfGfCfacuucgagcas(invAb) | 1445 |
| AD12590 | asGfsgsCfuCfgaaguGfcAfcCfaGfgcsg | 1112 | (NAG37)s(invAb)scgccugguGfCfAfcuucgagccus(invAb) | 1446 |
| AD12591 | asAfscsUfcUfucaguGfuUfuCfcAfgcsg | 1113 | (NAG37)s(invAb)scgcuggaaAfCfAfcugaagaguus(invAb) | 1447 |
| AD12592 | usAfscsAfgGfgucucCfcAfcUfuUfgasu | 1114 | (NAG37)s(invAb)saucaaaguGfGfGfagacccuguas(invAb) | 1448 |
| AD12593 | asCfsasCfaGfggucuCfcCfaCfuUfugsa | 1115 | (NAG37)s(invAb)sucaaagugGfGfAfgacccugugus(invAb) | 1449 |
| AD12594 | usGfsusAfcAfcagggUfcUfcCfcAfcusu | 1116 | (NAG37)s(invAb)saagugggaGfAfCfccuguguacas(invAb) | 1450 |
| AD12595 | asGfsgsUfaCfacaggGfuCfuCfcCfacsu | 1117 | (NAG37)s(invAb)sagugggagAfCfCfcuguguaccus(invAb) | 1451 |
| AD12596 | usAfsgsGfuAfcacagGfgUfcUfcCfcasc | 1118 | (NAG37)s(invAb)sgugggagaCfCfCfuguguaccuas(invAb) | 1452 |
| AD12625 | usGfsasAfcAfagucuGfcUfaUfuUfgasu | 1119 | (NAG37)s(invAb)saucaaauaGfCfAfgacuuguucas(invAb) | 1453 |
| AD12626 | usGfscsUfuUfaacuuUfcUfuCfuCfuasg | 1120 | (NAG37)s(invAb)scuagagaaGfAfAfaguuaaagcas(invAb) | 1454 |
| AD12627 | usUfsusGfcUfuuaacUfuUfcUfuCfucsu | 1121 | (NAG37)s(invAb)sagagaagaAfAfGfuuaaagcaaas(invAb) | 1455 |
| AD12628 | usGfsasAfaUfuacaaUfaUfuGfgGfccsu | 1122 | (NAG37)s(invAb)saggcccaaUfAfUfuguaauuucas(invAb) | 1456 |
| AD12629 | usAfsasCfuUfucguuCfuGfaAfgGfgusc | 1123 | (NAG37)s(invAb)sgacccuucAfGfAfacgaaaguuas(invAb) | 1457 |
| AD12630 | usCfsasUfaUfaacuuUfcGfuUfcUfgasa | 1124 | (NAG37)s(invAb)suucagaacGfAfAfaguuauaugas(invAb) | 1458 |
| AD12631 | usUfscsCfaUfaaucaGfuUfaAfaCfggsg | 1125 | (NAG37)s(invAb)scccguuuaAfCfUfgauuauggaas(invAb) | 1459 |
| AD12632 | usUfsasGfgAfaauccAfaUfgCfuGfucsu | 1126 | (NAG37)s(invAb)sagacagcaUfUfGfgauuuccuaas(invAb) | 1460 |
| AD12633 | usAfsasAfgGfacaaaAfuGfgCfaAfuasa | 1127 | (NAG37)s(invAb)suuauugccAfUfUfuuguccuuuas(invAb) | 1461 |
| AD12634 | usAfsasUfcAfaaggaCfaAfaAfuGfgcsa | 1128 | (NAG37)s(invAb)sugccauuuUfGfUfccuuugauuas(invAb) | 1462 |
| AD12953 | usUfsgsauCfacaagCfcAfaAfaccusg | 1129 | (NAG37)s(invAb)scagguuuuGfGfCfuugugaucaas(invAb) | 1429 |
| AD12954 | cPrpusUfsgsauCfacaagCfcAfaAfaccusg | 1130 | (NAG37)s(invAb)scagguuuuGfGfCfuugugaucaas(invAb) | 1429 |
| AD12955 | usUfsgsaucacaagCfcAfaAfaccusg | 1131 | (NAG37)s(invAb)scagguuuuGfGfCfuugugaucaas(invAb) | 1429 |
| AD12956 | cPrpusUfsgsaucacaagCfcAfaAfaccusg | 1132 | (NAG37)s(invAb)scagguuuuGfGfCfuugugaucaas(invAb) | 1429 |
| AD12957 | usCfscsuuUfaaaggUfuUfuCfaguasg | 1133 | (NAG37)s(invAb)scuacugaaAfAfCfcuuuaaaggas(invAb) | 1435 |
| AD12958 | cPrpusCfscsuuUfaaaggUfuUfuCfaguasg | 1134 | (NAG37)s(invAb)scuacugaaAfAfCfcuuuaaaggas(invAb) | 1435 |
| AD12959 | usCfscsuuuaaaggUfuUfuCfaguasg | 1135 | (NAG37)s(invAb)scuacugaaAfAfCfcuuuaaaggas(invAb) | 1435 |
| AD12960 | cPrpusCfscsuuuaaaggUfuUfuCfaguasg | 1136 | (NAG37)s(invAb)scuacugaaAfAfCfcuuuaaaggas(invAb) | 1435 |
| AD12961 | usCfscsuuuaaaggUfuUfuCfagugsg | 1137 | (NAG37)s(invAb)sccacugaaAfAfCfcuuuaaaggas(invAb) | 1463 |
| AD12962 | cPrpusCfscsuuuaaaggUfuUfuCfagugsg | 1138 | (NAG37)s(invAb)sccacugaaAfAfCfcuuuaaaggas(invAb) | 1463 |
| AD12972 | asGfsasaaGfaacuaUfuCfcAfuaausc | 1139 | (NAG37)s(invAb)sgauuauggAfAfUfaguucuuucus(invAb) | 1419 |
| AD12973 | cPrpasGfsasaaGfaacuaUfuCfcAfuaausc | 1140 | (NAG37)s(invAb)sgauuauggAfAfUfaguucuuucus(invAb) | 1419 |
| AD12974 | usGfsasaaGfaacuaUfuCfcAfuaausc | 1141 | (NAG37)s(invAb)sgauuauggAfAfUfaguucuuucas(invAb) | 1464 |
| AD12975 | cPrpusGfsasaaGfaacuaUfuCfcAfuaausc | 1142 | (NAG37)s(invAb)sgauuauggAfAfUfaguucuuucas(invAb) | 1464 |
| AD12976 | cPrpusGfaaaGfaacuaUfuCfcAfuaausc | 1143 | (NAG37)s(invAb)sgauuauggAfAfUfaguucuuucas(invAb) | 1464 |
| AD12977 | asGfsusguUfgucauUfuUfuGfagaasc | 1144 | (NAG37)s(invAb)sguucucaaAfAfAfugacaacacus(invAb) | 1411 |
| AD12978 | cPrpasGfsusguUfgucauUfuUfuGfagaasc | 1145 | (NAG37)s(invAb)sguucucaaAfAfAfugacaacacus(invAb) | 1411 |
| AD12979 | usGfsusguUfgucauUfuUfuGfagaasc | 1146 | (NAG37)s(invAb)sguucucaaAfAfAfugacaacacas(invAb) | 1465 |
| AD12980 | cPrpusGfsusguUfgucauUfuUfuGfagaasc | 1147 | (NAG37)s(invAb)sguucucaaAfAfAfugacaacacas(invAb) | 1465 |
| AD12981 | cPrpusGfsusguU_{UNA}gucauUfuUfuGfagaasc | 1148 | (NAG37)s(invAb)sguucucaaAfAfAfugacaacacas(invAb) | 1465 |
| AD12982 | cPrpusGfuguUfgucauUfuUfuGfagaasc | 1149 | (NAG37)s(invAb)sguucucaaAfAfAfugacaacacas(invAb) | 1465 |
| AD12983 | usGfsusguugucauUfuUfuGfagaasc | 1150 | (NAG37)s(invAb)sguucucaaAfAfAfugacaacacas(invAb) | 1465 |
| AD12984 | cPrpusGfsusguugucauUfuUfuGfagaasc | 1151 | (NAG37)s(invAb)sguucucaaAfAfAfugacaacacas(invAb) | 1465 |
| AD12985 | cPrpusGfuguugucauUfuUfuGfagaasc | 1152 | (NAG37)s(invAb)sguucucaaAfAfAfugacaacacas(invAb) | 1465 |
| AD13113 | usCfsasgaAfuccugUfcUfuGfucausu | 1153 | (NAG37)s(invAb)saaugacaaGfAfCfaggauucugas(invAb) | 1433 |
| AD13114 | cPrpusCfsasgaAfuccugUfcUfuGfucausu | 1154 | (NAG37)s(invAb)saaugacaaGfAfCfaggauucugas(invAb) | 1433 |
| AD13115 | usCfsasgaauccugUfcUfuGfucausu | 1155 | (NAG37)s(invAb)saaugacaaGfAfCfaggauucugas(invAb) | 1433 |
| AD13116 | cPrpusCfsasgaauccugUfcUfuGfucausu | 1156 | (NAG37)s(invAb)saaugacaaGfAfCfaggauucugas(invAb) | 1433 |
| AD13117 | cPrpusCfagaauccugUfcUfuGfucausu | 1157 | (NAG37)s(invAb)saaugacaaGfAfCfaggauucugas(invAb) | 1433 |
| AD13118 | usAfsasguuuagucAfaCfuUfcccasa | 1158 | (NAG37)s(invAb)suugggaagUfUfGfacuaaacuuas(invAb) | 1437 |
| AD13119 | cPrpusAfsasguuuagucAfaCfuUfcccasa | 1159 | (NAG37)s(invAb)suugggaagUfUfGfacuaaacuuas(invAb) | 1437 |
| AD13120 | usUfsusGfaUfcacaaGfcCfaAfaAfccsu | 1160 | (NAG37)s(invAb)sagguuuugGfCfUfugugaucaaas(invAb) | 1466 |
| AD13121 | usGfsusUfgAfucacaAfgCfcAfaAfacsc | 1161 | (NAG37)s(invAb)sgguuuuggCfUfUfgugaucaacas(invAb) | 1467 |
| AD13137 | asGfsasaAfgaacuaUfuCfcAfuaausc | 1162 | (NAG37)s(invAb)sgauuauggAfAfUfaguucuuucus(invAb) | 1419 |
| AD13138 | asGfsasaAfgaacuaUfuCfcAfuaausc | 1162 | (NAG37)s(invAb)sgauuauggAfaUfaGfuucuuucus(invAb) | 1468 |
| AD13139 | asGfsasaAfgaacuaUfuCfcAfuaausc | 1162 | (NAG37)s(invAb)sgauuauggAfaUfaguucuuucus(invAb) | 1469 |
| AD13140 | asGfsasaagAfacuaUfuCfcAfuaausc | 1163 | (NAG37)s(invAb)sgauuauggAfAfUfaguucuuucus(invAb) | 1419 |
| AD13141 | asGfsasaagAfacuaUfuCfcAfuaausc | 1163 | (NAG37)s(invAb)sgauuauggAfaUfaGfuucuuucus(invAb) | 1468 |
| AD13142 | asGfsasaagAfacuaUfuCfcAfuaausc | 1163 | (NAG37)s(invAb)sgauuauggAfaUfaguucuuucus(invAb) | 1469 |
| AD13143 | usAfsgsaaAfgaacuAfuUfcCfauaasu | 1164 | (NAG37)s(invAb)sauuauggaAfUfAfguucuuucuas(invAb) | 1470 |
| AD13144 | usAfsgsaaAfgaacuAfuUfcCfauagsu | 1165 | (NAG37)s(invAb)sacuauggaAfUfAfguucuuucuas(invAb) | 1471 |
| AD13145 | usAfsgsaaAfgaacuAfuUfcCfauagsc | 1166 | (NAG37)s(invAb)sgcuauggaAfUfAfguucuuucuas(invAb) | 1472 |
| AD13278 | asGfsusAfaGfaucugAfaAfcUfaGfucsa | 1167 | (NAG37)s(invAb)sugacuaguUfUfCfagaucuuacus(invAb) | 1473 |
| AD13279 | asGfsusUfaGfuaagaUfcUfgAfaAfcusa | 1168 | (NAG37)s(invAb)suaguuucaGfAfUfcuuacuaacus(invAb) | 1474 |
| AD13280 | usAfsasGfuUfaguaaGfaUfcUfgAfaasc | 1169 | (NAG37)s(invAb)sguuucagaUfCfUfuacuaacuuas(invAb) | 1475 |
| AD13281 | usCfsasUfaAfaacaaGfcAfcAfuGfcasc | 1170 | (NAG37)s(invAb)sgugcauguGfCfUfuguuuuaugas(invAb) | 1476 |
| AD13282 | usUfsgsAfaCfacuucGfcUfuUfgUfcusc | 1171 | (NAG37)s(invAb)sgagacaaaGfCfGfaaguguucaas(invAb) | 1477 |
| AD13283 | asGfsasUfuCfuagagGfuCfaCfuGfgasa | 1172 | (NAG37)s(invAb)suuccagugAfCfCfucuagaaucus(invAb) | 1478 |
| AD13284 | usGfsgsAfaUfaguguGfcAfaAfuGfcusc | 1173 | (NAG37)s(invAb)sgagcauuuGfCfAfcacuauuccas(invAb) | 1479 |
| AD13285 | usUfsgsAfuCfucaaaCfaUfcUfgUfcasu | 1174 | (NAG37)s(invAb)saugacagaUfGfUfuugagaucaas(invAb) | 1480 |
| AD13286 | usAfsgsCfaUfaaguuGfcCfaCfaCfagsa | 1175 | (NAG37)s(invAb)sucugugugGfCfAfacuuaugcuas(invAb) | 1481 |
| AD13287 | usCfsasCfaAfucaagGfuCfuGfgUfcusg | 1176 | (NAG37)s(invAb)scagaccagAfCfCfuugauugugas(invAb) | 1482 |
| AD13288 | asGfsasAfaGfagaguGfcUfuCfuCfccsu | 1177 | (NAG37)s(invAb)sagggagaaGfCfAfcucucuuucus(invAb) | 1483 |
| AD13289 | usGfsusUfaGfagagaGfuUfuGfgUfuusc | 1178 | (NAG37)s(invAb)sgaaaccaaAfCfUfcucucuaacas(invAb) | 1484 |
| AD13290 | usAfsasGfgUfuagagAfgAfgUfuUfggsu | 1179 | (NAG37)s(invAb)saccaaacuCfUfCfucuaaccuuas(invAb) | 1485 |
| AD13291 | usGfsgsUfuUfuguaaCfuUfaCfaUfcasc | 1180 | (NAG37)s(invAb)sgugauguaAfGfUfuacaaaaccas(invAb) | 1486 |
| AD13292 | usCfsasCfuUfucaucAfcAfgGfuGfgusu | 1181 | (NAG37)s(invAb)saaccaccuGfUfGfaugaaagugas(invAb) | 1487 |
| AD13293 | usGfsasUfuAfuaaauGfcAfcCfaGfucsa | 1182 | (NAG37)s(invAb)sugacugguGfCfAfuuuauaaucas(invAb) | 1488 |
| AD13294 | usAfsgsAfgUfuauagUfcAfgAfgCfugsu | 1183 | (NAG37)s(invAb)sacagcucuGfAfCfuauaacucuas(invAb) | 1489 |
| AD13295 | usUfsasGfgAfugcucUfgUfaAfaGfgusa | 1184 | (NAG37)s(invAb)suaccuuuaCfAfGfagcauccuaas(invAb) | 1490 |
| AD13296 | asGfsusUfaGfacuuaAfcUfuAfgUfucsc | 1185 | (NAG37)s(invAb)sggaacuaaGfUfUfaagucuaacus(invAb) | 1491 |
| AD13297 | asGfsusAfaAfuucaaUfcUfcUfcUfgasg | 1186 | (NAG37)s(invAb)scucagagaGfAfUfugaauuuacus(invAb) | 1492 |
| AD13298 | usGfsusUfgAfuguuaAfcCfaGfaGfcusg | 1187 | (NAG37)s(invAb)scagcucugGfUfUfaacaucaacas(invAb) | 1493 |
| AD13299 | asAfsasCfuAfugaugUfcAfgGfuCfaasc | 1188 | (NAG37)s(invAb)sguugaccuGfAfCfaucauaguuus(invAb) | 1494 |
| AD13300 | asCfsasUfuAfacgagAfuCfuGfuUfugsa | 1189 | (NAG37)s(invAb)sucaaacagAfUfCfucguuaaugus(invAb) | 1495 |
| AD13301 | usCfsasGfaAfuauagCfuUfuAfaGfaasc | 1190 | (NAG37)s(invAb)sguucuuaaAfGfCfuauauucugas(invAb) | 1496 |
| AD13322 | asGfsusAfgUfaggucCfuUfuGfuGfuasg | 1191 | (NAG37)s(invAb)scuacacaaAfGfGfaccuacuacus(invAb) | 1497 |
| AD13323 | usUfsgsCfuAfuuugaUfgAfgGfaCfgusc | 1192 | (NAG37)s(invAb)sgacguccuCfAfUfcaaauagcaas(invAb) | 1498 |
| AD13324 | asUfsgsUfaGfaggucUfcAfgUfuCfugsa | 1193 | (NAG37)s(invAb)sucagaacuGfAfGfaccucuacaus(invAb) | 1499 |
| AD13325 | asAfsasGfaAfaauguAfgAfgGfuCfucsa | 1194 | (NAG37)s(invAb)sugagaccuCfUfAfcauuuucuuus(invAb) | 1500 |
| AD13326 | asCfsgsAfaAfaauguGfaAfaAfuCfacsa | 1195 | (NAG37)s(invAb)sugugauuuUfCfAfcauuuuucgus(invAb) | 1501 |
| AD13327 | usUfsasCfuGfaagucCfuAfcUfaAfgusu | 1196 | (NAG37)s(invAb)saacuuaguAfGfGfacuucaguaas(invAb) | 1502 |
| AD13328 | asGfsasUfaAfacggaGfaAfgCfaGfgasg | 1197 | (NAG37)s(invAb)scuccugcuUfCfUfccguuuaucus(invAb) | 1503 |
| AD13329 | asCfsasUfuCfuaggaUfaUfuCfuUfccsa | 1198 | (NAG37)s(invAb)suggaagaaUfAfUfccuagaaugus(invAb) | 1504 |
| AD13330 | usAfsasCfuGfacauaUfgCfuUfuCfcusu | 1199 | (NAG37)s(invAb)saaggaaagCfAfUfaugucaguuas(invAb) | 1505 |
| AD13444 | usCfscsUfuUfaaaggUfuUfuCfaGfuasg | 1102 | (NAG37)s(invAb)scuacugaaAfAfCfcuuuaaaigas(invAb) | 1506 |
| AD13445 | cPrpusCfscsUfuUfaaaggUfuUfuCfaGfuasg | 1200 | (NAG37)s(invAb)scuacugaaAfAfCfcuuuaaaigas(invAb) | 1506 |
| AD13446 | usCfscsUfuuaaaggUfuUfuCfaguasg | 1201 | (NAG37)s(invAb)scuacugaaAfAfCfcuuuaaaigas(invAb) | 1506 |
| AD13447 | cPrpusCfscsUfuuaaaggUfuUfuCfaguasg | 1202 | (NAG37)s(invAb)scuacugaaAfAfCfcuuuaaaigas(invAb) | 1506 |
| AD13448 | usCfscsuuuAfaaggUfuUfuCfaguasg | 1203 | (NAG37)s(invAb)scuacugaaAfAfCfcuuuaaaggas(invAb) | 1435 |
| AD13449 | usCfscsuuuAfaaggUfuUfuCfaguasg | 1203 | (NAG37)s(invAb)scuacugaaAfaCfcUfuuaaaggas(invAb) | 1507 |
| AD13450 | usCfscsUfuuaaaggUfuUfuCfaguasg | 1201 | (NAG37)s(invAb)scuacugaaAfAfCfcuuuaaaggas(invAb) | 1435 |
| AD13451 | usCfscsUfuuaaaggUfuUfuCfaguasg | 1201 | (NAG37)s(invAb)scuacugaaAfaCfcUfuuaaaggas(invAb) | 1507 |
| AD13452 | usAfsgsGfuacacagGfgUfcUfcccasc | 1204 | (NAG37)s(invAb)sgugggagaCfCfCfuguguaccuas(invAb) | 1452 |
| AD13453 | usAfsgsguAfcacagGfgUfcUfcccasc | 1205 | (NAG37)s(invAb)sgugggagaCfCfCfuguguaccuas(invAb) | 1452 |
| AD13454 | usAfsgsguaCfacagGfgUfcUfcccasc | 1206 | (NAG37)s(invAb)sgugggagaCfCfCfuguguaccuas(invAb) | 1452 |
| AD13455 | cPrpusAfsgsguaCfacagGfgUfcUfcccasc | 1207 | (NAG37)s(invAb)sgugggagaCfCfCfuguguaccuas(invAb) | 1452 |
| AD13456 | usAfsgsguaCfacagGfgUfcUfcccasc | 1206 | (NAG37)s(invAb)sgugggagaCfcCfuGfuguaccuas(invAb) | 1508 |
| AD13457 | usAfsgsguaCfacagGfgUfcUfcccasc | 1206 | (NAG37)s(invAb)sgugggagaCfCfCfuguguaucuas(invAb) | 1509 |
| AD13458 | usAfsgsguaCfacagGfgUfcUfcccasu | 1208 | (NAG37)s(invAb)sgugggagaCfCfCfuguguaccuas(invAb) | 1452 |
| AD13459 | usAfsgsguaCfacagGfgUfcUfccuasc | 1209 | (NAG37)s(invAb)sgugggagaCfCfCfuguguaccuas(invAb) | 1452 |
| AD13507 | cPrpusCfaGfaauccugUfcUfuGfucausu | 1210 | (NAG37)s(invAb)saaugacaaGfAfCfaggauucugas(invAb) | 1433 |
| AD13508 | cPrpusCfagAfauccugUfcUfuGfucausu | 1211 | (NAG37)s(invAb)saaugacaaGfAfCfaggauucugas(invAb) | 1433 |
| AD13509 | cPrpusCfagaaUfccugUfcUfuGfucausu | 1212 | (NAG37)s(invAb)saaugacaaGfAfCfaggauucugas(invAb) | 1433 |
| AD13510 | cPrpasCfagaauccugUfcUfuGfucausu | 1213 | (NAG37)s(invAb)saaugacaaGfAfCfaggauucugus(invAb) | 1510 |
| AD13511 | asCfagaauccugUfcUfuGfucausu | 1214 | (NAG37)s(invAb)saaugacaaGfAfCfaggauucugus(invAb) | 1510 |
| AD13512 | cPrpusCfagaauccugUfcUfuGfucausu | 1157 | (NAG37)s(invAb)saaugacaaGfaCfaGfgauucugas(invAb) | 1511 |
| AD13513 | cPrpusCfagaauccugUfcUfuGfucgusu | 1215 | (NAG37)s(invAb)saacgacaaGfAfCfaggauucugas(invAb) | 1512 |
| AD13514 | cPrpusCfagaauccugUfcUfuGfucausc | 1216 | (NAG37)s(invAb)sgaugacaaGfAfCfaggauucugas(invAb) | 1513 |
| AD13515 | cPrpasCfaGfaauccugUfcUfuGfucausc | 1217 | (NAG37)s(invAb)sgaugacaaGfaCfaGfgauucugus(invAb) | 1514 |
| AD13516 | cPrpasCfaGfaauccugUfcUfuGfucausc | 1217 | (NAG37)s(invAb)sgaugacaaGfAfCfaggauucugus(invAb) | 1515 |
| AD13535 | asGfsgsCfaUfcuaauAfuUfcCfaGfgasc | 1218 | (NAG37)s(invAb)sguccuggaAfUfAfuuagaugccus(invAb) | 1516 |
| AD13536 | asGfscsAfuCfuaauaUfuCfcAfgGfacsa | 1219 | (NAG37)s(invAb)suguccuggAfAfUfauuagaugcus(invAb) | 1517 |
| AD13537 | asAfsgsGfcAfucuaaUfaUfuCfcAfggsa | 1220 | (NAG37)s(invAb)succuggaaUfAfUfuagaugccuus(invAb) | 1518 |
| AD13705 | cPrpusGfuguUfgucauUfuUfuGfagaasc | 1149 | (NAG37)s(invAb)sguucucaaAfaAfuGfacaacacas(invAb) | 1519 |
| AD13706 | cPrpusGfuguuGfucauUfuUfuGfagaasc | 1221 | (NAG37)s(invAb)sguucucaaAfAfAfugacaacacas(invAb) | 1465 |
| AD13707 | cPrpusGfuGfuugucauUfuUfuGfagaasc | 1222 | (NAG37)s(invAb)sguucucaaAfAfAfugacaacacas(invAb) | 1465 |
| AD13708 | usAfsasucaAfaggaCfaAfaAfuggcsa | 1223 | (NAG37)s(invAb)sugccauuuUfGfUfccuuugauuas(invAb) | 1462 |
| AD13709 | cPrpusAfaucaAfaggaCfaAfaAfuggcsa | 1224 | (NAG37)s(invAb)sugccauuuUfGfUfccuuugauuas(invAb) | 1462 |
| AD13710 | cPrpusAfaucaAfaggaCfaAfaAfuggcsa | 1224 | (NAG37)s(invAb)sugccauuuUfgUfcCfuuugauuas(invAb) | 1520 |
| AD13711 | usAfsasUfcaaaggaCfaAfaAfuggcsa | 1225 | (NAG37)s(invAb)sugccauuuUfGfUfccuuugauuas(invAb) | 1462 |
| AD13712 | cPrpusAfaUfcaaaggaCfaAfaAfuggcsa | 1226 | (NAG37)s(invAb)sugccauuuUfGfUfccuuugauuas(invAb) | 1462 |
| AD13804 | asCfagAfauccugUfcUfuGfucausu | 1227 | (NAG37)s(invAb)saaugacaaGfAfCfaggauucugus(invAb) | 1510 |
| AD13805 | asCfagAfauccugUfcUfuGfucgusu | 1228 | (NAG37)s(invAb)saacgacaaGfAfCfaggauucugus(invAb) | 1521 |
| AD13805.1 | isCfagAfauccugUfcUfuGfucgusu | 1229 | (NAG37)s(invAb)saacgacaaGfAfCfaggauucugus(invAb) | 1521 |
| AD13806 | asCfagAfauccugUfcUfuGfucausc | 1230 | (NAG37)s(invAb)sgaugacaaGfAfCfaggauucugus(invAb) | 1515 |
| AD13921 | usUfsasggaAfauccAfaUfgCfugucsu | 1231 | (NAG37)s(invAb)sagacagcaUfUfGfgauuuccuaas(invAb) | 1460 |
| AD13922 | cPrpusUfsasggaAfauccAfaUfgCfugucsu | 1232 | (NAG37)s(invAb)sagacagcaUfUfGfgauuuccuaas(invAb) | 1460 |
| AD13923 | usAfsusugaAfgcauUfgAfgAfcaccsa | 1233 | (NAG37)s(invAb)suggugucuCfAfAfugcuucaauas(invAb) | 1415 |
| AD13924 | cPrpusAfuugaAfgcauUfgAfgAfcaccsa | 1234 | (NAG37)s(invAb)suggugucuCfAfAfugcuucaauas(invAb) | 1415 |
| AD13925 | usAfsusugaAfgcauUfgAfgAfcaccsg | 1235 | (NAG37)s(invAb)scggugucuCfAfAfugcuucaauas(invAb) | 1522 |
| AD13926 | usAfsusugaAfgcauUfgAfgAfcaucsg | 1236 | (NAG37)s(invAb)scgaugucuCfAfAfugcuucaauas(invAb) | 1523 |
| AC003589 | cPrpusCfagAfauccugUfcUfuGfucgusu | 1237 | (NAG37)s(invAb)saacgacaaGfAfCfaggauucugas(invAb) | 1539 |
| AC003590 | cPrpusCfagAfauccugUfcUfuGfucausc | 1238 | (NAG37)s(invAb)sgaugacaaGfAfCfaggauucugas(invAb) | 1540 |
| AC003591 | cPrpusCfagAfauccugUfcUfuGfucgusc | 1239 | (NAG37)s(invAb)sgacgacaaGfAfCfaggauucugas(invAb) | 1524 |
| AC003592 | cPrpusCfagAfaUUNAccugUfcUfuGfucgusu | 1240 | (NAG37)s(invAb)saacgacaaGfAfCfaggauucugas(invAb) | 1539 |
| AC003593 | usUfuaAfcuuucuUfcUfcUfagccsu | 1241 | (NAG37)s(invAb)saggcuagaGfAfAfgaaaguuaaas(invAb) | 1525 |
| AC003625 | usAfsgsaAfuccuguCfuUfgUfcauusc | 1242 | (NAG37)s(invAb)sgaaugacaAfGfAfcaggauucuas(invAb) | 1526 |
| AC003626 | usUfscsaGfaauccuGfuCfuUfgucasc | 1243 | (NAG37)s(invAb)sgugacaagAfCfAfggauucugaas(invAb) | 1527 |
| AC003891 | isCfagAfauccugUfcUfuGfucgusu | 1244 | (NAG37)s(invAb)saacgacaaGfAfCfaggauucugus(invAb) | 1541 |
| AC004186 | asGfsgcauCfuaauauUfcCfaggsusu | 1245 | (NAG37)sccuggaAfuAfUfUfAfgaugccuus(invAb) | 1528 |
| AC006749 | usCfuuUfguguagGfcUfgCfacugsa | 1246 | (NAG37)s(invAb)sucagugcaGfCfCfuacacaaagas(invAb) | 1535 |
| AC006750 | asAfcuCfuucaguGfuUfuCfcagcsg | 1247 | (NAG37)s(invAb)scgcuggaaAfCfAfcugaagaguus(invAb) | 1536 |
| AC006751 | usAfggUfacacagGfgUfcUfcccasc | 1248 | (NAG37)s(invAb)sgugggagaCfCfCfuguguaccuas(invAb) | 1537 |
| AC007084 | cPrpusGfaaagaacuaguCfcauaausc | 1249 | (NAG37)s(invAb)sgauuauggAfAfUfaggucuuucas(invAb) | 1534 |
| AC007085 | cPrpusGfaaaGfaacuaUfuCfcAfuaasusc | 1250 | (NAG37)s(invAb)sgauuauggAfAfUfaguucuuucas(invAb) | 1538 |

(A^{2N}) = 2-aminoadenine-containing nucleotide; I = hypoxanthine (inosine) nucleotide

In some embodiments, a MARC1 RNAi agent is prepared or provided as a salt, mixed salt, or a free-acid. The RNAi agents described herein, upon delivery to a cell expressing a MARC1 gene, inhibit or knockdown expression of one or more MARC1 genes in vivo and/or in vitro.

### Targeting Ligands or Groups, Linking Groups, and Delivery Vehicles

In some embodiments, a MARC1 RNAi agent is conjugated to one or more non-nucleotide groups including, but not limited to, a targeting group, a linking group, a targeting ligand, a delivery polymer, or a delivery vehicle. The non-nucleotide group can enhance targeting, delivery or attachment of the RNAi agent. Examples of targeting groups and linking groups are provided in Table 7. The non-nucleotide group can be covalently linked to the 3′ and/or 5′ end of either the sense strand and/or the antisense strand. In some embodiments, a MARC1 RNAi agent contains a non-nucleotide group linked to the 3′ and/or 5′ end of the sense strand. In some embodiments, a non-nucleotide group is linked to the 5′ end of a MARC1 RNAi agent sense strand. A non-nucleotide group may be linked directly or indirectly to the RNAi agent via a linker/linking group. In some embodiments, a non-nucleotide group is linked to the RNAi agent via a labile, cleavable, or reversible bond or linker.

In some embodiments, a non-nucleotide group enhances the pharmacokinetic or biodistribution properties of an RNAi agent or conjugate to which it is attached to improve cell- or tissue-specific distribution and cell-specific uptake of the RNAi agent or conjugate. In some embodiments, a non-nucleotide group enhances endocytosis of the RNAi agent.

Targeting groups or targeting moieties enhance the pharmacokinetic or biodistribution properties of a conjugate or RNAi agent to which they are attached to improve cell-specific (including, in some cases, organ specific) distribution and cell-specific (or organ specific) uptake of the conjugate or RNAi agent. A targeting group can be monovalent, divalent, trivalent, tetravalent, or have higher valency for the target to which it is directed. Representative targeting groups include, without limitation, compounds with affinity to cell surface molecules, cell receptor ligands, haptens, antibodies, monoclonal antibodies, antibody fragments, and antibody mimics with affinity to cell surface molecules.

In some embodiments, a targeting group is linked to an RNAi agent using a linker, such as a PEG linker or one, two, or three abasic and/or ribitol (abasic ribose) residues, which can in some instances serve as linkers. In some embodiments, a targeting ligand comprises a galactose-derivative cluster.

The MARC1 RNAi agents described herein can be synthesized having a reactive group, such as an amino group (also referred to herein as an amine), at the 5′-terminus and/or the 3′-terminus. The reactive group can be used subsequently to attach a targeting moiety using methods typical in the art.

In some embodiments, a targeting group comprises an asialoglycoprotein receptor ligand. As used herein, an asialoglycoprotein receptor ligand is a ligand that contains a moiety having affinity for the asialoglycoprotein receptor. As noted herein, the asialoglycoprotein receptor is highly expressed on hepatocytes. In some embodiments, an asialoglycoprotein receptor ligand includes or consists of one or more galactose derivatives. As used herein, the term galactose derivative includes both galactose and derivatives of galactose having affinity for the asialoglycoprotein receptor that is equal to or greater than that of galactose. Galactose derivatives include, but are not limited to: galactose, galactosamine, N-formylgalactosamine, N-acetyl-galactosamine, N-propionyl-galactosamine, N-n-butanoyl-galactosamine, and N-iso-butanoylgalactos-amine (see for example: S.T. Iobst and K. Drickamer, J. B. C., 1996, 271, 6686). Galactose derivatives, and clusters of galactose derivatives, that are useful for in vivo targeting of oligonucleotides and other molecules to the liver are known in the art (see, for example, Baenziger and Fiete, 1980, Cell, 22, 611-620; Connolly et al., 1982, J. Biol. Chem., 257, 939-945).

Galactose derivatives have been used to target molecules to hepatocytes in vivo through their binding to the asialoglycoprotein receptor expressed on the surface of hepatocytes. Binding of asialoglycoprotein receptor ligands to the asialoglycoprotein receptor(s) facilitates cell-specific targeting to hepatocytes and endocytosis of the molecule into hepatocytes. Asialoglycoprotein receptor ligands can be monomeric (e.g., having a single galactose derivative, also referred to as monovalent or monodentate) or multimeric (e.g., having multiple galactose derivatives). The galactose derivative or galactose derivative cluster can be attached to the 3′ or 5′ end of the sense or antisense strand of the RNAi agent using methods known in the art.

The preparation of targeting ligands, such as galactose derivative clusters, is described in, for example, International Patent Application Publication No. WO 2018/044350 to Arrowhead Pharmaceuticals, Inc., and International Patent Application Publication No. WO 2017/156012 to Arrowhead Pharmaceuticals, Inc., the contents of both of which are incorporated by reference herein in their entirety.

As used herein, a galactose derivative cluster comprises a molecule having two to four terminal galactose derivatives. A terminal galactose derivative is attached to a molecule through its C-1 carbon. In some embodiments, the galactose derivative cluster is a galactose derivative trimer (also referred to as tri-antennary galactose derivative or tri-valent galactose derivative). In some embodiments, the galactose derivative cluster comprises N-acetyl-galactosamine moieties. In some embodiments, the galactose derivative cluster comprises three N-acetyl-galactosamine moieties. In some embodiments, the galactose derivative cluster is a galactose derivative tetramer (also referred to as tetra-antennary galactose derivative or tetra-valent galactose derivative). In some embodiments, the galactose derivative cluster comprises four N-acetyl-galactosamine moieties.

As used herein, a galactose derivative trimer contains three galactose derivatives, each linked to a central branch point. As used herein, a galactose derivative tetramer contains four galactose derivatives, each linked to a central branch point. The galactose derivatives can be attached to the central branch point through the C-1 carbons of the saccharides. In some embodiments, the galactose derivatives are linked to the branch point via linkers or spacers. In some embodiments, the linker or spacer is a flexible hydrophilic spacer, such as a PEG group (see. e.g., U.S. Pat. No. 5,885,968; Biessen et al. J. Med. Chem. 1995 Vol. 39 p. 1538-1546). In some embodiments, the PEG spacer is a PEG3 spacer. The branch point can be any small molecule which permits attachment of three galactose derivatives and further permits attachment of the branch point to the RNAi agent. An example of branch point group is a di-lysine or di-glutamate. Attachment of the branch point to the RNAi agent can occur through a linker or spacer. In some embodiments, the linker or spacer comprises a flexible hydrophilic spacer, such as, but not limited to, a PEG spacer. In some embodiments, the linker comprises a rigid linker, such as a cyclic group. In some embodiments, a galactose derivative comprises or consists of N-acetyl-galactosamine. In some embodiments, the galactose derivative cluster is comprised of a galactose derivative tetramer, which can be, for example, an N-acetyl-galactosamine tetramer.

Embodiments of the present disclosure include pharmaceutical compositions for delivering a MARC1 RNAi agent to a liver cell in vivo. Such pharmaceutical compositions can include, for example, a MARC1 RNAi agent conjugated to a galactose derivative cluster. In some embodiments, the galactose derivative cluster is comprised of a galactose derivative trimer, which can be, for example, an N-acetyl-galactosamine trimer, or galactose derivative tetramer, which can be, for example, an N-acetyl-galactosamine tetramer.

A targeting ligand or targeting group can be linked to the 3′ or 5′ end of a sense strand or an antisense strand of a MARC1 RNAi agent disclosed herein.

Targeting ligands include, but are not limited to (NAG37) and (NAG37) s as defined in Table 7. Other targeting groups and targeting ligands, including galactose cluster targeting ligands, are known in the art.

In some embodiments, a linking group is conjugated to the RNAi agent. The linking group facilitates covalent linkage of the agent to a targeting group, delivery polymer, or delivery vehicle. The linking group can be linked to the 3′ and/or the 5′ end of the RNAi agent sense strand or antisense strand. In some embodiments, the linking group is linked to the RNAi agent sense strand. In some embodiments, the linking group is conjugated to the 5′ or 3′ end of an RNAi agent sense strand. In some embodiments, a linking group is conjugated to the 5′ end of an RNAi agent sense strand. Examples of linking groups, can include, but are not limited to: reactive groups such a primary amines and alkynes, alkyl groups, abasic nucleotides, ribitol (abasic ribose), and/or PEG groups.

In some embodiments, a targeting group is linked internally to a nucleotide on the sense strand and/or the antisense strand of the RNAi agent. In some embodiments, a targeting group is linked to the RNAi agent via a linker.

A linker or linking group is a connection between two atoms that links one chemical group (such as an RNAi agent) or segment of interest to another chemical group (such as a targeting group or delivery polymer) or segment of interest via one or more covalent bonds. A labile linkage contains a labile bond. A linkage can optionally include a spacer that increases the distance between the two joined atoms. A spacer can further add flexibility and/or length to the linkage. Spacers include, but are not be limited to, alkyl groups, alkenyl groups, alkynyl groups, aryl groups, aralkyl groups, aralkenyl groups, and aralkynyl groups: each of which can contain one or more heteroatoms, heterocycles, amino acids, nucleotides, and saccharides. Spacer groups are well known in the art and the preceding list is not meant to limit the scope of the description.

In some embodiments, when two or more RNAi agents are included in a single composition, each of the RNAi agents may be linked to the same targeting group or two a different targeting groups (i.e., targeting groups having different chemical structure). In some embodiments, targeting groups are linked to the MARC1 RNAi agents disclosed herein without the use of an additional linker. In some embodiments, the targeting group itself is designed having a linker or other site to facilitate conjugation readily present. In some embodiments, when two or more MARC1 RNAi agents are included in a single molecule, each of the RNAi agents may utilize the same linker or different linkers (i.e., linkers having different chemical structures).

Any of the MARC1 RNAi agent nucleotide sequences listed in Tables 2, 3, 4, 5, or 6D, whether modified or unmodified, can contain 3′ and/or 5′ targeting group(s) or linking group(s). Any of the MARC1 RNAi agent sequences listed in Table 3, 4, or 6D, or are otherwise described herein, which contain a 3′ or 5′ targeting group or linking group, can alternatively contain no 3′ or 5′ targeting group or linking group, or can contain a different 3′ or 5′ targeting group or linking group including, but not limited to, those depicted in Table 7. Any of the MARC1 RNAi agent duplexes listed in Tables 6A, 6B, 6C, or 6D, whether modified or unmodified, can further comprise a targeting group or linking group, including, but not limited to, those depicted in Table 7, and the targeting group or linking group can be attached to the 3′ or 5′ terminus of either the sense strand or the antisense strand of the MARC1 RNAi agent duplex.

Examples of targeting groups and linking groups (which when combined can form targeting ligands) are provided in Table 7. Table 5 and Table 6D provide certain embodiments of MARC1 RNAi agent sense strands having a targeting group or linking group linked to the 5′ or 3′ end.

TABLE 7
Structures Representing Various Modified Nucleotides, Targeting Ligands or Targeting Groups,
Capping Residues, and Linking Groups

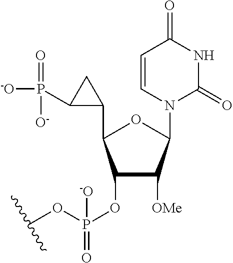

cPrpu

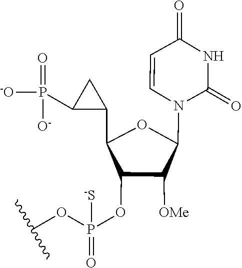

cPrpus

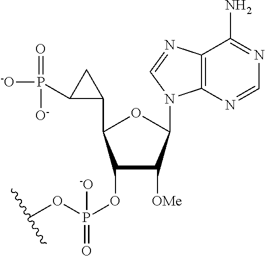

cPrpa

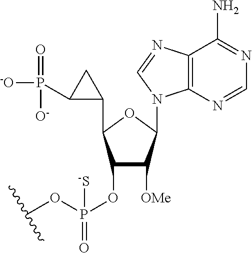

cPrpas

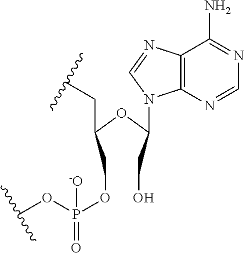

A_{UNA}

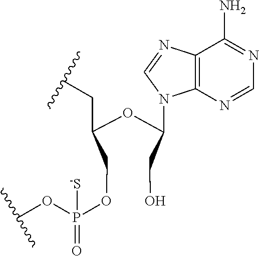

A_{UNA}s

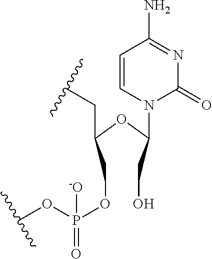

C_{UNA}

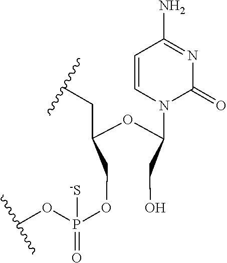

C_{UNA}s

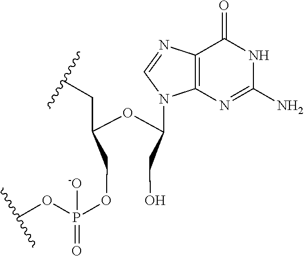

G_{UNA}

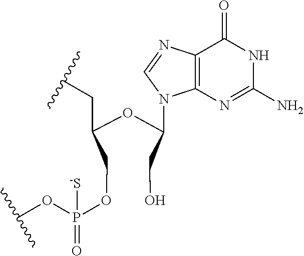

G_{UNA}s

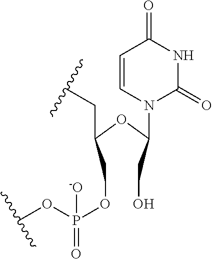

U_{UNA}

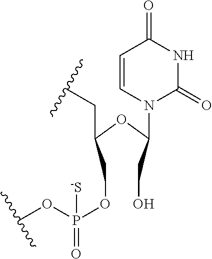

U_{UNA}s

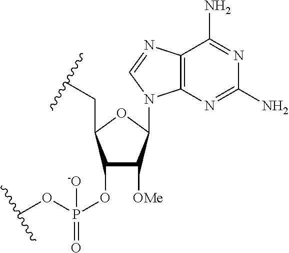

a_2N

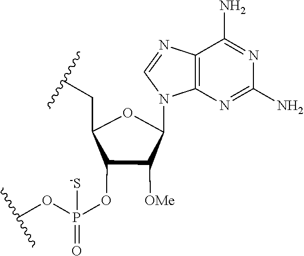

a_2Ns

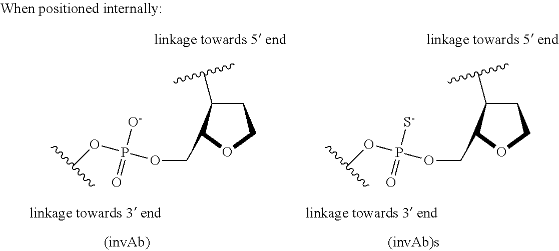

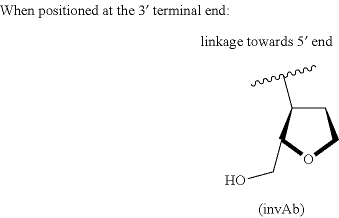

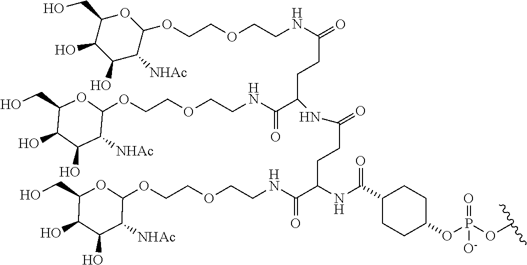 [SMILES: *OP(=O)([O-])O[C@H]1CC[C@@H](C(=O)NC(CCC(=O)NC(CCC(=O)NCCOCCOC2O[C@H](CO)[C@H](O)[C@H](O)[C@H]2NC(C)=O)C(=O)NCCOCCOC2O[C@H](CO)[C@H](O)[C@H](O)[C@H]2NC(C)=O)C(=O)NCCOCCOC2O[C@H](CO)[C@H](O)[C@H](O)[C@H]2NC(C)=O)CC1]

(NAG37)

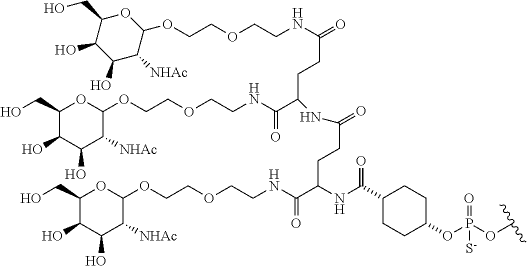 [SMILES: *OP(=O)([S-])O[C@H]1CC[C@@H](C(=O)NC(CCC(=O)NC(CCC(=O)NCCOCCOC2O[C@H](CO)[C@H](O)[C@H](O)[C@H]2NC(C)=O)C(=O)NCCOCCOC2O[C@H](CO)[C@H](O)[C@H](O)[C@H]2NC(C)=O)C(=O)NCCOCCOC2O[C@H](CO)[C@H](O)[C@H](O)[C@H]2NC(C)=O)CC1]

(NAG37)s

In each of the above structures in Table 7, NAG comprises an N-acetyl-galactosamine. In some embodiments, NAG as depicted in Table 7 above can comprise another galactose derivative that has affinity for the asialoglycoprotein receptor present on hepatocytes, as would be understood by a person of ordinary skill in the art to be attached in view of the structures above and description provided herein. Other linking groups known in the art may be used.

In some embodiments, a delivery vehicle can be used to deliver an RNAi agent to a cell or tissue. A delivery vehicle is a compound that improves delivery of the RNAi agent to a cell or tissue. A delivery vehicle can include, or consist of, but is not limited to: a polymer, such as an amphipathic polymer, a membrane active polymer, a peptide, a melittin peptide, a melittin-like peptide (MLP), a lipid, a reversibly modified polymer or peptide, or a reversibly modified membrane active polyamine. In some embodiments, the RNAi agents can be combined with lipids, nanoparticles, polymers, liposomes, micelles, DPCs or other delivery systems available in the art. The RNAi agents can also be chemically conjugated to targeting groups, lipids (including, but not limited to cholesterol and cholesteryl derivatives), nanoparticles, polymers, liposomes, micelles, DPCs (see, for example WO 2000/053722, WO 2008/0022309, WO 2011/104169, and WO 2012/083185, WO 2013/032829, WO 2013/158141, each of which is incorporated herein by reference), hydrogels, cyclodextrins, biodegradable nanocapsules, and bioadhesive microspheres, proteinaceous vectors, or other delivery systems suitable for nucleic acid or oligonucleotide delivery as known and available in the art.

### Pharmaceutical Compositions

The MARC1 RNAi agents disclosed herein can be prepared as pharmaceutical compositions or formulations (also referred to herein as “medicaments”). In some embodiments, pharmaceutical compositions include at least one MARC1 RNAi agent. These pharmaceutical compositions are particularly useful in the inhibition of the expression of the target mRNA in a target cell, a group of cells, a tissue, or an organism.

The pharmaceutical compositions can be used to treat a subject having a disease, disorder, or condition that would benefit from reduction in the level of the target MARC1 mRNA, or inhibition in expression of the target gene. The pharmaceutical compositions can be used to treat a subject at risk of developing a disease, disorder, symptom, or condition that would benefit from reduction of the level of the target mRNA or an inhibition in expression the target gene. In one embodiment, the method includes administering a MARC1 RNAi agent linked to a targeting ligand as described herein, to a subject to be treated. In some embodiments, one or more pharmaceutically acceptable excipients (including vehicles, carriers, diluents, and/or delivery polymers) are added to the pharmaceutical compositions that include a MARC1 RNAi agent, thereby forming a pharmaceutical formulation or medicament suitable for in vivo delivery to a subject, including a human.

The pharmaceutical compositions that include a MARC1 RNAi agent and methods disclosed herein decrease the level of the target mRNA in a cell, group of cells, group of cells, tissue, organ, or subject, including by administering to the subject a therapeutically effective amount of a herein described MARC1 RNAi agent, thereby inhibiting the expression or translation of MARC1 mRNA in the subject. In some embodiments, the subject has been previously identified as having a pathogenic upregulation of the target gene in hepatocytes. In some embodiments, the subject has been previously identified or diagnosed as having nonalcoholic steatohepatitis (NASH), nonalcoholic fatty liver disease (NAFLD), alcoholic fatty liver disease, autoimmune hepatitis, hepatic fibrosis, cirrhosis, elevated blood cholesterol levels, hypertriglyceridemia, liver disease, and/or other MARC1-related disease. In some embodiments, the subject would benefit from a reduction of MARC1 gene expression in the subject's liver.

In some embodiments, the described pharmaceutical compositions including a MARC1 RNAi agent are used for treating or managing clinical presentations associated nonalcoholic steatohepatitis (NASH), nonalcoholic fatty liver disease (NAFLD), alcoholic fatty liver disease, autoimmune hepatitis, hepatic fibrosis, cirrhosis, elevated blood cholesterol levels, hypertriglyceridemia, liver disease, and/or other MARC1-related disease. In some embodiments, a therapeutically (including prophylactically) effective amount of one or more of pharmaceutical compositions is administered to a subject in need of such treatment. In some embodiments, administration of any of the disclosed MARC1 RNAi agents can be used to decrease the number, severity, and/or frequency of symptoms of a disease in a subject.

The described pharmaceutical compositions that include a MARC1 RNAi agent can be used to treat at least one symptom in a subject having a disease or disorder that would benefit from reduction or inhibition in expression of MARC1 mRNA and/or a reduction in MARC1 protein levels. Measuring MARC1 levels can be conducted in accordance with established methods known in the art.

In some embodiments, the subject is administered a therapeutically effective amount of one or more pharmaceutical compositions that include a MARC1 RNAi agent thereby treating the symptom. In other embodiments, the subject is administered a prophylactically effective amount of one or more MARC1 RNAi agents, thereby preventing or inhibiting the at least one symptom.

The route of administration is the path by which a MARC1 RNAi agent is brought into contact with the body. In general, methods of administering drugs and oligonucleotides and nucleic acids for treatment of a mammal are well known in the art and can be applied to administration of the compositions described herein. The MARC1 RNAi agents disclosed herein can be administered via any suitable route in a preparation appropriately tailored to the particular route. Thus, herein described pharmaceutical compositions can be administered by injection, for example, intravenously, intramuscularly, intracutaneously, subcutaneously, intraarticularly, or intraperitoneally. In some embodiments, the herein described pharmaceutical compositions are administered via subcutaneous injection.

The pharmaceutical compositions including a MARC1 RNAi agent described herein can be delivered to a cell, group of cells, tissue, or subject using oligonucleotide delivery technologies known in the art. In general, any suitable method recognized in the art for delivering a nucleic acid molecule (in vitro or in vivo) can be adapted for use with the compositions described herein. For example, delivery can be by local administration, (e.g., direct injection, implantation, or topical administering), systemic administration, or subcutaneous, intravenous, intraperitoneal, or parenteral routes, including intracranial (e.g., intraventricular, intraparenchymal and intrathecal), intramuscular, transdermal, airway (aerosol), nasal, oral, rectal, or topical (including buccal and sublingual) administration. In certain embodiments, the compositions are administered by subcutaneous or intravenous infusion or injection.

In some embodiments, the pharmaceutical compositions described herein comprise one or more pharmaceutically acceptable excipients. The pharmaceutical compositions described herein are formulated for administration to a subject.

As used herein, a pharmaceutical composition or medicament includes a pharmacologically effective amount of at least one of the described therapeutic compounds and one or more pharmaceutically acceptable excipients. Pharmaceutically acceptable excipients (excipients) are substances other than the Active Pharmaceutical Ingredient (API, therapeutic product, e.g., MARC1 RNAi agent) that are intentionally included in the drug delivery system. Excipients do not exert or are not intended to exert a therapeutic effect at the intended dosage. Excipients can act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, detergents, dextran, dextrose, diluents, disintegrants, emulsifiers, extenders, fillers, flavors, glidants, humectants, lubricants, oils, polymers, preservatives, saline, salts, solvents, sugars, surfactants, suspending agents, sustained release matrices, sweeteners, thickening agents, tonicity agents, vehicles, water-repelling agents, and wetting agents.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water. CremophorR EL™ (BASF. Parsippany. NJ) or phosphate buffered saline (PBS). Suitable carriers should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle, which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

In some embodiments, pharmaceutical formulations that include the MARC1 RNAi agents disclosed herein suitable for subcutaneous administration can be prepared in an aqueous sodium phosphate buffer (e.g., the MARC1 RNAi agent formulated in 0.5 mM sodium phosphate monobasic. 0.5 mM sodium phosphate dibasic, in water). In some embodiments, pharmaceutical formulations that include the MARC1 RNAi agents disclosed herein suitable for subcutaneous administration can be prepared in water for injection (sterile water). MARC1 RNAi agents disclosed herein suitable for subcutaneous administration can be prepared in isotonic saline (0).9%).

Formulations suitable for intra-articular administration can be in the form of a sterile aqueous preparation of the drug that can be in microcrystalline form, for example, in the form of an aqueous microcrystalline suspension. Liposomal formulations or biodegradable polymer systems can also be used to present the drug for both intra-articular and ophthalmic administration.

Formulations suitable for oral administration of the MARC1 RNAi agents disclosed herein can also be prepared. In some embodiments, the MARC1 RNAi agents disclosed herein are administered orally. In some embodiments, the MARC1 RNAi agents disclosed herein are formulated in a capsule for oral administration.

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

The MARC1 RNAi agents can be formulated in compositions in dosage unit form for ease of administration and uniformity of dosage. Dosage unit form refers to physically discrete units suited as unitary dosages for the subject to be treated: each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the therapeutic effect to be achieved, and the limitations inherent in the art of compounding such an active compound for the treatment of individuals.

A pharmaceutical composition can contain other additional components commonly found in pharmaceutical compositions. Such additional components include, but are not limited to: anti-pruritics, astringents, local anesthetics, analgesics, antihistamines, or anti-inflammatory agents (e.g., acetaminophen. NSAIDs, diphenhydramine, etc.). It is also envisioned that cells, tissues, or isolated organs that express or comprise the herein defined RNAi agents may be used as “pharmaceutical compositions.” As used herein. “pharmacologically effective amount,” “therapeutically effective amount.” or simply “effective amount” refers to that amount of an RNAi agent to produce a pharmacological, therapeutic, or preventive result.

In some embodiments, the methods disclosed herein further comprise the step of administering a second therapeutic or treatment in addition to administering an RNAi agent disclosed herein. In some embodiments, the second therapeutic is another MARC1 RNAi agent (e.g., a MARC1 RNAi agent that targets a different sequence within the MARC1 target). In other embodiments, the second therapeutic can be a small molecule drug, an antibody, an antibody fragment, or an aptamer.

In some embodiments, the described MARC1 RNAi agent(s) are optionally combined with one or more additional therapeutics. The MARC1 RNAi agent and additional therapeutic(s) can be administered in a single composition or they can be administered separately. In some embodiments, the one or more additional therapeutics is administered separately in separate dosage forms from the RNAi agent (e.g., the MARC1 RNAi agent is administered by subcutaneous injection, while the additional therapeutic involved in the method of treatment dosing regimen is administered orally). In some embodiments, the described MARC1 RNAi agent(s) are administered to a subject in need thereof via subcutaneous injection, and the one or more optional additional therapeutics are administered orally, which together provide for a treatment regimen for diseases and conditions associated with nonalcoholic steatohepatitis (NASH), nonalcoholic fatty liver disease (NAFLD), alcoholic fatty liver disease, autoimmune hepatitis, hepatic fibrosis, cirrhosis, elevated blood cholesterol levels, hypertriglyceridemia, liver disease, and/or other MARC1-related disease. In some embodiments, the described MARC1 RNAi agent(s) are administered to a subject in need thereof via subcutaneous injection, and the one or more optional additional therapeutics are administered via a separate subcutaneous injection. In some embodiments, the MARC1 RNAi agent and one or more additional therapeutics are combined into a single dosage form (e.g., a “cocktail” formulated into a single composition for subcutaneous injection). The MARC1 RNAi agents, with or without the one or more additional therapeutics, can be combined with one or more excipients to form pharmaceutical compositions.

Generally, an effective amount of a MARC1 RNAi agent will be in the range of from about 0.1 to about 100 mg/kg of body weight/dose, e.g., from about 1.0 to about 50 mg/kg of body weight/dose. In some embodiments, an effective amount of an active compound will be in the range of from about 0.25 to about 5 mg/kg of body weight per dose. In some embodiments, an effective amount of an active ingredient will be in the range of from about 0.5 to about 4 mg/kg of body weight per dose. In some embodiments, an effective amount of a MARC1 RNAi agent may be a fixed dose. In some embodiments, the fixed dose is in the range of from about 5 mg to about 1,000 mg of MARC1 RNAi agent. In some embodiments, the fixed does is in the range of 10 to 400 mg of MARC1 RNAi agent. In some embodiments, the fixed does is in the range of 50 to 400 mg of MARC1 RNAi agent. Dosing may be weekly, bi-weekly, monthly, quarterly, or at any other interval depending on the dose of MARC1 RNAi agent administered, the activity level of the particular MARC1 RNAi agent, and the desired level of inhibition for the particular subject. The Examples herein show suitable levels for inhibition in certain animal species. The amount administered will depend on such variables as the overall health status of the patient or subject, the relative biological efficacy of the compound delivered, the formulation of the drug, the presence and types of excipients in the formulation, and the route of administration. Also, it is to be understood that the initial dosage administered can be increased beyond the above upper level to rapidly achieve the desired blood-level or tissue level, or the initial dosage can be smaller than the optimum.

For treatment of disease or for formation of a medicament or composition for treatment of a disease, the pharmaceutical compositions described herein including a MARC1 RNAi agent can be combined with an excipient or with a second therapeutic agent or treatment including, but not limited to: a second or other RNAi agent, a small molecule drug, an antibody, an antibody fragment, peptide and/or an aptamer.

The described MARC1 RNAi agents, when added to pharmaceutically acceptable excipients or adjuvants, can be packaged into kits, containers, packs, or dispensers. The pharmaceutical compositions described herein may be packaged in pre-filled syringes. pen injectors, autoinjectors, infusion bags/devices, or vials.

### Methods of Treatment and Inhibition of Expression

The MARC1 RNAi agents disclosed herein can be used to treat a subject (e.g., a human or other mammal) having a disease or disorder that would benefit from administration of the RNAi agent. In some embodiments, the RNAi agents disclosed herein can be used to treat a subject (e.g., a human) that would benefit from reduction and/or inhibition in expression of MARC1 mRNA and/or MARC1 protein levels, for example, a subject that has been diagnosed with or is suffering from symptoms related to nonalcoholic steatohepatitis (NASH), nonalcoholic fatty liver disease (NAFLD), alcoholic fatty liver disease, autoimmune hepatitis, hepatic fibrosis, cirrhosis, elevated blood cholesterol levels, hypertriglyceridemia, liver disease, and/or other MARC1-related disease.

In some embodiments, the subject is administered a therapeutically effective amount of any one or more MARC1 RNAi agents. Treatment of a subject can include therapeutic and/or prophylactic treatment. The subject is administered a therapeutically effective amount of any one or more MARC1 RNAi agents described herein. The subject may be an adult, adolescent, child, or infant. Administration of a pharmaceutical composition described herein can be to a human being or animal.

The MARC1 RNAi agents described herein can be used to treat at least one symptom in a subject having a MARC1-related disease or disorder, or having a disease or disorder that is mediated at least in part by MARC1 gene expression. In some embodiments, the MARC1 RNAi agents are used to treat or manage a clinical presentation of a subject with a disease or disorder that would benefit from or be mediated at least in part by a reduction in MARC1 mRNA or MARC1 protein levels. The subject is administered a therapeutically effective amount of one or more of the MARC1 RNAi agents or MARC1 RNAi agent-containing compositions described herein. In some embodiments, the methods disclosed herein comprise administering a composition comprising a MARC1 RNAi agent described herein to a subject to be treated. In some embodiments, the subject is administered a prophylactically effective amount of any one or more of the described MARC1 RNAi agents, thereby treating the subject by preventing or inhibiting the at least one symptom.

In certain embodiments, the present disclosure provides methods for treatment of diseases, disorders, conditions, or pathological states mediated at least in part by MARC1 gene expression, in a patient in need thereof, wherein the methods include administering to the patient any of the MARC1 RNAi agents described herein.

In some embodiments, the gene expression level and/or mRNA level of a MARC1 gene in a subject to whom a described MARC1 RNAi agent is administered is reduced by at least about 30%, 35%, 40%, 45%, 50%, 55%, 60%, 65%, 70%, 75%, 80%, 85%, 95%, 96%, 97%, 98%, 99%, or greater than 99% relative to the subject prior to being administered the MARC1 RNAi agent or to a subject not receiving the MARC1 RNAi agent. The MARC1 mRNA level in the subject may be reduced in a cell, group of cells, and/or tissue of the subject. In some embodiments, the MARC1 gene expression is inhibited by at least about 30%, 35%, 40%, 45% 50%, 55%, 60%, 65%, or greater than 65% in hepatocytes relative to the subject prior to being administered the MARC1 RNAi agent or to a subject not receiving the MARC1 RNAi agent.

In some embodiments, the MARC1 protein level in a subject to whom a described MARC1 RNAi agent has been administered is reduced by at least about 30%, 35%, 40%, 45%, 50%, 55%, 60%, 65%, 70%, 75%, 80%, 85%, 90%, 95%, 96%, 97%, 98%, 99%, or greater than 99% relative to the subject prior to being administered the MARC1 RNAi agent or to a subject not receiving the MARC1 RNAi agent. The protein level in the subject may be reduced in a cell, group of cells, tissue, blood, and/or other fluid of the subject.

A reduction in MARC1 mRNA levels and MARC1 protein levels can be assessed by any methods known in the art. As used herein, a reduction or decrease in MARC1 mRNA level and/or protein level are collectively referred to herein as a reduction or decrease in MARC1 or inhibiting or reducing the gene expression of MARC1. The Examples set forth herein illustrate known methods for assessing inhibition of MARC1 gene expression. The person of ordinary skill in the art would further know suitable methods for assessing inhibition of MARC1 gene expression in vivo and/or in vitro.

In some embodiments, disclosed herein are methods of treatment (including prophylactic or preventative treatment) of diseases, disorders, or symptoms caused by caused by nonalcoholic steatohepatitis (NASH), nonalcoholic fatty liver disease (NAFLD), alcoholic fatty liver disease, autoimmune hepatitis, hepatic fibrosis, cirrhosis, elevated blood cholesterol levels, hypertriglyceridemia, liver disease, and/or other MARC1-related disease, wherein the methods include administering to a subject in need thereof a therapeutically effective amount of a MARC1 RNAi agent that includes an antisense strand that is at least partially complementary to the portion of the MARC1 mRNA having the sequence in Table 1. In some embodiments, disclosed herein are methods of treatment (including prophylactic or preventative treatment) of diseases or symptoms caused by nonalcoholic steatohepatitis (NASH), nonalcoholic fatty liver disease (NAFLD), alcoholic fatty liver disease, autoimmune hepatitis, hepatic fibrosis, cirrhosis, elevated blood cholesterol levels, hypertriglyceridemia, liver disease, and/or other MARC1-related disease, wherein the methods include administering to a subject in need thereof a therapeutically effective amount of a MARC1 RNAi agent that includes an antisense strand comprising the sequence of any of the sequences in Tables 2, 3, or 6D, and a sense strand that comprises any of the sequences in Tables 2, 4, 5, or 6D, that is at least partially complementary to the antisense strand. In some embodiments, disclosed herein are methods of treatment (including prophylactic or preventative treatment) of diseases or symptoms caused by nonalcoholic steatohepatitis (NASH), nonalcoholic fatty liver disease (NAFLD), alcoholic fatty liver disease, autoimmune hepatitis, hepatic fibrosis, cirrhosis, elevated blood cholesterol levels, hypertriglyceridemia, liver disease, and/or other MARC1-related disease, wherein the methods include administering to a subject in need thereof a therapeutically effective amount of a MARC1 RNAi agent that includes a sense strand that comprises any of the sequences in Tables 2, 4, 5, or 6D, and an antisense strand comprising the sequence of any of the sequences in Tables 2, 3, or 6D that is at least partially complementary to the sense strand.

In some embodiments, disclosed herein are methods for inhibiting expression of a MARC1 gene in a cell, wherein the methods include administering to the cell a MARC1 RNAi agent that includes an antisense strand that is at least partially complementary to the portion of the MARC1 mRNA having the sequence in Table 1. In some embodiments, disclosed herein are methods of inhibiting expression of a MARC1 gene in a cell, wherein the methods include administering to a cell a MARC1 RNAi agent that includes an antisense strand comprising the sequence of any of the sequences in Tables 2, 3, or 6D, and a sense strand that comprises any of the sequences in Tables 2, 4, 5, or 6D that is at least partially complementary to the antisense strand. In some embodiments, disclosed herein are methods of inhibiting expression of a MARC1 gene in a cell, wherein the methods include administering a MARC1 RNAi agent that includes a sense strand that comprises any of the sequences in Tables 2, 4, 5, or 6D, and an antisense strand that includes the sequence of any of the sequences in Tables 2, 3, or 6D that is at least partially complementary to the sense strand.

The use of MARC1 RNAi agents provides methods for therapeutic (including prophylactic) treatment of diseases/disorders associated with nonalcoholic steatohepatitis (NASH), nonalcoholic fatty liver disease (NAFLD), alcoholic fatty liver disease, autoimmune hepatitis, hepatic fibrosis, cirrhosis, elevated blood cholesterol levels, hypertriglyceridemia, liver disease, and/or other MARC1-related disease. The described MARC1 RNAi agents mediate RNA interference to inhibit the expression of one or more genes necessary for production of MARC1 protein. MARC1 RNAi agents can also be used to treat or prevent various diseases, disorders, or conditions, including nonalcoholic steatohepatitis (NASH), nonalcoholic fatty liver disease (NAFLD), alcoholic fatty liver disease, autoimmune hepatitis, hepatic fibrosis, cirrhosis, elevated blood cholesterol levels, hypertriglyceridemia, liver disease, and/or other MARC1-related disease. Furthermore, compositions for delivery of MARC1 RNAi agents to liver cells, and specifically to hepatocytes. in vivo, are described.

### Cells, Tissues, Organs, and Non-Human Organisms

Cells, tissues, organs, and non-human organisms that include at least one of the MARC1 RNAi agents described herein are contemplated. The cell, tissue, organ, or non-human organism is made by delivering the RNAi agent to the cell, tissue, organ or non-human organism.

### Additional Illustrative Embodiments

Provided here are certain additional illustrative embodiments of the disclosed invention. These embodiments are illustrative only and do not limit the scope of the present disclosure or of the claims attached hereto.

Embodiment 1. An RNAi agent for inhibiting expression of a MARC1 gene, comprising: an antisense strand comprising a nucleotide sequence of at least 15 contiguous nucleotides differing by 0 or 1 nucleotides from 15 contiguous nucleotides of any one of the antisense strand sequences of Table 2, Table 3, or Table 6D; and a sense strand comprising a nucleotide sequence that is at least partially complementary to the antisense strand,

Embodiment 2. The RNAi agent of Embodiment 1, wherein the antisense strand comprises nucleotides 2-18 of any one of the sequences provided in Table 2, Table 3, or Table 6D.

Embodiment 3. The RNAi agent of Embodiment 1 or Embodiment 2, wherein the sense strand comprises a nucleotide sequence of at least 15 contiguous nucleotides differing by 0 or 1 nucleotides from 15 contiguous nucleotides of any one of the sense strand sequences of Table 2, Table 4, Table 5, or Table 6D, and wherein the sense strand has a region of at least 85% complementarity over at least 15 contiguous nucleotides to the antisense strand.

Embodiment 4. The RNAi agent of any one of Embodiments 1-3, wherein at least one nucleotide of the RNAi agent includes a modified internucleoside linkage.

Embodiment 5. The RNAi agent of any one of Embodiments 1-4, wherein all or substantially all of the nucleotides are modified nucleotides.

Embodiment 6. The RNAi agent of any one of Embodiments 4-5, wherein the modified nucleotides are independently selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.

Embodiment 7. The RNAi agent of Embodiment 5, wherein all or substantially all of the modified nucleotides are 2′-O-methyl nucleotides, 2′-fluoro nucleotides, or combinations thereof.

Embodiment 8. The RNAi agent of any one of Embodiments 1-7, wherein the antisense strand consists of or consists essentially of the nucleotide sequence of any one of the modified antisense strand sequences of Table 3.

Embodiment 9. The RNAi agent of any one of Embodiments 1-8 wherein the sense strand consists of, consists essentially of, or comprises the nucleotide sequence of any of the modified sense strand sequences of Table 4, Table 5, or Table 6D.

Embodiment 10. The RNAi agent of Embodiment 1, wherein the antisense strand comprises the nucleotide sequence of any one of the modified sequences of Table 3 or Table 6D, and the sense strand comprises the nucleotide sequence of any one of the modified sequences of Table 4, Table 5, or Table 6D.

Embodiment 11. The RNAi agent of any one of Embodiments 1-10, wherein the sense strand is between 18 and 30 nucleotides in length, and the antisense strand is between 18 and 30 nucleotides in length.

Embodiment 12. The RNAi agent of Embodiment 11, wherein the sense strand and the antisense strand are each between 18 and 27 nucleotides in length.

Embodiment 13. The RNAi agent of Embodiment 12, wherein the sense strand and the antisense strand are each between 18 and 24 nucleotides in length.

Embodiment 14. The RNAi agent of Embodiment 13, wherein the sense strand and the antisense strand are each 21 nucleotides in length.

Embodiment 15. The RNAi agent of Embodiment 14, wherein the RNAi agent has two blunt ends.

Embodiment 16. The RNAi agent of any one of Embodiments 1-15, wherein the sense strand comprises one or two terminal caps.

Embodiment 17. The RNAi agent of any one of Embodiments 1-16, wherein the sense strand comprises one or two inverted abasic residues.

Embodiment 18. The RNAi agent of Embodiment 1, wherein the RNAi agent is comprised of a sense strand and an antisense strand that form a duplex having the structure of any one of the duplexes in Table 6A and Table 6B.

Embodiment 19. The RNAi agent of Embodiment 18, wherein all or substantially all of the nucleotides are modified nucleotides.

Embodiment 20. The RNAi agent of Embodiment 1, comprising an antisense strand that consists of, consists essentially of, or comprises a nucleotide sequence that differs by 0 or 1 nucleotides from one of the following nucleotide sequences (5′→3′):

(SEQ ID NO: 1608)
UGAAAGAACUAUUCCAUAAUC;
(SEQ ID NO: 1657)
ACAGAAUCCUGUCUUGUCGUU;
(SEQ ID NO: 1580)
UCCUUUAAAGGUUUUCAGUAG;
or
(SEQ ID NO: 1659)
UAUUGAAGCAUUGAGACACCG.

Embodiment 21. The RNAi agent of any one of Embodiments 1-20, wherein the nucleotides of the antisense strand located at position 2 and position 14 from the 5′-end are 2′-fluoro modified nucleotides.

Embodiment 22. The RNAi agent of Embodiment 21, wherein the nucleotide of the antisense strand at position 2 is a 2′-fluoro uridine, and the nucleotide of the antisense strand at position 14 is a 2′-fluoro cytidine, and wherein the antisense strand comprises 3 or 4 phosphorothioate internucleoside linkages.

Embodiment 23. The RNAi agent of any one of Embodiments 1-22, wherein the sense strand consists of, consists essentially of, or comprises a nucleotide sequence that differs by 0 or 1 nucleotides from one of the following nucleotide sequences (5′→3′):

(SEQ ID NO: 1734)
GAUUAUGGAAUAGUUCUUUCA;
(SEQ ID NO: 1783)
AACGACAAGACAGGAUUCUGU;
(SEQ ID NO: 1774)
CUACUGAAAACCUUUAAAIGA;
or
(SEQ ID NO: 1784)
CGGUGUCUCAAUGCUUCAAUA

Embodiment 24. The RNAi agent of any one of Embodiments 20-23, wherein all or substantially all of the nucleotides are modified nucleotides.

Embodiment 25. The RNAi agent of Embodiment 1, comprising an antisense strand that comprises, consists of, or consists essentially of a modified nucleotide sequence that differs by 0 or 1 nucleotides from one of the following nucleotide sequences (5′→3′):

(SEQ ID NO: 1143)
cPrpusGfaaaGfaacuaUfuCfcAfuaausc;
(SEQ ID NO: 1228)
asCfagAfauccugUfcUfuGfucgusu;
(SEQ ID NO: 1229)
isCfagAfauccugUfcUfuGfucgusu;
(SEQ ID NO: 1200)
cPrpusCfscsUfuUfaaaggUfuUfuCfaGfuasg;
or
(SEQ ID NO: 1235)
usAfsusugaAfgcauUfgAfgAfcaccsg,

wherein a represents 2′-O-methyl adenosine, c represents 2′-O-methyl cytidine, g represents 2′-O-methyl guanosine, i represents 2′-O-methyl inosine; and u represents 2′-O-methyl uridine: Af, represents 2′-fluoro adenosine, Cf represents 2′-fluoro cytidine, Gf represents 2′-fluoro guanosine, and Uf represents 2′-fluoro uridine: cPrpu represents a 5′-cyclopropyl phosphonate-2′-O-methyl uridine: s represents a phosphorothioate linkage; and wherein all or substantially all of the nucleotides on the sense strand are modified nucleotides.

Embodiment 26. The RNAi agent of Embodiment 1, wherein the sense strand comprises, consists of, or consists essentially of a modified nucleotide sequence that differs by 0 or 1 nucleotides from one of the following nucleotide sequences (5′→3′):

(SEQ ID NO: 1319)
gauuauggAfAfUfaguucuuuca;
(SEQ ID NO: 1376)
aacgacaaGfAfCfaggauucugu;
(SEQ ID NO: 1361)
cuacugaaAfAfCfcuuuaaaiga;
or
(SEQ ID NO: 1377)
cggugucuCfAfAfugcuucaaua,

wherein a represents 2′-O-methyl adenosine, c represents 2′-O-methyl cytidine, g represents 2′-O-methyl guanosine, u represents 2′-O-methyl uridine, and i represents 2′-O-methyl inosine: Af, represents 2′-fluoro adenosine, Cf represents 2′-fluoro cytidine, Gf represents 2′-fluoro guanosine, and Uf represents 2′-fluoro uridine; s represents a phosphorothioate linkage; and wherein all or substantially all of the nucleotides on the antisense strand are modified nucleotides.

Embodiment 27. The RNAi agent of any one of Embodiments 20-26, wherein the sense strand further includes inverted abasic residues at the 3′ terminal end of the nucleotide sequence, at the 5′ end of the nucleotide sequence, or at both.

Embodiment 28. The RNAi agent of any one of Embodiments 1-27, wherein the RNAi agent is linked to a targeting ligand.

Embodiment 29. The RNAi agent of any one of Embodiments 1-28, wherein the targeting ligand comprises:

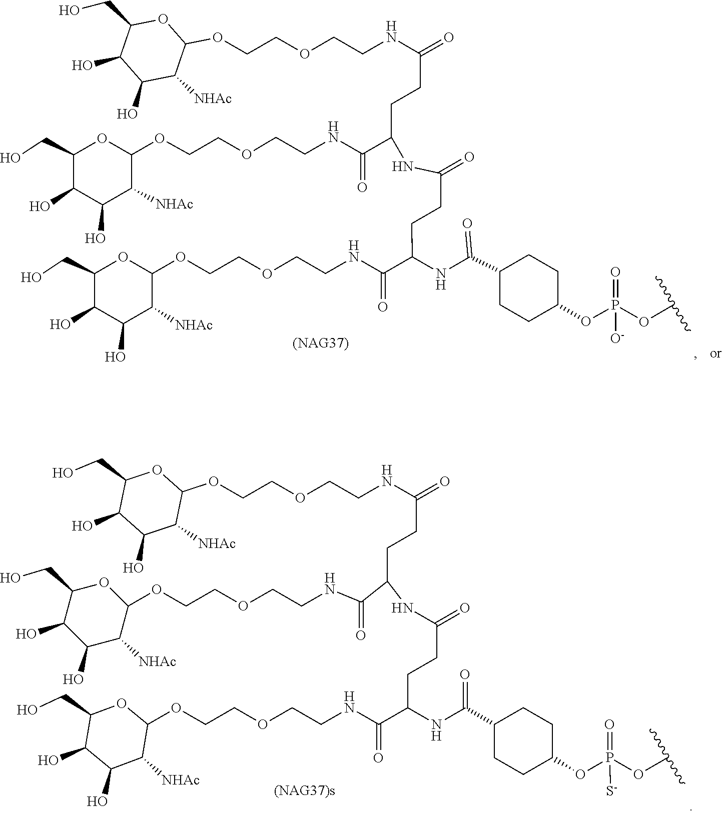

Embodiment 30. The RNAi agent of any one of Embodiments 1-29, wherein the targeting ligand is linked to the sense strand.

Embodiment 31. The RNAi agent of Embodiment 30, wherein the targeting ligand is linked to the 5′ terminal end of the sense strand.

Embodiment 32. A composition comprising the RNAi agent of any one of Embodiments 1-31, wherein the composition further comprises a pharmaceutically acceptable excipient.

Embodiment 33. The composition of Embodiment 32, further comprising a second RNAi agent capable of inhibiting the expression of a MARC1 gene.

Embodiment 34. The composition of any one of Embodiments 32-33, further comprising one or more additional therapeutics.

Embodiment 35. The composition of any one of Embodiments 32-34, wherein the composition is formulated for administration.

Embodiment 36. The composition of Embodiment 35, wherein the composition is delivered by subcutaneous injection.

Embodiment 37. The composition of any one of Embodiment 32-36, wherein the pharmaceutically acceptable excipient is a sodium phosphate buffer.

Embodiment 38. The composition of any one of Embodiment 32-36, wherein the pharmaceutically acceptable excipient is isotonic saline or water for injection.

Embodiment 39. A method for inhibiting expression of a MARC1 gene in a hepatocyte cell, the method comprising introducing into a cell of a subject an effective amount of an RNAi agent of any one of Embodiments 1-31 or the composition of any one of Embodiments 32-38.

Embodiment 40. The method of Embodiment 39, wherein the subject is a human subject.

Embodiment 41. The method of any one of Embodiments 39-40, wherein the MARC1 mRNA levels are reduced by at least about 50% in the hepatocyte cell or in the subject.

Embodiment 42. The method of any one of Embodiments 39-41, wherein the MARC1 protein levels are reduced by at least about 50% in the hepatocyte cell or in the subject.

Embodiment 43. A method of treating a MARC1-related disease, disorder, or symptom, the method comprising administering to a human subject in need thereof a therapeutically effective amount of the composition of any one of Embodiments 32-38.

Embodiment 44. The method of Embodiment 43, wherein the disease is hypertriglyceridemia, nonalcoholic steatohepatitis (NASH), alcoholic and nonalcoholic fatty liver disease (NAFLD), fatty liver disease, cirrhosis, elevated blood cholesterol levels, liver disease, autoimmune hepatitis and/or other MARC1-related disease.

Embodiment 45. The method of any one of Embodiments 39-44, wherein the level of serum MARC1 protein is decreased in the subject.

Embodiment 46. The method of any one of Embodiments 39-45, wherein the RNAi agent is administered to a human subject at a dose of about 0.05 mg/kg to about 5.0 mg/kg of body weight of the human subject.

Embodiment 47. Use of the RNAi agent of any one of Embodiments 1-31 or the composition according to any one of Embodiments 32-38, for the treatment of a disease, disorder, or symptom that is mediated at least in part by a reduction in MARC1 gene expression.

Embodiment 48. Use according to Embodiment 47, wherein the disease is nonalcoholic steatohepatitis (NASH), nonalcoholic fatty liver disease (NAFLD), alcoholic fatty liver disease, autoimmune hepatitis, hepatic fibrosis, cirrhosis, elevated blood cholesterol levels, hypertriglyceridemia, liver disease, and/or other MARC1-related disease.

Embodiment 49. Use of the RNAi agent of any one of Embodiments 1-31 or the composition according to any one of Embodiments 32-38, for the preparation of a pharmaceutical composition for treating a disease, disorder, or symptom that is mediated at least in part by a reduction in MARC1 gene expression.

The above provided embodiments and items are now illustrated with the following, non-limiting examples.

### EXAMPLES

### Example 1. Synthesis of MARC1 RNAi Agents

MARC1 RNAi agent duplexes shown in Tables 6A and 6B above, were synthesized in accordance with the following general procedures:

### A. Synthesis.

The sense and antisense strands of the RNAi agents were synthesized according to phosphoramidite technology on solid phase used in oligonucleotide synthesis. Such standard synthesis is generally known in the art. Depending on the scale, either a MerMade96E (R) (Bioautomation), a MerMade12® (Bioautomation), or an OP Pilot 100 (GE Healthcare) was used. Syntheses were performed on a solid support made of controlled pore glass (CPG, 500 Å or 600 Å, obtained from Prime Synthesis, Aston, PA, USA). The monomer positioned at the 3′ end of the respective strand was attached to the solid support as a starting point for synthesis. All RNA and 2′-modified RNA phosphoramidites were purchased from Thermo Fisher Scientific (Milwaukee, WI, USA) or Hongene Biotech (Shanghai, PRC). The 2′-O-methyl phosphoramidites included the following: (5′-O-dimethoxytrityl-N6-(benzoyl)-2′-O-methyl-adenosine-3′-O-(2-cyanoethyl-N,N-diisopropylamino) phosphoramidite, 5′-O-dimethoxy-trityl-N4-(acetyl)-2′-O-methyl-cytidine-3′-O-(2-cyanoethyl-N,N-diisopropyl-amino) phosphoramidite, (5′-O-dimethoxy trityl-N2-(isobutyryl)-2′-O-methyl-guanosine-3′-O-(2-cyanoethyl-N,N-diisopropylamino) phosphoramidite, and 5′-O-dimethoxytrityl-2′-O-methyl-uridine-3′-O-(2-cyanoethyl-N,N-diisopropylamino) phosphoramidite. The 2′-deoxy-2′-fluoro-phosphoramidites carried the same protecting groups as the 2′-O-methyl amidites. 5′-(4,4′-Dimethoxytrityl)-2′,3′-seco-uridine, 2′-benzoyl-3′-[(2-cyanoethyl)-(N,N-diisopropyl)]-phosphoramidite was also purchased from Thermo Fisher Scientific or Hongene Biotech. 5′-dimethoxytrityl-2′-O-methyl-inosine-3′-O-(2-cyanoethyl-N,N-diisopropylamino) phosphoramidites were purchased from Glen Research (Virginia) or Hongene Biotech. The cyclopropyl phosphonate phosphoramidites were synthesized in accordance with International Patent Application Publication No. WO 2017/214112 (see also Altenhofer et. al., Chem. Communications (Royal Soc. Chem.), 57 (55): 6808-6811 (July 2021)). The inverted abasic (3′-O-dimethoxytrityl-2′-deoxyribose-5′-O-(2-cyanoethyl-N,N-diisopropylamino) phosphoramidites were purchased from ChemGenes (Wilmington, MA, USA) or SAFC (St Louis, MO, USA). 5′-O-dimethoxytrityl-N2,N6-(phenoxyacetate)-2′-O-methyl-diaminopurine-3′-O-(2-cyanoethyl-N,N-diisopropylamino) phosphoramidites were obtained from ChemGenes or Hongene Biotech.

Targeting ligand-containing phosphoramidites were dissolved in anhydrous dichloromethane or anhydrous acetonitrile (50 mM), while all other amidites were dissolved in anhydrous acetonitrile (50 mM), or anhydrous dimethylformamide and molecular sieves (3A) were added. 5-Benzylthio-1H-tetrazole (BTT, 250 mM in acetonitrile), 5-Ethylthio-1H-tetrazole (ETT, 250 mM in acetonitrile), or 4,5-dicyanoimidazole (DCI) was used as activator solution. Coupling times were 12 min (RNA), 15 min (targeting ligand), 90 sec (2′OMe), and 60 sec (2′F). In order to introduce phosphorothioate linkages, a 100 mM solution of 3-phenyl 1,2,4-dithiazoline-5-one (POS, obtained from PolyOrg, Inc., Leominster, MA, USA) in anhydrous Acetonitrile was employed. Each of the MARC1 RNAi agent duplexes synthesized and tested in the following Examples utilized N-acetyl-galactosamine as “NAG” in the targeting ligand chemical structures represented in Table 7. (NAG37) and (NAG37) s targeting ligand phosphoramidite compounds can be synthesized in accordance with International Patent Application Publication No. WO 2018/044350 to Arrowhead Pharmaceuticals, Inc.

### B. Cleavage and Deprotection of Support Bound Oligomer

After finalization of the solid phase synthesis, the dried solid support was treated with a 1:1 volume solution of 40 wt. % methylamine in water and 28% ammonium hydroxide solution (Aldrich) for 1.5 hours at 30° C. The solution was evaporated and the solid residue was reconstituted in water (see below).

### Purification.

Crude oligomers were purified by anionic exchange HPLC using a TSKgel SuperQ-5 PW 13 μm column and Shimadzu LC-8 system. Buffer A was 20 mM Tris. 5 mM EDTA. pH 9.0) and contained 20% Acetonitrile and buffer B was the same as buffer A with the addition of 1.5 M sodium chloride. UV traces at 260 nm were recorded. Appropriate fractions were pooled then run on size exclusion HPLC using a GE Healthcare XK 26/40 column packed with Sephadex G-25 fine with a running buffer of filtered DI water or 100 mM ammonium bicarbonate, pH 6.7 and 20% Acetonitrile.

### D. Annealing.

Complementary strands were mixed by combining equimolar RNA solutions (sense and antisense) in 1×Phosphate-Buffered Saline (Corning, Cellgro) to form the RNAi agents. Some RNAi agents were lyophilized and stored at −15 to −25° C. Duplex concentration was determined by measuring the solution absorbance on a UV-Vis spectrometer in 1× Phosphate-Buffered Saline. The solution absorbance at 260 nm was then multiplied by a conversion factor and the dilution factor to determine the duplex concentration. The conversion factor used was either 0.050 mg/(mL·cm) or was calculated from an experimentally determined extinction coefficient.

### Example 2. hMARC1 SEAP Mouse Model

To evaluate MARC1 RNAi agents, a MARC1-SEAP mouse model was used. C57b16/Albino mice were transiently transfected in vivo with plasmid by hydrodynamic tail vein (HTV) injection. Mice were injected, via hydrodynamic tail vein (HTV), with plasmid pMIR1015 containing the 33-2500 region of the human MARC1 cDNA sequence (NCBI Reference Sequence: NM_022746.4 (Seq ID No. 1)) inserted into the 3′ UTR of the SEAP (secreted human placental alkaline phosphatase) reporter gene. 50 μg of the plasmid containing the hMARC1 cDNA in Ringer's solution in a total volume of 10% of the animal's body weight was injected, via HTV, to create MARC1-SEAP model mice. Following transfection with MARC1-SEAP, the mice were subsequently administered MARC1 RNAi agents. Inhibition of MARC1 expression by MARC1 RNAi agent results in concomitant inhibition of SEAP expression. SEAP expression levels were measured by Phospha-Light™ SEAP Reporter Gene Assay System (ThermoFisher Cat #T1016). Prior to treatment, SEAP expression levels in serum were measured and the mice were grouped according to average SEAP levels.

Analyses: SEAP levels may be measured at various times, both before and after administration of MARC1 RNAi agents.

i) Serum collection: Mice were anesthetized with 2-3% isoflurane and blood samples were collected from the submandibular area into serum separation tubes (Sarstedt AG & Co., Nümbrecht, Germany). Blood was allowed to coagulate at ambient temperature for 20 min. The tubes were centrifuged at 8,000×g for 3 min to separate the serum and stored at 4° C.

ii) Serum SEAP levels: Serum was collected and measured by the Phospha-Light™ SEAP Reporter Gene Assay System (ThermoFisher) according to the manufacturer's instructions. Serum SEAP levels for each animal was normalized to the control group of mice injected with saline in order to account for the non-treatment related decline in MARC1 sequence expression with this model. First, the SEAP level for each animal at a time point was divided by the pre-treatment level of expression in that animal (“pre-treatment”) in order to determine the ratio of expression “normalized to pre-treatment”. Expression at a specific time point was then normalized to the control group by dividing the “normalized to pre-treatment” ratio for an individual animal by the average “normalized to pre-treatment” ratio of all mice in the normal saline control group. Alternatively, in some Examples set forth herein, the serum SEAP levels for each animal were assessed by normalizing to pre-treatment levels only.

### Example 3. In Vivo Administration of MARC1 RNAi Agents in hMARC1-SEAP Mice

The hMARC1-SEAP model described in Example 2, above, was used. On Day 1, four (n=4) female C57bl/6 albino mice were dosed with either saline or MARC1 RNAi agents formulated in saline (at 2 mg/kg), via subcutaneous (SQ) injection. The dosing regimen is in accordance with Table 8 below.

TABLE 8
Dosing for mice of Example 3

| | | Targeted Position | |
|---|---|---|---|
| | Dose (RNAi | of MARC1 (Seq | |
| Group | Agent) | ID No. 1) | Dosing Route |
| 1 | Isotonic Saline | N/A | Day 1 SQ Injection |
| 2 | 2 mg/kg AD11764 | 325 | Day 1 SQ Injection |
| 3 | 2 mg/kg AD11765 | 536 | Day 1 SQ Injection |
| 4 | 2 mg/kg AD11766 | 609 | Day 1 SQ Injection |
| 5 | 2 mg/kg AD11767 | 611 | Day 1 SQ Injection |
| 6 | 2 mg/kg AD11768 | 636 | Day 1 SQ Injection |
| 7 | 2 mg/kg AD11769 | 640 | Day 1 SQ Injection |
| 8 | 2 mg/kg AD11770 | 644 | Day 1 SQ Injection |
| 9 | 2 mg/kg AD11771 | 710 | Day 1 SQ Injection |
| 10 | 2 mg/kg AD11772 | 841 | Day 1 SQ Injection |
| 11 | 2 mg/kg AD11773 | 932 | Day 1 SQ Injection |
| 12 | 2 mg/kg AD11774 | 940 | Day 1 SQ Injection |
| 13 | 2 mg/kg AD11775 | 945 | Day 1 SQ Injection |
| 14 | 2 mg/kg AD11776 | 954 | Day 1 SQ Injection |
| 15 | 2 mg/kg AD11777 | 1057 | Day 1 SQ Injection |
| 16 | 2 mg/kg AD11778 | 1089 | Day 1 SQ Injection |
| 17 | 2 mg/kg AD11779 | 1098 | Day 1 SQ Injection |
| 18 | 2 mg/kg AD11780 | 1102 | Day 1 SQ Injection |
| 19 | 2 mg/kg AD11781 | 1111 | Day 1 SQ Injection |
| 20 | 2 mg/kg AD11782 | 1190 | Day 1 SQ Injection |
| 21 | 2 mg/kg AD11783 | 1193 | Day 1 SQ Injection |
| 22 | 2 mg/kg AD11784 | 1282 | Day 1 SQ Injection |
| 23 | 2 mg/kg AD11785 | 1310 | Day 1 SQ Injection |
| 24 | 2 mg/kg AD11786 | 1313 | Day 1 SQ Injection |
| 25 | 2 mg/kg AD11787 | 1605 | Day 1 SQ Injection |
| 26 | 2 mg/kg AD11788 | 1635 | Day 1 SQ Injection |
| 27 | 2 mg/kg AD11789 | 1646 | Day 1 SQ Injection |
| 28 | 2 mg/kg AD11790 | 1648 | Day 1 SQ Injection |
| 29 | 2 mg/kg AD11791 | 1852 | Day 1 SQ Injection |
| 30 | 2 mg/kg AD11792 | 1897 | Day 1 SQ Injection |
| 31 | 2 mg/kg AD11793 | 1898 | Day 1 SQ Injection |
| 32 | 2 mg/kg AD11794 | 1955 | Day 1 SQ Injection |
| 33 | 2 mg/kg AD11795 | 1990 | Day 1 SQ Injection |

Serum was collected on Day −3, 1, 8, 15, 22, and 29. SEAP expression levels were determined pursuant to the procedure set forth in Example 2, above. Data from the experiment are shown in the following Table 9, with average SEAP reflecting the normalized average value of SEAP.

TABLE 9
Average SEAP normalized to pre-treatment and saline
control in hMARC1-SEAP mice of Example 3.

| | Day 8 | | Day 15 | | Day 22 | |
|---|---|---|---|---|---|---|
| | Avg | Std | Avg | Std | Avg | Std |
| Group ID | SEAP | Dev | SEAP | Dev | SEAP | Dev |
| 1. Isotonic Saline | 1.000 | 0.228 | 1.000 | 0.221 | 1.000 | 0.227 |
| 2. 2 mg/kg AD11764 | 1.417 | 0.470 | 1.331 | 0.425 | 1.557 | 0.467 |
| 3. 2 mg/kg AD11765 | 1.606 | 0.124 | 1.540 | 0.135 | 1.729 | 0.384 |
| 4. 2 mg/kg AD11766 | 1.007 | 0.134 | 1.098 | 0.186 | 1.389 | 0.087 |
| 5. 2 mg/kg AD11767 | 0.975 | 0.103 | 0.926 | 0.324 | 0.949 | 0.264 |
| 6. 2 mg/kg AD11768 | 1.091 | 0.310 | 1.007 | 0.302 | 1.168 | 0.314 |
| 7. 2 mg/kg AD11769 | 0.923 | 0.254 | 0.814 | 0.461 | 0.829 | 0.433 |
| 8. 2 mg/kg AD11770 | 0.992 | 0.022 | 0.984 | 0.158 | 0.989 | 0.085 |
| 9. 2 mg/kg AD11771 | 1.122 | 0.190 | 1.083 | 0.168 | 1.216 | 0.209 |
| 10. 2 mg/kg AD11772 | 1.047 | 0.152 | 0.881 | 0.333 | 0.785 | 0.357 |
| 11. 2 mg/kg AD11773 | 1.152 | 0.097 | 0.985 | 0.130 | 1.115 | 0.375 |
| 12. 2 mg/kg AD11774 | 0.824 | 0.241 | 0.815 | 0.374 | 0.844 | 0.526 |
| 13. 2 mg/kg AD11775 | 0.655 | 0.167 | 0.660 | 0.416 | 0.522 | 0.216 |
| 14. 2 mg/kg AD11776 | 0.959 | 0.247 | 0.862 | 0.273 | 1.087 | 0.408 |
| 15. 2 mg/kg AD11777 | 0.811 | 0.256 | 0.774 | 0.442 | 0.936 | 0.479 |
| 16. 2 mg/kg AD11778 | 0.542 | 0.122 | 0.474 | 0.261 | 0.354 | 0.243 |
| 17. 2 mg/kg AD11779 | 1.083 | 0.559 | 0.810 | 0.536 | 0.755 | 0.578 |
| 18. 2 mg/kg AD11780 | 0.998 | 0.310 | 0.910 | 0.465 | 0.973 | 0.511 |
| 19. 2 mg/kg AD11781 | 2.102 | 1.415 | 0.855 | 0.649 | 1.049 | 0.767 |
| 20. 2 mg/kg AD11782 | 1.156 | 0.379 | 0.464 | 0.215 | 0.486 | 0.222 |
| 21. 2 mg/kg AD11783 | 1.126 | 0.166 | 0.838 | 0.111 | 0.789 | 0.217 |
| 22. 2 mg/kg AD11784 | 0.973 | 0.209 | 0.774 | 0.306 | 0.696 | 0.379 |
| 23. 2 mg/kg AD11785 | 0.543 | 0.439 | 0.464 | 0.437 | 0.492 | 0.507 |
| 24. 2 mg/kg AD11786 | 0.730 | 0.369 | 0.458 | 0.308 | 0.433 | 0.253 |
| 25. 2 mg/kg AD11787 | 0.937 | 0.151 | 0.822 | 0.106 | 0.957 | 0.129 |
| 26. 2 mg/kg AD11788 | 1.015 | 0.215 | 0.683 | 0.168 | 0.721 | 0.149 |
| 27. 2 mg/kg AD11789 | 1.311 | 0.196 | 0.910 | 0.135 | 1.059 | 0.211 |
| 28. 2 mg/kg AD11790 | 1.100 | 0.207 | 0.825 | 0.210 | 1.073 | 0.366 |
| 29. 2 mg/kg AD11791 | 1.115 | 0.438 | 0.890 | 0.560 | 1.273 | 0.857 |
| 30. 2 mg/kg AD11792 | 0.946 | 0.201 | 0.876 | 0.120 | 1.020 | 0.252 |
| 31. 2 mg/kg AD11793 | 0.853 | 0.207 | 0.668 | 0.287 | 0.852 | 0.441 |
| 32. 2 mg/kg AD11794 | 1.095 | 0.357 | 0.785 | 0.323 | 0.997 | 0.513 |
| 33. 2 mg/kg AD11795 | 1.117 | 0.084 | 0.973 | 0.199 | 1.154 | 0.174 |

Groups 5, 7, 8, 12-16, 18, 22-25, 30, and 31 showed reduction in SEAP at Day 8. Groups 5, 7, 8, and 10-33 showed reduction in SEAP at Day 15. Groups 5, 7, 8, 10, 12, 13, 15-18, 20-26, 31, and 32 showed reduction in SEAP at Day 22.

### Example 4. In Vivo Administration of MARC1 RNAi Agents in hMARC1-SEAP Mice

The hMARC1-SEAP model described in Example 2, above, was used. On Day 1, four (n-4) female C57bl/6 albino mice were dosed with either saline or MARC1 RNAi agents formulated in saline (at 2 mg/kg, 4 mg/kg, or 6 mg/kg), via subcutaneous (SQ) injection, at 200 μL per 20 g (10 mL/kg) body weight injection volume. The dosing regimen is in accordance with Table 10 below.

TABLE 10
Dosing for mice of Example 4

| | | Targeted Position | |
|---|---|---|---|
| | Dose (RNAi | of MARC1 (Seq | |
| Group | Agent) | ID No. 1) | Dosing Route |
| 1 | Isotonic Saline | N/A | Day 1 SQ Injection |
| 2 | 6 mg/kg AD11778 | 1089 | Day 1 SQ Injection |
| 3 | 4 mg/kg AD11778 | 1089 | Day 1 SQ Injection |
| 4 | 2 mg/kg AD11778 | 1089 | Day 1 SQ Injection |
| 5 | 6 mg/kg AD11785 | 1310 | Day 1 SQ Injection |
| 6 | 4 mg/kg AD11785 | 1310 | Day 1 SQ Injection |
| 7 | 2 mg/kg AD11785 | 1310 | Day 1 SQ Injection |
| 8 | 6 mg/kg AD11786 | 1313 | Day 1 SQ Injection |
| 9 | 4 mg/kg AD11786 | 1313 | Day 1 SQ Injection |
| 10 | 2 mg/kg AD11786 | 1313 | Day 1 SQ Injection |
| 11 | 4 mg/kg AD11782 | 1190 | Day 1 SQ Injection |

Serum was collected on Day −5, 1, 8, 15, 22, and 29. SEAP expression levels were determined pursuant to the procedure set forth in Example 2, above. Data from the experiment are shown in the following Table 11, with average SEAP reflecting the normalized average value of SEAP.

TABLE 11
Average SEAP normalized to pre-treatment and saline
control in MARC1-SEAP mice of Example 4.

| | Day 8 | | Day 15 | | Day 22 | |
|---|---|---|---|---|---|---|
| | Avg | Std | Avg | Std | Avg | Std |
| Group ID | SEAP | Dev | SEAP | Dev | SEAP | Dev |
| 1. Isotonic Saline | 1.000 | 0.212 | 1.000 | 0.335 | 1.000 | 0.182 |
| 2. 6 mg/kg AD11778 | 0.241 | 0.054 | 0.107 | 0.018 | 0.075 | 0.025 |
| 3. 4 mg/kg AD11778 | 0.409 | 0.074 | 0.209 | 0.083 | 0.234 | 0.122 |
| 4. 2 mg/kg AD11778 | 0.425 | 0.084 | 0.207 | 0.058 | 0.228 | 0.089 |
| 5. 6 mg/kg AD11785 | 0.252 | 0.135 | 0.140 | 0.103 | 0.162 | 0.127 |
| 6. 4 mg/kg AD11785 | 0.418 | 0.098 | 0.214 | 0.115 | 0.249 | 0.219 |
| 7. 2 mg/kg AD11785 | 0.597 | 0.107 | 0.383 | 0.093 | 0.441 | 0.138 |
| 8. 6 mg/kg AD11786 | 0.272 | 0.109 | 0.164 | 0.152 | 0.187 | 0.220 |
| 9. 4 mg/kg AD11786 | 0.286 | 0.025 | 0.200 | 0.045 | 0.194 | 0.063 |
| 10. 2 mg/kg AD11786 | 0.433 | 0.118 | 0.371 | 0.139 | 0.377 | 0.164 |
| 11. 4 mg/kg AD11782 | 0.614 | 0.089 | 0.318 | 0.075 | 0.313 | 0.180 |

Day 29

| | Avg | Std |
|---|---|---|
| Group ID | SEAP | Dev |
| 1. Isotonic Saline | 1.000 | 0.250 |
| 2. 6 mg/kg AD11778 | 0.134 | 0.078 |
| 3. 4 mg/kg AD11778 | 0.419 | 0.345 |
| 4. 2 mg/kg AD11778 | 0.238 | 0.099 |
| 5. 6 mg/kg AD11785 | 0.218 | 0.113 |
| 6. 4 mg/kg AD11785 | 0.342 | 0.322 |
| 7. 2 mg/kg AD11785 | 0.505 | 0.131 |
| 8. 6 mg/kg AD11786 | 0.247 | 0.271 |
| 9. 4 mg/kg AD11786 | 0.273 | 0.082 |
| 10. 2 mg/kg AD11786 | 0.485 | 0.231 |
| 11. 4 mg/kg AD11782 | 0.369 | 0.128 |

Groups 2-11 showed reduction in SEAP at all time points.

### Example 5. In Vivo Administration of MARC1 RNAi Agents in hMARC1-SEAP Mice

The hMARC1-SEAP model described in Example 2, above, was used. On Day 1, four (n=4) female C57bl/6 albino mice were dosed with either saline or MARC1 RNAi agents formulated in saline (at 2 mg/kg), via subcutaneous (SQ) injection, at 200 μl per 20 g (10 mL/kg) body weight injection volume. The dosing regimen is in accordance with Table 12 below.

TABLE 12
Dosing for mice of Example 5

| | | Targeted Position | |
|---|---|---|---|
| | Dose (RNAi | of MARC1 (Seq | |
| Group | Agent) | ID No. 1) | Dosing Route |
| 1 | Isotonic Saline | N/A | Day 1 SQ Injection |
| 2 | 2 mg/kg AD12363 | 305 | Day 1 SQ Injection |
| 3 | 2 mg/kg AD12364 | 761 | Day 1 SQ Injection |
| 4 | 2 mg/kg AD12365 | 956 | Day 1 SQ Injection |
| 5 | 2 mg/kg AD12366 | 1109 | Day 1 SQ Injection |
| 6 | 2 mg/kg AD12367 | 1275 | Day 1 SQ Injection |
| 7 | 2 mg/kg AD12368 | 1633 | Day 1 SQ Injection |
| 8 | 2 mg/kg AD12369 | 1817 | Day 1 SQ Injection |
| 9 | 2 mg/kg AD12370 | 1900 | Day 1 SQ Injection |
| 10 | 2 mg/kg AD12371 | 1954 | Day 1 SQ Injection |
| 11 | 2 mg/kg AD12372 | 1633 | Day 1 SQ Injection |
| 12 | 2 mg/kg AD11786 | 1313 | Day 1 SQ Injection |

Serum was collected on Day −9. 1. 8. 15, and 22. SEAP expression levels were determined pursuant to the procedure set forth in Example 2, above. Data from the experiment are shown in the following Table 13, with average SEAP reflecting the normalized average value of SEAP.

TABLE 13
Average SEAP normalized to pre-treatment and saline
control in MARC1-SEAP mice of Example 5.

| | Day 8 | | Day 15 | | Day 22 | |
|---|---|---|---|---|---|---|
| | Avg | Std | Avg | Std | Avg | Std |
| Group ID | SEAP | Dev | SEAP | Dev | SEAP | Dev |
| 1. Isotonic Saline | 1.000 | 0.087 | 1.000 | 0.302 | 1.000 | 0.299 |
| 2. 2 mg/kg AD12363 | 0.778 | 0.196 | 0.551 | 0.180 | 0.703 | 0.219 |
| 3. 2 mg/kg AD12364 | 0.887 | 0.032 | 0.661 | 0.191 | 0.878 | 0.402 |
| 4. 2 mg/kg AD12365 | 0.918 | 0.246 | 0.723 | 0.224 | 0.801 | 0.231 |
| 5. 2 mg/kg AD12366 | 0.732 | 0.198 | 0.598 | 0.258 | 0.601 | 0.142 |
| 6. 2 mg/kg AD12367 | 0.622 | 0.133 | 0.374 | 0.074 | 0.517 | 0.182 |
| 7. 2 mg/kg AD12368 | 0.800 | 0.096 | 0.529 | 0.047 | 0.713 | 0.185 |
| 8. 2 mg/kg AD12369 | 0.970 | 0.262 | 0.835 | 0.111 | 0.887 | 0.171 |
| 9. 2 mg/kg AD12370 | 0.747 | 0.215 | 0.508 | 0.251 | 0.634 | 0.225 |
| 10. 2 mg/kg AD12371 | 0.639 | 0.150 | 0.355 | 0.193 | 0.704 | 0.161 |
| 11. 2 mg/kg AD12372 | 0.813 | 0.209 | 0.704 | 0.184 | 0.764 | 0.249 |
| 12. 2 mg/kg AD11786 | 0.393 | 0.051 | 0.582 | 0.114 | 0.327 | 0.099 |

Groups 2-12 showed reduction in SEAP at all time points.

### Example 6. In Vivo Administration of MARC1 RNAi Agents in hMARC1-SEAP Mice

The hMARC1-SEAP model described in Example 2, above, was used. On Day 1, four (n-4) female C57bl/6 albino mice were dosed with either saline or MARC1 RNAi agents formulated in saline (at 2 mg/kg), via subcutaneous (SQ) injection, at 200 μL per 20 g (10 mL/kg) body weight injection volume. The dosing regimen is in accordance with Table 14 below.

TABLE 14
Dosing for mice of Example 6

| | | Targeted Position | |
|---|---|---|---|
| | Dose (RNAi | of MARC1 (Seq | |
| Group | Agent) | ID No. 1) | Dosing Route |
| 1 | Isotonic Saline | N/A | Day 1 SQ Injection |
| 2 | 2 mg/kg AD11786 | 1313 | Day 1 SQ Injection |
| 3 | 2 mg/kg AD12291 | 1313 | Day 1 SQ Injection |
| 4 | 2 mg/kg AD12292 | 1313 | Day 1 SQ Injection |
| 5 | 2 mg/kg AD12972 | 1313 | Day 1 SQ Injection |
| 6 | 2 mg/kg AD12973 | 1313 | Day 1 SQ Injection |
| 7 | 2 mg/kg AD12974 | 1313 | Day 1 SQ Injection |
| 8 | 2 mg/kg AD12975 | 1313 | Day 1 SQ Injection |
| 9 | 2 mg/kg AD12976 | 1313 | Day 1 SQ Injection |
| 10 | 2 mg/kg AD12591 | 901 | Day 1 SQ Injection |

Serum was collected on Day −14,−7, 1, 8, 15, 22, and 29. SEAP expression levels were determined pursuant to the procedure set forth in Example 2, above. Data from the experiment are shown in the following Table 15, with average SEAP reflecting the normalized average value of SEAP.

TABLE 15
Average SEAP normalized to pre-treatment and saline
control in MARC1-SEAP mice of Example 6.

| | Day 8 | | Day 15 | | Day 22 | |
|---|---|---|---|---|---|---|
| | Avg | Std | Avg | Std | Avg | Std |
| Group ID | SEAP | Dev | SEAP | Dev | SEAP | Dev |
| 1. Isotonic Saline | 1.000 | 0.152 | 1.000 | 0.188 | 1.000 | 0.334 |
| 2. 2 mg/kg AD11786 | 0.591 | 0.085 | 0.475 | 0.152 | 0.395 | 0.171 |
| 3. 2 mg/kg AD12291 | 0.625 | 0.041 | 0.392 | 0.094 | 0.387 | 0.231 |
| 4. 2 mg/kg AD12292 | 0.823 | 0.079 | 0.614 | 0.094 | 0.433 | 0.077 |
| 5. 2 mg/kg AD12972 | 0.703 | 0.140 | 0.584 | 0.220 | 0.548 | 0.229 |
| 6. 2 mg/kg AD12973 | 0.627 | 0.109 | 0.376 | 0.052 | 0.275 | 0.045 |
| 7. 2 mg/kg AD12974 | 0.633 | 0.037 | 0.493 | 0.145 | 0.446 | 0.152 |
| 8. 2 mg/kg AD12975 | 0.573 | 0.057 | 0.409 | 0.141 | 0.303 | 0.134 |
| 9. 2 mg/kg AD12976 | 0.493 | 0.095 | 0.341 | 0.100 | 0.283 | 0.121 |
| 10. 2 mg/kg AD12591 | 0.849 | 0.131 | 0.776 | 0.184 | 0.514 | 0.078 |

Day 29

| | Avg | Std |
|---|---|---|
| Group ID | SEAP | Dev |
| 1. Isotonic Saline | 1.000 | 0.214 |
| 2. 2 mg/kg AD11786 | 0.508 | 0.160 |
| 3. 2 mg/kg AD12291 | 0.444 | 0.333 |
| 4. 2 mg/kg AD12292 | 0.657 | 0.135 |
| 5. 2 mg/kg AD12972 | 0.567 | 0.258 |
| 6. 2 mg/kg AD12973 | 0.337 | 0.065 |
| 7. 2 mg/kg AD12974 | 0.525 | 0.185 |
| 8. 2 mg/kg AD12975 | 0.382 | 0.210 |
| 9. 2 mg/kg AD12976 | 0.351 | 0.152 |
| 10. 2 mg/kg AD12591 | 0.658 | 0.163 |

Groups 2-10 showed reduction in SEAP at all time points.

### Example 7. In Vivo Administration of MARC1 RNAI Agents in hMARC1-SEAP Mice

The hMARC1-SEAP model described in Example 2, above, was used. On Day 1, four (n=4) female C57bl/6 albino mice were dosed with either saline or MARC1 RNAi agents formulated in saline (at 2 mg/kg), via subcutaneous (SQ) injection, at 200 μL per 20 g (10 mL/kg) body weight injection volume. The dosing regimen is in accordance with Table 16 below.

TABLE 16
Dosing for mice of Example 7

| | | Targeted Position | |
|---|---|---|---|
| | Dose (RNAi | of MARC1 (Seq | |
| Group | Agent) | ID No. 1) | Dosing Route |
| 1 | Isotonic Saline | N/A | Day 1 SQ Injection |
| 2 | 2 mg/kg AD11786 | 1313 | Day 1 SQ Injection |
| 3 | 2 mg/kg AD12367 | 1275 | Day 1 SQ Injection |
| 4 | 2 mg/kg AD13113 | 1275 | Day 1 SQ Injection |
| 5 | 2 mg/kg AD13114 | 1275 | Day 1 SQ Injection |
| 6 | 2 mg/kg AD13115 | 1275 | Day 1 SQ Injection |
| 7 | 2 mg/kg AD13116 | 1275 | Day 1 SQ Injection |
| 8 | 2 mg/kg AD13117 | 1275 | Day 1 SQ Injection |
| 9 | 2 mg/kg AD12371 | 1954 | Day 1 SQ Injection |
| 10 | 2 mg/kg AD13118 | 1954 | Day 1 SQ Injection |
| 11 | 2 mg/kg AD13119 | 1954 | Day 1 SQ Injection |
| 12 | 2 mg/kg AD12596 | 1014 | Day 1 SQ Injection |

Serum was collected on Day −14.−7, 1, 8, 15, 22, and 29. SEAP expression levels were determined pursuant to the procedure set forth in Example 2, above. Data from the experiment are shown in the following Table 17, with average SEAP reflecting the normalized average value of SEAP.

TABLE 17
Average SEAP normalized to pre-treatment and saline
control in MARC1-SEAP mice of Example 7.

| | Day 8 | | Day 15 | | Day 22 | |
|---|---|---|---|---|---|---|
| | Avg | Std | Avg | Std | Avg | Std |
| Group ID | SEAP | Dev | SEAP | Dev | SEAP | Dev |
| 1. Isotonic Saline | 1.000 | 0.182 | 1.000 | 0.322 | 1.000 | 0.373 |
| 2. 2 mg/kg AD11786 | 0.881 | 0.210 | 0.875 | 0.558 | 0.711 | 0.598 |
| 3. 2 mg/kg AD12367 | 1.103 | 0.095 | 1.260 | 0.308 | 1.241 | 0.481 |
| 4. 2 mg/kg AD13113 | 0.906 | 0.248 | 1.020 | 0.408 | 0.896 | 0.401 |
| 5. 2 mg/kg AD13114 | 0.936 | 0.138 | 0.947 | 0.447 | 0.714 | 0.307 |
| 6. 2 mg/kg AD13115 | 1.026 | 0.118 | 1.175 | 0.157 | 1.033 | 0.151 |
| 7. 2 mg/kg AD13116 | 0.890 | 0.032 | 0.886 | 0.214 | 0.767 | 0.170 |
| 8. 2 mg/kg AD13117 | 0.645 | 0.155 | 0.626 | 0.310 | 0.616 | 0.428 |
| 9. 2 mg/kg AD12371 | 1.381 | 0.131 | 1.744 | 0.574 | 1.492 | 0.524 |
| 10. 2 mg/kg AD13118 | 1.083 | 0.374 | 1.026 | 0.328 | 1.010 | 0.300 |
| 11. 2 mg/kg AD13119 | 1.311 | 0.128 | 1.592 | 0.111 | 1.469 | 0.141 |
| 12. 2 mg/kg AD12596 | 1.260 | 0.273 | 1.562 | 0.242 | 1.399 | 0.352 |

Groups 2, 4, 5, 7, and 8 showed reduction in SEAP at Day 8. Groups 2, 5, 7, and 8 showed reduction in SEAP at Day 15. Groups 2, 4, 5, 7, and 8 showed reduction in SEAP at Day 22.

### Example 8. In Vivo Administration of MARC1 RNAi Agents in hMARC1-SEAP Mice

The hMARC1-SEAP model described in Example 2, above, was used. On Day 1, four (n=4) female C57bl/6 albino mice were dosed with either saline or MARC1 RNAi agents formulated in saline (at 2 mg/kg), via subcutaneous (SQ) injection, at 200 μL per 20 g (10 mL/kg) body weight injection volume. The dosing regimen is in accordance with Table 18 below.

TABLE 18
Dosing for mice of Example 8

| | | Targeted Position | |
|---|---|---|---|
| | Dose (RNAi | of MARC1 (Seq ID | |
| Group | Agent) | No. 1) | Dosing Route |
| 1 | Isotonic Saline | N/A | Day 1 SQ Injection |
| 2 | 2 mg/kg AD11786 | 1313 | Day 1 SQ Injection |
| 3 | 2 mg/kg AD12976 | 1313 | Day 1 SQ Injection |
| 4 | 2 mg/kg AD13322 | 419 | Day 1 SQ Injection |
| 5 | 2 mg/kg AD13323 | 601 | Day 1 SQ Injection |
| 6 | 2 mg/kg AD13324 | 1124 | Day 1 SQ Injection |
| 7 | 2 mg/kg AD13325 | 1131 | Day 1 SQ Injection |
| 8 | 2 mg/kg AD13326 | 1157 | Day 1 SQ Injection |
| 9 | 2 mg/kg AD13327 | 1246 | Day 1 SQ Injection |
| 10 | 2 mg/kg AD13328 | 1332 | Day 1 SQ Injection |
| 11 | 2 mg/kg AD13329 | 1432 | Day 1 SQ Injection |
| 12 | 2 mg/kg AD13330 | 1842 | Day 1 SQ Injection |

Serum was collected on Day −14,−7, 1, 8, 15, 22, and 29. SEAP expression levels were determined pursuant to the procedure set forth in Example 2, above. Data from the experiment are shown in the following Table 19, with average SEAP reflecting the normalized average value of SEAP.

TABLE 19
Average SEAP normalized to pre-treatment and saline
control in MARC1-SEAP mice of Example 8.

| | Day 8 | | Day 15 | | Day 22 | |
|---|---|---|---|---|---|---|
| | Avg | Std | Avg | Std | Avg | Std |
| Group ID | SEAP | Dev | SEAP | Dev | SEAP | Dev |
| 1. Isotonic Saline | 1.000 | 0.221 | 1.000 | 0.421 | 1.000 | 0.323 |
| 2. 2 mg/kg AD11786 | 0.529 | 0.035 | 0.451 | 0.111 | 0.429 | 0.144 |
| 3. 2 mg/kg AD12976 | 0.334 | 0.107 | 0.231 | 0.129 | 0.246 | 0.193 |
| 4. 2 mg/kg AD13322 | 0.568 | 0.158 | 0.824 | 0.913 | 0.625 | 0.293 |
| 5. 2 mg/kg AD13323 | 0.918 | 0.141 | 0.722 | 0.189 | 0.758 | 0.237 |
| 6. 2 mg/kg AD13324 | 0.599 | 0.123 | 0.506 | 0.079 | 0.566 | 0.149 |
| 7. 2 mg/kg AD13325 | 0.488 | 0.110 | 0.510 | 0.212 | 0.598 | 0.200 |
| 8. 2 mg/kg AD13326 | 0.660 | 0.171 | 0.796 | 0.196 | 0.624 | 0.071 |
| 9. 2 mg/kg AD13327 | 0.701 | 0.092 | 0.592 | 0.271 | 0.948 | 0.568 |
| 10. 2 mg/kg AD13328 | 0.785 | 0.155 | 0.612 | 0.128 | 0.548 | 0.224 |
| 11. 2 mg/kg AD13329 | 0.570 | 0.066 | 0.542 | 0.138 | 0.584 | 0.180 |
| 12. 2 mg/kg AD13330 | 0.552 | 0.117 | 0.588 | 0.089 | 0.520 | 0.092 |

Groups 2-12 showed reduction in SEAP at all time points.

### Example 9. In Vivo Administration of MARC1 RNAi Agents in hMARC1-SEAP Mice

The hMARC1-SEAP model described in Example 2, above, was used. On Day 1, four (n=4) female C57bl/6 albino mice were dosed with either saline or MARC1 RNAi agents formulated in saline (at 2 mg/kg), via subcutaneous (SQ) injection, at 200 μL per 20 g (10 mL/kg) body weight injection volume. The dosing regimen is in accordance with Table 20 below

TABLE 20
Dosing for mice of Example 9

| | | Targeted Position | |
|---|---|---|---|
| | Dose (RNAi | of MARC1 (Seq ID | |
| Group | Agent) | No. 1) | Dosing Route |
| 1 | Isotonic Saline | N/A | Day 1 SQ Injection |
| 2 | 2 mg/kg AD12976 | 1313 | Day 1 SQ Injection |
| 3 | 2 mg/kg AD13535 | 1059 | Day 1 SQ Injection |
| 4 | 2 mg/kg AD13536 | 1058 | Day 1 SQ Injection |
| 5 | 2 mg/kg AD13537 | 1060 | Day 1 SQ Injection |
| 6 | 2 mg/kg AD12982 | 1089 | Day 1 SQ Injection |
| 7 | 2 mg/kg AD13117 | 1275 | Day 1 SQ Injection |
| 8 | 2 mg/kg AD12960 | 1817 | Day 1 SQ Injection |
| 9 | 2 mg/kg AD12287 | 1190 | Day 1 SQ Injection |
| 10 | 2 mg/kg AD12289 | 1310 | Day 1 SQ Injection |

Serum was collected on Day −7, 1, 8, 15, and 22. SEAP expression levels were determined pursuant to the procedure set forth in Example 2, above. Data from the experiment are shown in the following Table 21, with average SEAP reflecting the normalized average value of SEAP.

TABLE 21
Average SEAP normalized to pre-treatment and saline
control in MARC1-SEAP mice of Example 9.

| | Day 8 | | Day 15 | | Day 22 | |
|---|---|---|---|---|---|---|
| | Avg | Std | Avg | Std | Avg | Std |
| Group ID | SEAP | Dev | SEAP | Dev | SEAP | Dev |
| 1. Isotonic Saline | 1.000 | 0.134 | 1.000 | 0.296 | 1.000 | 0.476 |
| 2. 2 mg/kg AD12976 | 0.507 | 0.120 | 0.266 | 0.147 | 0.261 | 0.186 |
| 3. 2 mg/kg AD13535 | 0.976 | 0.234 | 0.790 | 0.311 | 0.797 | 0.396 |
| 4. 2 mg/kg AD13536 | 0.731 | 0.242 | 0.535 | 0.222 | 0.591 | 0.238 |
| 5. 2 mg/kg AD13537 | 0.372 | 0.104 | 0.229 | 0.092 | 0.208 | 0.062 |
| 6. 2 mg/kg AD12982 | 0.487 | 0.051 | 0.234 | 0.062 | 0.164 | 0.046 |
| 7. 2 mg/kg AD13117 | 0.482 | 0.198 | 0.290 | 0.178 | 0.349 | 0.290 |
| 8. 2 mg/kg AD12960 | 0.803 | 0.123 | 0.673 | 0.047 | 0.655 | 0.128 |
| 9. 2 mg/kg AD12287 | 0.535 | 0.096 | 0.307 | 0.095 | 0.256 | 0.108 |
| 10. 2 mg/kg AD12289 | 0.727 | 0.087 | 0.320 | 0.097 | 0.310 | 0.074 |

Groups 2-10 showed reduction in SEAP at all time points.

### Example 10. In Vivo Administration of MARC1 RNAi Agents in hMARC1-SEAP Mice

The hMARC1-SEAP model described in Example 2, above, was used. On Day 1, four (n=4) female C57bl/6 albino mice were dosed with either saline or MARC1 RNAi agents formulated in saline (at 2 mg/kg), via subcutaneous (SQ) injection, at 200 μL per 20 g (10 mL/kg) body weight injection volume. The dosing regimen is in accordance with Table 22 below.

TABLE 22
Dosing for mice of Example 10

| | | Targeted Position | |
|---|---|---|---|
| | Dose (RNAi | of MARC1 (Seq ID | |
| Group | Agent) | No. 1) | Dosing Route |
| 1 | Isotonic Saline | N/A | Day 1 SQ Injection |
| 2 | 2 mg/kg AD11778 | 1089 | Day 1 SQ Injection |
| 3 | 2 mg/kg AD12982 | 1089 | Day 1 SQ Injection |
| 4 | 2 mg/kg AD13705 | 1089 | Day 1 SQ Injection |
| 5 | 2 mg/kg AD13706 | 1089 | Day 1 SQ Injection |
| 6 | 2 mg/kg AD13707 | 1089 | Day 1 SQ Injection |
| 7 | 2 mg/kg AD12634 | 1931 | Day 1 SQ Injection |
| 8 | 2 mg/kg AD13708 | 1931 | Day 1 SQ Injection |
| 9 | 2 mg/kg AD13709 | 1931 | Day 1 SQ Injection |
| 10 | 2 mg/kg AD13710 | 1931 | Day 1 SQ Injection |
| 11 | 2 mg/kg AD13711 | 1931 | Day 1 SQ Injection |
| 12 | 2 mg/kg AD13712 | 1931 | Day 1 SQ Injection |

Serum was collected on Day −7, 1, 8, 15, and 22. SEAP expression levels were determined pursuant to the procedure set forth in Example 2, above. Data from the experiment are shown in the following Table 23, with average SEAP reflecting the normalized average value of SEAP.

TABLE 23
Average SEAP normalized to pre-treatment and saline
control in MARC1-SEAP mice of Example 10.

| | Day 8 | | Day 15 | | Day 22 | |
|---|---|---|---|---|---|---|
| | Avg | Std | Avg | Std | Avg | Std |
| Group ID | SEAP | Dev | SEAP | Dev | SEAP | Dev |
| 1. Isotonic Saline | 1.000 | 0.312 | 1.000 | 0.306 | 1.000 | 0.169 |
| 2. 2 mg/kg AD11778 | 0.510 | 0.109 | 0.348 | 0.057 | 0.619 | 0.266 |
| 3. 2 mg/kg AD12982 | 0.407 | 0.090 | 0.237 | 0.068 | 0.272 | 0.162 |
| 4. 2 mg/kg AD13705 | 0.485 | 0.027 | 0.260 | 0.057 | 0.316 | 0.104 |
| 5. 2 mg/kg AD13706 | 0.457 | 0.159 | 0.219 | 0.141 | 0.299 | 0.221 |
| 6. 2 mg/kg AD13707 | 0.597 | 0.038 | 0.368 | 0.094 | 0.452 | 0.186 |
| 7. 2 mg/kg AD12634 | 0.632 | 0.069 | 0.665 | 0.277 | 0.772 | 0.438 |
| 8. 2 mg/kg AD13708 | 0.566 | 0.094 | 0.461 | 0.089 | 0.614 | 0.155 |
| 9. 2 mg/kg AD13709 | 0.562 | 0.044 | 0.306 | 0.131 | 0.373 | 0.226 |
| 10. 2 mg/kg AD13710 | 0.415 | 0.131 | 0.259 | 0.194 | 0.323 | 0.184 |
| 11. 2 mg/kg AD13711 | 0.556 | 0.112 | 0.506 | 0.155 | 0.467 | 0.128 |
| 12. 2 mg/kg AD13712 | 0.391 | 0.108 | 0.325 | 0.154 | 0.262 | 0.065 |

Groups 2-12 showed reduction in SEAP at all time points.

### Example 11. In Vivo Administration of MARC1 RNAi Agents in hMARC1-SEAP Mice

The hMARC1-SEAP model described in Example 2, above, was used. On Day 1, four (n=4) female C57bl/6 albino mice were dosed with either saline or RNAi agents formulated in saline (at 2 mg/kg), via subcutaneous (SQ) injection, at 200 μL per 20 g (10 mL/kg) body weight injection volume. The dosing regimen is in accordance with Table 24 below.

TABLE 24
Dosing for mice of Example 11

| | | Targeted Position | |
|---|---|---|---|
| | Dose (RNAi | of MARC1 (Seq ID | |
| Group | Agent) | No. 1) | Dosing Route |
| 1 | Isotonic Saline | N/A | Day 1 SQ Injection |
| 2 | 2 mg/kg AD11786 | 1313 | Day 1 SQ Injection |
| 3 | 2 mg/kg AD12976 | 1313 | Day 1 SQ Injection |
| 4 | 2 mg/kg AD12367 | 1275 | Day 1 SQ Injection |
| 5 | 2 mg/kg AD13117 | 1275 | Day 1 SQ Injection |
| 6 | 2 mg/kg AD13508 | 1275 | Day 1 SQ Injection |
| 7 | 2 mg/kg AD13511 | 1275 | Day 1 SQ Injection |
| 8 | 2 mg/kg AD13510 | 1275 | Day 1 SQ Injection |
| 9 | 2 mg/kg AD13514 | 1275 | Day 1 SQ Injection |
| 10 | 2 mg/kg AD13804 | 1275 | Day 1 SQ Injection |
| 11 | 2 mg/kg AD13805 | 1275 | Day 1 SQ Injection |
| 12 | 2 mg/kg AD13806 | 1275 | Day 1 SQ Injection |

Serum was collected on Day −7, 1, 8, 15, and 22. SEAP expression levels were determined pursuant to the procedure set forth in Example 2, above. Data from the experiment are shown in the following Table 25, with average SEAP reflecting the normalized average value of SEAP.

TABLE 25
Average SEAP normalized to pre-treatment and saline
control in MARC1-SEAP mice of Example 11.

| | Day 8 | | Day 15 | | Day 22 | |
|---|---|---|---|---|---|---|
| | Avg | Std | Avg | Std | Avg | Std |
| Group ID | SEAP | Dev | SEAP | Dev | SEAP | Dev |
| 1. Isotonic Saline | 1.000 | 0.316 | 1.000 | 0.380 | 1.000 | 0.370 |
| 2. 2 mg/kg AD11786 | 0.911 | 0.235 | 0.635 | 0.357 | 0.691 | 0.471 |
| 3. 2 mg/kg AD12976 | 0.638 | 0.207 | 0.426 | 0.168 | 0.481 | 0.267 |
| 4. 2 mg/kg AD12367 | 0.844 | 0.239 | 0.714 | 0.297 | 0.799 | 0.349 |
| 5. 2 mg/kg AD13117 | 0.801 | 0.325 | 0.662 | 0.439 | 0.651 | 0.417 |
| 6. 2 mg/kg AD13508 | 0.829 | 0.232 | 0.551 | 0.145 | 0.595 | 0.161 |
| 7. 2 mg/kg AD13511 | 0.736 | 0.197 | 0.607 | 0.232 | 0.672 | 0.404 |
| 8. 2 mg/kg AD13510 | 0.750 | 0.040 | 0.558 | 0.100 | 0.477 | 0.067 |
| 9. 2 mg/kg AD13514 | 0.486 | 0.124 | 0.354 | 0.203 | 0.358 | 0.236 |
| 10. 2 mg/kg AD13804 | 0.596 | 0.052 | 0.472 | 0.051 | 0.414 | 0.290 |
| 11. 2 mg/kg AD13805 | 0.496 | 0.120 | 0.339 | 0.116 | 0.355 | 0.125 |
| 12. 2 mg/kg AD13806 | 0.670 | 0.126 | 0.465 | 0.124 | 0.472 | 0.079 |

Groups 2-12 showed reduction in SEAP at all time points.

### Example 12. In Vivo Administration of MARC1 RNAI Agents in hMARC1-SEAP Mice

The hMARC1-SEAP model described in Example 2, above, was used. On Day 1, four (n-4) female C57bl/6 albino mice were dosed with either saline or RNAi agents formulated in saline (at 2 mg/kg), via subcutaneous (SQ) injection, at 200 μL per 20 g (10 mL/kg) body weight injection volume. The dosing regimen is in accordance with Table 26 below.

TABLE 26
Dosing for mice of Example 12

| | | Targeted Position | |
|---|---|---|---|
| | Dose (RNAi | of MARC1 (Seq ID | |
| Group | Agent) | No. 1) | Dosing Route |
| 1 | Isotonic Saline | N/A | Day 1 SQ Injection |
| 2 | 2 mg/kg AD12976 | 1313 | Day 1 SQ Injection |
| 3 | 2 mg/kg AD12960 | 1817 | Day 1 SQ Injection |
| 4 | 2 mg/kg AD13447 | 1817 | Day 1 SQ Injection |
| 5 | 2 mg/kg AD13117 | 1275 | Day 1 SQ Injection |
| 6 | 2 mg/kg AD12287 | 1190 | Day 1 SQ Injection |
| 7 | 2 mg/kg AD13923 | 1190 | Day 1 SQ Injection |
| 8 | 2 mg/kg AD13924 | 1190 | Day 1 SQ Injection |
| 9 | 2 mg/kg AD13925 | 1190 | Day 1 SQ Injection |
| 10 | 2 mg/kg AD13926 | 1190 | Day 1 SQ Injection |

Serum was collected on Day −7, 1, 8, 15, and 22. SEAP expression levels were determined pursuant to the procedure set forth in Example 2, above. Data from the experiment are shown in the following Table 27, with average SEAP reflecting the normalized average value of SEAP.

TABLE 27
Average SEAP normalized to pre-treatment and saline
control in MARC1-SEAP mice of Example 12.

| | Day 8 | | Day 15 | | Day 22 | |
|---|---|---|---|---|---|---|
| | Avg | Std | Avg | Std | Avg | Std |
| Group ID | SEAP | Dev | SEAP | Dev | SEAP | Dev |
| 1. Isotonic Saline | 1.000 | 0.296 | 1.000 | 0.225 | 1.000 | 0.163 |
| 2. 2 mg/kg AD12976 | 0.514 | 0.058 | 0.577 | 0.079 | 0.247 | 0.108 |
| 3. 2 mg/kg AD12960 | 0.560 | 0.049 | 0.645 | 0.122 | 0.269 | 0.108 |
| 4. 2 mg/kg AD13447 | 0.495 | 0.098 | 0.582 | 0.175 | 0.287 | 0.054 |
| 5. 2 mg/kg AD13117 | 0.442 | 0.068 | 0.530 | 0.094 | 0.191 | 0.080 |
| 6. 2 mg/kg AD12287 | 0.703 | 0.062 | 0.817 | 0.081 | 0.268 | 0.056 |
| 7. 2 mg/kg AD13923 | 0.641 | 0.210 | 0.780 | 0.229 | 0.360 | 0.283 |
| 8. 2 mg/kg AD13924 | 0.628 | 0.092 | 0.739 | 0.106 | 0.418 | 0.068 |
| 9. 2 mg/kg AD13925 | 0.566 | 0.282 | 0.555 | 0.142 | 0.243 | 0.077 |
| 10. 2 mg/kg AD13926 | 0.513 | 0.061 | 0.664 | 0.078 | 0.341 | 0.086 |

Groups 2-10 showed reduction in SEAP at all time points.

### Example 13. In Vivo Administration of MARC1 RNAi Agents in hMARC1-SEAP Mice

The hMARC1-SEAP model described in Example 2, above, was used. On Day 1, four (n-4) female C57bl/6 albino mice were dosed with either saline or RNAi agents formulated in saline (at 2 mg/kg), via subcutaneous (SQ) injection, at 250 uL per 25 g body weight injection volume. The dosing regimen is in accordance with Table 28 below.

TABLE 28
Dosing for mice of Example 13

| | | Targeted Position | |
|---|---|---|---|
| | Dose (RNAi | of MARC1 (Seq ID | |
| Group | Agent) | No. 1) | Dosing Route |
| 1 | Isotonic Saline | N/A | Day 1 SQ Injection |
| 2 | 2 mg/kg AD11786 | 1313 | Day 1 SQ Injection |
| 3 | 2 mg/kg AD12976 | 1313 | Day 1 SQ Injection |
| 4 | 2 mg/kg AD12369 | 1817 | Day 1 SQ Injection |
| 5 | 2 mg/kg AD13447 | 1817 | Day 1 SQ Injection |
| 6 | 2 mg/kg AD12367 | 1275 | Day 1 SQ Injection |
| 7 | 2 mg/kg AD13805 | 1275 | Day 1 SQ Injection |
| 8 | 2 mg/kg AD11782 | 1190 | Day 1 SQ Injection |
| 9 | 2 mg/kg AD13925 | 1190 | Day 1 SQ Injection |

Serum was collected on Day −7, 1, 8, 15, and 22. SEAP expression levels were determined pursuant to the procedure set forth in Example 2, above. Data from the experiment are shown in the following Table 29, with average SEAP reflecting the normalized average value of SEAP.

TABLE 29
Average SEAP normalized to pre-treatment and saline
control in MARC1-SEAP mice of Example 13.

| | Day 8 | | Day 15 | | Day 22 | |
|---|---|---|---|---|---|---|
| | Avg | Std | Avg | Std | Avg | Std |
| Group ID | SEAP | Dev | SEAP | Dev | SEAP | Dev |
| 1. Isotonic Saline | 1.000 | 0.673 | 1.000 | 0.779 | 1.000 | 0.998 |
| 2. 2 mg/kg AD11786 | 0.745 | 0.349 | 0.433 | 0.195 | 0.405 | 0.122 |
| 3. 2 mg/kg AD12976 | 0.499 | 0.057 | 0.391 | 0.083 | 0.323 | 0.138 |
| 4. 2 mg/kg AD12369 | 0.837 | 0.105 | 0.704 | 0.127 | 0.906 | 0.175 |
| 5. 2 mg/kg AD13447 | 0.887 | 0.070 | 0.983 | 0.144 | 1.081 | 0.187 |
| 6. 2 mg/kg AD12367 | 0.825 | 0.148 | 0.565 | 0.145 | 0.659 | 0.113 |
| 7. 2 mg/kg AD13805 | 0.474 | 0.085 | 0.237 | 0.048 | 0.317 | 0.091 |
| 8. 2 mg/kg AD11782 | 0.666 | 0.111 | 0.571 | 0.022 | 0.618 | 0.228 |
| 9. 2 mg/kg AD13925 | 0.518 | 0.113 | 0.430 | 0.166 | 0.564 | 0.169 |

Groups 2-9 showed reduction in SEAP at Day 8 and Day 15. Groups 2-4 and 6-9 showed reduction in SEAP at Day 22.

### Example 14. MARC1-GLuc AAV Mouse Model

To evaluate certain MARC1 RNAi agents. a MARC1-GLuc (Gaussia Luciferase) AAV (Adeno-associated virus) mouse model was used. Six to eight-week-old male C57BL/6 mice were transduced with MARC1-GLuc AAV serotype 8, administered at least 14 days prior to administration of an MARC1 RNAi agent or control. The genome of the MARC1-GLuc AAV contains the 33-2500 region of the human MARC1 cDNA sequence (GenBank NM_022746.4 (SEQ ID NO:1)) inserted into the 3′ UTR of the GLuc reporter gene sequence. 5E12 to 1E13 GC/kg of the respective virus in PBS in a total volume of 250 uL per 25 g of animal's body weight was injected into mice via the tail vein to create MARC1-GLuc AAV model mice. Inhibition of expression of MARC1 by MARC1 RNAi agents result in concomitant inhibition of GLuc expression, which is measured. Prior to administration of a treatment (between day −7 and day 1 pre-dose). GLuc expression levels in serum were measured by the Pierce™ Gaussia Luciferase Glow Assay Kit (Thermo Fisher Scientific. Catalog #16161), and the mice were grouped according to average GLuc levels.

Mice were anesthetized with 2-3% isoflurane and blood samples were collected from the submandibular area into serum separation tubes (Sarstedt AG & Co., Nümbrecht. Germany). Blood was allowed to coagulate at ambient temperature for 20 min. The tubes were centrifuged at 8.000 xg for 3 min to separate the serum and stored at 4° C. Serum was collected and measured by the Pierce™ Gaussia Luciferase Glow Assay Kit according to the manufacturer's instructions. Serum GLuc levels for each animal can be normalized to the control group of mice injected with vehicle control in order to account for the non-treatment related shift in MARC1 expression with this model. To do so. first. the GLuc level for each animal at a time point was divided by the pre-treatment level of expression in that animal (Day 1) in order to determine the ratio of expression “normalized to pre-treatment”. Expression at a specific time point was then normalized to the control group by dividing the “normalized to pre-treatment” ratio for an individual animal by the average “normalized to pre-treatment” ratio of all mice in the normal vehicle control group. Alternatively, the serum GLuc levels for each animal was assessed by normalizing to pre-treatment levels only.

### Example 15. In Vivo Testing of MARC1 RNAi Agents in MARC1-GLuc AAV Mice

The MARC1-GLUC AAV mouse model described in Example 14, above, using the MARC1-GLuc AAV containing the 33-2500 region of the human MARC1 cDNA sequence was used. On Day 1, four (n=4) male C57bl/6 mice were dosed with either saline or RNAi agents formulated in saline (at 2 mg/kg), via subcutaneous (SQ) injection, at 250 uL per 25 g body weight injection volume. The injections were performed between the skin and muscle (i.e. subcutaneous injections). The dosing regimen is in accordance with Table 30 below.

TABLE 30
Dosing for mice of Example 15

| | | Targeted | |
|---|---|---|---|
| | | Position | |
| | Dose (RNAi | (Seq ID | |
| Group | Agent) | No. 1) | Dosing Regimen |
| 1 | Saline | N/A | Single SQ injection on day 1 |
| 2 | 2 mg/kg AD12363 | 305 | Single SQ injection on day 1 |
| 3 | 2 mg/kg AD12364 | 761 | Single SQ injection on day 1 |
| 4 | 2 mg/kg AD12365 | 956 | Single SQ injection on day 1 |
| 5 | 2 mg/kg AD12366 | 1109 | Single SQ injection on day 1 |
| 6 | 2 mg/kg AD12367 | 1275 | Single SQ injection on day 1 |
| 7 | 2 mg/kg AD12368 | 1633 | Single SQ injection on day 1 |
| 8 | 2 mg/kg AD12369 | 1817 | Single SQ injection on day 1 |
| 9 | 2 mg/kg AD12370 | 1900 | Single SQ injection on day 1 |
| 10 | 2 mg/kg AD12371 | 1954 | Single SQ injection on day 1 |
| 11 | 2 mg/kg AD11786 | 1313 | Single SQ injection on day 1 |

Each of the MARC1 RNAi agents included modified nucleotides that were conjugated at the 5′ terminal end of the sense strand to a targeting ligand that included three N-acetyl-galactosamine groups (tridentate ligand) having the modified sequences as set forth in the duplex structures herein. (See Tables 3, 4, 5A, 5B, 5C, and 6 for specific modifications and structure information related to the MARC1 RNAi agents, including (NAG37) s ligand). The MARC1 RNAi agent AD12363 (Group 2) included nucleotide sequences that were designed to inhibit expression of an MARC1 gene at position 305 of the gene; the MARC1 RNAi agent AD12364 (Group 3) included nucleotide sequences that were designed to inhibit expression of an MARC1 gene at position 761 of the gene: the MARC1 RNAi agent AD12365 (Group 4) included nucleotide sequences that were designed to inhibit expression of an MARC1 gene at position 956 of the gene: the MARC1 RNAi agent AD12366 (Group 5) included nucleotide sequences that were designed to inhibit expression of an MARC1 gene at position 1109 of the gene: the MARC1 RNAi agent AD12367 (Group 6) included nucleotide sequences that were designed to inhibit expression of an MARC1 gene at position 1275 of the gene: the MARC1 RNAi agent AD12368 (Group 7) included nucleotide sequences that were designed to inhibit expression of an MARC1 gene at position 1633 of the gene; the MARC1 RNAi agent AD12369 (Group 8) included nucleotide sequences that were designed to inhibit expression of an MARC1 gene at position 1817 of the gene; the MARC1 RNAi agent AD12370 (Group 9) included nucleotide sequences that were designed to inhibit expression of an MARC1 gene at position 1900 of the gene; the MARC1 RNAi agent AD12371 (Group 10) included nucleotide sequences that were designed to inhibit expression of an MARC1 gene at position 1954 of the gene; the MARC1 RNAi agent AD11786 (Group 11) included nucleotide sequences that were designed to inhibit expression of an MARC1 gene at position 1313 of the gene. (See, e.g., SEQ ID NO:1 and Table 2 for the MARC1 gene referenced).

Serum was collected on Day 1, 8, 15, and 22. GLuc expression levels were determined pursuant to the procedure set forth in Example 14, above. Data from the experiment are shown in the following Table 31, with average GLuc reflecting the normalized average value of GLuc.

TABLE 31
Average GLuc normalized to pre-treatment and saline
control in MARC1-GLuc-AAV mice of Example 15.

| | Day 8 | | Day 15 | | Day 22 | |
|---|---|---|---|---|---|---|
| | Avg | Std | Avg | Std | Avg | Std |
| Group ID | GLuc | Dev | GLuc | Dev | GLuc | Dev |
| 1. Saline | 1.000 | 0.576 | 1.000 | 0.170 | 1.000 | 0.273 |
| 2. 2 mg/kg AD12363 | 0.301 | 0.147 | 1.351 | 0.378 | 0.997 | 0.193 |
| 3. 2 mg/kg AD12364 | 0.348 | 0.065 | 1.297 | 0.100 | 1.395 | 0.191 |
| 4. 2 mg/kg AD12365 | 0.381 | 0.065 | 0.927 | 0.136 | 1.279 | 0.169 |
| 5. 2 mg/kg AD12366 | 0.425 | 0.112 | 1.066 | 0.240 | 1.205 | 0.173 |
| 6. 2 mg/kg AD12367 | 0.294 | 0.064 | 0.956 | 0.162 | 1.039 | 0.144 |
| 7. 2 mg/kg AD12368 | 0.483 | 0.231 | 1.355 | 0.334 | 1.405 | 0.663 |
| 8. 2 mg/kg AD12369 | 0.346 | 0.122 | 1.007 | 0.199 | 1.379 | 0.291 |
| 9. 2 mg/kg AD12370 | 0.344 | 0.056 | 0.938 | 0.126 | 1.190 | 0.190 |
| 10. 2 mg/kg AD12371 | 0.358 | 0.080 | 1.126 | 0.078 | 1.717 | 0.249 |
| 11. 2 mg/kg AD11786 | 0.300 | 0.031 | 0.930 | 0.260 | 1.023 | 0.194 |

Groups 2-11 showed reduction in GLuc at Day 8. Groups 4, 6, 9, and 11 showed reduction in GLuc at Day 15. Group 2 showed reduction in GLuc at Day 22.

### Example 16. In Vivo Administration of MARC1 RNAi Agents in Rats

MARC1 RNAi agents were tested in Sprague Dawley rats for inhibition of MARC1. On Day 1, four (n=4) male Sprague Dawley rat animals were dosed with either saline or RNAi agents formulated in saline (at 3 mg/kg), via subcutaneous (SQ) injection, at 1000 μL per 25 g (4 mL/kg) body weight injection volume. The dosing regimen is in accordance with Table 32 below.

TABLE 32
Dosing for mice of Example 16

| | | Targeted Position | |
|---|---|---|---|
| | Dose (RNAi | of MARC1 (Seq ID | |
| Group | Agent) | No. 1) | Dosing Route |
| 1 | Saline | N/A | Day 1 SQ Injection |
| 2 | 3 mg/kg AD12583 | 405 | Day 1 SQ Injection |
| 3 | 3 mg/kg AD12584 | 406 | Day 1 SQ Injection |
| 4 | 3 mg/kg AD12585 | 409 | Day 1 SQ Injection |
| 5 | 3 mg/kg AD12586 | 411 | Day 1 SQ Injection |
| 6 | 3 mg/kg AD12587 | 564 | Day 1 SQ Injection |
| 7 | 3 mg/kg AD12588 | 565 | Day 1 SQ Injection |
| 8 | 3 mg/kg AD12589 | 566 | Day 1 SQ Injection |
| 9 | 3 mg/kg AD12590 | 567 | Day 1 SQ Injection |
| 10 | 3 mg/kg AD12591 | 901 | Day 1 SQ Injection |
| 11 | 3 mg/kg AD12592 | 1008 | Day 1 SQ Injection |
| 12 | 3 mg/kg AD12593 | 1009 | Day 1 SQ Injection |
| 13 | 3 mg/kg AD12594 | 1012 | Day 1 SQ Injection |
| 14 | 3 mg/kg AD12595 | 1013 | Day 1 SQ Injection |
| 15 | 3 mg/kg AD12596 | 1014 | Day 1 SQ Injection |

The MARC1 RNAi agents' targeted gene positions of Groups 2-15 are cross-reactive with human MARC1.

On Day 8 post dosing, animals were sacrificed, and liver tissue was collected. Expression of rat MARC1 was determined using qPCR, with rat β-actin as control. Average MARC1 expression for each animal in liver tissue was normalized relative to pre-dose and control group 1 (Saline). Data from the experiment are shown in the following Table 33.

TABLE 33
Average relative expression of MARC1 in
rat liver, at Day 8, of Example 16.

| | Day 8 | | |
|---|---|---|---|
| | Rel Exp | | |
| Group ID | rMARC1 | Low | High |
| 1. Saline | 1.000 | 0.144 | 0.168 |
| 2. 3 mg/kg AD12583 | 1.031 | 0.278 | 0.380 |
| 3. 3 mg/kg AD12584 | 0.627 | 0.160 | 0.215 |
| 4. 3 mg/kg AD12585 | 0.436 | 0.071 | 0.084 |
| 5. 3 mg/kg AD12586 | 1.471 | 0.341 | 0.444 |
| 6. 3 mg/kg AD12587 | 1.158 | 0.297 | 0.400 |
| 7. 3 mg/kg AD12588 | 1.191 | 0.242 | 0.303 |
| 8. 3 mg/kg AD12589 | 0.874 | 0.152 | 0.184 |
| 9. 3 mg/kg AD12590 | 0.549 | 0.067 | 0.077 |
| 10. 3 mg/kg AD12591 | 0.369 | 0.104 | 0.144 |
| 11. 3 mg/kg AD12592 | 1.321 | 0.169 | 0.194 |
| 12. 3 mg/kg AD12593 | 1.051 | 0.162 | 0.191 |
| 13. 3 mg/kg AD12594 | 1.149 | 0.225 | 0.280 |
| 14. 3 mg/kg AD12595 | 0.561 | 0.145 | 0.195 |
| 15. 3 mg/kg AD12596 | 0.405 | 0.103 | 0.139 |

Groups 3, 4, 8-10, 14, and 15 showed reduction in rMARC1 at Day 8.

### Example 17. In Vivo Administration of MARC1 RNAi Agents in Rats

MARC1 RNAi agents were tested in Sprague Dawley rats for inhibition of MARC1. On Day 1, four (n=4) male Sprague Dawley rat animals were dosed with either saline or RNAi agents formulated in saline (at 3 mg/kg), via subcutaneous (SQ) injection, at 1000 ML per 25 g (4 mL/kg) body weight injection volume. The dosing regimen is in accordance with Table 34 below.

TABLE 34
Dosing for mice of Example 17

| | | Targeted Position | |
|---|---|---|---|
| | Dose (RNAi | of MARC1 (Seq ID | |
| Group | Agent) | No. 1) | Dosing Route |
| 1 | Saline | N/A | Day 1 SQ Injection |
| 2 | 3 mg/kg AD12596 | 1014 | Day 1 SQ Injection |
| 3 | 3 mg/kg AD13452 | 1014 | Day 1 SQ Injection |
| 4 | 3 mg/kg AD13453 | 1014 | Day 1 SQ Injection |
| 5 | 3 mg/kg AD13454 | 1014 | Day 1 SQ Injection |
| 6 | 3 mg/kg AD13455 | 1014 | Day 1 SQ Injection |
| 7 | 3 mg/kg AD13456 | 1014 | Day 1 SQ Injection |
| 8 | 3 mg/kg AD13457 | 1014 | Day 1 SQ Injection |
| 9 | 3 mg/kg AD13458 | 1014 | Day 1 SQ Injection |
| 10 | 3 mg/kg AD13459 | 1014 | Day 1 SQ Injection |

The MARC1 RNAi agents' targeted gene positions of Groups 2-10 are cross-reactive with human MARC1.

On Day 8 post dosing, animals were sacrificed, and liver tissue was collected. Expression of rat MARC1 was determined using qPCR, with rat β-actin as control. Average MARC1 expression for each animal in liver tissue was normalized relative to pre-dose and control group 1 (Saline). Data from the experiment are shown in the following Table 35.

TABLE 35
Average relative expression of MARC1 in
rat liver, at Day 8, of Example 17.

| | Day 8 | | |
|---|---|---|---|
| | Rel Exp | | |
| Group ID | rMARC1 | Low | High |
| 1. Saline | 1.000 | 0.132 | 0.151 |
| 2. 3 mg/kg AD12596 | 0.559 | 0.246 | 0.439 |
| 3. 3 mg/kg AD13452 | 0.437 | 0.083 | 0.102 |
| 4. 3 mg/kg AD13453 | 0.469 | 0.113 | 0.149 |
| 5. 3 mg/kg AD13454 | 0.361 | 0.075 | 0.094 |
| 6. 3 mg/kg AD13455 | 0.486 | 0.138 | 0.192 |
| 7. 3 mg/kg AD13456 | 0.414 | 0.061 | 0.072 |
| 8. 3 mg/kg AD13457 | 0.394 | 0.136 | 0.208 |
| 9. 3 mg/kg AD13458 | 0.573 | 0.160 | 0.222 |
| 10. 3 mg/kg AD13459 | 0.368 | 0.056 | 0.066 |

Groups 2-10 showed reduction in rMARC1 at Day 8.

### Example 18. In Vivo Administration of MARC1 RNAi Agents in Cynomolgus Monkeys

MARC1 RNAi agents were tested in Cynomolgus monkeys for inhibition of MARC1. Liver biopsies were collected from all animals on Day −7 and used as internal reference samples for the purpose of normalization. On Day 1, four groups with three (n=3) animals per group of female Cynomolgus macaques (non-naïve) monkey animals were dosed with RNAi agents formulated in saline (at 3 mg/kg), via subcutaneous (SQ) injection with syringe and needle in the mid-scapular region, at 0.3 mL/kg body weight injection volume. On Days 15 and 43, additional liver biopsies were collected. All animals were fasted for at least 12 but not more than 18 hours prior to sedation and collection of liver biopsies. The dosing regimen is in accordance with Table 36 below.

TABLE 36
Dosing for mice of Example 18

| | | Targeted Position | |
|---|---|---|---|
| | Dose (RNAi | of MARC1 (Seq ID | |
| Group | Agent) | No. 1) | Dosing Route |
| 1 | 3 mg/kg AD11786 | 1313 | Day 1 SQ Injection |
| 2 | 3 mg/kg AD11778 | 1089 | Day 1 SQ Injection |
| 3 | 3 mg/kg AD12363 | 305 | Day 1 SQ Injection |
| 4 | 3 mg/kg AD12369 | 1817 | Day 1 SQ Injection |

Before each SQ injection, the test animals were first sedated. Sedation was accomplished using Ketamine HCl (10 mg/kg), administered as an intramuscular (IM) injection (none was injected into the quadriceps). Individual doses of MARC1 RNAi agents were calculated based on the body weights recorded on each day of dosing.

For each animal, liver biopsy samples (approximately 200 mg each (160 to 240 mg: ±10%)) were collected for exploratory gene knockdown analysis.

Serum blood was collected on Day −7, Day 1, Day 15, Day 29, and Day 43, prior to liver biopsy sample collections or dose administration when applicable, and from any animals found in moribund condition or sacrificed at an unscheduled interval.

Expression of cyno MARC1 was determined using qPCR, with cyno ARLI as control. Average MARC1 expression for each animal in liver tissue was normalized relative to Day −7 sample level for each individual test animal. Mean relative expression was then calculated for each group from individual normalized values. Data from the experiment are shown in the following Table 37.

TABLE 37
Average MARC1 normalized to pre-treatment
Cynomolgus monkeys, of Example 18.

| | Day −7 (Pre-Dose) | | | Day 15 | | |
|---|---|---|---|---|---|---|
| | Rel Exp | | | Rel Exp | | |
| Group ID | MARC1 | Low | High | MARC1 | Low | High |
| 1. 3 mg/kg AD11786 | 1.000 | 0.446 | 0.805 | 0.446 | 0.137 | 0.199 |
| 2. 3 mg/kg AD11778 | 1.000 | 0.497 | 0.987 | 0.824 | 0.255 | 0.369 |
| 3. 3 mg/kg AD12363 | 1.000 | 0.601 | 1.504 | 0.836 | 0.515 | 1.339 |
| 4. 3 mg/kg AD12369 | 1.000 | 0.125 | 0.142 | 0.548 | 0.164 | 0.235 |

| | Day 43 | | |
|---|---|---|---|
| | Rel Exp | | |
| Group ID | MARC1 | Low | High |
| 1. 3 mg/kg AD11786 | 0.355 | 0.159 | 0.289 |
| 2. 3 mg/kg AD11778 | 0.715 | 0.309 | .554 |
| 3. 3 mg/kg AD12363 | 0.926 | 0.510 | 1.133 |
| 4. 3 mg/kg AD12369 | 0.228 | 0.079 | 0.120 |

Groups 1˜4 showed reduction in MARC1 by Day 15 and Day 43 post-dose.

MARC1 protein levels were quantified via a scheduled LC-MS/MS assay. For this, cynomolgus liver samples were homogenized using RIPA Lysis and Extraction Buffer (Thermo Scientific). Proteins were extracted using a magnetic bead protocol and trypsin-digested for 20 hours in the presence of internal standards. LC-MS/MS-based peptide quantitation was performed, and area-under-the-response-curve for two MARC1-specific peptides (sequences listed below), the corresponding internal standards as well as a specific peptide for the SLC25A3 protein quantified. SLC25A3 is a phosphate carrier protein that was chosen as a normalizing protein for its juxtaposition to MARC1 protein in the mitochondrial membrane. SLC25A3-normalized MARC1 protein concentrations in the liver of the Cynomolgus monkey test animals are shown in the following Table 38.

TABLE 38
MARC1 protein levels in Cynomolgus monkey liver, relative to pre-dose and
SLC25A3 expression, of Example 18.

| | | Day −7 (Pre-Dose) | | |
|---|---|---|---|---|
| Group ID | Peptide sequence | Rel MARC1 | Low | High |
| 1. 3 mg/kg AD11786 | DLLLPIK | 1.00 | 0.87 | 1.13 |
| | SPLFGQYFVLENPGTIK | 1.00 | 0.81 | 1.19 |
| 2. 3 mg/kg AD11778 | DLLLPIK | 1.00 | 0.83 | 1.17 |
| | SPLFGQYFVLENPGTIK | 1.00 | 0.66 | 1.34 |
| | DLLLPIK | 1.00 | 0.83 | 1.17 |
| 3. 3 mg/kg AD12363 | SPLFGQYFVLENPGTIK | 1.00 | 0.54 | 1.46 |
| 4. 3 mg/kg AD12369 | DLLLPIK | 1.00 | 0.89 | 1.11 |
| | SPLFGQYFVLENPGTIK | 1.00 | 0.79 | 1.21 |
| | | Day 15 | | |
| Group ID | Peptide sequence | Rel MARC1 | Low | High |
| 1. 3 mg/kg AD11786 | DLLLPIK | 0.63 | 0.49 | 0.90 |
| | SPLFGQYFVLENPGTIK | 0.61 | 0.29 | 1.07 |
| 2. 3 mg/kg AD11778 | DLLLPIK | 0.89 | 0.77 | 1.00 |
| | SPLFGQYFVLENPGTIK | 1.15 | 0.47 | 1.72 |
| 3. 3 mg/kg AD12363 | DLLLPIK | 0.97 | 0.70 | 1.17 |
| | SPLFGQYFVLENPGTIK | 1.06 | 0.50 | 1.81 |
| 4. 3 mg/kg AD12369 | DLLLPIK | 0.64 | 0.57 | 0.71 |
| | SPLFGQYFVLENPGTIK | 0.68 | 0.56 | 0.76 |
| | | Day 43 | | |
| Group ID | Peptide sequence | Rel MARC1 | Low | High |
| 1. 3 mg/kg AD11786 | DLLLPIK | 0.42 | 0.32 | 0.62 |
| | SPLFGQYFVLENPGTIK | 0.68 | 0.47 | 1.03 |
| 2. 3 mg/kg AD11778 | DLLLPIK | 0.65 | 0.58 | 0.72 |
| | SPLFGQYFVLENPGTIK | 1.17 | 0.67 | 1.63 |
| 3. 3 mg/kg AD12363 | DLLLPIK | 1.14 | 0.81 | 1.46 |
| | SPLFGQYFVLENPGTIK | 2.12 | 0.86 | 2.87 |
| 4. 3 mg/kg AD12369 | DLLLPIK | 0.37 | 0.34 | 0.42 |
| | SPLFGQYFVLENPGTIK | 0.42 | 0.27 | 0.66 |

Group 4 (3 mg/kg AD12369) showed time-dependent >50% MARC1 protein knockdown.

### Example 19. In Vivo Administration of MARC1 RNAi Agents in Cynomolgus Monkeys

MARC1 RNAi agents were tested in Cynomolgus monkeys for inhibition of MARC1. On Day 1 and Day 29, four groups with three (n=3) animals per group of male Cynomolgus macaques (non-naïve) monkey animals were dosed with RNAi agents formulated in saline (at 3 mg/kg), via subcutaneous (SQ) injection, at 0.3 mL/kg body weight injection volume. The dosing regimen is in accordance with Table 39 below.

TABLE 39
Dosing for mice of Example 19

| | Dose (RNAi | | Dose | # Animals |
|---|---|---|---|---|
| Group | Agent) | Dosing Route | Volume | (n=) |
| 1 | 3.0 mg/kg AD12976 | Day 1 and 29 SQ | 0.3 ml/kg | n = 3 |
| | | Injection | | |
| 2 | 3.0 mg/kg AD13805 | Day 1 and 29 SQ | 0.3 ml/kg | n = 3 |
| | | Injection | | |
| 3 | 3.0 mg/kg AD13445 | Day 1 and 29 SQ | 0.3 ml/kg | n = 3 |
| | | Injection | | |
| 4 | 3.0 mg/kg AD13925 | Day 1 and 29 SQ | 0.3 ml/kg | n = 3 |
| | | Injection | | |

The test animals were weighed and dosed on Day 1 and Day 29, via subcutaneous (SQ) administration, with a syringe and needle in the mid-scapular region.

Liver biopsies were collected on Day −7 (pre-dose), 15, 29, and 43. Liver biopsies were collected as a sedated procedure. Animals were fasted overnight (at least 12 hours but less than 18 hours) prior to each liver biopsy collection. For each animal, liver biopsy samples (˜40 mg each, 30-60 mg, +/−10%) were collected for gene knockdown analysis. For Group 2 animals only, on Day 15, an additional liver biopsy (˜40 mg each, 30 to 60 mg: +10%) was collected. Liver biopsies were also collected from any animals found in moribund condition or sacrificed at an unscheduled internal, if applicable.

Blood was collected on Day −7 (pre-dose), 1, 15, 29, and 43, prior to liver biopsy sample collections or RNAi agent dose administration when applicable. Blood was also collected from any animals found in moribund condition or sacrificed at an unscheduled internal, if applicable. Animals were fasted overnight (at least 12 hours but less than 24 hours) for scheduled collections. Fasting duration was consistent between collections (+/−1 hour). Animals were not fasted for unscheduled collections. The blood collection site was femoral vein, with saphenous vein as alternative collection site.

Sedation was accomplished using Ketamine HCl (10 mg/kg), administered as an intramuscular (IM) injection (none was injected into the quadriceps).

Expression of cMARC1 was determined using qPCR, with cyno ARLI as endogenous control. Average MARC1 expression for each animal in liver tissue was normalized relative to Day −7 sample level for each individual test animal. Mean relative expression was then calculated for each group from individual normalized values. Data from the experiment are shown in the following Table 40.

TABLE 40
Average MARC1 normalized to pre-treatment
Cynomolgus monkeys, of Example 19.

| | Day −7 (Pre-Dose) | | | Day 15 | | |
|---|---|---|---|---|---|---|
| | Rel Exp | | | Rel Exp | | |
| Group ID | MARC1 | Low | High | MARC1 | Low | High |
| 1. 3.0 mg/kg AD12976 | 1.000 | 0.666 | 1.991 | 0.801 | 0.411 | 0.844 |
| 2. 3.0 mg/kg AD13805 | 1.000 | 0.600 | 1.499 | 0.712 | 0.352 | 0.696 |
| 3. 3.0 mg/kg AD13445 | 1.000 | 0.650 | 1.855 | 0.849 | 0.430 | 0.869 |
| 4. 3.0 mg/kg AD13925 | 1.000 | 0.644 | 1.805 | 0.553 | 0.378 | 1.188 |
| | Day −29 | | | Day 43 | | |
| | Rel Exp | | | Rel Exp | | |
| Group ID | MARC1 | Low | High | MARC1 | Low | High |
| 1. 3.0 mg/kg AD12976 | 0.920 | 0.420 | 0.771 | 0.495 | 0.279 | 0.638 |
| 2. 3.0 mg/kg AD13805 | 0.388 | 0.208 | 0.447 | 0.250 | 0.142 | 0.327 |
| 3. 3.0 mg/kg AD13445 | 1.024 | 0.601 | 1.453 | 0.886 | 0.529 | 1.312 |
| 4. 3.0 mg/kg AD13925 | 0.302 | 0.190 | 0.510 | 0.235 | 0.143 | 0.368 |

Groups 1˜4 showed reduction in MARC1 by Day 15 and Day 43 post-dose.

MARC1 protein levels were quantified via a scheduled LC-MS/MS assay. For this, cynomolgus liver samples were homogenized using RIPA Lysis and Extraction Buffer (Thermo Scientific). Proteins were extracted using a magnetic bead protocol and trypsin-digested for 20 hours in the presence of internal standards. LC-MS/MS-based peptide quantitation was performed, and area-under-the-response-curve for two MARC1-specific peptides (sequences listed below), the corresponding internal standards as well as a specific peptide for the SLC25A3 protein quantified. SLC25A3 is a phosphate carrier protein that was chosen as a normalizing protein for its juxtaposition to MARC1 protein in the mitochondrial membrane. SLC25A3-normalized MARC1 protein concentrations in the liver of the Cynomolgus monkey test animals are shown in the following Table 41 and Table 42. Table 41 and Table 42 show MARC1 protein levels as quantified by their respective peptide sequences in accordance with LC-MS/MS assay.

TABLE 41
MARC1 protein levels in Cynomolgus monkey liver (quantified
using peptide sequence: DLLLPIK), relative to pre-
dose and SLC25A3 expression, of Example 19.

| | Day −7 | | Day 15 | |
|---|---|---|---|---|
| | Rel Exp | | Rel Exp | |
| Group ID | MARC1 | Std Dev | MARC1 | Std Dev |
| 1. 3.0 mg/kg AD12976 | 1.000 | 0.000 | 0.787 | 0.119 |
| 2. 3.0 mg/kg AD13805 | 1.000 | 0.000 | 0.536 | 0.232 |
| 3. 3.0 mg/kg AD13445 | 1.000 | 0.000 | 1.048 | 0.271 |
| 4. 3.0 mg/kg AD13925 | 1.000 | 0.000 | 0.475 | 0.030 |
| | Day 29 | | Day 43 | |
| | Rel Exp | | Rel Exp | |
| Group ID | MARC1 | Std Dev | MARC1 | Std Dev |
| 1. 3.0 mg/kg AD12976 | 0.744 | 0.123 | 0.642 | 0.153 |
| 2. 3.0 mg/kg AD13805 | 0.449 | 0.147 | 0.281 | 0.101 |
| 3. 3.0 mg/kg AD13445 | 0.874 | 0.188 | 0.943 | 0.057 |
| 4. 3.0 mg/kg AD13925 | 0.400 | 0.109 | 0.323 | 0.057 |

MARC1 RNAi agents showed MARC1 protein inhibition out to at least 43 days post dose. Groups 1, 2, and 4 showed reduction in MARC1 at all time points, while Group 3 showed negligible reduction at all time points. More specifically, AD13805 achieved approximately 71% inhibition (0.281) of MARC1 at Day 43, after 2×3.0 mg/kg dose.

TABLE 42
MARC1 protein levels in Cynomolgus monkey liver (quantified
using peptide sequence: SPLFGQYFVLENPGTIK), relative
to pre-dose and SLC25A3 expression, of Example 19.

| | Day −7 | | Day 15 | |
|---|---|---|---|---|
| | Rel Exp | | Rel Exp | |
| Group ID | MARC1 | Std Dev | MARC1 | Std Dev |
| 1. 3.0 mg/kg AD12976 | 1.000 | 0.000 | 0.820 | 0.056 |
| 2. 3.0 mg/kg AD13805 | 1.000 | 0.000 | 0.481 | 0.135 |
| 3. 3.0 mg/kg AD13445 | 1.000 | 0.000 | 0.969 | 0.083 |
| 4. 3.0 mg/kg AD13925 | 1.000 | 0.000 | 0.544 | 0.047 |
| | Day 29 | | Day 43 | |
| | Rel Exp | | Rel Exp | |
| Group ID | MARC1 | Std Dev | MARC1 | Std Dev |
| 1. 3.0 mg/kg AD12976 | 0.719 | 0.142 | 0.642 | 0.191 |
| 2. 3.0 mg/kg AD13805 | 0.383 | 0.069 | 0.245 | 0.065 |
| 3. 3.0 mg/kg AD13445 | 0.875 | 0.143 | 0.740 | 0.107 |
| 4. 3.0 mg/kg AD13925 | 0.363 | 0.059 | 0.286 | 0.019 |

MARC1 RNAi agents showed MARC1 protein inhibition out to at least 43 days post dose. Groups 1, 2, and 4 showed reduction in MARC1 at all time points, while Group 3 showed less significant reduction at all time points (Day 15 reduction is negligible). More specifically, AD13805 achieved approximately 75% inhibition (0.245) of MARC1 at Day 43, after 2×3.0 mg/kg dose.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An RNAi agent for inhibiting expression of a MARC1 gene, comprising:
an antisense strand comprising a nucleotide sequence of at least 15 contiguous nucleotides differing by 0 or 1 nucleotides from 15 contiguous nucleotides of any one of the antisense strand sequences of Table 2, Table 3, or Table 6D; and
a sense strand comprising a nucleotide sequence that is at least partially complementary to the antisense strand.

2. The RNAi agent of claim 1, wherein the antisense strand comprises nucleotides 2-18 of any one of the sequences provided in Table 2, Table 3, or Table 6D.

3. The RNAi agent of claim 1 or claim 2, wherein the sense strand comprises a nucleotide sequence of at least 15 contiguous nucleotides differing by 0 or 1 nucleotides from 15 contiguous nucleotides of any one of the sense strand sequences of Table 2, Table 4, Table 5, or Table 6D, and wherein the sense strand has a region of at least 85% complementarity over at least 15 contiguous nucleotides to the antisense strand.

4. The RNAi agent of any one of claims 1-3, wherein at least one nucleotide of the RNAi agent includes a modified internucleoside linkage.

5. The RNAi agent of any one of claims 1-4, wherein all or substantially all of the nucleotides are modified nucleotides.

6. The RNAi agent of any one of claims 4-5, wherein the modified nucleotides are independently selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.

7. The RNAi agent of claim 5, wherein all or substantially all of the modified nucleotides are 2′-O-methyl nucleotides, 2′-fluoro nucleotides, or combinations thereof.

8. The RNAi agent of any one of claims 1-7, wherein the antisense strand consists of or consists essentially of the nucleotide sequence of any one of the modified antisense strand sequences of Table 3 or Table 6D.

9. The RNAi agent of any one of claims 1-8 wherein the sense strand consists of, consists essentially of, or comprises the nucleotide sequence of any of the modified sense strand sequences of Table 4, Table 5, or Table 6D.

10. The RNAi agent of claim 1, wherein the antisense strand comprises the nucleotide sequence of any one of the modified sequences of Table 3, and the sense strand comprises the nucleotide sequence of any one of the modified sequences of Table 4, Table 5, or Table 6D.

11. The RNAi agent of any one of claims 1-10, wherein the sense strand is between 18 and 30 nucleotides in length, and the antisense strand is between 18 and 30 nucleotides in length.

12. The RNAi agent of claim 11, wherein the sense strand and the antisense strand are each between 18 and 27 nucleotides in length.

13. The RNAi agent of claim 12, wherein the sense strand and the antisense strand are each between 18 and 24 nucleotides in length.

14. The RNAi agent of claim 13, wherein the sense strand and the antisense strand are each 21 nucleotides in length.

15. The RNAi agent of claim 14, wherein the RNAi agent has two blunt ends.

16. The RNAi agent of any one of claims 1-15, wherein the sense strand comprises one or two terminal caps.

17. The RNAi agent of any one of claims 1-16, wherein the sense strand comprises one or two inverted abasic residues.

18. The RNAi agent of claim 1, wherein the RNAi agent is comprised of a sense strand and an antisense strand that form a duplex having the structure of any one of the duplexes in Table 6A and Table 6B.

19. The RNAi agent of claim 18, wherein all or substantially all of the nucleotides are modified nucleotides.

20. The RNAi agent of claim 1, comprising an antisense strand that consists of, consists essentially of, or comprises a nucleotide sequence that differs by 0 or 1 nucleotides from one of the following nucleotide sequences (5′→3′):
(SEQ ID NO: 1608)
UGAAAGAACUAUUCCAUAAUC;
(SEQ ID NO: 1657)
ACAGAAUCCUGUCUUGUCGUU;
(SEQ ID NO: 1580)
UCCUUUAAAGGUUUUCAGUAG;
or
(SEQ ID NO: 1659)
UAUUGAAGCAUUGAGACACCG.

21. The RNAi agent of any one of claims 1-20, wherein the nucleotides of the antisense strand located at position 2 and position 14 from the 5′-end are 2′-fluoro modified nucleotides.

22. The RNAi agent of claim 21, wherein the nucleotide of the antisense strand at position 2 is a 2′-fluoro uridine, and the nucleotide of the antisense strand at position 14 is a 2′-fluoro cytidine, and wherein the antisense strand comprises 3 or 4 phosphorothioate internucleoside linkages.

23. The RNAi agent of any one of claims 1-22, wherein the sense strand consists of, consists essentially of, or comprises a nucleotide sequence that differs by 0 or 1 nucleotides from one of the following nucleotide sequences (5′→3′):
(SEQ ID NO: 1734)
GAUUAUGGAAUAGUUCUUUCA;
(SEQ ID NO: 1783)
AACGACAAGACAGGAUUCUGU;
(SEQ ID NO: 1774)
CUACUGAAAACCUUUAAAIGA;
or
(SEQ ID NO: 1784)
CGGUGUCUCAAUGCUUCAAUA

24. The RNAi agent of any one of claims 20-23, wherein all or substantially all of the nucleotides are modified nucleotides.

25. The RNAi agent of claim 1, comprising an antisense strand that comprises, consists of, or consists essentially of a modified nucleotide sequence that differs by 0 or 1 nucleotides from one of the following nucleotide sequences (5′→3′):
(SEQ ID NO: 1143)
cPrpusGfaaaGfaacuaUfuCfcAfuaausc;
(SEQ ID NO: 1228)
asCfagAfauccugUfcUfuGfucgusu;
(SEQ ID NO: 1229)
isCfagAfauccugUfcUfuGfucgusu;
(SEQ ID NO: 1200)
cPrpusCfscsUfuUfaaaggUfuUfuCfaGfuasg;
or
(SEQ ID NO: 1235)
usAfsusugaAfgcauUfgAfgAfcaccsg,
wherein a represents 2′-O-methyl adenosine, c represents 2′-O-methyl cytidine, g represents 2′-O-methyl guanosine, i represents 2′-O-methyl inosine; and u represents 2′-O-methyl uridine: Af, represents 2′-fluoro adenosine, Cf represents 2′-fluoro cytidine, Gf represents 2′-fluoro guanosine, and Uf represents 2′-fluoro uridine: cPrpu represents a 5′-cyclopropyl phosphonate-2′-O-methyl uridine: s represents a phosphorothioate linkage; and wherein all or substantially all of the nucleotides on the sense strand are modified nucleotides.

26. The RNAi agent of claim 1, wherein the sense strand comprises, consists of, or consists essentially of a modified nucleotide sequence that differs by 0 or 1 nucleotides from one of the following nucleotide sequences (5′→3′):
(SEQ ID NO: 1319)
gauuauggAfAfUfaguucuuuca;
(SEQ ID NO: 1376)
aacgacaaGfAfCfaggauucugu;
(SEQ ID NO: 1361)
cuacugaaAfAfCfcuuuaaaiga;
or
(SEQ ID NO: 1377)
cggugucuCfAfAfugcuucaaua,
wherein a represents 2′-O-methyl adenosine, c represents 2′-O-methyl cytidine, g represents 2′-O-methyl guanosine, u represents 2′-O-methyl uridine, and i represents 2′-O-methyl inosine; Af, represents 2′-fluoro adenosine, Cf represents 2′-fluoro cytidine, Gf represents 2′-fluoro guanosine, and Uf represents 2′-fluoro uridine; s represents a phosphorothioate linkage; and wherein all or substantially all of the nucleotides on the antisense strand are modified nucleotides.

27. The RNAi agent of any one of claims 20-26, wherein the sense strand further includes inverted abasic residues at the 3′ terminal end of the nucleotide sequence, at the 5′ end of the nucleotide sequence, or at both.

28. The RNAi agent of any one of claims 1-27, wherein the RNAi agent is linked to a targeting ligand.

29. The RNAi agent of any one of claims 1-28, wherein the sense strand comprises:

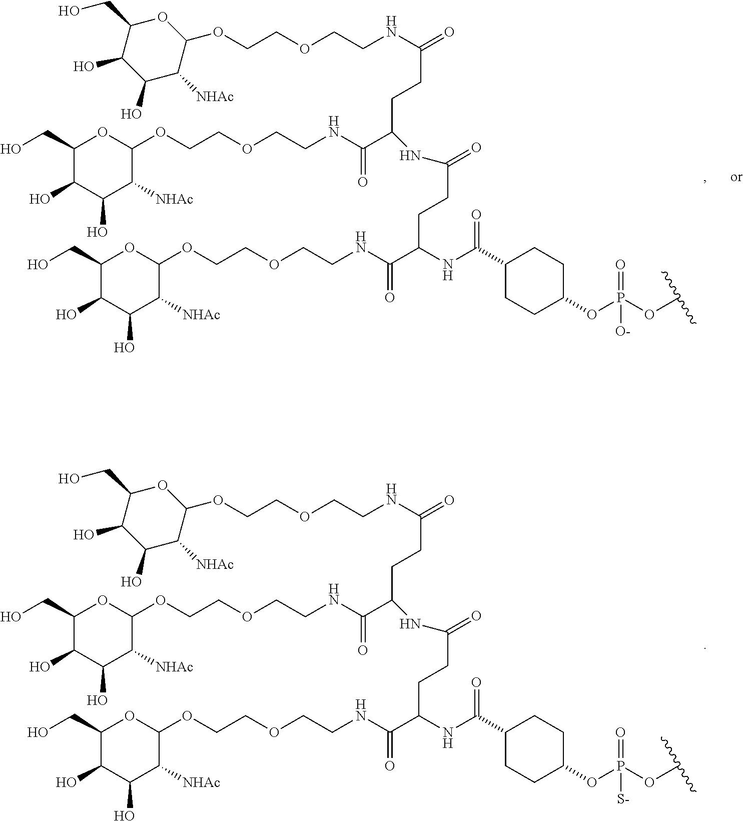

30. The RNAi agent of any one of claims 1-29, wherein the targeting ligand is linked to the sense strand.

31. The RNAi agent of claim 30, wherein the targeting ligand is linked to the 5′ terminal end of the sense strand.

32. A composition comprising the RNAi agent of any one of claims 1-31, wherein the composition further comprises a pharmaceutically acceptable excipient.

33. The composition of claim 32, further comprising a second RNAi agent capable of inhibiting the expression of a MARC1 gene.

34. The composition of any one of claims 32-33, further comprising one or more additional therapeutics.

35. The composition of any one of claims 32-34, wherein the composition is formulated for administration.

36. The composition of claim 35, wherein the composition is delivered by subcutaneous injection.

37. The composition of any one of claim 32-36, wherein the pharmaceutically acceptable excipient is a sodium phosphate buffer.

38. The composition of any one of claim 32-36, wherein the pharmaceutically acceptable excipient is isotonic saline or water for injection.

39. A method for inhibiting expression of a MARC1 gene in a hepatocyte cell, the method comprising introducing into a cell of a subject an effective amount of an RNAi agent of any one of claims 1-31 or the composition of any one of claims 32-38.

40. The method of claim 39, wherein the subject is a human subject.

41. The method of any one of claims 39-40, wherein the MARC1 mRNA levels are reduced by at least about 50% in the hepatocyte cell or in the subject.

42. The method of any one of claims 39-41, wherein the MARC1 protein levels are reduced by at least about 50% in the hepatocyte cell or in the subject.

43. A method of treating a MARC1-related disease, disorder, or symptom, the method comprising administering to a human subject in need thereof a therapeutically effective amount of the composition of any one of claims 32-38.

44. The method of claim 43, wherein the disease is nonalcoholic steatohepatitis (NASH), nonalcoholic fatty liver disease (NAFLD), alcoholic fatty liver disease, autoimmune hepatitis, hepatic fibrosis, cirrhosis, elevated blood cholesterol levels, hypertriglyceridemia, liver disease, and/or other MARC1-related disease.

45. The method of any one of claims 39-44, wherein the level of serum MARC1 protein is decreased in the subject.

46. The method of any one of claims 39-45, wherein the RNAi agent is administered to a human subject at a dose of about 0.05 mg/kg to about 5.0 mg/kg of body weight of the human subject.

47. Use of the RNAi agent of any one of claims 1-31 or the composition according to any one of claims 32-38, for the treatment of a disease, disorder, or symptom that is mediated at least in part by a reduction in MARC1 gene expression.

48. Use according to claim 47, wherein the disease is nonalcoholic steatohepatitis (NASH), nonalcoholic fatty liver disease (NAFLD), alcoholic fatty liver disease, autoimmune hepatitis, hepatic fibrosis, cirrhosis, elevated blood cholesterol levels, hypertriglyceridemia, liver disease, and/or other MARC1-related disease.

49. Use of the RNAi agent of any one of claims 1-31 or the composition according to any one of claims 32-38, for the preparation of a pharmaceutical composition for treating a disease, disorder, or symptom that is mediated at least in part by a reduction in MARC1 gene expression.

50. Use according to any one of claims 47-49, wherein the RNAi agent is administered to a human subject at a dose of about 0.05 mg/kg to about 5.0 mg/kg of body weight of the human subject.
